SEQUENCE LISTING (77 sequences)

SEQ ID NO: 1 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 2 - DNA, 2695 bp, Homo sapiens
caaataaaagcgatggcgattgggctgccgcgtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtcaaaaaaaaaa

SEQ ID NO: 3 - DNA, 572 bp, Homo sapiens
gtacctgtctataaggagtcctgcttatcacaatgaatgttctcctgggcagcgttgtgatctttgccaccttcgtgactttatgcaatgcatcatgctatttcatacctaatgagggagttccaggagattcaaccaggaaatgcatggatctcaaaggaaacaaacacccaataaactcggagtggcagactgacaactgtgagacatgcacttgctacgaaacagaaatttcatgttgcacccttgtttctacacctgtgggttatgacaaagacaactgccaaagaatcttcaagaaggaggactgcaagtatatcgtggtggagaagaaggacccaaaaaagacctgttctgtcagtgaatggataatctaatgtgcttctagtaggcacagggctcccaggccaggcctcattctcctctggcctctaatagtcaatgattgtgtagccatgcctatcagtaaaaagatttttgagcaaacacttgaatatgtgtgtccttttaatttataatttatgtatgcattgatttaatacacattataaagaatacaaaacatttaaaaa

SEQ ID NO: 4 - DNA, 420 bp, Homo sapiens
gtacctgtctataaggagtcctgcttatcacaatgaatgttctcctgggcagcgttgtgatctttgccaccttcgtgactttatgcaatgcatcatgctatttcatacctaatgagggagttccaggagattcaaccaggatgtttctacacctgtgggttatgacaaagacaactgccaaagaatcttcaagaaggaggactgcaagtatatcgtggtggagaagaaggacccaaaaaagacctgttctgtcagtgaatggataatctaatgtgcttctagtaggcacagggctcccaggccaggcctcattctcctctggcctctaatagtcaatgattgtgtagccatgcctatcagtaaaaagatttttgagcaaacacttgaatatgtgtgtccttttaatttataatttatgta

SEQ ID NO: 5 - DNA, 30000 bp, Homo sapiens
ctctaaaactaaaaacaatgtttgtcaggagttacaaaccatgaccaactaattatggggaatcataaaatatgactgtatgagatcttgatggtttacaaagtgtacccactgttaatcactttaaacattaatgaacttaaaaatgaatttacggagattggaatgtttctttcctgttgtattagttggctcaggctgccataacaaaataccacagactgggaggcttaagtaacagaaattcatttctcacagttctgggggctggaagtccacgatcaaggtgcaggaaaggcaggcttcattctgaggcccctctcttggctcacatgtggccaccctcccactgcgtgctcacatgacctctttgtgctcctggaaagagggtgtgggggacagagggaaagagaaggagagggaactctctggtgtctcgtctttcaaggaccctaacctgggccactttggcccaggcactgtggggtggggggttgtggctgctctgctctgagtggccaagataaagcaacagaaaaatgtccaaagctgtgcagcaaagacaagccaccgaacagggatctgctcatcagtgtggggacctccaagtcggccaccctggaggcaagcccccacagagcccatgcaaggtggcagcagcagaagaagggaattgtccctgtccttggcacattcctcaccgacctggtgatgctggacactgcgatgaatggtaatgtggatgagaatatgatggactcccagaaaaggagacccagctgctcaggtggctgcaaatcattacagccttcatcctggggaggaactgggggcctggttctgggtcagagagcagcccagtgagggtgagagctacagcctgtcctgccagctggatccccagtcccggtcaaccagtaatcaaggctgagcagatcaggcttcccggagctggtcttgggaagccagccctggggtgagttggctcctgctgtggtactgagacaatattgtcataaattcaatgcgcccttgtatccctttttcttttttatctgtctacatctataatcactatgcatactagtctttgttagtgtttctattcaacttaatagagatatgttatacttaaaaaaaaaaaaaaaatgggccgggcgcagtggctcatgcctgtaatcccagcactttgggaggccgaggtgggtggatcatgaggtcaggagatcgagaccatcctgattaacgtggtgaaaccccgtctctactaaaaatacaaaaaaaattagccgggcgtggtggcaggcgcctgtagtcccagctactcaggaggctgaggcaggagaatggcgtgaacccgggaggcagagcttgcagtgagccgagatggcgccactgcactccagcctgggtccagcctgggcaacaagagtaaaaccctgtctcaaaaaaaaaaaaaaaaagataaataataaaaaataaaatataaataaaaaagcaaactgcagactctcagcacaaatgatttcatccacctctggcactaagagacttcaacaaacactgttgtttctaacagttcatggccacatccttaggatgatgacttcagcccccttaagtttctgcctaataaaattcaatgctgcccaaagaatttatgatttgttccaggcaaaacctgaatataggcccccgaacccccttttcctaaaggatttactttagaaaactggcagttagaaatcctttctctgtctcttggaaatgtatcttctataacacaggaatgtctttcccaaggacctgggagccatcctttcgaaatgtaatcattaggtaggacagagcctctgtctcccaatcttggtggtagggtaggaccctgatattgataagcaccagttcacaatttgataagcacaagttcacaatctggtcagtgtactcattgaccaaacctccctcccctcaactagcatccttcagtacttttcccttaacacatcctagcatttaaaaagcctcttgccttctggtttggtagaattgagctcagttaatgctgaagtgtctcttccctgccacagtagtctgaataaaatctgtcttgacatttttgacaagtgtctggtgcaaaatttttctttaacaatagcaagtgttgaaattggatagtgtaagttttttttttagctttgttctttttcagaatatttaggctatcctaggtcattcgcttttctatataaattttagcaaaagcttatcaatttccacaaaaacctgctggactattttttttgtttttgtttttgagaccaagtctcactctgtcatccaggctggagtgtggtggcacaatcttggctcactgcaacctccgcctcccaagttcaagcgattctcctgcctcagcctccctagtagctgggattacaggcagatgccaccatgcccagataatttttgtatttttagtagagtcactgtttcaccatgttggccaggctggtcttgaacacctgaactcaggtgatccacctgcctcggcctcccaagtgctgggattacaggcgtgagccactgcacccagccaggactgactttttaattttttcccttgatttcgcagtatttggaatggactcactttcttaggaacgacattaattttttttaaattggcagaaatttatcaataaattgggggaggactgtcatcttaacaatagagtcttttaatctatgaacatgggattatttaaatcagggattagcaaattttctgcaaagaaccagatagtaaaaatatttgtggtattgtgggctatattcaactttgctttgtagtgtaaaagcagtgttggcccggcgcggtggctcatgcttgtaatcccagcactttgggaggctgaggcgggtggactacctaaggtcaggagttcaagaccagctggctaacatggagaaaccccgttactactaaaaatacaaaaaaaatagccaggcatagtggcgcacgcctgtaattccaactattcgggaggctgagacaggagaatcgcttgaacctgggaggtggatgttgcagtgagctgagatcacaccattgcactccagcttgggcaacaagagcaaaactccatctcaaaaaaaaaaaaattgtgtttcaataaaactttatttacaaaacaaacagggggccagttcagggccatggactgtagtttgctgcctctaatttagaacttcttcaatttctctgagcaaagttttatagtttttattgtaagtcttgcatttctttcattaaactcatttctaagtattttattcttttaggtgttactgtgaatgaaattttgtttccttaatttcattttcacattgtttattactagtatgtagaaatacaattgatttttacatatcggccttatatacttgaaccttgtttgctaaactcatttattaattccagtaaaattttttgatagattacttgggattttctgcatacagatcatgtggtctacaaataaacacagttttacttttttcttttcattctagatgtctttaatttacctttctctctctttctccctctaccccctttttaagttgccctggctatgaccagtagtacaatgttgaatggaagtggtaagagtagacattcttgcctgtttcctaatcttgagtgggaaacattctgtctttcatcattaagtacattaactgtaggttgtctgtagttgccctctatcagattgaagaagtttacttttattcctagtttgttgagagtttttaccacgaatgtatgttggctattgtcaaatgcttctgtatctattgaaatgacaatgtgatgtattttagtctattaatctagtacattacattcactgattttgaggttttgttgtttttaattaattaattttaaaaacctcaccatatactagagaaagcattacttgatttttggatgtcacaccaaccttgcatttctgggataaatcatacttggtcatggtgtataatcttttgtacatgttgatggatttttgtttgctaatatcttattgagtatgtttgcatctctattcatgagagatattgttctataatttttgtttattttggtgtgtttgtttagctttgatattggggtaacactggcctcattaaaggagttggaacatgatctatccttttttattttctgaaagatttttgtgaaagacataatatttcttctcgtcttattgtgtcgtgtcgtgtcctgtcctgtctttttgaggtggagttttgctcttgttgcccaggcgggagtgcaatggcatgatctcggcccattgcaacctccacctcccaggttcaggtgatgctcctgcctcccaagtagctgggattacaggtatgcaccaccagacctggctaattttttttttttttttttttttgagacaaagtcttgctctgtcacccaggcgggagtgcagtaacgtgatcttggctcactgcaagctccgcctcccgggttcacgccattctcctacttcagcctcctgaatagctgggactacaggcacccgcgaccacacctaactttttttgtatttttagtagagacggggtttcaccacgttagccaggatgatctcgatctcctgacctcatgatccacccgtctcggcctcccaacgtgctgggattacagttatgagccaccatgccctgcctaattttgtatttttagtagtgatggggtttcaccatgttgatcaggctggttttgaactcctgacctcaggccatccacccgcctttgcctccaatttggctaatttttttattttgtagagatggagttctcactgtgttgtccaggctgctcttgaatttctggcctcaagcaatcctcctacctgggcctcccaaagttctgggattataggtataagccactactcctggcctaaaaatgtattttttatacatatgtctctgtcaaaattgggataaagagaaacagactggcagtggtatgcatttcaaggaaaataggagaaagcctatttacttaggaaagtgaggctcataagtacttcatatacaaacatcatacctgcttcactgggttgtgttacctgacttgccccgagcatttgaacttgctacccccaatagagaattgactccctcattagccaaagaggaatctgagacctggtgagggtgagcacctggccagtcatcagtaagctccttttcatcccacccaccggcggtgattagtaaccatgtctgacaggcatttactttcctcctggtgagtagctggaaaagagagtagtttggaaatatttggaaatagcaccgtttgtctctcacatatgtgatctggaagccccctgcctgcttcacattgtcctgcctttccagactgaaccagtgttcattttacatatgttgagtgatggctcatgtctccctaaaaggtataaaaccaagctgtgctctgactaccttgggcacatgttgtcagaacctcctgagggtgtgtcacggtagcatatcctcaaccttggcgacttggtgctaacgctgtactaaattaccaggctggttaacatctattggaatttcttggtgccaaaggctgtgggagacctgaacacattgtcaagggaggaggaaggggaaggagaagggctgtgggatctcccgcatgaaaggccccatgctggtggtagcccccacctgctgctctggggctcaaaggtgcttagatctcttcacttgagtgcagatgctgccagccctttgggcaggtgtgtgagggaggcagccctggctgtaaagagagcaggggcttggctgggcgtggtggctcacgcctgtaatcccagcactttgggaggccgaggtggcagatcatttgaggtcaggagtttgtgaccagcctggccaacatggtgaaaccccatctctactaaaaaaaaaatacagaaattagctgggcatggtggcatgtacctgtaatcccagctacgcaggaggctgaggcaggagaatcacttgaacccaggaggcggaggttgcagtgagctgagatagcgccactgcactccagcccgggcgacagcgcgagactccatctcaaaaaaaaaaaaaaaaaaaaaaaaaagagagtactggcttttgggtcattaggactattatataactgaattgtgtcaggcctctgagctgaagctcagctattataacccctgtgacctgcacatatatgtccagatggcctgcaggaaccaagaagtctggagcagccaaaaaacccacaaagtaaaacagccagttcctgccttaactgagtaaccaaaattacaacattttaccattgtgacttgtccctgccctaccttagctgatcaaccaactttgtgacattcttcttctggacaatgagtcttatgatctccccactatgtaccttgtgaccccctcctctgctaacaatagataaccacgttttactgtaactttccattacctacccaactcctataaagcaatcccttccccatctccctttgctgactcttttcggactcagcccacctgcacccaagtgaattaaaagttttattcctcatacaaagcctgtttggtggtttcttcacatggacgtgcttgacaaattggggtcggcttgcccagtgcagttaagtccagatatccacactgaagttgcagcactagaaaggaaggtgtttattcacagagccctgagcaagggggaccaggcagcgcatgctcaaaacctgaactctctgatggctggcagatgtgaaccctgaaaatctgagacaagtgtcaatttagaaagtttattttgccaaggttgaggacatgcccccgtgacacaccctcaggaggttctgacgacatgtgcccaaggtggtcagagcacagcttggttttataccttttagggagacatgagccatcaatcaacatatgtaaaatgaacattggtttggtctggaaaggcaggacaacttgaagcaggcagtgggcttccagatcacagataggtgagagacaaacggttgcattcttttgagtttctgattagcctttccaaaggaggcaatcagatatgcatttatctcagtgagcagagggatgactttgaatagaatgcgaggcagatttgccctaagcagttcccagcttgaattttctctttaacttagtgatttgggagggccaacatagtttcctttcacacaggtaagggtttttaaaggcggggtaaattttcaggaaagcagcagaagttagataaaattgtaaatcagtacatgaagggtacacattggtttggcctaaaaaaggtgagatatcttgaagcaggggcttacaggtcataggtggattcagagattctttgatttgtagttggttcaggaggcgatgccttgcctgaaaattcagggttaacagaaaagaatattagctttagctcatgggtgtggctttcaggagcctccggaagaaatttagaatgaaagaaggtggtcagagtgcagtcctcagctccctctatctgaggtctgcaggccagcagatccgtttggtgggggtccacatggaggtccaggtttctgaatgacaaactcaaggacgtgtgttaagatgttatctttagttcctaaaactaaacatctccggactctaacttccttggctattgttttgggctactattaccttcctgcctaacaagttacttacttacttctcagggctagctggatgcctgaaatttcccttgaaggcactcaagattttcctttatttccatgcttgatggtggcgttcggggtggtggcacaggtccctgcttcatctcaggcctgggtttaaattctcactctttctcaccatctgaaaaccctgaatagttgtttccatcctgaaacctcaatgccccactggtaaagtgggcgagagaaggcctgttttgttgtgttgctgggaaatccctgaggtaatgcatgtgtggacatgagaggctccagctcccttcccacctctcagccctccaccctggggtggttctggaaggcactgctcatggtaaggacagaaggttgctggctggcgttcttctgtctgattcagtggctgcatttacttagcatgtgctctatgaggagaataagggcctgtgatgctggtatagatcagttcagatatactggtgggtacgtggctgctgcccaacactcctgagtgcccccactcagcatgttggatcctggcccctccagacctcaacatcatccagaaacagaaggaatagagcctcgcacagtagggtcccccagactgacttctggcagctgaactgaagggtcagtggttgagccatactggttgttcaacactttgaataccacatccagaaagaaaatacaagctccctgtcacggtagcctccttgtgactgtgttacattcaacacaagacaaacatcagcttcacaaaagggagcagtctctgggacaatccattgaaagcgtgagaggcaggtagttcctgggttctgtgcatagttgctttaagctgtaaacacacaatccttgaccaaataccctttaatccacttgacaaacaagaagcttccgggcctccaaggggatgcccaagtctcccttttctctgggacaaaacctggttttgtttcttaggtttctcagcggctaatctggaacatgtttgcttttgccccgtttctttgtcccagtgttttttctgctgttttcacacctctctggataagctttccaagttccttacagaaaaccagaaagattattcaccagcagtcttcctggtccctggcttgtcagcctcaatgcattccagtgcactcttgtgacactgtatccagagtagctgtggctcacagcatttacagtgttctttctcgctattctattagtggcttccttttccaacccagttctttgcctggatatccaggatctctatcccaccgtcaccctcactccatatagggggttcatcctacagcatttctgttgctgtaaaaagaccctctactgcatcctttggcatggagaatttgtcttattgtatttttgaagcaagagattatgcagaaatgaatccatgggtgggggcagagtctgtctttctggtttctgcagagtgaaaactctcataatgtctgtcctagactaagccccgcagtccagaagctggtccacctctggacttaacacttgagctgaaaacgtctctttttgcccatgctcattttagttcggtttccaacagaacagaatcctgacgaataccaaaacgggtcctgccacagggtatcacaaagattgggctctacaaagtaaaattgccagggtataagtggaccctcagggaggctggcttatgccaggctgggaattgggcagtcctcgaacactgaacacttttccatctgtcccttcatgcctgcttgtcttggggaggctggcttttatggaggggtccctgggaaggaatgggggccgatggggaggggaatgggatccgtattgcgtatttggggtgaggaagggacccagagggaaagtggaaagaacaggagccggtggagggcaatgtgcagggtaggggtgggggtgcagttttcacctctggtctctgtgagatacgaaataagattgtgttctgcgtatgaagtaggcagttgggtgagaggtttaagagaagagatcatggtttggaaaggctctagagcacgatgggagaggaagtggatgagagataagtcatcagcttgaagactggtaaggggagaccccaccgatcctggtgttaaatcaagtttagcctaaagccttctccttacatattttaaattcaccctaatggtttctctgtacatagtgaactgtaagctaactgcacgtgtaaacaggctgtcacctactctcgtaccaagtagccgagtctcagttaatcacagcagccagacttcaaccactcacaggcggccagctgttgaaactggactcaaataagccaagcaccatgccatcaccaatccgtctgtttctgtacctcacttccagtttctgtacatcactttcctttttctgtccataaatctttgactatgaggcagtgcaggagtctctctgaacttatttttgcctgggggctgcctgattcacacaaatggttctttgctcaatcaaactctgttaaatttaatttgtctaacgttcttttaacactgggaatgatggcagggcacggtggcccatgcttgtaatcccagcactttgggagaccaaggcaggcagatcacttgaggtcaggcgttcaagaccaacctgggcaatggtgaaaccctgtctctactaaaaatacaaaaattagccaggcatggtggtgcatgcctatagccccagctactgggaaggctgaagcacaagaatcgcttgtgcccgggaggtggaggttgcagtgagctgagattgcagcactgcactccatcctgggcgacagagcaagactctgtctcagaaaaaaacaaaaaacaaaacaaaacaaaaaaccctggaaatgaatggcagctcctgccacagcatcagcccaagggctgtgtgactccatggatcttgaggtgttctttggggcttgtagtgctgacccacaggtggggatagtgtggctgtagtcagttaaagtttaaaatctctacatgtcaaaacatatcatttgagaggtaaaaatataaatgaggaaaatttctcattccttagttctaactccagaatgggtgtgctgaccatgaaaaaaatcagggaaatattttaacatgtgtgacacttgaagattaatatctttaatattagaagccttaactaatccagaagaaaaatataaataacccaaacagaaaaatagctaaaggacatgagtgaacagggtactcacaaaagaagaaaaacaattggtcaatacatatacaaaaaaaggtctaggctgtgcacggtggctcacgcctgtaatcccagcactttggaagcccgaggggggcagatcacgaggtcaggagatcaagaccatcctgactaacatggtgaaaccccgtctctattaaaaatacaaaaaattagccgggtgtgatggagggtgcctgtagttccagctactcgggaggctgaggcaggagaagggcgtgaacctggaaggtggagcttgtactgagctgaaattgcaccattgcactgcagcctgggagacagagcgagactccgtctcaaaaaaaaaaaaaaaaaaaaaaaaaaaggtctaatcttgccaattagcaaagaaaaataaattaagacaaagcatttcccacatatcatagtaacaggtgataatctaagagaattataattttcttttacccagaaattctacctttgtgattttttttttttttgagatggagtttcgctcttgttgcccaggttggagtgcaatggcgtgatctcagctcacagcaaccttcgtctcccggtttcaagtgattctcctgcctcaccctccggagtagctgggattacaggcatgctccaccacgcccggctaattttgtatttttagtagagacggggtttctccatgttggtcaggctggtctcgaactcgtgacctcacgtgatccacccacctcgtccttccaaagtgctgggattacaggtgcgagccacctcacccggcctctatgaatttatcttaaagaaattagtataagtgtgggaaatttttatctgaaggacctggatttcaacagcgtttcttttttttttttttgagacagagtctcgctctctcgcccaggctggagtgcagtggcgcgatctccgctcactgcaagctccgccttccgggttcacgccattctcctgcgtcagccttccgagtagctgggactacaggcgcccgccatgacgcccggctaatttttttgtatttttagtagagacggggtttcaccatgttagccaggatggtctcgatctcctgacctcgtgatccgcccgtctcggcctcccaaagtgctgggattacaggtgtgagccaccatgcccagcccagtgtttctaattatagaaaaaaaagtcagaaacaacctgagtgttcaacaagaggtacctgaccaaataaatgatggtccatatcattccatggaataaaatgtagccattaaaatgataatcagcctgggcatggtggcttatgcctgtaatcccaacactttgggaggaggaggcaggagcatcacttgagcccaggagttcaagaccaggctgggcagcatagcaagaccctgtctctacaaaaaaaatctaaaaattagctgggtgtggtggcacgctcctgtagtcccacctacttgggaggcttaggtgggaggatcaattgaatctaggaattggaggtaacagtgagctataatggtgccactgcactccagcctgagcagcagagcaagactatgtctttaaaaaaaattaaaataaataaaaagaaaaataaataattaaaagaaattaagttggttccatctataaggatgacagttgaggtaggatcaacactgaaaactgatcaggcaggtttggacagagctggctgtcacacacccggagagcactgctcatttcatttgccttcctccctgtgggcagctggccacacctgctctgagcatccctttctcaacccctggggagcagtagatgagctcatttcagcagtgatatgggttggtcttgccaggatactttctacttgggaatacctgagataacccaggtgaaatcctgttctgggcagctgctttgcaccaggcaccctgctggtcactggaaggcacacagacaggaataggtcacagtcactgtctgggtcacaaagttcttaactgcaaacaatagaatttactctaggtcatttgaggtggaaagagatttgttagagaattcaagcctctactaacggccagagacacaggctttcttggcacaagttcaggtagagcactgaaaccacactgctgcttggctccagcaagaccaccttgctaccagtcacagtcacagcccccatgcactgacactgcacactggactccaagaaacccttgccctaagagccagaaccctccacaccatcattcctagggagattctgcatgatgccagtgtccttggaccctctctgaagaggactttttgagaggagagtgtttgtggagccgaggtttgtgcctgtaccctggaggtaagcaggtgagggcagcaaatcctagttcctctcaatgtaaggcagaacttcccacttcacactgtgggaagttcttcaaacttggagttttaaaagaatccaaggggctacagtgcatgatgaatgtgccccgatttcctggaacttagagtcgatgaacacttaacttatgcaggtaaaggcagaagtgtgtggcaccgttggtgagtgaggaggcagccagacagcactacagctcttctaaggagcaggagagtgacctcaagctgtgttaggggtggtgaggctgattttagggagggtagaggatggtgctggaaagggccactgtggccagctcagatgagcttcaaggatcccctggttaggagcatgaactctcatctgtgggcagagggatctcaggttaaattttaggaactgtagcaagctgtggcgaggagtgaggggactttttgggataaaattgtcttatgagctcaattgcatcccctaaaaattcattggtttaagccctaacccccaatatcccagaatgtgactgtaatggagacacggcctttaaagaggtggcttaggtaaaatgagatagttagagtggaccctaatccaatacagctagtgtccttatcagaagaggaaatttggacacagagagagatgccagggatgcatgccatgtaagaggcagcaagagcatggccatctgcaaaccaaagagagagggctcagaggaaaacaagccttccccaatggcttgacctcacatttctagtctccagaactgtgagcaaataaatttctttagtcaaagccacccagttgcagtgttttgttgtggtggcagagcaaacagataaagtagtaagtccctccagcaccctaggaggttctgcttgttgccattccccaaaggccatcctagccatgatgagggcaggtgcggttgtttgggaggagaccactgtggaacaaagagaaacaaggcttgcttgatgcagcagggagtagactacagattcaagaagggctctagaaaggtccagcaattcagcttgtcagggaaccccgagctgtatgtagttcttgctctcctgtgccctgcaggatggcttgacccatttctgttctcatgtcagaccctaagggcagggtcatctgggccacagacccaggcccggtgggcctggtgggctgacctgtgggctattcttatgccttcaggaaacccacctttaccacctggctgttgtgactggcatcgctgaggctgtttctttgtgtataaaatcgaataaggacacatcacgtgctagcaatccaccagcatagggcgaaggctcagcaaagagaagttctctcctcctctgctcttttaggtcagcagcaaatgcagatcggggtggggacaaggtaaacacataacttgggtggagatgtgattgattaataactcataaatcatctgaaaccatactttccttttgatagtcaaccctctgtaaacactcaatgtgttctcaccttgttatcattcccaatgatgtcgaatgcgtggttgccctctccagtataaaagtttgatgcagctttgcctggatgtacctgtctataaggagtcctgcttatcacaatggtaggtaactggttttatatataaaggctagaaaagcaaaagagaatatgtattcctattttaatatgcgtgtgcttctaccacatagcattgaatggtaaatgtgacctagttaggaaccttatgttattacctgcaattcaatgtgcagagtatgagagtctgagagcttgcgaaaattgtaatgagtttagcactagttctgttctctaagatctaagttgtttttccttccagtaattctggggttcaattagatttcataagtgtctctaattcttgggtcctatgtgagtaatttgaaattaaaaataatttcagagctacatgactgcatcgtttttgagatgagggaactgagcctggggaggggaagaagctgccactggggtctcaccgcaagcctgtgaatgagctgagataggaacccaggcattcctctctccctgccaggggctcccaaacttggctgtgcagtggaaactccgggggaattttgaagaacactgacacccatcttccaccccgacactctgatttaattgggctgcagtgagaacagagcatcaatttaaaaagctgcccaggtcattttaatactcaagtttgggaaccatttccctaggcccttttcactgtatcatgtgttaaggaaaaatcttgggtttggggctgtgtttgttggtttatttgtttgtttttgagacaggatctcactctgttgcccaggctggagtgcagtggtgcagtcatgattcactgcagcctggaccttcctgggctcaggtgatcctcccatctcagcttcccaagtagctgggaccacaggcgagtgccaccatgcccgactaatttttgtatttgttttagagataggattttgctatgctgcccaggctggtcttgaactcctgggctcatgcaatctgcccacctcagcctcccaaagtggtgggattactggtgtgaaccactgtgcctggccaagaaagaattttgcttcaaaatttataaaaacccatgcaggcttttcttgtgtcttaaacttgatctttcttaaaacattctttactaacgctggggactgaactgggctgctctgggggctgcaagggagatgaggcaagaaacagccagccaagtgggaggagccaagggaggtgggagagggtcctcaggaggggaccccgggcctcctctgggccactcggcaagacaaacttgctctcagaaactgtccggaggcagaactggggatggtaaaacaacagtggggtgggaggtcctccccatctccactagacaggctcctccagcctatactctatgcagcctcacagtgaacttagcaagctgcagagctggcaatacctctgcctgcctaagatcctgaggacccccaactctttatttatttatttatttattttattattgttatttgtagtagagatgagtttttactatgttgcccaggctggtctctaactcttgggcccaagtgatcctcccaccttggcttcccaaagtgctgggattacaggtgtgagaaattatgcctggccagacccccaactcttgatcatgggataacccttccaggagcagtaatttacctaaagagatacaacttttttttttttttttttttttttttttctgagacggagtctctctgtcgcccaggctggagtgcagtggcgcaatctcggctcactgcaagctccacctcccgggttcacgccattctcctgcctcagcctcctgagtagctgggactacaggcgcccgccaccacgcccagctaattttttgtatttttagtagagacggggtttcaccatgttagccaggatggtctcgatctcctgacctcatgatccacccgcctcggcctcccaaagtgctgggattacaggcgtgagccaccgcgcccagccaagagacacaacttctaaggatactgccagcctgcttcacctcaaggctcgctgtaccatctagaaaatcctagcccattctggcgtcgtggtgtaatctcctgcacacatcaggagctcagggttactaaatgcaggaaaggaagacacaaagggaaggaaggctggtggcagaatagaagacaggaatgaaaaaagctttagagcctctctactcaggggtggacctcagaccagcagcgtgggcatcacctgggagcttccgtgtaagtctaaagacctactgaagcagaatctgcaggttaattaacaggctgtctggttgggcgcggtggctcacgcccataatcccagcactttgggaggccgaggcgggcggatcatttgaggtcaggtatttgagaccagcctggccaacatgatgaaactacgtctctactacaaatacaaaaattagccaggtgtggtggtgcactcctgtaatcccagctactcaggaggctgaggcaggagaatcacttgaacccgggaggcggaggttgcagtgagccaagattgcatcactgaactctactctaggcgatagaacgagattccgtcccaaaaagaaacaaacaaacagacaaacaaaacaaaacaaaaaaaactcccaggaatttatatgcacattaaagcagcactgggttggtcattgttcctgggttctcacctgggagactaggtaaagacactgaggcactggctcctagattgtgtttcctatgagctccctggataactctaatggtcacccaaatgggagaacccctctttttacagggaaggaaaactgagtttcagagagagagagagagagagacagaaagggagggagggagggagggagagagagagagagacagcattaggctatttctgagatactgtcaagtggataatctcaaaattaaataatttgctctctgctgtcaccatgtttgggctctcccccagtggttcaatctcctgcctctgctctagagctgcaggagacttggggattttgcacatgtcacttgtcctgggatgcagccctgcttgtccctgttgactctcctgctttgtgactcagtgaaaccagatccccaagattccagtgatccccagtaccccaggagccctctttctgcatgaagggagtaaaaatgctgtcaacaaggctggctttttggggggaaaagtttaggtttccacaatgatttcgagctgaaggctactcagaagaatgaaacagaacaagtgccactccaagtcctggcttgagctgaatgtatcaaggcgttgtgtcatttttaaagaatctttctttctttgtggctataaaactcatatattttattgtagaaaatttagaaaataagaggaaaagtccaaagaagaaaattgtcataatctccccatccaaagatgacttgttatttggtacaaaaccctcagtgtgttctgagcatctctctccgcatcatgtatttcatagcctgcttcatttcatgtcgtgacatgtggtgatcaattgctcatgtgacttcaccttctaccttgctggctcaccaggtgtttttctgagtcactgttctccactggctctgttcttgggcattttgtttctttccatttttccactgcagtgagcatcgttgtggaggagtctgtctctgtctctgtctctgtgccatttttaggatcacatcttggagatgaaaaggtgaaatcaaatgttgtaaacggttttaagaatgtggatatatagtagcaaattgttctccttaacgattgttcaaagtaagtcattcttgtgcatatcacctcccatagatacctctatttccctctgggtattataaattaaatgattattctcaccatcccattatgcttctatttcatgctacatatttacatttcttttgttatcagtaagttgaactttttcatgtgtttattcatcatttctgtgtgtgtgtgtctgtgtgtgaaatgccactttatttctcttgccctttttctgtttaagatgtttttcttctcccattggtatgtaatgatattgttctgaaaatttgatatctttctaatttcagttatgttcttatgacgtttatattctcaaatctatttttcttttatgcttagaataagtatttatgtatatatgctgtgattattttatgatcttattgtaaaaacttaaaccttgaatccttttagaatttatttcactaatggggactcaaataggaaattaactttttttcttccccaaatagttcaccacttgttctaatagcatttattgaataatcgctctccacagatctcaaagaacatgtttattttataataaacttctacatatatttatactttctgggttttgtttttttcttttttaccctatccaactacctttggaattttctgggttttctattttgatctctgtgtttgcctttttttttttaaagacagggtttcactctgttgcccaggctggagtgcagtggtgctatcatggctcactgcagcctcaaccttcctgggctcaagtgattctccacctcagcctcccaagtagcagagactacagggacacaccaccatacccacctaacttttgtactttttgtagagatgggttttcaccatgttgcctaggctggtcttgaactcctgagctcaagcgatgtgcctgcctcagcttcccaaattgctgagattacagaattgagacactgtgccctccctatctttctattcatatatgagtaacatactacttcaagtagcgtcacttcataattaattttagcaatcttacagaataaatgccttcattcccattgtcaaaagacaaaattacaacaaatttagtttaaagatcttaattgattttttttttttttttttttttgagacggagtctcactctcttgcccaggctagagtgcggtggcacaatcttggctcactgcaacctccgcctcccaggctcaagtgattctcccacctcagcctcccgagtacctgggattactggcgtgtgccgccacgcccagctaattttttggtattttagtagagaatgggtttcatcatgttgcccagggtggtttcaattgcctgagttcaggcaatccacccacctccgcctcccaaagtgctaggattacaggcatgagccaccacgcctggccctcaattaacttttacttgtgagtctataatcagggaacacttcattccataaaatagatcgagtgttctgcagagctgaccagagaagtttggctttttttttttttgagacggagtctcgctctgtcacccaggctggagtgcagtggccagatctcggctcactgcaaggtccgcctcctgggttcgtgccattctcctgcctcagactcccgagtagctgggactacaggcacccaccaccacacccggctaattttttgtatttttagtagagacggggtttcaccgtgttagccaggatggtctcgatctcctgacctcatgatccacccgccttggcctcccagagtgctgggatcacaggcgtgagccaccgtgcctggcagagaagtttggctttatagacagaaaagagccagaaaaagcagaaacagagaacaaaaagcaaattggtcttttcgaagtgactttgcttataagttaaagcagaggggactttctcatcatgtcagctaaaactggcctgttgggcatttggctattacctctcactctcttctaatttcttagaaggttggttaagcaacttagttttggcttagtggcagagaacttcagcatgggcgactgcattttggtttggtctgttgggcccagtgcagctcagtccaaacaaatggcctcctcaacattttttttaacaccatcttccctttcaaaagttcccagaaattaccatgttaaggtttgtttgtttatttagctcatctcaaaaagatgacaaagtcaactagatacttttccttgttctacaatttcagcctagattaactggtttgttcagttatgagctccacatcataacctctcagcagagcccagccgagatcctggtacacagcactcagtcaaggattatcctttgaattaattcattctccaccctgacctttcttcacattacagaatgttctcctgggcagcgttgtgatctttgccaccttcgtgactttatgcaatgcatcatgctatttcatacctaatgagggagttccaggagattcaaccaggagtaagtcttggcttttcaatgtttattatgttattgcagcctggtagatggacctgtctgcagatgaaagcctttgtgtttctgtttgtttgtttctttgttttttgagatagtcatgctctgtctcccaggctggagtgcagtggcaccatttcagctcactgcaaactcctcctcccgggttccagtgattctacctcctcagcctcccaagtagctgggactacaggtgtgtgccatcatgcctggctaatttttgtatttttggtaaagatggggctttgccacgttggccaggctggtctcgaactcctgacctcaggtcatctgcccgccacagcctcccaaagtgttgggattacaggtgtgagccactgtgcctggcctcacttgggcagccttctaagaacacagtagcctgggccctgtacccagagatactgatttcattgttctagggtaaggctgagacatgggtggctttcaagtacccaagatgtttcacatgtgcacccagggttgaaaaccaccgatgcaaagcaaagggaagtgtagaagtgagattctcatccagagataatccagtaggaaaaaatgtggtcccaagtacccacaggctcacatggctatctaattcaataaaagggccttattttttcttgaaatatctgttctgaaataaaacttagaaggatataagctttctgtaagaaaacattatcagtagcatgaactgaacagtactggaattagccgtgggtgaatacaggagcaccattcttagatacctttaaaataattgagctcctctctctctgggaggggtgtagatagtaggggcaggactcaatgtcctgataggccttgttctcattgcatgacttgcactgatgatgttgtgtgtattattttagaatgcatggatctcaaaggaaacaaacacccaataaactcggagtggcagactgacaactgtgagacatgcacttgctacgaaacagaaatttcatgttgcaccctgtaagtctcaagccagtgctggccaagagggggacatagctgttctctgaggaacagctactccgtcttgcaaaattccaatcttcaggggtttagtttttgttttagttttttatagactttatatagagtagttttagattcacagcaaaactgagcagaggtacagaaatttcccatgtgcctcctgccctcacacatgcacgacctccccgttatcaacatcgtccaccacagtggtgtatttgttatgactgatgaacctaccttgcacatcattatcacctgagtccatagtttatgttagggcttcctcttggtgttgtggtattggggggttagttttcagtcctatttctttctttttaaaaaattgtgggccagtcacaatggctcacgcctgtaatcccagcattttggggggccgaagcgggcggatcacgaggtcaggagatcgagaccctcctagctaacacgtgaaaccccgtctctactaaaaataccaaaaaaattagccgggcttggtggcgggtgcctgtagtcccagctacttgggaggctgaggcaggagaatggcgtgaacccgggaggcagagcttgcagtgagccgagatcgcaccactgcactccagcctgggcgacagagcgagactgtgtctaaaaaaaaaaaaaattgcggtaaaatatacgtaacgtaaaatgtaccatgttaacaattttaaatgtatactttagtggcattaggtccattcacattgttgtggcatcactaccatccacctccagaacttgttcatcctgcaaaactgaagctctatacccattaagcagtcactctccatccccttctccccagcccctggcaaccaccatttcacttctgtctctatgattttgactactctaggtccctcatataagtggaatcatacagtaattgcttttttgagactggctcatttcacttagcataatatcctcatggttcatctgtgttgacgcttgcatcaacattgccttcttgttcgaggctgaatcatgttctgtaatatggagagaccacattttgcatctccattcatctgttgatgaacacttgggttgttttccacctcaatctcatttcttttctgttgtttttgaaacttgataagtagctttattccagatgggctcctgaagacaggaaagagtatctaattgtctgacactattccagagattggcccggtcatcacctcagtctgggccttgtagatttgtagaaatagcagtggctctgagcataggcagagggatcagatgtgaggggtaagggaggatctgtagtgtgccttgcaccataaaacttatgctgcttcactgctcaagtttaactcacgtcagcccaaaggctatgcagatgttttctgctgggttgctatggacaccagaagtctagaacttgagcaggcggaacacagagtacccgcagtgccgtcctaatttacctgtttcctcagcagctatgtccataacagctacctaactcccaaccccccaaatccatctaacctcttcatgtagccttggcttgtagtgttctgcaggaacaggaatggatctgatggcagagcattggtgtcaaggggcatttcgcaaacttgtctgatcacagactcacctgggaagcttgatgaaatacagattcctgggctccatcccagaactgattgatcagaatctgtaggggaggggccaaggaatctgaattttaacaggtgcctcgagagtcttatgaggacactgcgagtaaagaagcaagcacttgacttagagcagaacacctgagtcctactgaacttgaagtgcctggagagttccagccacatttatcgagcaggcctcatgctaggtgcctaatggaacagaggaatattggactgggagatggctggctgctcagagcaggctcctgggcatcgcattggccagatcactgggtattacagctgaaccttgattttgtggctccagaaatttgacattgtctttatatcaagtgttgatgtcactctggtggagtgtgattgtatcccagccaggtgaaggactggtgactggtccctccctcacataggagcgtggctccctggtggctaaaatgtccatgtcacccagatttggcatccctgccctcttctaagattctccactgtaggctgggattctcgtggtcctggtgtggaaacgagccaggctgctccttgcagtgaagagggaagaacctcactgttggagtcagagtaacttgagtctgaatctccattcccttgtatactagctgtgtgaccctgagtcagtttctgaatgtctctgagcctctgtttcctcacattataattatgtggctatgataattaaatgagattctgtgtatatattctttaacccaacaaacacttaatgccttctctgtgccaattctgtgtcctgtgatcctgggacagtaactgacacctggtcaggtttcaaggtttcaaggtttctactgccatgttgcctgttccatttggcacactgctccatgcaggggaaatgaatcaacaccttcttccctggaattgcctcacctcctttgtttttctcatgaacttttttctcctccatggaaaaatacagactttgattaagctttttctggaagaagtttttggacccactgcaccctctatgggacgctgcccagatctcaaggtgctcggtggcagtggagcctgaggctgggggcccaggcagaggctcccaggtagggcatgggagatgtcctgggctccacgctgcccgggcccagggctggctccctgacttgggtgaggaaaagggagtgggagacaaaaggccaaggtcatccagccactgactctggttagctgaatacccgatgttgacagtttctccctccctctctctctctgctgtttcctctatagagctaagtctggtttgcattgaaataagcatgcagtaatatgccttttgccttaccaacttgtatttgcacacaggcagcacagattcaccatttcacttcctaccatgaactaaaagaattgtatctcttcaacaaacaccactttatttttaaccttttttttttattgtggtaaaatgtatactacataaaatgtgccagtgtaaccattttaaaacatacaattcagcggcattaagtacattcacagtactatgcaaccataaacactatttccaaatctttttaatgactccaatctgaaactctatatccattaaacaataactcagattccctgtcccccaaacctgtggtaatctcaagtctactttctgtctctatgaatttgcctactctagatatttcacataaatacaatcacactgtgtttgtcttttcgtgtctggcttatttcactcagcatgttgtcaaggttcattcatgttgtagcatgtatcagtacttcattcttgtttctgccagaataatattccatggcatggtcataccacattttgcttatccatttatttgttgaacacttgagttgcttctgcctgttggataaagtaagtaatgttgttctgaacattggcatacagtgtttgtcaatatacaaacattggcatataatgtttgagtccctgctttcaattcttttgggatatatacccaggagtagctttctgggtcatatggtaattttttgtttaacttattgaggagctgccaagctgttttccacagcagctgcaccattttacattcctaccagccagcaatcacctttaaacatgaaaaacatgaaaggttttaaatgatatttcccaattttctaagtaaacatatgaattatagaatcattgggaagcataagagaaaaaagaagaaatacctactcataatcccaactgtgtaaaggtaataactattaacattctatttttatttatttatttatttgtattaaaaaaattaagacagggtctcactatgttgcccaagctggtctcaaactcctgggatcaagctgttgtctcacctcagccccaagtagctgggattacaggcatgtgccaccatgcctgggcttctatcatcatttattttcttttttttttttttgagatggaatcttgctctgtcgcccaggatggagtgcagtggtgtgatctctgctcactgcaagctccgcctcctgggttcacgccattctcctgtctcagcctcctgagtagctgggactacaggcgcctgcaaccacgcccggctaattttttgtatttttagtggagacggggtttcaccggtttagccaggatggtctcgatctcctgacctcgtgatccgcccgcctcggcctcccaaggtgctgggattacaggcgtgagacacagcgcccggccctatcatcatttctttatgtctcttttctatatgttttttcttttacatagtggatatcaaattttatatcacagttttgtgggctgatttttccacttaatattctagcactgggctgggcatggtgtctcacgcctgtaatcccagcactttgggaggacaaggtgggtggatcatttgaggtcaggagttcgagacctgcctggccaacatggtgagacctcccccgcgccgacccgccatctctactaaaaatataaaaattagctgggtgtagtggtgtgctcctgtaatcccagctactcaagaggctgaggcaggagaatcgcttaaacctgggagacggaggctgaagtgagctgagatcgtgccactgcactccatcctgggtgacagagtgaaattcatctcaaaaaattaaagtaataatagtaataatacataaatattctagcattagtatcatctcatctcatcagaactcttcaaaatatttgtatgtactgcagctcaattagaggtggtctctgactttcatcagcttctccctgggctccatgacactggcctggagtgactcattgctctggttggttgagagagctcctttgccaacaggcctccaagtcagggctgggatttgtttcctttccacattctagcaacaatatgctggccacttcctgaacagggagggtgggaggagccagcatggaacaagctgccactttctagagtagccagacttgcccctgggcctgtcagcacctactgatgaccttctgtgcctgcaggatggaatgtaggggtgagctgtgtgactctatggtacagactggtccaggttcctggaggtcacactctggggctgccagccaaagggtgcccttaagttggagtgtgggtgacttcctgaacagagctggcactggggagggggtgctcggggtcctttctcttctaacaggccccagttttgtctcaatgacagtgtttctacacctgtgggttatgacaaagacaactgccaaagaatcttcaagaaggaggactgcaagtatatcgtggtggagaagaaggacccaaaaaagacctgttctgtcagtgaatggataatctaatgtgcttctagtaggcacagggctcccaggccaggcctcattctcctctggcctctaatagtcaatgattgtgtagccatgcctatcagtaaaaagatttttgagcaaacacttgaatatgtgtgtccttttaatttataatttatgtatgcattgatttaatacacattataaagaatacaaaacatttaaaaagggagacaaaactacatatgaatggccgggcacagtggctcatgcctataatcccagcactttgggaggccaaggcaggcggatcatttgaggtcaggggatcgagaacagcctggccaacatagtgaaaccccgtctccactgaaatacaaaattagcgtggtggcaggcacctgtagtcccagctactcgggaggctgaggcaggagaatcacttgaacccgggaggcggaggttgcagtgagccaagatcacatcagtgcactccagcctgggtgacagagtgagactccgtctcaaaaaacaagcaaacaaacaaacaaacccaaacccaaacaaaacaaaacaaaacaaaatacatatgaataaaagttctatttttcctaaactctataataggtcattgtgcatcccttactcaggggaccactgccactcttactgtttctgatgtctgcactctgctgcccgtgtcctgctgtctggtgaacgatgaaaaaaatgtgtgtcttcaagctggaattagatcccttcttttctttacggtgttcctgtttatccctccttcctcttcatcaggaagtgtcttctatgatgtgtcccccacattcctctgtgtttgcttgaggtcaaagcagcttagaacaagaaacagcttagaacaagcctgtaaaacagcttagaacaagcctgaagaaagtgcccccaagaccacatgcagcccgaggggaattaaaggccaggggctctgccttgcagatggagagtggcctgggtgcaaggccccagcatcagctacctcttgctacacacagaccaccgaaaacttagtggcttaagacaacgacaacattgattctcttttaactgtttctgtgttaaagaacatctgttcactcaacaggtctgtactgagcacctgggccaggcagtgctctagacctggggatacagcagtgaaccagagagatttcaggagagaaccctgttctcctggggcttggactcagctgtgtgtacgtgacagatcagaaatgagcaaacagtcatctgttgaacaatcagtgggcaactgataaagagctttattttcccctgtcccctatctgtttaaacatgctatgtgggccagttgagcaatctagacaggtcccttagctttcttgttcaccttcagataatttaaaaaagtggtctgagttacattttgagttacacaaagtctcattccaggggctgcatctgaattgagagggaatgggggacaggagagttaagggctccttctgcctgaggtcatgttctaggctgacttcctcatgggacttttgtgggcttctctctccacagctccccttccactcacagcctatgactgttatcttagcttctgcatcgtaatctatgtctcctattgttttctaatgaggttgccttgccccttgtagcagagtcactcttggatgagatccataaaaccctgctatgatcacctcctttgctgcggaatgtggggaagcagttggcaactgagtatggaggacctctgtcaaaactgagacagaaagaagtgctatttaacatctagtcacaagtaaataatgtgggttataatctgggctgtaggctggacgtggtggctcatgcttgtaatcctagatcaggtgggaggatcgcttgtgcccaggactttgagaccagtctgggtaacacagcaagaccctgtctctacaaaaaaatttaaaaaccgggcatcatggcgcatgtctgtaatcccagctactcagcaggttgaggcaggagaatcgcttgaacacaggaggcagaggttacagcaagccgagattgcgccactgcgcctgagcgacagagtcagaccctgtctcaaagaaaaaagaaaagaaaaaaagaatctgggttctgccatttatttatttactcaattttttttttttttt

SEQ ID NO: 6 - DNA, 30000 bp, Homo sapiens
ttttaaaattaaaaataatgtttgttaggagttataaattatgattaattaattatggggaattataaaatatgattgtatgagattttgatggtttataaagtgtatttattgttaattattttaaatattaatgaatttaaaaatgaatttacggagattggaatgttttttttttgttgtattagttggtttaggttgttataataaaatattatagattgggaggtttaagtaatagaaatttattttttatagttttgggggttggaagtttacgattaaggtgtaggaaaggtaggttttattttgaggttttttttttggtttatatgtggttatttttttattgcgtgtttatatgatttttttgtgtttttggaaagagggtgtgggggatagagggaaagagaaggagagggaattttttggtgtttcgttttttaaggattttaatttgggttattttggtttaggtattgtggggtggggggttgtggttgttttgttttgagtggttaagataaagtaatagaaaaatgtttaaagttgtgtagtaaagataagttatcgaatagggatttgtttattagtgtggggatttttaagtcggttattttggaggtaagtttttatagagtttatgtaaggtggtagtagtagaagaagggaattgtttttgtttttggtatattttttatcgatttggtgatgttggatattgcgatgaatggtaatgtggatgagaatatgatggatttttagaaaaggagatttagttgtttaggtggttgtaaattattatagtttttattttggggaggaattgggggtttggttttgggttagagagtagtttagtgagggtgagagttatagtttgttttgttagttggatttttagtttcggttaattagtaattaaggttgagtagattaggtttttcggagttggttttgggaagttagttttggggtgagttggtttttgttgtggtattgagataatattgttataaatttaatgcgtttttgtattttttttttttttttatttgtttatatttataattattatgtatattagtttttgttagtgtttttatttaatttaatagagatatgttatatttaaaaaaaaaaaaaaaatgggtcgggcgtagtggtttatgtttgtaattttagtattttgggaggtcgaggtgggtggattatgaggttaggagatcgagattattttgattaacgtggtgaaatttcgtttttattaaaaatataaaaaaaattagtcgggcgtggtggtaggcgtttgtagttttagttatttaggaggttgaggtaggagaatggcgtgaattcgggaggtagagtttgtagtgagtcgagatggcgttattgtattttagtttgggtttagtttgggtaataagagtaaaattttgttttaaaaaaaaaaaaaaaaagataaataataaaaaataaaatataaataaaaaagtaaattgtagatttttagtataaatgattttatttatttttggtattaagagattttaataaatattgttgtttttaatagtttatggttatatttttaggatgatgattttagtttttttaagtttttgtttaataaaatttaatgttgtttaaagaatttatgatttgttttaggtaaaatttgaatataggttttcgaattttttttttttaaaggatttattttagaaaattggtagttagaaattttttttttgttttttggaaatgtattttttataatataggaatgttttttttaaggatttgggagttattttttcgaaatgtaattattaggtaggatagagtttttgttttttaattttggtggtagggtaggattttgatattgataagtattagtttataatttgataagtataagtttataatttggttagtgtatttattgattaaatttttttttttttaattagtattttttagtattttttttttaatatattttagtatttaaaaagttttttgttttttggtttggtagaattgagtttagttaatgttgaagtgttttttttttgttatagtagtttgaataaaatttgttttgatatttttgataagtgtttggtgtaaaatttttttttaataatagtaagtgttgaaattggatagtgtaagttttttttttagttttgtttttttttagaatatttaggttattttaggttattcgtttttttatataaattttagtaaaagtttattaatttttataaaaatttgttggattattttttttgtttttgtttttgagattaagttttattttgttatttaggttggagtgtggtggtataattttggtttattgtaattttcgttttttaagtttaagcgatttttttgttttagtttttttagtagttgggattataggtagatgttattatgtttagataatttttgtatttttagtagagttattgttttattatgttggttaggttggttttgaatatttgaatttaggtgatttatttgtttcggttttttaagtgttgggattataggcgtgagttattgtatttagttaggattgattttttaatttttttttttgatttcgtagtatttggaatggatttatttttttaggaacgatattaattttttttaaattggtagaaatttattaataaattgggggaggattgttattttaataatagagttttttaatttatgaatatgggattatttaaattagggattagtaaattttttgtaaagaattagatagtaaaaatatttgtggtattgtgggttatatttaattttgttttgtagtgtaaaagtagtgttggttcggcgcggtggtttatgtttgtaattttagtattttgggaggttgaggcgggtggattatttaaggttaggagtttaagattagttggttaatatggagaaatttcgttattattaaaaatataaaaaaaatagttaggtatagtggcgtacgtttgtaattttaattattcgggaggttgagataggagaatcgtttgaatttgggaggtggatgttgtagtgagttgagattatattattgtattttagtttgggtaataagagtaaaattttattttaaaaaaaaaaaaattgtgttttaataaaattttatttataaaataaatagggggttagtttagggttatggattgtagtttgttgtttttaatttagaatttttttaatttttttgagtaaagttttatagtttttattgtaagttttgtattttttttattaaatttatttttaagtattttatttttttaggtgttattgtgaatgaaattttgtttttttaattttatttttatattgtttattattagtatgtagaaatataattgatttttatatatcggttttatatatttgaattttgtttgttaaatttatttattaattttagtaaaattttttgatagattatttgggattttttgtatatagattatgtggtttataaataaatatagttttatttttttttttttattttagatgtttttaatttatttttttttttttttttttttttattttttttttaagttgttttggttatgattagtagtataatgttgaatggaagtggtaagagtagatatttttgtttgttttttaattttgagtgggaaatattttgttttttattattaagtatattaattgtaggttgtttgtagttgttttttattagattgaagaagtttatttttatttttagtttgttgagagtttttattacgaatgtatgttggttattgttaaatgtttttgtatttattgaaatgataatgtgatgtattttagtttattaatttagtatattatatttattgattttgaggttttgttgtttttaattaattaattttaaaaattttattatatattagagaaagtattatttgatttttggatgttatattaattttgtatttttgggataaattatatttggttatggtgtataattttttgtatatgttgatggatttttgtttgttaatattttattgagtatgtttgtatttttatttatgagagatattgttttataatttttgtttattttggtgtgtttgtttagttttgatattggggtaatattggttttattaaaggagttggaatatgatttatttttttttattttttgaaagatttttgtgaaagatataatattttttttcgttttattgtgtcgtgtcgtgttttgttttgtttttttgaggtggagttttgtttttgttgtttaggcgggagtgtaatggtatgatttcggtttattgtaatttttattttttaggtttaggtgatgtttttgttttttaagtagttgggattataggtatgtattattagatttggttaattttttttttttttttttttttgagataaagttttgttttgttatttaggcgggagtgtagtaacgtgattttggtttattgtaagtttcgtttttcgggtttacgttatttttttattttagttttttgaatagttgggattataggtattcgcgattatatttaattttttttgtatttttagtagagacggggttttattacgttagttaggatgatttcgattttttgattttatgatttattcgtttcggttttttaacgtgttgggattatagttatgagttattatgttttgtttaattttgtatttttagtagtgatggggttttattatgttgattaggttggttttgaatttttgattttaggttatttattcgtttttgtttttaatttggttaatttttttattttgtagagatggagtttttattgtgttgtttaggttgtttttgaatttttggttttaagtaatttttttatttgggttttttaaagttttgggattataggtataagttattatttttggtttaaaaatgtattttttatatatatgtttttgttaaaattgggataaagagaaatagattggtagtggtatgtattttaaggaaaataggagaaagtttatttatttaggaaagtgaggtttataagtattttatatataaatattatatttgttttattgggttgtgttatttgatttgtttcgagtatttgaatttgttatttttaatagagaattgatttttttattagttaaagaggaatttgagatttggtgagggtgagtatttggttagttattagtaagtttttttttattttatttatcggcggtgattagtaattatgtttgataggtatttattttttttttggtgagtagttggaaaagagagtagtttggaaatatttggaaatagtatcgtttgttttttatatatgtgatttggaagttttttgtttgttttatattgttttgtttttttagattgaattagtgtttattttatatatgttgagtgatggtttatgtttttttaaaaggtataaaattaagttgtgttttgattattttgggtatatgttgttagaattttttgagggtgtgttacggtagtatatttttaattttggcgatttggtgttaacgttgtattaaattattaggttggttaatatttattggaattttttggtgttaaaggttgtgggagatttgaatatattgttaagggaggaggaaggggaaggagaagggttgtgggatttttcgtatgaaaggttttatgttggtggtagtttttatttgttgttttggggtttaaaggtgtttagatttttttatttgagtgtagatgttgttagttttttgggtaggtgtgtgagggaggtagttttggttgtaaagagagtaggggtttggttgggcgtggtggtttacgtttgtaattttagtattttgggaggtcgaggtggtagattatttgaggttaggagtttgtgattagtttggttaatatggtgaaattttatttttattaaaaaaaaaatatagaaattagttgggtatggtggtatgtatttgtaattttagttacgtaggaggttgaggtaggagaattatttgaatttaggaggcggaggttgtagtgagttgagatagcgttattgtattttagttcgggcgatagcgcgagattttattttaaaaaaaaaaaaaaaaaaaaaaaaaagagagtattggtttttgggttattaggattattatataattgaattgtgttaggtttttgagttgaagtttagttattataatttttgtgatttgtatatatatgtttagatggtttgtaggaattaagaagtttggagtagttaaaaaatttataaagtaaaatagttagtttttgttttaattgagtaattaaaattataatattttattattgtgatttgtttttgttttattttagttgattaattaattttgtgatattttttttttggataatgagttttatgatttttttattatgtattttgtgatttttttttttgttaataatagataattacgttttattgtaattttttattatttatttaatttttataaagtaattttttttttatttttttttgttgatttttttcggatttagtttatttgtatttaagtgaattaaaagttttattttttatataaagtttgtttggtggtttttttatatggacgtgtttgataaattggggtcggtttgtttagtgtagttaagtttagatatttatattgaagttgtagtattagaaaggaaggtgtttatttatagagttttgagtaagggggattaggtagcgtatgtttaaaatttgaattttttgatggttggtagatgtgaattttgaaaatttgagataagtgttaatttagaaagtttattttgttaaggttgaggatatgttttcgtgatatatttttaggaggttttgacgatatgtgtttaaggtggttagagtatagtttggttttatattttttagggagatatgagttattaattaatatatgtaaaatgaatattggtttggtttggaaaggtaggataatttgaagtaggtagtgggtttttagattatagataggtgagagataaacggttgtatttttttgagtttttgattagtttttttaaaggaggtaattagatatgtatttattttagtgagtagagggatgattttgaatagaatgcgaggtagatttgttttaagtagtttttagtttgaattttttttttaatttagtgatttgggagggttaatatagtttttttttatataggtaagggtttttaaaggcggggtaaatttttaggaaagtagtagaagttagataaaattgtaaattagtatatgaagggtatatattggtttggtttaaaaaaggtgagatattttgaagtaggggtttataggttataggtggatttagagattttttgatttgtagttggtttaggaggcgatgttttgtttgaaaatttagggttaatagaaaagaatattagttttagtttatgggtgtggtttttaggagttttcggaagaaatttagaatgaaagaaggtggttagagtgtagtttttagttttttttatttgaggtttgtaggttagtagattcgtttggtgggggtttatatggaggtttaggtttttgaatgataaatttaaggacgtgtgttaagatgttatttttagtttttaaaattaaatattttcggattttaatttttttggttattgttttgggttattattatttttttgtttaataagttatttatttattttttagggttagttggatgtttgaaattttttttgaaggtatttaagatttttttttatttttatgtttgatggtggcgttcggggtggtggtataggtttttgttttattttaggtttgggtttaaatttttatttttttttattatttgaaaattttgaatagttgtttttattttgaaattttaatgttttattggtaaagtgggcgagagaaggtttgttttgttgtgttgttgggaaatttttgaggtaatgtatgtgtggatatgagaggttttagttttttttttattttttagttttttattttggggtggttttggaaggtattgtttatggtaaggatagaaggttgttggttggcgtttttttgtttgatttagtggttgtatttatttagtatgtgttttatgaggagaataagggtttgtgatgttggtatagattagtttagatatattggtgggtacgtggttgttgtttaatatttttgagtgtttttatttagtatgttggattttggtttttttagattttaatattatttagaaatagaaggaatagagtttcgtatagtagggttttttagattgatttttggtagttgaattgaagggttagtggttgagttatattggttgtttaatattttgaatattatatttagaaagaaaatataagttttttgttacggtagtttttttgtgattgtgttatatttaatataagataaatattagttttataaaagggagtagtttttgggataatttattgaaagcgtgagaggtaggtagtttttgggttttgtgtatagttgttttaagttgtaaatatataatttttgattaaatattttttaatttatttgataaataagaagttttcgggtttttaaggggatgtttaagttttttttttttttgggataaaatttggttttgttttttaggttttttagcggttaatttggaatatgtttgtttttgtttcgtttttttgttttagtgttttttttgttgtttttatatttttttggataagttttttaagttttttatagaaaattagaaagattatttattagtagtttttttggtttttggtttgttagttttaatgtattttagtgtatttttgtgatattgtatttagagtagttgtggtttatagtatttatagtgttttttttcgttattttattagtggtttttttttttaatttagttttttgtttggatatttaggatttttattttatcgttatttttattttatatagggggtttattttatagtatttttgttgttgtaaaaagattttttattgtattttttggtatggagaatttgttttattgtatttttgaagtaagagattatgtagaaatgaatttatgggtgggggtagagtttgtttttttggtttttgtagagtgaaaatttttataatgtttgttttagattaagtttcgtagtttagaagttggtttatttttggatttaatatttgagttgaaaacgtttttttttgtttatgtttattttagttcggtttttaatagaatagaattttgacgaatattaaaacgggttttgttatagggtattataaagattgggttttataaagtaaaattgttagggtataagtggatttttagggaggttggtttatgttaggttgggaattgggtagttttcgaatattgaatatttttttatttgtttttttatgtttgtttgttttggggaggttggtttttatggaggggtttttgggaaggaatgggggtcgatggggaggggaatgggattcgtattgcgtatttggggtgaggaagggatttagagggaaagtggaaagaataggagtcggtggagggtaatgtgtagggtaggggtgggggtgtagtttttatttttggtttttgtgagatacgaaataagattgtgttttgcgtatgaagtaggtagttgggtgagaggtttaagagaagagattatggtttggaaaggttttagagtacgatgggagaggaagtggatgagagataagttattagtttgaagattggtaaggggagattttatcgattttggtgttaaattaagtttagtttaaagttttttttttatatattttaaatttattttaatggtttttttgtatatagtgaattgtaagttaattgtacgtgtaaataggttgttatttattttcgtattaagtagtcgagttttagttaattatagtagttagattttaattatttataggcggttagttgttgaaattggatttaaataagttaagtattatgttattattaattcgtttgtttttgtattttatttttagtttttgtatattatttttttttttttgtttataaatttttgattatgaggtagtgtaggagtttttttgaatttatttttgtttgggggttgtttgatttatataaatggttttttgtttaattaaattttgttaaatttaatttgtttaacgtttttttaatattgggaatgatggtagggtacggtggtttatgtttgtaattttagtattttgggagattaaggtaggtagattatttgaggttaggcgtttaagattaatttgggtaatggtgaaattttgtttttattaaaaatataaaaattagttaggtatggtggtgtatgtttatagttttagttattgggaaggttgaagtataagaatcgtttgtgttcgggaggtggaggttgtagtgagttgagattgtagtattgtattttattttgggcgatagagtaagattttgttttagaaaaaaataaaaaataaaataaaataaaaaattttggaaatgaatggtagtttttgttatagtattagtttaagggttgtgtgattttatggattttgaggtgttttttggggtttgtagtgttgatttataggtggggatagtgtggttgtagttagttaaagtttaaaatttttatatgttaaaatatattatttgagaggtaaaaatataaatgaggaaaattttttattttttagttttaattttagaatgggtgtgttgattatgaaaaaaattagggaaatattttaatatgtgtgatatttgaagattaatatttttaatattagaagttttaattaatttagaagaaaaatataaataatttaaatagaaaaatagttaaaggatatgagtgaatagggtatttataaaagaagaaaaataattggttaatatatatataaaaaaaggtttaggttgtgtacggtggtttacgtttgtaattttagtattttggaagttcgaggggggtagattacgaggttaggagattaagattattttgattaatatggtgaaatttcgtttttattaaaaatataaaaaattagtcgggtgtgatggagggtgtttgtagttttagttattcgggaggttgaggtaggagaagggcgtgaatttggaaggtggagtttgtattgagttgaaattgtattattgtattgtagtttgggagatagagcgagatttcgttttaaaaaaaaaaaaaaaaaaaaaaaaaaaggtttaattttgttaattagtaaagaaaaataaattaagataaagtattttttatatattatagtaataggtgataatttaagagaattataatttttttttatttagaaattttatttttgtgattttttttttttttgagatggagtttcgtttttgttgtttaggttggagtgtaatggcgtgattttagtttatagtaattttcgtttttcggttttaagtgatttttttgttttatttttcggagtagttgggattataggtatgttttattacgttcggttaattttgtatttttagtagagacggggtttttttatgttggttaggttggtttcgaattcgtgattttacgtgatttatttatttcgtttttttaaagtgttgggattataggtgcgagttattttattcggtttttatgaatttattttaaagaaattagtataagtgtgggaaatttttatttgaaggatttggattttaatagcgttttttttttttttttttgagatagagtttcgttttttcgtttaggttggagtgtagtggcgcgattttcgtttattgtaagtttcgtttttcgggtttacgttatttttttgcgttagtttttcgagtagttgggattataggcgttcgttatgacgttcggttaatttttttgtatttttagtagagacggggttttattatgttagttaggatggtttcgattttttgatttcgtgattcgttcgtttcggttttttaaagtgttgggattataggtgtgagttattatgtttagtttagtgtttttaattatagaaaaaaaagttagaaataatttgagtgtttaataagaggtatttgattaaataaatgatggtttatattattttatggaataaaatgtagttattaaaatgataattagtttgggtatggtggtttatgtttgtaattttaatattttgggaggaggaggtaggagtattatttgagtttaggagtttaagattaggttgggtagtatagtaagattttgtttttataaaaaaaatttaaaaattagttgggtgtggtggtacgtttttgtagttttatttatttgggaggtttaggtgggaggattaattgaatttaggaattggaggtaatagtgagttataatggtgttattgtattttagtttgagtagtagagtaagattatgtttttaaaaaaaattaaaataaataaaaagaaaaataaataattaaaagaaattaagttggttttatttataaggatgatagttgaggtaggattaatattgaaaattgattaggtaggtttggatagagttggttgttatatattcggagagtattgtttattttatttgtttttttttttgtgggtagttggttatatttgttttgagtattttttttttaatttttggggagtagtagatgagtttattttagtagtgatatgggttggttttgttaggatattttttatttgggaatatttgagataatttaggtgaaattttgttttgggtagttgttttgtattaggtattttgttggttattggaaggtatatagataggaataggttatagttattgtttgggttataaagtttttaattgtaaataatagaatttattttaggttatttgaggtggaaagagatttgttagagaatttaagtttttattaacggttagagatataggtttttttggtataagtttaggtagagtattgaaattatattgttgtttggttttagtaagattattttgttattagttatagttatagtttttatgtattgatattgtatattggattttaagaaatttttgttttaagagttagaattttttatattattatttttagggagattttgtatgatgttagtgtttttggattttttttgaagaggattttttgagaggagagtgtttgtggagtcgaggtttgtgtttgtattttggaggtaagtaggtgagggtagtaaattttagttttttttaatgtaaggtagaattttttattttatattgtgggaagttttttaaatttggagttttaaaagaatttaaggggttatagtgtatgatgaatgtgtttcgattttttggaatttagagtcgatgaatatttaatttatgtaggtaaaggtagaagtgtgtggtatcgttggtgagtgaggaggtagttagatagtattatagtttttttaaggagtaggagagtgattttaagttgtgttaggggtggtgaggttgattttagggagggtagaggatggtgttggaaagggttattgtggttagtttagatgagttttaaggattttttggttaggagtatgaatttttatttgtgggtagagggattttaggttaaattttaggaattgtagtaagttgtggcgaggagtgaggggattttttgggataaaattgttttatgagtttaattgtattttttaaaaatttattggtttaagttttaatttttaatattttagaatgtgattgtaatggagatacggtttttaaagaggtggtttaggtaaaatgagatagttagagtggattttaatttaatatagttagtgtttttattagaagaggaaatttggatatagagagagatgttagggatgtatgttatgtaagaggtagtaagagtatggttatttgtaaattaaagagagagggtttagaggaaaataagttttttttaatggtttgattttatatttttagtttttagaattgtgagtaaataaatttttttagttaaagttatttagttgtagtgttttgttgtggtggtagagtaaatagataaagtagtaagtttttttagtattttaggaggttttgtttgttgttattttttaaaggttattttagttatgatgagggtaggtgcggttgtttgggaggagattattgtggaataaagagaaataaggtttgtttgatgtagtagggagtagattatagatttaagaagggttttagaaaggtttagtaatttagtttgttagggaatttcgagttgtatgtagtttttgtttttttgtgttttgtaggatggtttgatttatttttgtttttatgttagattttaagggtagggttatttgggttatagatttaggttcggtgggtttggtgggttgatttgtgggttatttttatgtttttaggaaatttatttttattatttggttgttgtgattggtatcgttgaggttgtttttttgtgtataaaatcgaataaggatatattacgtgttagtaatttattagtatagggcgaaggtttagtaaagagaagttttttttttttttgtttttttaggttagtagtaaatgtagatcggggtggggataaggtaaatatataatttgggtggagatgtgattgattaataatttataaattatttgaaattatattttttttttgatagttaattttttgtaaatatttaatgtgtttttattttgttattatttttaatgatgtcgaatgcgtggttgttttttttagtataaaagtttgatgtagttttgtttggatgtatttgtttataaggagttttgtttattataatggtaggtaattggttttatatataaaggttagaaaagtaaaagagaatatgtatttttattttaatatgcgtgtgtttttattatatagtattgaatggtaaatgtgatttagttaggaattttatgttattatttgtaatttaatgtgtagagtatgagagtttgagagtttgcgaaaattgtaatgagtttagtattagttttgttttttaagatttaagttgtttttttttttagtaattttggggtttaattagattttataagtgtttttaatttttgggttttatgtgagtaatttgaaattaaaaataattttagagttatatgattgtatcgtttttgagatgagggaattgagtttggggaggggaagaagttgttattggggttttatcgtaagtttgtgaatgagttgagataggaatttaggtatttttttttttttgttaggggtttttaaatttggttgtgtagtggaaatttcgggggaattttgaagaatattgatatttattttttatttcgatattttgatttaattgggttgtagtgagaatagagtattaatttaaaaagttgtttaggttattttaatatttaagtttgggaattatttttttaggttttttttattgtattatgtgttaaggaaaaattttgggtttggggttgtgtttgttggtttatttgtttgtttttgagataggattttattttgttgtttaggttggagtgtagtggtgtagttatgatttattgtagtttggatttttttgggtttaggtgatttttttattttagttttttaagtagttgggattataggcgagtgttattatgttcgattaatttttgtatttgttttagagataggattttgttatgttgtttaggttggttttgaatttttgggtttatgtaatttgtttattttagttttttaaagtggtgggattattggtgtgaattattgtgtttggttaagaaagaattttgttttaaaatttataaaaatttatgtaggttttttttgtgttttaaatttgattttttttaaaatattttttattaacgttggggattgaattgggttgttttgggggttgtaagggagatgaggtaagaaatagttagttaagtgggaggagttaagggaggtgggagagggtttttaggaggggatttcgggttttttttgggttattcggtaagataaatttgtttttagaaattgttcggaggtagaattggggatggtaaaataatagtggggtgggaggttttttttatttttattagataggtttttttagtttatattttatgtagttttatagtgaatttagtaagttgtagagttggtaatatttttgtttgtttaagattttgaggatttttaattttttatttatttatttatttattttattattgttatttgtagtagagatgagtttttattatgttgtttaggttggtttttaatttttgggtttaagtgatttttttattttggttttttaaagtgttgggattataggtgtgagaaattatgtttggttagatttttaatttttgattatgggataatttttttaggagtagtaatttatttaaagagatataatttttttttttttttttttttttttttttttgagacggagtttttttgtcgtttaggttggagtgtagtggcgtaatttcggtttattgtaagttttatttttcgggtttacgttatttttttgttttagttttttgagtagttgggattataggcgttcgttattacgtttagttaattttttgtatttttagtagagacggggttttattatgttagttaggatggtttcgattttttgattttatgatttattcgtttcggttttttaaagtgttgggattataggcgtgagttatcgcgtttagttaagagatataatttttaaggatattgttagtttgttttattttaaggttcgttgtattatttagaaaattttagtttattttggcgtcgtggtgtaattttttgtatatattaggagtttagggttattaaatgtaggaaaggaagatataaagggaaggaaggttggtggtagaatagaagataggaatgaaaaaagttttagagtttttttatttaggggtggattttagattagtagcgtgggtattatttgggagttttcgtgtaagtttaaagatttattgaagtagaatttgtaggttaattaataggttgtttggttgggcgcggtggtttacgtttataattttagtattttgggaggtcgaggcgggcggattatttgaggttaggtatttgagattagtttggttaatatgatgaaattacgtttttattataaatataaaaattagttaggtgtggtggtgtatttttgtaattttagttatttaggaggttgaggtaggagaattatttgaattcgggaggcggaggttgtagtgagttaagattgtattattgaattttattttaggcgatagaacgagatttcgttttaaaaagaaataaataaatagataaataaaataaaataaaaaaaatttttaggaatttatatgtatattaaagtagtattgggttggttattgtttttgggtttttatttgggagattaggtaaagatattgaggtattggtttttagattgtgttttttatgagttttttggataattttaatggttatttaaatgggagaatttttttttttatagggaaggaaaattgagttttagagagagagagagagagagatagaaagggagggagggagggagggagagagagagagagatagtattaggttatttttgagatattgttaagtggataattttaaaattaaataatttgttttttgttgttattatgtttgggtttttttttagtggtttaattttttgtttttgttttagagttgtaggagatttggggattttgtatatgttatttgttttgggatgtagttttgtttgtttttgttgatttttttgttttgtgatttagtgaaattagatttttaagattttagtgatttttagtattttaggagttttttttttgtatgaagggagtaaaaatgttgttaataaggttggttttttggggggaaaagtttaggtttttataatgatttcgagttgaaggttatttagaagaatgaaatagaataagtgttattttaagttttggtttgagttgaatgtattaaggcgttgtgttatttttaaagaatttttttttttttgtggttataaaatttatatattttattgtagaaaatttagaaaataagaggaaaagtttaaagaagaaaattgttataatttttttatttaaagatgatttgttatttggtataaaatttttagtgtgttttgagtattttttttcgtattatgtattttatagtttgttttattttatgtcgtgatatgtggtgattaattgtttatgtgattttattttttattttgttggtttattaggtgtttttttgagttattgttttttattggttttgtttttgggtattttgtttttttttatttttttattgtagtgagtatcgttgtggaggagtttgtttttgtttttgtttttgtgttatttttaggattatattttggagatgaaaaggtgaaattaaatgttgtaaacggttttaagaatgtggatatatagtagtaaattgttttttttaacgattgtttaaagtaagttatttttgtgtatattattttttatagatatttttatttttttttgggtattataaattaaatgattatttttattattttattatgtttttattttatgttatatatttatattttttttgttattagtaagttgaatttttttatgtgtttatttattatttttgtgtgtgtgtgtttgtgtgtgaaatgttattttattttttttgttttttttttgtttaagatgtttttttttttttattggtatgtaatgatattgttttgaaaatttgatatttttttaattttagttatgtttttatgacgtttatatttttaaatttattttttttttatgtttagaataagtatttatgtatatatgttgtgattattttatgattttattgtaaaaatttaaattttgaatttttttagaatttattttattaatggggatttaaataggaaattaatttttttttttttttaaatagtttattatttgttttaatagtatttattgaataatcgttttttatagattttaaagaatatgtttattttataataaatttttatatatatttatattttttgggttttgttttttttttttttattttatttaattatttttggaattttttgggttttttattttgatttttgtgtttgtttttttttttttaaagatagggttttattttgttgtttaggttggagtgtagtggtgttattatggtttattgtagttttaatttttttgggtttaagtgattttttattttagttttttaagtagtagagattatagggatatattattatatttatttaatttttgtattttttgtagagatgggtttttattatgttgtttaggttggttttgaatttttgagtttaagcgatgtgtttgttttagttttttaaattgttgagattatagaattgagatattgtgttttttttatttttttatttatatatgagtaatatattattttaagtagcgttattttataattaattttagtaattttatagaataaatgtttttatttttattgttaaaagataaaattataataaatttagtttaaagattttaattgattttttttttttttttttttttgagacggagttttatttttttgtttaggttagagtgcggtggtataattttggtttattgtaattttcgttttttaggtttaagtgatttttttattttagtttttcgagtatttgggattattggcgtgtgtcgttacgtttagttaattttttggtattttagtagagaatgggttttattatgttgtttagggtggttttaattgtttgagtttaggtaatttatttattttcgttttttaaagtgttaggattataggtatgagttattacgtttggtttttaattaatttttatttgtgagtttataattagggaatattttattttataaaatagatcgagtgttttgtagagttgattagagaagtttggttttttttttttttgagacggagtttcgttttgttatttaggttggagtgtagtggttagatttcggtttattgtaaggttcgttttttgggttcgtgttatttttttgttttagattttcgagtagttgggattataggtatttattattatattcggttaattttttgtatttttagtagagacggggttttatcgtgttagttaggatggtttcgattttttgattttatgatttattcgttttggttttttagagtgttgggattataggcgtgagttatcgtgtttggtagagaagtttggttttatagatagaaaagagttagaaaaagtagaaatagagaataaaaagtaaattggttttttcgaagtgattttgtttataagttaaagtagaggggatttttttattatgttagttaaaattggtttgttgggtatttggttattattttttatttttttttaattttttagaaggttggttaagtaatttagttttggtttagtggtagagaattttagtatgggcgattgtattttggtttggtttgttgggtttagtgtagtttagtttaaataaatggtttttttaatattttttttaatattatttttttttttaaaagtttttagaaattattatgttaaggtttgtttgtttatttagtttattttaaaaagatgataaagttaattagatatttttttttgttttataattttagtttagattaattggtttgtttagttatgagttttatattataattttttagtagagtttagtcgagattttggtatatagtatttagttaaggattattttttgaattaatttattttttattttgatttttttttatattatagaatgtttttttgggtagcgttgtgatttttgttattttcgtgattttatgtaatgtattatgttattttatatttaatgagggagttttaggagatttaattaggagtaagttttggttttttaatgtttattatgttattgtagtttggtagatggatttgtttgtagatgaaagtttttgtgtttttgtttgtttgtttttttgttttttgagatagttatgttttgttttttaggttggagtgtagtggtattattttagtttattgtaaattttttttttcgggttttagtgattttatttttttagttttttaagtagttgggattataggtgtgtgttattatgtttggttaatttttgtatttttggtaaagatggggttttgttacgttggttaggttggtttcgaatttttgattttaggttatttgttcgttatagttttttaaagtgttgggattataggtgtgagttattgtgtttggttttatttgggtagttttttaagaatatagtagtttgggttttgtatttagagatattgattttattgttttagggtaaggttgagatatgggtggtttttaagtatttaagatgttttatatgtgtatttagggttgaaaattatcgatgtaaagtaaagggaagtgtagaagtgagatttttatttagagataatttagtaggaaaaaatgtggttttaagtatttataggtttatatggttatttaatttaataaaagggttttatttttttttgaaatatttgttttgaaataaaatttagaaggatataagttttttgtaagaaaatattattagtagtatgaattgaatagtattggaattagtcgtgggtgaatataggagtattatttttagatatttttaaaataattgagtttttttttttttgggaggggtgtagatagtaggggtaggatttaatgttttgataggttttgtttttattgtatgatttgtattgatgatgttgtgtgtattattttagaatgtatggattttaaaggaaataaatatttaataaattcggagtggtagattgataattgtgagatatgtatttgttacgaaatagaaattttatgttgtattttgtaagttttaagttagtgttggttaagagggggatatagttgttttttgaggaatagttatttcgttttgtaaaattttaatttttaggggtttagtttttgttttagttttttatagattttatatagagtagttttagatttatagtaaaattgagtagaggtatagaaattttttatgtgttttttgtttttatatatgtacgattttttcgttattaatatcgtttattatagtggtgtatttgttatgattgatgaatttattttgtatattattattatttgagtttatagtttatgttagggtttttttttggtgttgtggtattggggggttagtttttagttttatttttttttttttaaaaaattgtgggttagttataatggtttacgtttgtaattttagtattttggggggtcgaagcgggcggattacgaggttaggagatcgagatttttttagttaatacgtgaaatttcgtttttattaaaaatattaaaaaaattagtcgggtttggtggcgggtgtttgtagttttagttatttgggaggttgaggtaggagaatggcgtgaattcgggaggtagagtttgtagtgagtcgagatcgtattattgtattttagtttgggcgatagagcgagattgtgtttaaaaaaaaaaaaaattgcggtaaaatatacgtaacgtaaaatgtattatgttaataattttaaatgtatattttagtggtattaggtttatttatattgttgtggtattattattatttatttttagaatttgtttattttgtaaaattgaagttttatatttattaagtagttattttttatttttttttttttagtttttggtaattattattttatttttgtttttatgattttgattattttaggttttttatataagtggaattatatagtaattgtttttttgagattggtttattttatttagtataatatttttatggtttatttgtgttgacgtttgtattaatattgtttttttgttcgaggttgaattatgttttgtaatatggagagattatattttgtatttttatttatttgttgatgaatatttgggttgttttttattttaattttattttttttttgttgtttttgaaatttgataagtagttttattttagatgggtttttgaagataggaaagagtatttaattgtttgatattattttagagattggttcggttattattttagtttgggttttgtagatttgtagaaatagtagtggttttgagtataggtagagggattagatgtgaggggtaagggaggatttgtagtgtgttttgtattataaaatttatgttgttttattgtttaagtttaatttacgttagtttaaaggttatgtagatgttttttgttgggttgttatggatattagaagtttagaatttgagtaggcggaatatagagtattcgtagtgtcgttttaatttatttgtttttttagtagttatgtttataatagttatttaatttttaattttttaaatttatttaatttttttatgtagttttggtttgtagtgttttgtaggaataggaatggatttgatggtagagtattggtgttaaggggtatttcgtaaatttgtttgattatagatttatttgggaagtttgatgaaatatagatttttgggttttattttagaattgattgattagaatttgtaggggaggggttaaggaatttgaattttaataggtgtttcgagagttttatgaggatattgcgagtaaagaagtaagtatttgatttagagtagaatatttgagttttattgaatttgaagtgtttggagagttttagttatatttatcgagtaggttttatgttaggtgtttaatggaatagaggaatattggattgggagatggttggttgtttagagtaggtttttgggtatcgtattggttagattattgggtattatagttgaattttgattttgtggttttagaaatttgatattgtttttatattaagtgttgatgttattttggtggagtgtgattgtattttagttaggtgaaggattggtgattggttttttttttatataggagcgtggttttttggtggttaaaatgtttatgttatttagatttggtatttttgtttttttttaagattttttattgtaggttgggattttcgtggttttggtgtggaaacgagttaggttgttttttgtagtgaagagggaagaattttattgttggagttagagtaatttgagtttgaatttttatttttttgtatattagttgtgtgattttgagttagtttttgaatgtttttgagtttttgtttttttatattataattatgtggttatgataattaaatgagattttgtgtatatattttttaatttaataaatatttaatgttttttttgtgttaattttgtgttttgtgattttgggatagtaattgatatttggttaggttttaaggttttaaggtttttattgttatgttgtttgttttatttggtatattgttttatgtaggggaaatgaattaatatttttttttttggaattgttttattttttttgttttttttatgaattttttttttttttatggaaaaatatagattttgattaagttttttttggaagaagtttttggatttattgtattttttatgggacgttgtttagattttaaggtgttcggtggtagtggagtttgaggttgggggtttaggtagaggtttttaggtagggtatgggagatgttttgggttttacgttgttcgggtttagggttggttttttgatttgggtgaggaaaagggagtgggagataaaaggttaaggttatttagttattgattttggttagttgaatattcgatgttgatagtttttttttttttttttttttttgttgttttttttatagagttaagtttggtttgtattgaaataagtatgtagtaatatgttttttgttttattaatttgtatttgtatataggtagtatagatttattattttattttttattatgaattaaaagaattgtatttttttaataaatattattttatttttaattttttttttttattgtggtaaaatgtatattatataaaatgtgttagtgtaattattttaaaatatataatttagcggtattaagtatatttatagtattatgtaattataaatattatttttaaatttttttaatgattttaatttgaaattttatatttattaaataataatttagattttttgttttttaaatttgtggtaattttaagtttattttttgtttttatgaatttgtttattttagatattttatataaatataattatattgtgtttgttttttcgtgtttggtttattttatttagtatgttgttaaggtttatttatgttgtagtatgtattagtattttatttttgtttttgttagaataatattttatggtatggttatattatattttgtttatttatttatttgttgaatatttgagttgtttttgtttgttggataaagtaagtaatgttgttttgaatattggtatatagtgtttgttaatatataaatattggtatataatgtttgagtttttgtttttaatttttttgggatatatatttaggagtagttttttgggttatatggtaattttttgtttaatttattgaggagttgttaagttgttttttatagtagttgtattattttatatttttattagttagtaattatttttaaatatgaaaaatatgaaaggttttaaatgatattttttaattttttaagtaaatatatgaattatagaattattgggaagtataagagaaaaaagaagaaatatttatttataattttaattgtgtaaaggtaataattattaatattttatttttatttatttatttatttgtattaaaaaaattaagatagggttttattatgttgtttaagttggttttaaatttttgggattaagttgttgttttattttagttttaagtagttgggattataggtatgtgttattatgtttgggtttttattattatttatttttttttttttttttttgagatggaattttgttttgtcgtttaggatggagtgtagtggtgtgatttttgtttattgtaagtttcgttttttgggtttacgttatttttttgttttagttttttgagtagttgggattataggcgtttgtaattacgttcggttaattttttgtatttttagtggagacggggttttatcggtttagttaggatggtttcgattttttgatttcgtgattcgttcgtttcggttttttaaggtgttgggattataggcgtgagatatagcgttcggttttattattatttttttatgttttttttttatatgtttttttttttatatagtggatattaaattttatattatagttttgtgggttgatttttttatttaatattttagtattgggttgggtatggtgttttacgtttgtaattttagtattttgggaggataaggtgggtggattatttgaggttaggagttcgagatttgtttggttaatatggtgagatttttttcgcgtcgattcgttatttttattaaaaatataaaaattagttgggtgtagtggtgtgtttttgtaattttagttatttaagaggttgaggtaggagaatcgtttaaatttgggagacggaggttgaagtgagttgagatcgtgttattgtattttattttgggtgatagagtgaaatttattttaaaaaattaaagtaataatagtaataatatataaatattttagtattagtattattttattttattagaattttttaaaatatttgtatgtattgtagtttaattagaggtggtttttgatttttattagtttttttttgggttttatgatattggtttggagtgatttattgttttggttggttgagagagtttttttgttaataggtttttaagttagggttgggatttgttttttttttatattttagtaataatatgttggttattttttgaatagggagggtgggaggagttagtatggaataagttgttattttttagagtagttagatttgtttttgggtttgttagtatttattgatgattttttgtgtttgtaggatggaatgtaggggtgagttgtgtgattttatggtatagattggtttaggtttttggaggttatattttggggttgttagttaaagggtgtttttaagttggagtgtgggtgattttttgaatagagttggtattggggagggggtgttcggggtttttttttttttaataggttttagttttgttttaatgatagtgtttttatatttgtgggttatgataaagataattgttaaagaatttttaagaaggaggattgtaagtatatcgtggtggagaagaaggatttaaaaaagatttgttttgttagtgaatggataatttaatgtgtttttagtaggtatagggtttttaggttaggttttatttttttttggtttttaatagttaatgattgtgtagttatgtttattagtaaaaagatttttgagtaaatatttgaatatgtgtgtttttttaatttataatttatgtatgtattgatttaatatatattataaagaatataaaatatttaaaaagggagataaaattatatatgaatggtcgggtatagtggtttatgtttataattttagtattttgggaggttaaggtaggcggattatttgaggttaggggatcgagaatagtttggttaatatagtgaaatttcgtttttattgaaatataaaattagcgtggtggtaggtatttgtagttttagttattcgggaggttgaggtaggagaattatttgaattcgggaggcggaggttgtagtgagttaagattatattagtgtattttagtttgggtgatagagtgagatttcgttttaaaaaataagtaaataaataaataaatttaaatttaaataaaataaaataaaataaaatatatatgaataaaagttttattttttttaaattttataataggttattgtgtattttttatttaggggattattgttatttttattgtttttgatgtttgtattttgttgttcgtgttttgttgtttggtgaacgatgaaaaaaatgtgtgtttttaagttggaattagatttttttttttttttacggtgtttttgtttattttttttttttttttattaggaagtgttttttatgatgtgttttttatatttttttgtgtttgtttgaggttaaagtagtttagaataagaaatagtttagaataagtttgtaaaatagtttagaataagtttgaagaaagtgtttttaagattatatgtagttcgaggggaattaaaggttaggggttttgttttgtagatggagagtggtttgggtgtaaggttttagtattagttattttttgttatatatagattatcgaaaatttagtggtttaagataacgataatattgatttttttttaattgtttttgtgttaaagaatatttgtttatttaataggtttgtattgagtatttgggttaggtagtgttttagatttggggatatagtagtgaattagagagattttaggagagaattttgtttttttggggtttggatttagttgtgtgtacgtgatagattagaaatgagtaaatagttatttgttgaataattagtgggtaattgataaagagttttatttttttttgttttttatttgtttaaatatgttatgtgggttagttgagtaatttagataggttttttagtttttttgtttatttttagataatttaaaaaagtggtttgagttatattttgagttatataaagttttattttaggggttgtatttgaattgagagggaatgggggataggagagttaagggttttttttgtttgaggttatgttttaggttgatttttttatgggatttttgtgggtttttttttttatagttttttttttatttatagtttatgattgttattttagtttttgtatcgtaatttatgttttttattgttttttaatgaggttgttttgttttttgtagtagagttatttttggatgagatttataaaattttgttatgattattttttttgttgcggaatgtggggaagtagttggtaattgagtatggaggatttttgttaaaattgagatagaaagaagtgttatttaatatttagttataagtaaataatgtgggttataatttgggttgtaggttggacgtggtggtttatgtttgtaattttagattaggtgggaggatcgtttgtgtttaggattttgagattagtttgggtaatatagtaagattttgtttttataaaaaaatttaaaaatcgggtattatggcgtatgtttgtaattttagttatttagtaggttgaggtaggagaatcgtttgaatataggaggtagaggttatagtaagtcgagattgcgttattgcgtttgagcgatagagttagattttgttttaaagaaaaaagaaaagaaaaaaagaatttgggttttgttatttatttatttatttaattttttttttttttt

SEQ ID NO: 7 - DNA, 29999 bp, Homo sapiens
aaaaaaaaaaaaaaattgagtaaataaataaatggcagaacccagattctttttttcttttcttttttctttgagacagggtctgactctgtcgctcaggcgcagtggcgcaatctcggcttgctgtaacctctgcctcctgtgttcaagcgattctcctgcctcaacctgctgagtagctgggattacagacatgcgccatgatgcccggtttttaaatttttttgtagagacagggtcttgctgtgttacccagactggtctcaaagtcctgggcacaagcgatcctcccacctgatctaggattacaagcatgagccaccacgtccagcctacagcccagattataacccacattatttacttgtgactagatgttaaatagcacttctttctgtctcagttttgacagaggtcctccatactcagttgccaactgcttccccacattccgcagcaaaggaggtgatcatagcagggttttatggatctcatccaagagtgactctgctacaaggggcaaggcaacctcattagaaaacaataggagacatagattacgatgcagaagctaagataacagtcataggctgtgagtggaaggggagctgtggagagagaagcccacaaaagtcccatgaggaagtcagcctagaacatgacctcaggcagaaggagcccttaactctcctgtcccccattccctctcaattcagatgcagcccctggaatgagactttgtgtaactcaaaatgtaactcagaccacttttttaaattatctgaaggtgaacaagaaagctaagggacctgtctagattgctcaactggcccacatagcatgtttaaacagataggggacaggggaaaataaagctctttatcagttgcccactgattgttcaacagatgactgtttgctcatttctgatctgtcacgtacacacagctgagtccaagccccaggagaacagggttctctcctgaaatctctctggttcactgctgtatccccaggtctagagcactgcctggcccaggtgctcagtacagacctgttgagtgaacagatgttctttaacacagaaacagttaaaagagaatcaatgttgtcgttgtcttaagccactaagttttcggtggtctgtgtgtagcaagaggtagctgatgctggggccttgcacccaggccactctccatctgcaaggcagagcccctggcctttaattcccctcgggctgcatgtggtcttgggggcactttcttcaggcttgttctaagctgttttacaggcttgttctaagctgtttcttgttctaagctgctttgacctcaagcaaacacagaggaatgtgggggacacatcatagaagacacttcctgatgaagaggaaggagggataaacaggaacaccgtaaagaaaagaagggatctaattccagcttgaagacacacatttttttcatcgttcaccagacagcaggacacgggcagcagagtgcagacatcagaaacagtaagagtggcagtggtcccctgagtaagggatgcacaatgacctattatagagtttaggaaaaatagaacttttattcatatgtattttgttttgttttgttttgtttgggtttgggtttgtttgtttgtttgcttgttttttgagacggagtctcactctgtcacccaggctggagtgcactgatgtgatcttggctcactgcaacctccgcctcccgggttcaagtgattctcctgcctcagcctcccgagtagctgggactacaggtgcctgccaccacgctaattttgtatttcagtggagacggggtttcactatgttggccaggctgttctcgatcccctgacctcaaatgatccgcctgccttggcctcccaaagtgctgggattataggcatgagccactgtgcccggccattcatatgtagttttgtctccctttttaaatgttttgtattctttataatgtgtattaaatcaatgcatacataaattataaattaaaaggacacacatattcaagtgtttgctcaaaaatctttttactgataggcatggctacacaatcattgactattagaggccagaggagaatgaggcctggcctgggagccctgtgcctactagaagcacattagattatccattcactgacagaacaggtcttttttgggtccttcttctccaccacgatatacttgcagtcctccttcttgaagattctttggcagttgtctttgtcataacccacaggtgtagaaacactgtcattgagacaaaactggggcctgttagaagagaaaggaccccgagcaccccctccccagtgccagctctgttcaggaagtcacccacactccaacttaagggcaccctttggctggcagccccagagtgtgacctccaggaacctggaccagtctgtaccatagagtcacacagctcacccctacattccatcctgcaggcacagaaggtcatcagtaggtgctgacaggcccaggggcaagtctggctactctagaaagtggcagcttgttccatgctggctcctcccaccctccctgttcaggaagtggccagcatattgttgctagaatgtggaaaggaaacaaatcccagccctgacttggaggcctgttggcaaaggagctctctcaaccaaccagagcaatgagtcactccaggccagtgtcatggagcccagggagaagctgatgaaagtcagagaccacctctaattgagctgcagtacatacaaatattttgaagagttctgatgagatgagatgatactaatgctagaatatttatgtattattactattattactttaattttttgagatgaatttcactctgtcacccaggatggagtgcagtggcacgatctcagctcacttcagcctccgtctcccaggtttaagcgattctcctgcctcagcctcttgagtagctgggattacaggagcacaccactacacccagctaatttttatatttttagtagagatggcgggtcggcgcgggggaggtctcaccatgttggccaggcaggtctcgaactcctgacctcaaatgatccacccaccttgtcctcccaaagtgctgggattacaggcgtgagacaccatgcccagcccagtgctagaatattaagtggaaaaatcagcccacaaaactgtgatataaaatttgatatccactatgtaaaagaaaaaacatatagaaaagagacataaagaaatgatgatagggccgggcgctgtgtctcacgcctgtaatcccagcaccttgggaggccgaggcgggcggatcacgaggtcaggagatcgagaccatcctggctaaaccggtgaaaccccgtctccactaaaaatacaaaaaattagccgggcgtggttgcaggcgcctgtagtcccagctactcaggaggctgagacaggagaatggcgtgaacccaggaggcggagcttgcagtgagcagagatcacaccactgcactccatcctgggcgacagagcaagattccatctcaaaaaaaaaaaaaagaaaataaatgatgatagaagcccaggcatggtggcacatgcctgtaatcccagctacttggggctgaggtgagacaacagcttgatcccaggagtttgagaccagcttgggcaacatagtgagaccctgtcttaatttttttaatacaaataaataaataaataaaaatagaatgttaatagttattacctttacacagttgggattatgagtaggtatttcttcttttttctcttatgcttcccaatgattctataattcatatgtttacttagaaaattgggaaatatcatttaaaacctttcatgtttttcatgtttaaaggtgattgctggctggtaggaatgtaaaatggtgcagctgctgtggaaaacagcttggcagctcctcaataagttaaacaaaaaattaccatatgacccagaaagctactcctgggtatatatcccaaaagaattgaaagcagggactcaaacattatatgccaatgtttgtatattgacaaacactgtatgccaatgttcagaacaacattacttactttatccaacaggcagaagcaactcaagtgttcaacaaataaatggataagcaaaatgtggtatgaccatgccatggaatattattctggcagaaacaagaatgaagtactgatacatgctacaacatgaatgaaccttgacaacatgctgagtgaaataagccagacacgaaaagacaaacacagtgtgattgtatttatgtgaaatatctagagtaggcaaattcatagagacagaaagtagacttgagattaccacaggtttgggggacagggaatctgagttattgtttaatggatatagagtttcagattggagtcattaaaaagatttggaaatagtgtttatggttgcatagtactgtgaatgtacttaatgccgctgaattgtatgttttaaaatggttacactggcacattttatgtagtatacattttaccacaataaaaaaaaaaggttaaaaataaagtggtgtttgttgaagagatacaattcttttagttcatggtaggaagtgaaatggtgaatctgtgctgcctgtgtgcaaatacaagttggtaaggcaaaaggcatattactgcatgcttatttcaatgcaaaccagacttagctctatagaggaaacagcagagagagagagggagggagaaactgtcaacatcgggtattcagctaaccagagtcagtggctggatgaccttggccttttgtctcccactcccttttcctcacccaagtcagggagccagccctgggcccgggcagcgtggagcccaggacatctcccatgccctacctgggagcctctgcctgggcccccagcctcaggctccactgccaccgagcaccttgagatctgggcagcgtcccatagagggtgcagtgggtccaaaaacttcttccagaaaaagcttaatcaaagtctgtatttttccatggaggagaaaaaagttcatgagaaaaacaaaggaggtgaggcaattccagggaagaaggtgttgattcatttcccctgcatggagcagtgtgccaaatggaacaggcaacatggcagtagaaaccttgaaaccttgaaacctgaccaggtgtcagttactgtcccaggatcacaggacacagaattggcacagagaaggcattaagtgtttgttgggttaaagaatatatacacagaatctcatttaattatcatagccacataattataatgtgaggaaacagaggctcagagacattcagaaactgactcagggtcacacagctagtatacaagggaatggagattcagactcaagttactctgactccaacagtgaggttcttccctcttcactgcaaggagcagcctggctcgtttccacaccaggaccacgagaatcccagcctacagtggagaatcttagaagagggcagggatgccaaatctgggtgacatggacattttagccaccagggagccacgctcctatgtgagggagggaccagtcaccagtccttcacctggctgggatacaatcacactccaccagagtgacatcaacacttgatataaagacaatgtcaaatttctggagccacaaaatcaaggttcagctgtaatacccagtgatctggccaatgcgatgcccaggagcctgctctgagcagccagccatctcccagtccaatattcctctgttccattaggcacctagcatgaggcctgctcgataaatgtggctggaactctccaggcacttcaagttcagtaggactcaggtgttctgctctaagtcaagtgcttgcttctttactcgcagtgtcctcataagactctcgaggcacctgttaaaattcagattccttggcccctcccctacagattctgatcaatcagttctgggatggagcccaggaatctgtatttcatcaagcttcccaggtgagtctgtgatcagacaagtttgcgaaatgccccttgacaccaatgctctgccatcagatccattcctgttcctgcagaacactacaagccaaggctacatgaagaggttagatggatttggggggttgggagttaggtagctgttatggacatagctgctgaggaaacaggtaaattaggacggcactgcgggtactctgtgttccgcctgctcaagttctagacttctggtgtccatagcaacccagcagaaaacatctgcatagcctttgggctgacgtgagttaaacttgagcagtgaagcagcataagttttatggtgcaaggcacactacagatcctcccttacccctcacatctgatccctctgcctatgctcagagccactgctatttctacaaatctacaaggcccagactgaggtgatgaccgggccaatctctggaatagtgtcagacaattagatactctttcctgtcttcaggagcccatctggaataaagctacttatcaagtttcaaaaacaacagaaaagaaatgagattgaggtggaaaacaacccaagtgttcatcaacagatgaatggagatgcaaaatgtggtctctccatattacagaacatgattcagcctcgaacaagaaggcaatgttgatgcaagcgtcaacacagatgaaccatgaggatattatgctaagtgaaatgagccagtctcaaaaaagcaattactgtatgattccacttatatgagggacctagagtagtcaaaatcatagagacagaagtgaaatggtggttgccaggggctggggagaaggggatggagagtgactgcttaatgggtatagagcttcagttttgcaggatgaacaagttctggaggtggatggtagtgatgccacaacaatgtgaatggacctaatgccactaaagtatacatttaaaattgttaacatggtacattttacgttacgtatattttaccgcaattttttttttttttagacacagtctcgctctgtcgcccaggctggagtgcagtggtgcgatctcggctcactgcaagctctgcctcccgggttcacgccattctcctgcctcagcctcccaagtagctgggactacaggcacccgccaccaagcccggctaatttttttggtatttttagtagagacggggtttcacgtgttagctaggagggtctcgatctcctgacctcgtgatccgcccgcttcggccccccaaaatgctgggattacaggcgtgagccattgtgactggcccacaattttttaaaaagaaagaaataggactgaaaactaaccccccaataccacaacaccaagaggaagccctaacataaactatggactcaggtgataatgatgtgcaaggtaggttcatcagtcataacaaatacaccactgtggtggacgatgttgataacggggaggtcgtgcatgtgtgagggcaggaggcacatgggaaatttctgtacctctgctcagttttgctgtgaatctaaaactactctatataaagtctataaaaaactaaaacaaaaactaaacccctgaagattggaattttgcaagacggagtagctgttcctcagagaacagctatgtccccctcttggccagcactggcttgagacttacagggtgcaacatgaaatttctgtttcgtagcaagtgcatgtctcacagttgtcagtctgccactccgagtttattgggtgtttgtttcctttgagatccatgcattctaaaataatacacacaacatcatcagtgcaagtcatgcaatgagaacaaggcctatcaggacattgagtcctgcccctactatctacacccctcccagagagagaggagctcaattattttaaaggtatctaagaatggtgctcctgtattcacccacggctaattccagtactgttcagttcatgctactgataatgttttcttacagaaagcttatatccttctaagttttatttcagaacagatatttcaagaaaaaataaggcccttttattgaattagatagccatgtgagcctgtgggtacttgggaccacattttttcctactggattatctctggatgagaatctcacttctacacttccctttgctttgcatcggtggttttcaaccctgggtgcacatgtgaaacatcttgggtacttgaaagccacccatgtctcagccttaccctagaacaatgaaatcagtatctctgggtacagggcccaggctactgtgttcttagaaggctgcccaagtgaggccaggcacagtggctcacacctgtaatcccaacactttgggaggctgtggcgggcagatgacctgaggtcaggagttcgagaccagcctggccaacgtggcaaagccccatctttaccaaaaatacaaaaattagccaggcatgatggcacacacctgtagtcccagctacttgggaggctgaggaggtagaatcactggaacccgggaggaggagtttgcagtgagctgaaatggtgccactgcactccagcctgggagacagagcatgactatctcaaaaaacaaagaaacaaacaaacagaaacacaaaggctttcatctgcagacaggtccatctaccaggctgcaataacataataaacattgaaaagccaagacttactcctggttgaatctcctggaactccctcattaggtatgaaatagcatgatgcattgcataaagtcacgaaggtggcaaagatcacaacgctgcccaggagaacattctgtaatgtgaagaaaggtcagggtggagaatgaattaattcaaaggataatccttgactgagtgctgtgtaccaggatctcggctgggctctgctgagaggttatgatgtggagctcataactgaacaaaccagttaatctaggctgaaattgtagaacaaggaaaagtatctagttgactttgtcatctttttgagatgagctaaataaacaaacaaaccttaacatggtaatttctgggaacttttgaaagggaagatggtgttaaaaaaaatgttgaggaggccatttgtttggactgagctgcactgggcccaacagaccaaaccaaaatgcagtcgcccatgctgaagttctctgccactaagccaaaactaagttgcttaaccaaccttctaagaaattagaagagagtgagaggtaatagccaaatgcccaacaggccagttttagctgacatgatgagaaagtcccctctgctttaacttataagcaaagtcacttcgaaaagaccaatttgctttttgttctctgtttctgctttttctggctcttttctgtctataaagccaaacttctctgccaggcacggtggctcacgcctgtgatcccagcactctgggaggccaaggcgggtggatcatgaggtcaggagatcgagaccatcctggctaacacggtgaaaccccgtctctactaaaaatacaaaaaattagccgggtgtggtggtgggtgcctgtagtcccagctactcgggagtctgaggcaggagaatggcacgaacccaggaggcggaccttgcagtgagccgagatctggccactgcactccagcctgggtgacagagcgagactccgtctcaaaaaaaaaaaaagccaaacttctctggtcagctctgcagaacactcgatctattttatggaatgaagtgttccctgattatagactcacaagtaaaagttaattgagggccaggcgtggtggctcatgcctgtaatcctagcactttgggaggcggaggtgggtggattgcctgaactcaggcaattgaaaccaccctgggcaacatgatgaaacccattctctactaaaataccaaaaaattagctgggcgtggcggcacacgccagtaatcccaggtactcgggaggctgaggtgggagaatcacttgagcctgggaggcggaggttgcagtgagccaagattgtgccaccgcactctagcctgggcaagagagtgagactccgtctcaaaaaaaaaaaaaaaaaaaaaatcaattaagatctttaaactaaatttgttgtaattttgtcttttgacaatgggaatgaaggcatttattctgtaagattgctaaaattaattatgaagtgacgctacttgaagtagtatgttactcatatatgaatagaaagatagggagggcacagtgtctcaattctgtaatctcagcaatttgggaagctgaggcaggcacatcgcttgagctcaggagttcaagaccagcctaggcaacatggtgaaaacccatctctacaaaaagtacaaaagttaggtgggtatggtggtgtgtccctgtagtctctgctacttgggaggctgaggtggagaatcacttgagcccaggaaggttgaggctgcagtgagccatgatagcaccactgcactccagcctgggcaacagagtgaaaccctgtctttaaaaaaaaaaaggcaaacacagagatcaaaatagaaaacccagaaaattccaaaggtagttggatagggtaaaaaagaaaaaaacaaaacccagaaagtataaatatatgtagaagtttattataaaataaacatgttctttgagatctgtggagagcgattattcaataaatgctattagaacaagtggtgaactatttggggaagaaaaaaagttaatttcctatttgagtccccattagtgaaataaattctaaaaggattcaaggtttaagtttttacaataagatcataaaataatcacagcatatatacataaatacttattctaagcataaaagaaaaatagatttgagaatataaacgtcataagaacataactgaaattagaaagatatcaaattttcagaacaatatcattacataccaatgggagaagaaaaacatcttaaacagaaaaagggcaagagaaataaagtggcatttcacacacagacacacacacacagaaatgatgaataaacacatgaaaaagttcaacttactgataacaaaagaaatgtaaatatgtagcatgaaatagaagcataatgggatggtgagaataatcatttaatttataatacccagagggaaatagaggtatctatgggaggtgatatgcacaagaatgacttactttgaacaatcgttaaggagaacaatttgctactatatatccacattcttaaaaccgtttacaacatttgatttcaccttttcatctccaagatgtgatcctaaaaatggcacagagacagagacagagacagactcctccacaacgatgctcactgcagtggaaaaatggaaagaaacaaaatgcccaagaacagagccagtggagaacagtgactcagaaaaacacctggtgagccagcaaggtagaaggtgaagtcacatgagcaattgatcaccacatgtcacgacatgaaatgaagcaggctatgaaatacatgatgcggagagagatgctcagaacacactgagggttttgtaccaaataacaagtcatctttggatggggagattatgacaattttcttctttggacttttcctcttattttctaaattttctacaataaaatatatgagttttatagccacaaagaaagaaagattctttaaaaatgacacaacgccttgatacattcagctcaagccaggacttggagtggcacttgttctgtttcattcttctgagtagccttcagctcgaaatcattgtggaaacctaaacttttccccccaaaaagccagccttgttgacagcatttttactcccttcatgcagaaagagggctcctggggtactggggatcactggaatcttggggatctggtttcactgagtcacaaagcaggagagtcaacagggacaagcagggctgcatcccaggacaagtgacatgtgcaaaatccccaagtctcctgcagctctagagcagaggcaggagattgaaccactgggggagagcccaaacatggtgacagcagagagcaaattatttaattttgagattatccacttgacagtatctcagaaatagcctaatgctgtctctctctctctctccctccctccctccctccctttctgtctctctctctctctctctctgaaactcagttttccttccctgtaaaaagaggggttctcccatttgggtgaccattagagttatccagggagctcataggaaacacaatctaggagccagtgcctcagtgtctttacctagtctcccaggtgagaacccaggaacaatgaccaacccagtgctgctttaatgtgcatataaattcctgggagttttttttgttttgttttgtttgtctgtttgtttgtttctttttgggacggaatctcgttctatcgcctagagtagagttcagtgatgcaatcttggctcactgcaacctccgcctcccgggttcaagtgattctcctgcctcagcctcctgagtagctgggattacaggagtgcaccaccacacctggctaatttttgtatttgtagtagagacgtagtttcatcatgttggccaggctggtctcaaatacctgacctcaaatgatccgcccgcctcggcctcccaaagtgctgggattatgggcgtgagccaccgcgcccaaccagacagcctgttaattaacctgcagattctgcttcagtaggtctttagacttacacggaagctcccaggtgatgcccacgctgctggtctgaggtccacccctgagtagagaggctctaaagcttttttcattcctgtcttctattctgccaccagccttccttccctttgtgtcttcctttcctgcatttagtaaccctgagctcctgatgtgtgcaggagattacaccacgacgccagaatgggctaggattttctagatggtacagcgagccttgaggtgaagcaggctggcagtatccttagaagttgtgtctcttggctgggcgcggtggctcacgcctgtaatcccagcactttgggaggccgaggcgggtggatcatgaggtcaggagatcgagaccatcctggctaacatggtgaaaccccgtctctactaaaaatacaaaaaattagctgggcgtggtggcgggcgcctgtagtcccagctactcaggaggctgaggcaggagaatggcgtgaacccgggaggtggagcttgcagtgagccgagattgcgccactgcactccagcctgggcgacagagagactccgtctcagaaaaaaaaaaaaaaaaaaaaaaaaaaaagttgtatctctttaggtaaattactgctcctggaagggttatcccatgatcaagagttgggggtctggccaggcataatttctcacacctgtaatcccagcactttgggaagccaaggtgggaggatcacttgggcccaagagttagagaccagcctgggcaacatagtaaaaactcatctctactacaaataacaataataaaataaataaataaataaataaagagttgggggtcctcaggatcttaggcaggcagaggtattgccagctctgcagcttgctaagttcactgtgaggctgcatagagtataggctggaggagcctgtctagtggagatggggaggacctcccaccccactgttgttttaccatccccagttctgcctccggacagtttctgagagcaagtttgtcttgccgagtggcccagaggaggcccggggtcccctcctgaggaccctctcccacctcccttggctcctcccacttggctggctgtttcttgcctcatctcccttgcagcccccagagcagcccagttcagtccccagcgttagtaaagaatgttttaagaaagatcaagtttaagacacaagaaaagcctgcatgggtttttataaattttgaagcaaaattctttcttggccaggcacagtggttcacaccagtaatcccaccactttgggaggctgaggtgggcagattgcatgagcccaggagttcaagaccagcctgggcagcatagcaaaatcctatctctaaaacaaatacaaaaattagtcgggcatggtggcactcgcctgtggtcccagctacttgggaagctgagatgggaggatcacctgagcccaggaaggtccaggctgcagtgaatcatgactgcaccactgcactccagcctgggcaacagagtgagatcctgtctcaaaaacaaacaaataaaccaacaaacacagccccaaacccaagatttttccttaacacatgatacagtgaaaagggcctagggaaatggttcccaaacttgagtattaaaatgacctgggcagctttttaaattgatgctctgttctcactgcagcccaattaaatcagagtgtcggggtggaagatgggtgtcagtgttcttcaaaattcccccggagtttccactgcacagccaagtttgggagcccctggcagggagagaggaatgcctgggttcctatctcagctcattcacaggcttgcggtgagaccccagtggcagcttcttcccctccccaggctcagttccctcatctcaaaaacgatgcagtcatgtagctctgaaattatttttaatttcaaattactcacataggacccaagaattagagacacttatgaaatctaattgaaccccagaattactggaaggaaaaacaacttagatcttagagaacagaactagtgctaaactcattacaattttcgcaagctctcagactctcatactctgcacattgaattgcaggtaataacataaggttcctaactaggtcacatttaccattcaatgctatgtggtagaagcacacgcatattaaaataggaatacatattctcttttgcttttctagcctttatatataaaaccagttacctaccattgtgataagcaggactccttatagacaggtacatccaggcaaagctgcatcaaacttttatactggagagggcaaccacgcattcgacatcattgggaatgataacaaggtgagaacacattgagtgtttacagagggttgactatcaaaaggaaagtatggtttcagatgatttatgagttattaatcaatcacatctccacccaagttatgtgtttaccttgtccccaccccgatctgcatttgctgctgacctaaaagagcagaggaggagagaacttctctttgctgagccttcgccctatgctggtggattgctagcacgtgatgtgtccttattcgattttatacacaaagaaacagcctcagcgatgccagtcacaacagccaggtggtaaaggtgggtttcctgaaggcataagaatagcccacaggtcagcccaccaggcccaccgggcctgggtctgtggcccagatgaccctgcccttagggtctgacatgagaacagaaatgggtcaagccatcctgcagggcacaggagagcaagaactacatacagctcggggttccctgacaagctgaattgctggacctttctagagcccttcttgaatctgtagtctactccctgctgcatcaagcaagccttgtttctctttgttccacagtggtctcctcccaaacaaccgcacctgccctcatcatggctaggatggcctttggggaatggcaacaagcagaacctcctagggtgctggagggacttactactttatctgtttgctctgccaccacaacaaaacactgcaactgggtggctttgactaaagaaatttatttgctcacagttctggagactagaaatgtgaggtcaagccattggggaaggcttgttttcctctgagccctctctctttggtttgcagatggccatgctcttgctgcctcttacatggcatgcatccctggcatctctctctgtgtccaaatttcctcttctgataaggacactagctgtattggattagggtccactctaactatctcattttacctaagccacctctttaaaggccgtgtctccattacagtcacattctgggatattgggggttagggcttaaaccaatgaatttttaggggatgcaattgagctcataagacaattttatcccaaaaagtcccctcactcctcgccacagcttgctacagttcctaaaatttaacctgagatccctctgcccacagatgagagttcatgctcctaaccaggggatccttgaagctcatctgagctggccacagtggccctttccagcaccatcctctaccctccctaaaatcagcctcaccacccctaacacagcttgaggtcactctcctgctccttagaagagctgtagtgctgtctggctgcctcctcactcaccaacggtgccacacacttctgcctttacctgcataagttaagtgttcatcgactctaagttccaggaaatcggggcacattcatcatgcactgtagccccttggattcttttaaaactccaagtttgaagaacttcccacagtgtgaagtgggaagttctgccttacattgagaggaactaggatttgctgccctcacctgcttacctccagggtacaggcacaaacctcggctccacaaacactctcctctcaaaaagtcctcttcagagagggtccaaggacactggcatcatgcagaatctccctaggaatgatggtgtggagggttctggctcttagggcaagggtttcttggagtccagtgtgcagtgtcagtgcatgggggctgtgactgtgactggtagcaaggtggtcttgctggagccaagcagcagtgtggtttcagtgctctacctgaacttgtgccaagaaagcctgtgtctctggccgttagtagaggcttgaattctctaacaaatctctttccacctcaaatgacctagagtaaattctattgtttgcagttaagaactttgtgacccagacagtgactgtgacctattcctgtctgtgtgccttccagtgaccagcagggtgcctggtgcaaagcagctgcccagaacaggatttcacctgggttatctcaggtattcccaagtagaaagtatcctggcaagaccaacccatatcactgctgaaatgagctcatctactgctccccaggggttgagaaagggatgctcagagcaggtgtggccagctgcccacagggaggaaggcaaatgaaatgagcagtgctctccgggtgtgtgacagccagctctgtccaaacctgcctgatcagttttcagtgttgatcctacctcaactgtcatccttatagatggaaccaacttaatttcttttaattatttatttttctttttatttattttaattttttttaaagacatagtcttgctctgctgctcaggctggagtgcagtggcaccattatagctcactgttacctccaattcctagattcaattgatcctcccacctaagcctcccaagtaggtgggactacaggagcgtgccaccacacccagctaatttttagattttttttgtagagacagggtcttgctatgctgcccagcctggtcttgaactcctgggctcaagtgatgctcctgcctcctcctcccaaagtgttgggattacaggcataagccaccatgcccaggctgattatcattttaatggctacattttattccatggaatgatatggaccatcatttatttggtcaggtacctcttgttgaacactcaggttgtttctgacttttttttctataattagaaacactgggctgggcatggtggctcacacctgtaatcccagcactttgggaggccgagacgggcggatcacgaggtcaggagatcgagaccatcctggctaacatggtgaaaccccgtctctactaaaaatacaaaaaaattagccgggcgtcatggcgggcgcctgtagtcccagctactcggaaggctgacgcaggagaatggcgtgaacccggaaggcggagcttgcagtgagcggagatcgcgccactgcactccagcctgggcgagagagcgagactctgtctcaaaaaaaaaaaaaagaaacgctgttgaaatccaggtccttcagataaaaatttcccacacttatactaatttctttaagataaattcatagaggccgggtgaggtggctcgcacctgtaatcccagcactttggaaggacgaggtgggtggatcacgtgaggtcacgagttcgagaccagcctgaccaacatggagaaaccccgtctctactaaaaatacaaaattagccgggcgtggtggagcatgcctgtaatcccagctactccggagggtgaggcaggagaatcacttgaaaccgggagacgaaggttgctgtgagctgagatcacgccattgcactccaacctgggcaacaagagcgaaactccatctcaaaaaaaaaaaaaatcacaaaggtagaatttctgggtaaaagaaaattataattctcttagattatcacctgttactatgatatgtgggaaatgctttgtcttaatttatttttctttgctaattggcaagattagacctttttttttttttttttttttttttttgagacggagtctcgctctgtctcccaggctgcagtgcaatggtgcaatttcagctcagtacaagctccaccttccaggttcacgcccttctcctgcctcagcctcccgagtagctggaactacaggcaccctccatcacacccggctaattttttgtatttttaatagagacggggtttcaccatgttagtcaggatggtcttgatctcctgacctcgtgatctgcccccctcgggcttccaaagtgctgggattacaggcgtgagccaccgtgcacagcctagacctttttttgtatatgtattgaccaattgtttttcttcttttgtgagtaccctgttcactcatgtcctttagctatttttctgtttgggttatttatatttttcttctggattagttaaggcttctaatattaaagatattaatcttcaagtgtcacacatgttaaaatatttccctgatttttttcatggtcagcacacccattctggagttagaactaaggaatgagaaattttcctcatttatatttttacctctcaaatgatatgttttgacatgtagagattttaaactttaactgactacagccacactatccccacctgtgggtcagcactacaagccccaaagaacacctcaagatccatggagtcacacagcccttgggctgatgctgtggcaggagctgccattcatttccagggttttttgttttgttttgttttttgtttttttctgagacagagtcttgctctgtcgcccaggatggagtgcagtgctgcaatctcagctcactgcaacctccacctcccgggcacaagcgattcttgtgcttcagccttcccagtagctggggctataggcatgcaccaccatgcctggctaatttttgtatttttagtagagacagggtttcaccattgcccaggttggtcttgaacgcctgacctcaagtgatctgcctgccttggtctcccaaagtgctgggattacaagcatgggccaccgtgccctgccatcattcccagtgttaaaagaacgttagacaaattaaatttaacagagtttgattgagcaaagaaccatttgtgtgaatcaggcagcccccaggcaaaaataagttcagagagactcctgcactgcctcatagtcaaagattatggacagaaaaaggaaagtgatgtacagaaactggaagtgaggtacagaaacagacggattggtgatggcatggtgcttggcttatttgagtccagtttcaacagctggccgcctgtgagtggttgaagtctggctgctgtgattaactgagactcggctacttggtacgagagtaggtgacagcctgtttacacgtgcagttagcttacagttcactatgtacagagaaaccattagggtgaatttaaaatatgtaaggagaaggctttaggctaaacttgatttaacaccaggatcggtggggtctccccttaccagtcttcaagctgatgacttatctctcatccacttcctctcccatcgtgctctagagcctttccaaaccatgatctcttctcttaaacctctcacccaactgcctacttcatacgcagaacacaatcttatttcgtatctcacagagaccagaggtgaaaactgcacccccacccctaccctgcacattgccctccaccggctcctgttctttccactttccctctgggtcccttcctcaccccaaatacgcaatacggatcccattcccctccccatcggcccccattccttcccagggacccctccataaaagccagcctccccaagacaagcaggcatgaagggacagatggaaaagtgttcagtgttcgaggactgcccaattcccagcctggcataagccagcctccctgagggtccacttataccctggcaattttactttgtagagcccaatctttgtgataccctgtggcaggacccgttttggtattcgtcaggattctgttctgttggaaaccgaactaaaatgagcatgggcaaaaagagacgttttcagctcaagtgttaagtccagaggtggaccagcttctggactgcggggcttagtctaggacagacattatgagagttttcactctgcagaaaccagaaagacagactctgcccccacccatggattcatttctgcataatctcttgcttcaaaaatacaataagacaaattctccatgccaaaggatgcagtagagggtctttttacagcaacagaaatgctgtaggatgaaccccctatatggagtgagggtgacggtgggatagagatcctggatatccaggcaaagaactgggttggaaaaggaagccactaatagaatagcgagaaagaacactgtaaatgctgtgagccacagctactctggatacagtgtcacaagagtgcactggaatgcattgaggctgacaagccagggaccaggaagactgctggtgaataatctttctggttttctgtaaggaacttggaaagcttatccagagaggtgtgaaaacagcagaaaaaacactgggacaaagaaacggggcaaaagcaaacatgttccagattagccgctgagaaacctaagaaacaaaaccaggttttgtcccagagaaaagggagacttgggcatccccttggaggcccggaagcttcttgtttgtcaagtggattaaagggtatttggtcaaggattgtgtgtttacagcttaaagcaactatgcacagaacccaggaactacctgcctctcacgctttcaatggattgtcccagagactgctcccttttgtgaagctgatgtttgtcttgtgttgaatgtaacacagtcacaaggaggctaccgtgacagggagcttgtattttctttctggatgtggtattcaaagtgttgaacaaccagtatggctcaaccactgacccttcagttcagctgccagaagtcagtctgggggaccctactgtgcgaggctctattccttctgtttctggatgatgttgaggtctggaggggccaggatccaacatgctgagtgggggcactcaggagtgttgggcagcagccacgtacccaccagtatatctgaactgatctataccagcatcacaggcccttattctcctcatagagcacatgctaagtaaatgcagccactgaatcagacagaagaacgccagccagcaaccttctgtccttaccatgagcagtgccttccagaaccaccccagggtggagggctgagaggtgggaagggagctggagcctctcatgtccacacatgcattacctcagggatttcccagcaacacaacaaaacaggccttctctcgcccactttaccagtggggcattgaggtttcaggatggaaacaactattcagggttttcagatggtgagaaagagtgagaatttaaacccaggcctgagatgaagcagggacctgtgccaccaccccgaacgccaccatcaagcatggaaataaaggaaaatcttgagtgccttcaagggaaatttcaggcatccagctagccctgagaagtaagtaagtaacttgttaggcaggaaggtaatagtagcccaaaacaatagccaaggaagttagagtccggagatgtttagttttaggaactaaagataacatcttaacacacgtccttgagtttgtcattcagaaacctggacctccatgtggacccccaccaaacggatctgctggcctgcagacctcagatagagggagctgaggactgcactctgaccaccttctttcattctaaatttcttccggaggctcctgaaagccacacccatgagctaaagctaatattcttttctgttaaccctgaattttcaggcaaggcatcgcctcctgaaccaactacaaatcaaagaatctctgaatccacctatgacctgtaagcccctgcttcaagatatctcaccttttttaggccaaaccaatgtgtacccttcatgtactgatttacaattttatctaacttctgctgctttcctgaaaatttaccccgcctttaaaaacccttacctgtgtgaaaggaaactatgttggccctcccaaatcactaagttaaagagaaaattcaagctgggaactgcttagggcaaatctgcctcgcattctattcaaagtcatccctctgctcactgagataaatgcatatctgattgcctcctttggaaaggctaatcagaaactcaaaagaatgcaaccgtttgtctctcacctatctgtgatctggaagcccactgcctgcttcaagttgtcctgcctttccagaccaaaccaatgttcattttacatatgttgattgatggctcatgtctccctaaaaggtataaaaccaagctgtgctctgaccaccttgggcacatgtcgtcagaacctcctgagggtgtgtcacgggggcatgtcctcaaccttggcaaaataaactttctaaattgacacttgtctcagattttcagggttcacatctgccagccatcagagagttcaggttttgagcatgcgctgcctggtcccccttgctcagggctctgtgaataaacaccttcctttctagtgctgcaacttcagtgtggatatctggacttaactgcactgggcaagccgaccccaatttgtcaagcacgtccatgtgaagaaaccaccaaacaggctttgtatgaggaataaaacttttaattcacttgggtgcaggtgggctgagtccgaaaagagtcagcaaagggagatggggaagggattgctttataggagttgggtaggtaatggaaagttacagtaaaacgtggttatctattgttagcagaggagggggtcacaaggtacatagtggggagatcataagactcattgtccagaagaagaatgtcacaaagttggttgatcagctaaggtagggcagggacaagtcacaatggtaaaatgttgtaattttggttactcagttaaggcaggaactggctgttttactttgtgggttttttggctgctccagacttcttggttcctgcaggccatctggacatatatgtgcaggtcacaggggttataatagctgagcttcagctcagaggcctgacacaattcagttatataatagtcctaatgacccaaaagccagtactctcttttttttttttttttttttttttttgagatggagtctcgcgctgtcgcccgggctggagtgcagtggcgctatctcagctcactgcaacctccgcctcctgggttcaagtgattctcctgcctcagcctcctgcgtagctgggattacaggtacatgccaccatgcccagctaatttctgtattttttttttagtagagatggggtttcaccatgttggccaggctggtcacaaactcctgacctcaaatgatctgccacctcggcctcccaaagtgctgggattacaggcgtgagccaccacgcccagccaagcccctgctctctttacagccagggctgcctccctcacacacctgcccaaagggctggcagcatctgcactcaagtgaagagatctaagcacctttgagccccagagcagcaggtgggggctaccaccagcatggggcctttcatgcgggagatcccacagcccttctccttccccttcctcctcccttgacaatgtgttcaggtctcccacagcctttggcaccaagaaattccaatagatgttaaccagcctggtaatttagtacagcgttagcaccaagtcgccaaggttgaggatatgctaccgtgacacaccctcaggaggttctgacaacatgtgcccaaggtagtcagagcacagcttggttttataccttttagggagacatgagccatcactcaacatatgtaaaatgaacactggttcagtctggaaaggcaggacaatgtgaagcaggcagggggcttccagatcacatatgtgagagacaaacggtgctatttccaaatatttccaaactactctcttttccagctactcaccaggaggaaagtaaatgcctgtcagacatggttactaatcaccgccggtgggtgggatgaaaaggagcttactgatgactggccaggtgctcaccctcaccaggtctcagattcctctttggctaatgagggagtcaattctctattgggggtagcaagttcaaatgctcggggcaagtcaggtaacacaacccagtgaagcaggtatgatgtttgtatatgaagtacttatgagcctcactttcctaagtaaataggctttctcctattttccttgaaatgcataccactgccagtctgtttctctttatcccaattttgacagagacatatgtataaaaaatacatttttaggccaggagtagtggcttatacctataatcccagaactttgggaggcccaggtaggaggattgcttgaggccagaaattcaagagcagcctggacaacacagtgagaactccatctctacaaaataaaaaaattagccaaattggaggcaaaggcgggtggatggcctgaggtcaggagttcaaaaccagcctgatcaacatggtgaaaccccatcactactaaaaatacaaaattaggcagggcatggtggctcataactgtaatcccagcacgttgggaggccgagacgggtggatcatgaggtcaggagatcgagatcatcctggctaacgtggtgaaaccccgtctctactaaaaatacaaaaaaagttaggtgtggtcgcgggtgcctgtagtcccagctattcaggaggctgaagtaggagaatggcgtgaacccgggaggcggagcttgcagtgagccaagatcacgttactgcactcccgcctgggtgacagagcaagactttgtctcaaaaaaaaaaaaaaaaaaaaaattagccaggtctggtggtgcatacctgtaatcccagctacttgggaggcaggagcatcacctgaacctgggaggtggaggttgcaatgggccgagatcatgccattgcactcccgcctgggcaacaagagcaaaactccacctcaaaaagacaggacaggacacgacacgacacaataagacgagaagaaatattatgtctttcacaaaaatctttcagaaaataaaaaaggatagatcatgttccaactcctttaatgaggccagtgttaccccaatatcaaagctaaacaaacacaccaaaataaacaaaaattatagaacaatatctctcatgaatagagatgcaaacatactcaataagatattagcaaacaaaaatccatcaacatgtacaaaagattatacaccatgaccaagtatgatttatcccagaaatgcaaggttggtgtgacatccaaaaatcaagtaatgctttctctagtatatggtgaggtttttaaaattaattaattaaaaacaacaaaacctcaaaatcagtgaatgtaatgtactagattaatagactaaaatacatcacattgtcatttcaatagatacagaagcatttgacaatagccaacatacattcgtggtaaaaactctcaacaaactaggaataaaagtaaacttcttcaatctgatagagggcaactacagacaacctacagttaatgtacttaatgatgaaagacagaatgtttcccactcaagattaggaaacaggcaagaatgtctactcttaccacttccattcaacattgtactactggtcatagccagggcaacttaaaaagggggtagagggagaaagagagagaaaggtaaattaaagacatctagaatgaaaagaaaaaagtaaaactgtgtttatttgtagaccacatgatctgtatgcagaaaatcccaagtaatctatcaaaaaattttactggaattaataaatgagtttagcaaacaaggttcaagtatataaggccgatatgtaaaaatcaattgtatttctacatactagtaataaacaatgtgaaaatgaaattaaggaaacaaaatttcattcacagtaacacctaaaagaataaaatacttagaaatgagtttaatgaaagaaatgcaagacttacaataaaaactataaaactttgctcagagaaattgaagaagttctaaattagaggcagcaaactacagtccatggccctgaactggccccctgtttgttttgtaaataaagttttattgaaacacaatttttttttttttgagatggagttttgctcttgttgcccaagctggagtgcaatggtgtgatctcagctcactgcaacatccacctcccaggttcaagcgattctcctgtctcagcctcccgaatagttggaattacaggcgtgcgccactatgcctggctattttttttgtatttttagtagtaacggggtttctccatgttagccagctggtcttgaactcctgaccttaggtagtccacccgcctcagcctcccaaagtgctgggattacaagcatgagccaccgcgccgggccaacactgcttttacactacaaagcaaagttgaatatagcccacaataccacaaatatttttactatctggttctttgcagaaaatttgctaatccctgatttaaataatcccatgttcatagattaaaagactctattgttaagatgacagtcctcccccaatttattgataaatttctgccaatttaaaaaaaattaatgtcgttcctaagaaagtgagtccattccaaatactgcgaaatcaagggaaaaaattaaaaagtcagtcctggctgggtgcagtggctcacgcctgtaatcccagcacttgggaggccgaggcaggtggatcacctgagttcaggtgttcaagaccagcctggccaacatggtgaaacagtgactctactaaaaatacaaaaattatctgggcatggtggcatctgcctgtaatcccagctactagggaggctgaggcaggagaatcgcttgaacttgggaggcggaggttgcagtgagccaagattgtgccaccacactccagcctggatgacagagtgagacttggtctcaaaaacaaaaacaaaaaaaatagtccagcaggtttttgtggaaattgataagcttttgctaaaatttatatagaaaagcgaatgacctaggatagcctaaatattctgaaaaagaacaaagctaaaaaaaaaacttacactatccaatttcaacacttgctattgttaaagaaaaattttgcaccagacacttgtcaaaaatgtcaagacagattttattcagactactgtggcagggaagagacacttcagcattaactgagctcaattctaccaaaccagaaggcaagaggctttttaaatgctaggatgtgttaagggaaaagtactgaaggatgctagttgaggggagggaggtttggtcaatgagtacactgaccagattgtgaacttgtgcttatcaaattgtgaactggtgcttatcaatatcagggtcctaccctaccaccaagattgggagacagaggctctgtcctacctaatgattacatttcgaaaggatggctcccaggtccttgggaaagacattcctgtgttatagaagatacatttccaagagacagagaaaggatttctaactgccagttttctaaagtaaatcctttaggaaaagggggttcgggggcctatattcaggttttgcctggaacaaatcataaattctttgggcagcattgaattttattaggcagaaacttaagggggctgaagtcatcatcctaaggatgtggccatgaactgttagaaacaacagtgtttgttgaagtctcttagtgccagaggtggatgaaatcatttgtgctgagagtctgcagtttgcttttttatttatattttattttttattatttatctttttttttttttttttgagacagggttttactcttgttgcccaggctggacccaggctggagtgcagtggcgccatctcggctcactgcaagctctgcctcccgggttcacgccattctcctgcctcagcctcctgagtagctgggactacaggcgcctgccaccacgcccggctaattttttttgtatttttagtagagacggggtttcaccacgttaatcaggatggtctcgatctcctgacctcatgatccacccacctcggcctcccaaagtgctgggattacaggcatgagccactgcgcccggcccattttttttttttttttaagtataacatatctctattaagttgaatagaaacactaacaaagactagtatgcatagtgattatagatgtagacagataaaaaagaaaaagggatacaagggcgcattgaatttatgacaatattgtctcagtaccacagcaggagccaactcaccccagggctggcttcccaagaccagctccgggaagcctgatctgctcagccttgattactggttgaccgggactggggatccagctggcaggacaggctgtagctctcaccctcactgggctgctctctgacccagaaccaggcccccagttcctccccaggatgaaggctgtaatgatttgcagccacctgagcagctgggtctccttttctgggagtccatcatattctcatccacattaccattcatcgcagtgtccagcatcaccaggtcggtgaggaatgtgccaaggacagggacaattcccttcttctgctgctgccaccttgcatgggctctgtgggggcttgcctccagggtggccgacttggaggtccccacactgatgagcagatccctgttcggtggcttgtctttgctgcacagctttggacatttttctgttgctttatcttggccactcagagcagagcagccacaaccccccaccccacagtgcctgggccaaagtggcccaggttagggtccttgaaagacgagacaccagagagttccctctccttctctttccctctgtcccccacaccctctttccaggagcacaaagaggtcatgtgagcacgcagtgggagggtggccacatgtgagccaagagaggggcctcagaatgaagcctgcctttcctgcaccttgatcgtggacttccagcccccagaactgtgagaaatgaatttctgttacttaagcctcccagtctgtggtattttgttatggcagcctgagccaactaatacaacaggaaagaaacattccaatctccgtaaattcatttttaagttcattaatgtttaaagtgattaacagtgggtacactttgtaaaccatcaagatctcatacagtcatattttatgattccccataattagttggtcatggtttgtaactcctgacaaacattgtttttagttttagag

SEQ ID NO: 8 - DNA, 30000 bp, Homo sapiens
aaaaaaaaaaaaaaattgagtaaataaataaatggtagaatttagattttttttttttttttttttttttttgagatagggtttgattttgtcgtttaggcgtagtggcgtaatttcggtttgttgtaatttttgttttttgtgtttaagcgatttttttgttttaatttgttgagtagttgggattatagatatgcgttatgatgttcggtttttaaatttttttgtagagatagggttttgttgtgttatttagattggttttaaagttttgggtataagcgatttttttatttgatttaggattataagtatgagttattacgtttagtttatagtttagattataatttatattatttatttgtgattagatgttaaatagtatttttttttgttttagttttgatagaggttttttatatttagttgttaattgtttttttatatttcgtagtaaaggaggtgattatagtagggttttatggattttatttaagagtgattttgttataaggggtaaggtaattttattagaaaataataggagatatagattacgatgtagaagttaagataatagttataggttgtgagtggaaggggagttgtggagagagaagtttataaaagttttatgaggaagttagtttagaatatgattttaggtagaaggagtttttaatttttttgttttttatttttttttaatttagatgtagtttttggaatgagattttgtgtaatttaaaatgtaatttagattatttttttaaattatttgaaggtgaataagaaagttaagggatttgtttagattgtttaattggtttatatagtatgtttaaatagataggggataggggaaaataaagttttttattagttgtttattgattgtttaatagatgattgtttgtttatttttgatttgttacgtatatatagttgagtttaagttttaggagaatagggtttttttttgaaatttttttggtttattgttgtatttttaggtttagagtattgtttggtttaggtgtttagtatagatttgttgagtgaatagatgttttttaatatagaaatagttaaaagagaattaatgttgtcgttgttttaagttattaagttttcggtggtttgtgtgtagtaagaggtagttgatgttggggttttgtatttaggttattttttatttgtaaggtagagtttttggtttttaatttttttcgggttgtatgtggttttgggggtattttttttaggtttgttttaagttgttttataggtttgttttaagttgttttttgttttaagttgttttgattttaagtaaatatagaggaatgtgggggatatattatagaagatattttttgatgaagaggaaggagggataaataggaatatcgtaaagaaaagaagggatttaattttagtttgaagatatatattttttttatcgtttattagatagtaggatacgggtagtagagtgtagatattagaaatagtaagagtggtagtggttttttgagtaagggatgtataatgatttattatagagtttaggaaaaatagaatttttatttatatgtattttgttttgttttgttttgtttgggtttgggtttgtttgtttgtttgtttgttttttgagacggagttttattttgttatttaggttggagtgtattgatgtgattttggtttattgtaattttcgtttttcgggtttaagtgatttttttgttttagtttttcgagtagttgggattataggtgtttgttattacgttaattttgtattttagtggagacggggttttattatgttggttaggttgttttcgattttttgattttaaatgattcgtttgttttggttttttaaagtgttgggattataggtatgagttattgtgttcggttatttatatgtagttttgtttttttttttaaatgttttgtattttttataatgtgtattaaattaatgtatatataaattataaattaaaaggatatatatatttaagtgtttgtttaaaaatttttttattgataggtatggttatataattattgattattagaggttagaggagaatgaggtttggtttgggagttttgtgtttattagaagtatattagattatttatttattgatagaataggttttttttgggttttttttttttattacgatatatttgtagtttttttttttgaagattttttggtagttgtttttgttataatttataggtgtagaaatattgttattgagataaaattggggtttgttagaagagaaaggatttcgagtattttttttttagtgttagttttgtttaggaagttatttatattttaatttaagggtattttttggttggtagttttagagtgtgatttttaggaatttggattagtttgtattatagagttatatagtttatttttatattttattttgtaggtatagaaggttattagtaggtgttgataggtttaggggtaagtttggttattttagaaagtggtagtttgttttatgttggttttttttattttttttgtttaggaagtggttagtatattgttgttagaatgtggaaaggaaataaattttagttttgatttggaggtttgttggtaaaggagtttttttaattaattagagtaatgagttattttaggttagtgttatggagtttagggagaagttgatgaaagttagagattatttttaattgagttgtagtatatataaatattttgaagagttttgatgagatgagatgatattaatgttagaatatttatgtattattattattattattttaattttttgagatgaattttattttgttatttaggatggagtgtagtggtacgattttagtttattttagttttcgttttttaggtttaagcgatttttttgttttagttttttgagtagttgggattataggagtatattattatatttagttaatttttatatttttagtagagatggcgggtcggcgcgggggaggttttattatgttggttaggtaggtttcgaatttttgattttaaatgatttatttattttgtttttttaaagtgttgggattataggcgtgagatattatgtttagtttagtgttagaatattaagtggaaaaattagtttataaaattgtgatataaaatttgatatttattatgtaaaagaaaaaatatatagaaaagagatataaagaaatgatgatagggtcgggcgttgtgttttacgtttgtaattttagtattttgggaggtcgaggcgggcggattacgaggttaggagatcgagattattttggttaaatcggtgaaatttcgtttttattaaaaatataaaaaattagtcgggcgtggttgtaggcgtttgtagttttagttatttaggaggttgagataggagaatggcgtgaatttaggaggcggagtttgtagtgagtagagattatattattgtattttattttgggcgatagagtaagattttattttaaaaaaaaaaaaaagaaaataaatgatgatagaagtttaggtatggtggtatatgtttgtaattttagttatttggggttgaggtgagataatagtttgattttaggagtttgagattagtttgggtaatatagtgagattttgttttaatttttttaatataaataaataaataaataaaaatagaatgttaatagttattatttttatatagttgggattatgagtaggtattttttttttttttttttatgttttttaatgattttataatttatatgtttatttagaaaattgggaaatattatttaaaattttttatgttttttatgtttaaaggtgattgttggttggtaggaatgtaaaatggtgtagttgttgtggaaaatagtttggtagttttttaataagttaaataaaaaattattatatgatttagaaagttatttttgggtatatattttaaaagaattgaaagtagggatttaaatattatatgttaatgtttgtatattgataaatattgtatgttaatgtttagaataatattatttattttatttaataggtagaagtaatttaagtgtttaataaataaatggataagtaaaatgtggtatgattatgttatggaatattattttggtagaaataagaatgaagtattgatatatgttataatatgaatgaattttgataatatgttgagtgaaataagttagatacgaaaagataaatatagtgtgattgtatttatgtgaaatatttagagtaggtaaatttatagagatagaaagtagatttgagattattataggtttgggggatagggaatttgagttattgtttaatggatatagagttttagattggagttattaaaaagatttggaaatagtgtttatggttgtatagtattgtgaatgtatttaatgtcgttgaattgtatgttttaaaatggttatattggtatattttatgtagtatatattttattataataaaaaaaaaaggttaaaaataaagtggtgtttgttgaagagatataatttttttagtttatggtaggaagtgaaatggtgaatttgtgttgtttgtgtgtaaatataagttggtaaggtaaaaggtatattattgtatgtttattttaatgtaaattagatttagttttatagaggaaatagtagagagagagagggagggagaaattgttaatatcgggtatttagttaattagagttagtggttggatgattttggttttttgttttttatttttttttttttatttaagttagggagttagttttgggttcgggtagcgtggagtttaggatattttttatgttttatttgggagtttttgtttgggtttttagttttaggttttattgttatcgagtattttgagatttgggtagcgttttatagagggtgtagtgggtttaaaaattttttttagaaaaagtttaattaaagtttgtatttttttatggaggagaaaaaagtttatgagaaaaataaaggaggtgaggtaattttagggaagaaggtgttgatttattttttttgtatggagtagtgtgttaaatggaataggtaatatggtagtagaaattttgaaattttgaaatttgattaggtgttagttattgttttaggattataggatatagaattggtatagagaaggtattaagtgtttgttgggttaaagaatatatatatagaattttatttaattattatagttatataattataatgtgaggaaatagaggtttagagatatttagaaattgatttagggttatatagttagtatataagggaatggagatttagatttaagttattttgattttaatagtgaggtttttttttttttattgtaaggagtagtttggttcgtttttatattaggattacgagaattttagtttatagtggagaattttagaagagggtagggatgttaaatttgggtgatatggatattttagttattagggagttacgtttttatgtgagggagggattagttattagttttttatttggttgggatataattatattttattagagtgatattaatatttgatataaagataatgttaaatttttggagttataaaattaaggtttagttgtaatatttagtgatttggttaatgcgatgtttaggagtttgttttgagtagttagttattttttagtttaatatttttttgttttattaggtatttagtatgaggtttgttcgataaatgtggttggaattttttaggtattttaagtttagtaggatttaggtgttttgttttaagttaagtgtttgtttttttattcgtagtgtttttataagattttcgaggtatttgttaaaatttagattttttggttttttttttatagattttgattaattagttttgggatggagtttaggaatttgtattttattaagttttttaggtgagtttgtgattagataagtttgcgaaatgttttttgatattaatgttttgttattagatttatttttgtttttgtagaatattataagttaaggttatatgaagaggttagatggatttggggggttgggagttaggtagttgttatggatatagttgttgaggaaataggtaaattaggacggtattgcgggtattttgtgtttcgtttgtttaagttttagatttttggtgtttatagtaatttagtagaaaatatttgtatagtttttgggttgacgtgagttaaatttgagtagtgaagtagtataagttttatggtgtaaggtatattatagatttttttttattttttatatttgatttttttgtttatgtttagagttattgttatttttataaatttataaggtttagattgaggtgatgatcgggttaatttttggaatagtgttagataattagatatttttttttgtttttaggagtttatttggaataaagttatttattaagttttaaaaataatagaaaagaaatgagattgaggtggaaaataatttaagtgtttattaatagatgaatggagatgtaaaatgtggtttttttatattatagaatatgatttagtttcgaataagaaggtaatgttgatgtaagcgttaatatagatgaattatgaggatattatgttaagtgaaatgagttagttttaaaaaagtaattattgtatgattttatttatatgagggatttagagtagttaaaattatagagatagaagtgaaatggtggttgttaggggttggggagaaggggatggagagtgattgtttaatgggtatagagttttagttttgtaggatgaataagttttggaggtggatggtagtgatgttataataatgtgaatggatttaatgttattaaagtatatatttaaaattgttaatatggtatattttacgttacgtatattttatcgtaattttttttttttttagatatagtttcgttttgtcgtttaggttggagtgtagtggtgcgatttcggtttattgtaagttttgtttttcgggtttacgttatttttttgttttagttttttaagtagttgggattataggtattcgttattaagttcggttaatttttttggtatttttagtagagacggggttttacgtgttagttaggagggtttcgattttttgatttcgtgattcgttcgtttcggttttttaaaatgttgggattataggcgtgagttattgtgattggtttataattttttaaaaagaaagaaataggattgaaaattaattttttaatattataatattaagaggaagttttaatataaattatggatttaggtgataatgatgtgtaaggtaggtttattagttataataaatatattattgtggtggacgatgttgataacggggaggtcgtgtatgtgtgagggtaggaggtatatgggaaatttttgtatttttgtttagttttgttgtgaatttaaaattattttatataaagtttataaaaaattaaaataaaaattaaatttttgaagattggaattttgtaagacggagtagttgttttttagagaatagttatgttttttttttggttagtattggtttgagatttatagggtgtaatatgaaatttttgtttcgtagtaagtgtatgttttatagttgttagtttgttatttcgagtttattgggtgtttgttttttttgagatttatgtattttaaaataatatatataatattattagtgtaagttatgtaatgagaataaggtttattaggatattgagttttgtttttattatttatattttttttagagagagaggagtttaattattttaaaggtatttaagaatggtgtttttgtatttatttacggttaattttagtattgtttagtttatgttattgataatgtttttttatagaaagtttatatttttttaagttttattttagaatagatattttaagaaaaaataaggtttttttattgaattagatagttatgtgagtttgtgggtatttgggattatatttttttttattggattatttttggatgagaattttatttttatatttttttttgttttgtatcggtggtttttaattttgggtgtatatgtgaaatattttgggtatttgaaagttatttatgttttagttttattttagaataatgaaattagtatttttgggtatagggtttaggttattgtgtttttagaaggttgtttaagtgaggttaggtatagtggtttatatttgtaattttaatattttgggaggttgtggcgggtagatgatttgaggttaggagttcgagattagtttggttaacgtggtaaagttttatttttattaaaaatataaaaattagttaggtatgatggtatatatttgtagttttagttatttgggaggttgaggaggtagaattattggaattcgggaggaggagtttgtagtgagttgaaatggtgttattgtattttagtttgggagatagagtatgattattttaaaaaataaagaaataaataaatagaaatataaaggtttttatttgtagataggtttatttattaggttgtaataatataataaatattgaaaagttaagatttatttttggttgaattttttggaatttttttattaggtatgaaatagtatgatgtattgtataaagttacgaaggtggtaaagattataacgttgtttaggagaatattttgtaatgtgaagaaaggttagggtggagaatgaattaatttaaaggataatttttgattgagtgttgtgtattaggatttcggttgggttttgttgagaggttatgatgtggagtttataattgaataaattagttaatttaggttgaaattgtagaataaggaaaagtatttagttgattttgttatttttttgagatgagttaaataaataaataaattttaatatggtaatttttgggaatttttgaaagggaagatggtgttaaaaaaaatgttgaggaggttatttgtttggattgagttgtattgggtttaatagattaaattaaaatgtagtcgtttatgttgaagttttttgttattaagttaaaattaagttgtttaattaattttttaagaaattagaagagagtgagaggtaatagttaaatgtttaataggttagttttagttgatatgatgagaaagttttttttgttttaatttataagtaaagttatttcgaaaagattaatttgttttttgttttttgtttttgttttttttggtttttttttgtttataaagttaaatttttttgttaggtacggtggtttacgtttgtgattttagtattttgggaggttaaggcgggtggattatgaggttaggagatcgagattattttggttaatacggtgaaatttcgtttttattaaaaatataaaaaattagtcgggtgtggtggtgggtgtttgtagttttagttattcgggagtttgaggtaggagaatggtacgaatttaggaggcggattttgtagtgagtcgagatttggttattgtattttagtttgggtgatagagcgagatttcgttttaaaaaaaaaaaaagttaaatttttttggttagttttgtagaatattcgatttattttatggaatgaagtgttttttgattatagatttataagtaaaagttaattgagggttaggcgtggtggtttatgtttgtaattttagtattttgggaggcggaggtgggtggattgtttgaatttaggtaattgaaattattttgggtaatatgatgaaatttattttttattaaaatattaaaaaattagttgggcgtggcggtatacgttagtaattttaggtattcgggaggttgaggtgggagaattatttgagtttgggaggcggaggttgtagtgagttaagattgtgttatcgtattttagtttgggtaagagagtgagatttcgttttaaaaaaaaaaaaaaaaaaaaaattaattaagatttttaaattaaatttgttgtaattttgttttttgataatgggaatgaaggtatttattttgtaagattgttaaaattaattatgaagtgacgttatttgaagtagtatgttatttatatatgaatagaaagatagggagggtatagtgttttaattttgtaattttagtaatttgggaagttgaggtaggtatatcgtttgagtttaggagtttaagattagtttaggtaatatggtgaaaatttatttttataaaaagtataaaagttaggtgggtatggtggtgtgtttttgtagtttttgttatttgggaggttgaggtggagaattatttgagtttaggaaggttgaggttgtagtgagttatgatagtattattgtattttagtttgggtaatagagtgaaattttgtttttaaaaaaaaaaaggtaaatatagagattaaaatagaaaatttagaaaattttaaaggtagttggatagggtaaaaaagaaaaaaataaaatttagaaagtataaatatatgtagaagtttattataaaataaatatgttttttgagatttgtggagagcgattatttaataaatgttattagaataagtggtgaattatttggggaagaaaaaaagttaattttttatttgagtttttattagtgaaataaattttaaaaggatttaaggtttaagtttttataataagattataaaataattatagtatatatatataaatatttattttaagtataaaagaaaaatagatttgagaatataaacgttataagaatataattgaaattagaaagatattaaatttttagaataatattattatatattaatgggagaagaaaaatattttaaatagaaaaagggtaagagaaataaagtggtattttatatatagatatatatatatagaaatgatgaataaatatatgaaaaagtttaatttattgataataaaagaaatgtaaatatgtagtatgaaatagaagtataatgggatggtgagaataattatttaatttataatatttagagggaaatagaggtatttatgggaggtgatatgtataagaatgatttattttgaataatcgttaaggagaataatttgttattatatatttatatttttaaaatcgtttataatatttgattttatttttttatttttaagatgtgattttaaaaatggtatagagatagagatagagatagatttttttataacgatgtttattgtagtggaaaaatggaaagaaataaaatgtttaagaatagagttagtggagaatagtgatttagaaaaatatttggtgagttagtaaggtagaaggtgaagttatatgagtaattgattattatatgttacgatatgaaatgaagtaggttatgaaatatatgatgcggagagagatgtttagaatatattgagggttttgtattaaataataagttatttttggatggggagattatgataatttttttttttggatttttttttttattttttaaattttttataataaaatatatgagttttatagttataaagaaagaaagattttttaaaaatgatataacgttttgatatatttagtttaagttaggatttggagtggtatttgttttgttttatttttttgagtagtttttagttcgaaattattgtggaaatttaaatttttttttttaaaaagttagttttgttgatagtatttttattttttttatgtagaaagagggtttttggggtattggggattattggaattttggggatttggttttattgagttataaagtaggagagttaatagggataagtagggttgtattttaggataagtgatatgtgtaaaatttttaagttttttgtagttttagagtagaggtaggagattgaattattgggggagagtttaaatatggtgatagtagagagtaaattatttaattttgagattatttatttgatagtattttagaaatagtttaatgttgtttttttttttttttttttttttttttttttttttttttgtttttttttttttttttttttgaaatttagttttttttttttgtaaaaagaggggtttttttatttgggtgattattagagttatttagggagtttataggaaatataatttaggagttagtgttttagtgtttttatttagttttttaggtgagaatttaggaataatgattaatttagtgttgttttaatgtgtatataaatttttgggagttttttttgttttgttttgtttgtttgtttgtttgtttttttttgggacggaatttcgttttatcgtttagagtagagtttagtgatgtaattttggtttattgtaattttcgtttttcgggtttaagtgatttttttgttttagttttttgagtagttgggattataggagtgtattattatatttggttaatttttgtatttgtagtagagacgtagttttattatgttggttaggttggttttaaatatttgattttaaatgattcgttcgtttcggttttttaaagtgttgggattatgggcgtgagttatcgcgtttaattagatagtttgttaattaatttgtagattttgttttagtaggtttttagatttatacggaagtttttaggtgatgtttacgttgttggtttgaggtttatttttgagtagagaggttttaaagttttttttatttttgttttttattttgttattagttttttttttttttgtgttttttttttttgtatttagtaattttgagtttttgatgtgtgtaggagattatattacgacgttagaatgggttaggattttttagatggtatagcgagttttgaggtgaagtaggttggtagtatttttagaagttgtgttttttggttgggcgcggtggtttacgtttgtaattttagtattttgggaggtcgaggcgggtggattatgaggttaggagatcgagattattttggttaatatggtgaaatttcgtttttattaaaaatataaaaaattagttgggcgtggtggcgggcgtttgtagttttagttatttaggaggttgaggtaggagaatggcgtgaattcgggaggtggagtttgtagtgagtcgagattgcgttattgtattttagtttgggcgatagagagatttcgttttagaaaaaaaaaaaaaaaaaaaaaaaaaaaagttgtatttttttaggtaaattattgtttttggaagggttattttatgattaagagttgggggtttggttaggtataattttttatatttgtaattttagtattttgggaagttaaggtgggaggattatttgggtttaagagttagagattagtttgggtaatatagtaaaaatttatttttattataaataataataataaaataaataaataaataaataaagagttgggggtttttaggattttaggtaggtagaggtattgttagttttgtagtttgttaagtttattgtgaggttgtatagagtataggttggaggagtttgtttagtggagatggggaggattttttattttattgttgttttattatttttagttttgttttcggatagtttttgagagtaagtttgttttgtcgagtggtttagaggaggttcggggtttttttttgaggatttttttttattttttttggttttttttatttggttggttgttttttgttttattttttttgtagtttttagagtagtttagtttagtttttagcgttagtaaagaatgttttaagaaagattaagtttaagatataagaaaagtttgtatgggtttttataaattttgaagtaaaatttttttttggttaggtatagtggtttatattagtaattttattattttgggaggttgaggtgggtagattgtatgagtttaggagtttaagattagtttgggtagtatagtaaaattttatttttaaaataaatataaaaattagtcgggtatggtggtattcgtttgtggttttagttatttgggaagttgagatgggaggattatttgagtttaggaaggtttaggttgtagtgaattatgattgtattattgtattttagtttgggtaatagagtgagattttgttttaaaaataaataaataaattaataaatatagttttaaatttaagatttttttttaatatatgatatagtgaaaagggtttagggaaatggtttttaaatttgagtattaaaatgatttgggtagttttttaaattgatgttttgtttttattgtagtttaattaaattagagtgtcggggtggaagatgggtgttagtgttttttaaaattttttcggagtttttattgtatagttaagtttgggagtttttggtagggagagaggaatgtttgggtttttattttagtttatttataggtttgcggtgagattttagtggtagtttttttttttttttaggtttagtttttttattttaaaaacgatgtagttatgtagttttgaaattatttttaattttaaattatttatataggatttaagaattagagatatttatgaaatttaattgaattttagaattattggaaggaaaaataatttagattttagagaatagaattagtgttaaatttattataattttcgtaagtttttagatttttatattttgtatattgaattgtaggtaataatataaggtttttaattaggttatatttattatttaatgttatgtggtagaagtatacgtatattaaaataggaatatatatttttttttgtttttttagtttttatatataaaattagttatttattattgtgataagtaggattttttatagataggtatatttaggtaaagttgtattaaatttttatattggagagggtaattacgtattcgatattattgggaatgataataaggtgagaatatattgagtgtttatagagggttgattattaaaaggaaagtatggttttagatgatttatgagttattaattaattatatttttatttaagttatgtgtttattttgtttttatttcgatttgtatttgttgttgatttaaaagagtagaggaggagagaatttttttttgttgagttttcgttttatgttggtggattgttagtacgtgatgtgtttttattcgattttatatataaagaaatagttttagcgatgttagttataatagttaggtggtaaaggtgggttttttgaaggtataagaatagtttataggttagtttattaggtttatcgggtttgggtttgtggtttagatgattttgtttttagggtttgatatgagaatagaaatgggttaagttattttgtagggtataggagagtaagaattatatatagttcggggttttttgataagttgaattgttggatttttttagagttttttttgaatttgtagtttattttttgttgtattaagtaagttttgtttttttttgttttatagtggtttttttttaaataatcgtatttgtttttattatggttaggatggtttttggggaatggtaataagtagaattttttagggtgttggagggatttattattttatttgtttgttttgttattataataaaatattgtaattgggtggttttgattaaagaaatttatttgtttatagttttggagattagaaatgtgaggttaagttattggggaaggtttgtttttttttgagttttttttttttggtttgtagatggttatgtttttgttgttttttatatggtatgtatttttggtatttttttttgtgtttaaatttttttttttgataaggatattagttgtattggattagggtttattttaattattttattttatttaagttatttttttaaaggtcgtgtttttattatagttatattttgggatattgggggttagggtttaaattaatgaatttttaggggatgtaattgagtttataagataattttattttaaaaagtttttttatttttcgttatagtttgttatagtttttaaaatttaatttgagatttttttgtttatagatgagagtttatgtttttaattaggggatttttgaagtttatttgagttggttatagtggttttttttagtattattttttattttttttaaaattagttttattatttttaatatagtttgaggttatttttttgttttttagaagagttgtagtgttgtttggttgtttttttatttattaacggtgttatatatttttgtttttatttgtataagttaagtgtttatcgattttaagttttaggaaatcggggtatatttattatgtattgtagttttttggatttttttaaaattttaagtttgaagaattttttatagtgtgaagtgggaagttttgttttatattgagaggaattaggatttgttgtttttatttgtttatttttagggtataggtataaatttcggttttataaatattttttttttaaaaagttttttttagagagggtttaaggatattggtattatgtagaatttttttaggaatgatggtgtggagggttttggtttttagggtaagggttttttggagtttagtgtgtagtgttagtgtatgggggttgtgattgtgattggtagtaaggtggttttgttggagttaagtagtagtgtggttttagtgttttatttgaatttgtgttaagaaagtttgtgtttttggtcgttagtagaggtttgaattttttaataaatttttttttattttaaatgatttagagtaaattttattgtttgtagttaagaattttgtgatttagatagtgattgtgatttatttttgtttgtgtgttttttagtgattagtagggtgtttggtgtaaagtagttgtttagaataggattttatttgggttattttaggtatttttaagtagaaagtattttggtaagattaatttatattattgttgaaatgagtttatttattgttttttaggggttgagaaagggatgtttagagtaggtgtggttagttgtttatagggaggaaggtaaatgaaatgagtagtgtttttcgggtgtgtgatagttagttttgtttaaatttgtttgattagtttttagtgttgattttattttaattgttatttttatagatggaattaatttaattttttttaattatttatttttttttttatttattttaattttttttaaagatatagttttgttttgttgtttaggttggagtgtagtggtattattatagtttattgttatttttaatttttagatttaattgatttttttatttaagttttttaagtaggtgggattataggagcgtgttattatatttagttaatttttagattttttttgtagagatagggttttgttatgttgtttagtttggttttgaatttttgggtttaagtgatgtttttgttttttttttttaaagtgttgggattataggtataagttattatgtttaggttgattattattttaatggttatattttattttatggaatgatatggattattatttatttggttaggtattttttgttgaatatttaggttgtttttgatttttttttttataattagaaatattgggttgggtatggtggtttatatttgtaattttagtattttgggaggtcgagacgggcggattacgaggttaggagatcgagattattttggttaatatggtgaaatttcgtttttattaaaaatataaaaaaattagtcgggcgttatggcgggcgtttgtagttttagttattcggaaggttgacgtaggagaatggcgtgaattcggaaggcggagtttgtagtgagcggagatcgcgttattgtattttagtttgggcgagagagcgagattttgttttaaaaaaaaaaaaaagaaacgttgttgaaatttaggttttttagataaaaattttttatatttatattaatttttttaagataaatttatagaggtcgggtgaggtggttcgtatttgtaattttagtattttggaaggacgaggtgggtggattacgtgaggttacgagttcgagattagtttgattaatatggagaaatttcgtttttattaaaaatataaaattagtcgggcgtggtggagtatgtttgtaattttagttatttcggagggtgaggtaggagaattatttgaaatcgggagacgaaggttgttgtgagttgagattacgttattgtattttaatttgggtaataagagcgaaattttattttaaaaaaaaaaaaaattataaaggtagaatttttgggtaaaagaaaattataatttttttagattattatttgttattatgatatgtgggaaatgttttgttttaatttatttttttttgttaattggtaagattagatttttttttttttttttttttttttttttgagacggagtttcgttttgttttttaggttgtagtgtaatggtgtaattttagtttagtataagttttattttttaggtttacgttttttttttgttttagtttttcgagtagttggaattataggtattttttattatattcggttaattttttgtatttttaatagagacggggttttattatgttagttaggatggttttgattttttgatttcgtgatttgtttttttcgggtttttaaagtgttgggattataggcgtgagttatcgtgtatagtttagatttttttttgtatatgtattgattaattgtttttttttttttgtgagtattttgtttatttatgttttttagttatttttttgtttgggttatttatattttttttttggattagttaaggtttttaatattaaagatattaatttttaagtgttatatatgttaaaatatttttttgattttttttatggttagtatatttattttggagttagaattaaggaatgagaaattttttttatttatatttttattttttaaatgatatgttttgatatgtagagattttaaattttaattgattatagttatattatttttatttgtgggttagtattataagttttaaagaatattttaagatttatggagttatatagtttttgggttgatgttgtggtaggagttgttatttatttttagggttttttgttttgttttgttttttgtttttttttgagatagagttttgttttgtcgtttaggatggagtgtagtgttgtaattttagtttattgtaatttttatttttcgggtataagcgatttttgtgttttagtttttttagtagttggggttataggtatgtattattatgtttggttaatttttgtatttttagtagagatagggttttattattgtttaggttggttttgaacgtttgattttaagtgatttgtttgttttggttttttaaagtgttgggattataagtatgggttatcgtgttttgttattatttttagtgttaaaagaacgttagataaattaaatttaatagagtttgattgagtaaagaattatttgtgtgaattaggtagtttttaggtaaaaataagtttagagagatttttgtattgttttatagttaaagatttatggatagaaaaaggaaagtgatgtatagaaattggaagtgaggtatagaaatagacggattggtgatggtatggtgtttggtttatttgagtttagttttaatagttggtcgtttgtgagtggttgaagtttggttgttgtgattaattgagattcggttatttggtacgagagtaggtgatagtttgtttatacgtgtagttagtttatagtttattatgtatagagaaattattagggtgaatttaaaatatgtaaggagaaggttttaggttaaatttgatttaatattaggatcggtggggtttttttttattagtttttaagttgatgatttattttttatttatttttttttttatcgtgttttagagtttttttaaattatgatttttttttttaaattttttatttaattgtttattttatacgtagaatataattttatttcgtattttatagagattagaggtgaaaattgtatttttatttttattttgtatattgttttttatcggtttttgttttttttattttttttttgggttttttttttattttaaatacgtaatacggattttatttttttttttatcggtttttatttttttttagggatttttttataaaagttagtttttttaagataagtaggtatgaagggatagatggaaaagtgtttagtgttcgaggattgtttaatttttagtttggtataagttagtttttttgagggtttatttatattttggtaattttattttgtagagtttaatttttgtgatattttgtggtaggattcgttttggtattcgttaggattttgttttgttggaaatcgaattaaaatgagtatgggtaaaaagagacgtttttagtttaagtgttaagtttagaggtggattagtttttggattgcggggtttagtttaggatagatattatgagagtttttattttgtagaaattagaaagatagattttgtttttatttatggatttatttttgtataattttttgttttaaaaatataataagataaattttttatgttaaaggatgtagtagagggtttttttatagtaatagaaatgttgtaggatgaattttttatatggagtgagggtgacggtgggatagagattttggatatttaggtaaagaattgggttggaaaaggaagttattaatagaatagcgagaaagaatattgtaaatgttgtgagttatagttattttggatatagtgttataagagtgtattggaatgtattgaggttgataagttagggattaggaagattgttggtgaataatttttttggttttttgtaaggaatttggaaagtttatttagagaggtgtgaaaatagtagaaaaaatattgggataaagaaacggggtaaaagtaaatatgttttagattagtcgttgagaaatttaagaaataaaattaggttttgttttagagaaaagggagatttgggtatttttttggaggttcggaagttttttgtttgttaagtggattaaagggtatttggttaaggattgtgtgtttatagtttaaagtaattatgtatagaatttaggaattatttgttttttacgtttttaatggattgttttagagattgtttttttttgtgaagttgatgtttgttttgtgttgaatgtaatatagttataaggaggttatcgtgatagggagtttgtattttttttttggatgtggtatttaaagtgttgaataattagtatggtttaattattgattttttagtttagttgttagaagttagtttgggggattttattgtgcgaggttttattttttttgtttttggatgatgttgaggtttggaggggttaggatttaatatgttgagtgggggtatttaggagtgttgggtagtagttacgtatttattagtatatttgaattgatttatattagtattataggtttttattttttttatagagtatatgttaagtaaatgtagttattgaattagatagaagaacgttagttagtaattttttgtttttattatgagtagtgttttttagaattattttagggtggagggttgagaggtgggaagggagttggagttttttatgtttatatatgtattattttagggattttttagtaatataataaaataggttttttttcgtttattttattagtggggtattgaggttttaggatggaaataattatttagggtttttagatggtgagaaagagtgagaatttaaatttaggtttgagatgaagtagggatttgtgttattatttcgaacgttattattaagtatggaaataaaggaaaattttgagtgtttttaagggaaattttaggtatttagttagttttgagaagtaagtaagtaatttgttaggtaggaaggtaatagtagtttaaaataatagttaaggaagttagagttcggagatgtttagttttaggaattaaagataatattttaatatacgtttttgagtttgttatttagaaatttggatttttatgtggatttttattaaacggatttgttggtttgtagattttagatagagggagttgaggattgtattttgattatttttttttattttaaatttttttcggaggtttttgaaagttatatttatgagttaaagttaatatttttttttgttaattttgaatttttaggtaaggtatcgttttttgaattaattataaattaaagaatttttgaatttatttatgatttgtaagtttttgttttaagatattttattttttttaggttaaattaatgtgtattttttatgtattgatttataattttatttaatttttgttgtttttttgaaaatttatttcgtttttaaaaatttttatttgtgtgaaaggaaattatgttggtttttttaaattattaagttaaagagaaaatttaagttgggaattgtttagggtaaatttgtttcgtattttatttaaagttatttttttgtttattgagataaatgtatatttgattgttttttttggaaaggttaattagaaatttaaaagaatgtaatcgtttgttttttatttatttgtgatttggaagtttattgtttgttttaagttgttttgtttttttagattaaattaatgtttattttatatatgttgattgatggtttatgtttttttaaaaggtataaaattaagttgtgttttgattattttgggtatatgtcgttagaattttttgagggtgtgttacgggggtatgtttttaattttggtaaaataaattttttaaattgatatttgttttagatttttagggtttatatttgttagttattagagagtttaggttttgagtatgcgttgtttggttttttttgtttagggttttgtgaataaatatttttttttttagtgttgtaattttagtgtggatatttggatttaattgtattgggtaagtcgattttaatttgttaagtacgtttatgtgaagaaattattaaataggttttgtatgaggaataaaatttttaatttatttgggtgtaggtgggttgagttcgaaaagagttagtaaagggagatggggaagggattgttttataggagttgggtaggtaatggaaagttatagtaaaacgtggttatttattgttagtagaggagggggttataaggtatatagtggggagattataagatttattgtttagaagaagaatgttataaagttggttgattagttaaggtagggtagggataagttataatggtaaaatgttgtaattttggttatttagttaaggtaggaattggttgttttattttgtgggttttttggttgttttagattttttggtttttgtaggttatttggatatatatgtgtaggttataggggttataatagttgagttttagtttagaggtttgatataatttagttatataatagttttaatgatttaaaagttagtatttttttttttttttttttttttttttttttgagatggagtttcgcgttgtcgttcgggttggagtgtagtggcgttattttagtttattgtaattttcgttttttgggtttaagtgatttttttgttttagttttttgcgtagttgggattataggtatatgttattatgtttagttaatttttgtattttttttttagtagagatggggttttattatgttggttaggttggttataaatttttgattttaaatgatttgttatttcggttttttaaagtgttgggattataggcgtgagttattacgtttagttaagtttttgttttttttatagttagggttgttttttttatatatttgtttaaagggttggtagtatttgtatttaagtgaagagatttaagtatttttgagttttagagtagtaggtgggggttattattagtatggggttttttatgcgggagattttatagttttttttttttttttttttttttttttgataatgtgtttaggttttttatagtttttggtattaagaaattttaatagatgttaattagtttggtaatttagtatagcgttagtattaagtcgttaaggttgaggatatgttatcgtgatatatttttaggaggttttgataatatgtgtttaaggtagttagagtatagtttggttttatattttttagggagatatgagttattatttaatatatgtaaaatgaatattggtttagtttggaaaggtaggataatgtgaagtaggtagggggtttttagattatatatgtgagagataaacggtgttatttttaaatatttttaaattatttttttttttagttatttattaggaggaaagtaaatgtttgttagatatggttattaattatcgtcggtgggtgggatgaaaaggagtttattgatgattggttaggtgtttatttttattaggttttagatttttttttggttaatgagggagttaattttttattgggggtagtaagtttaaatgttcggggtaagttaggtaatataatttagtgaagtaggtatgatgtttgtatatgaagtatttatgagttttatttttttaagtaaataggtttttttttattttttttgaaatgtatattattgttagtttgtttttttttattttaattttgatagagatatatgtataaaaaatatatttttaggttaggagtagtggtttatatttataattttagaattttgggaggtttaggtaggaggattgtttgaggttagaaatttaagagtagtttggataatatagtgagaattttatttttataaaataaaaaaattagttaaattggaggtaaaggcgggtggatggtttgaggttaggagtttaaaattagtttgattaatatggtgaaattttattattattaaaaatataaaattaggtagggtatggtggtttataattgtaattttagtacgttgggaggtcgagacgggtggattatgaggttaggagatcgagattattttggttaacgtggtgaaatttcgtttttattaaaaatataaaaaaagttaggtgtggtcgcgggtgtttgtagttttagttatttaggaggttgaagtaggagaatggcgtgaattcgggaggcggagtttgtagtgagttaagattacgttattgtattttcgtttgggtgatagagtaagattttgttttaaaaaaaaaaaaaaaaaaaaaattagttaggtttggtggtgtatatttgtaattttagttatttgggaggtaggagtattatttgaatttgggaggtggaggttgtaatgggtcgagattatgttattgtattttcgtttgggtaataagagtaaaattttattttaaaaagataggataggatacgatacgatataataagacgagaagaaatattatgttttttataaaaattttttagaaaataaaaaaggatagattatgttttaatttttttaatgaggttagtgttattttaatattaaagttaaataaatatattaaaataaataaaaattatagaataatattttttatgaatagagatgtaaatatatttaataagatattagtaaataaaaatttattaatatgtataaaagattatatattatgattaagtatgatttattttagaaatgtaaggttggtgtgatatttaaaaattaagtaatgttttttttagtatatggtgaggtttttaaaattaattaattaaaaataataaaattttaaaattagtgaatgtaatgtattagattaatagattaaaatatattatattgttattttaatagatatagaagtatttgataatagttaatatatattcgtggtaaaaatttttaataaattaggaataaaagtaaatttttttaatttgatagagggtaattatagataatttatagttaatgtatttaatgatgaaagatagaatgttttttatttaagattaggaaataggtaagaatgtttatttttattatttttatttaatattgtattattggttatagttagggtaatttaaaaagggggtagagggagaaagagagagaaaggtaaattaaagatatttagaatgaaaagaaaaaagtaaaattgtgtttatttgtagattatatgatttgtatgtagaaaattttaagtaatttattaaaaaattttattggaattaataaatgagtttagtaaataaggtttaagtatataaggtcgatatgtaaaaattaattgtatttttatatattagtaataaataatgtgaaaatgaaattaaggaaataaaattttatttatagtaatatttaaaagaataaaatatttagaaatgagtttaatgaaagaaatgtaagatttataataaaaattataaaattttgtttagagaaattgaagaagttttaaattagaggtagtaaattatagtttatggttttgaattggttttttgtttgttttgtaaataaagttttattgaaatataatttttttttttttgagatggagttttgtttttgttgtttaagttggagtgtaatggtgtgattttagtttattgtaatatttattttttaggtttaagcgatttttttgttttagtttttcgaatagttggaattataggcgtgcgttattatgtttggttattttttttgtatttttagtagtaacggggtttttttatgttagttagttggttttgaatttttgattttaggtagtttattcgttttagttttttaaagtgttgggattataagtatgagttatcgcgtcgggttaatattgtttttatattataaagtaaagttgaatatagtttataatattataaatatttttattatttggttttttgtagaaaatttgttaatttttgatttaaataattttatgtttatagattaaaagattttattgttaagatgatagtttttttttaatttattgataaatttttgttaatttaaaaaaaattaatgtcgtttttaagaaagtgagtttattttaaatattgcgaaattaagggaaaaaattaaaaagttagttttggttgggtgtagtggtttacgtttgtaattttagtatttgggaggtcgaggtaggtggattatttgagtttaggtgtttaagattagtttggttaatatggtgaaatagtgattttattaaaaatataaaaattatttgggtatggtggtatttgtttgtaattttagttattagggaggttgaggtaggagaatcgtttgaatttgggaggcggaggttgtagtgagttaagattgtgttattatattttagtttggatgatagagtgagatttggttttaaaaataaaaataaaaaaaatagtttagtaggtttttgtggaaattgataagtttttgttaaaatttatatagaaaagcgaatgatttaggatagtttaaatattttgaaaaagaataaagttaaaaaaaaaatttatattatttaattttaatatttgttattgttaaagaaaaattttgtattagatatttgttaaaaatgttaagatagattttatttagattattgtggtagggaagagatattttagtattaattgagtttaattttattaaattagaaggtaagaggttttttaaatgttaggatgtgttaagggaaaagtattgaaggatgttagttgaggggagggaggtttggttaatgagtatattgattagattgtgaatttgtgtttattaaattgtgaattggtgtttattaatattagggttttattttattattaagattgggagatagaggttttgttttatttaatgattatatttcgaaaggatggtttttaggtttttgggaaagatatttttgtgttatagaagatatatttttaagagatagagaaaggatttttaattgttagttttttaaagtaaattttttaggaaaagggggttcgggggtttatatttaggttttgtttggaataaattataaattttttgggtagtattgaattttattaggtagaaatttaagggggttgaagttattattttaaggatgtggttatgaattgttagaaataatagtgtttgttgaagttttttagtgttagaggtggatgaaattatttgtgttgagagtttgtagtttgtttttttatttatattttattttttattatttatttttttttttttttttttgagatagggttttatttttgttgtttaggttggatttaggttggagtgtagtggcgttatttcggtttattgtaagttttgtttttcgggtttacgttatttttttgttttagttttttgagtagttgggattataggcgtttgttattacgttcggttaattttttttgtatttttagtagagacggggttttattacgttaattaggatggtttcgattttttgattttatgatttatttatttcggttttttaaagtgttgggattataggtatgagttattgcgttcggtttattttttttttttttttaagtataatatatttttattaagttgaatagaaatattaataaagattagtatgtatagtgattatagatgtagatagataaaaaagaaaaagggatataagggcgtattgaatttatgataatattgttttagtattatagtaggagttaatttattttagggttggttttttaagattagtttcgggaagtttgatttgtttagttttgattattggttgatcgggattggggatttagttggtaggataggttgtagtttttatttttattgggttgttttttgatttagaattaggtttttagtttttttttaggatgaaggttgtaatgatttgtagttatttgagtagttgggtttttttttttgggagtttattatatttttatttatattattatttatcgtagtgtttagtattattaggtcggtgaggaatgtgttaaggatagggataattttttttttttgttgttgttattttgtatgggttttgtgggggtttgtttttagggtggtcgatttggaggtttttatattgatgagtagatttttgttcggtggtttgtttttgttgtatagttttggatatttttttgttgttttattttggttatttagagtagagtagttataattttttattttatagtgtttgggttaaagtggtttaggttagggtttttgaaagacgagatattagagagttttttttttttttttttttttttgttttttatattttttttttaggagtataaagaggttatgtgagtacgtagtgggagggtggttatatgtgagttaagagaggggttttagaatgaagtttgttttttttgtattttgatcgtggatttttagtttttagaattgtgagaaatgaatttttgttatttaagttttttagtttgtggtattttgttatggtagtttgagttaattaatataataggaaagaaatattttaattttcgtaaatttatttttaagtttattaatgtttaaagtgattaatagtgggtatattttgtaaattattaagattttatatagttatattttatgattttttataattagttggttatggtttgtaatttttgataaatattgtttttagttttagag

SEQ ID NO: 9 - DNA, 21 bp, Artificial Sequence
  Chemically Synthesized - primer
gtttaggttggagtgtagtgg

SEQ ID NO: 10 - DNA, 25 bp, Artificial Sequence
  Chemically Synthesized - primer
atcctaactaacataataaaacccc

SEQ ID NO: 11 - DNA, 27 bp, Artificial Sequence
  Chemically Synthesized - primer
agtttttttatttaggggtggatttta

SEQ ID NO: 12 - DNA, 27 bp, Artificial Sequence
  Chemically Synthesized - primer
ccaaactaatctcaaatacctaacctc

SEQ ID NO: 13 - DNA, 25 bp, Artificial Sequence
  Chemically Synthesized - primer
gttatttaggcgggagtgtagtaac

SEQ ID NO: 14 - DNA, 25 bp, Artificial Sequence
  Chemically Synthesized - primer
taaatcataaaatcaaaaaatcgaa

SEQ ID NO: 15 - DNA, 25 bp, Artificial Sequence
  Chemically Synthesized - primer
tatttaggtgggagtgtagtaatgt

SEQ ID NO: 16 - DNA, 25 bp, Artificial Sequence
  Chemically Synthesized - primer
taaatcataaaatcaaaaaatcaaa

SEQ ID NO: 17 - DNA, 24 bp, Artificial Sequence
  Chemically Synthesized - primer
aaatcataaaatcaaaaaatcgaa

SEQ ID NO: 18 - DNA, 24 bp, Artificial Sequence
  Chemically Synthesized - primer
aaatcataaaatcaaaaaatcaaa

SEQ ID NO: 19 - DNA, 24 bp, Artificial Sequence
  Chemically Synthesized - primer
ttatttaggcgggagtgtagtaac

SEQ ID NO: 20 - DNA, 26 bp, Artificial Sequence
  Chemically Synthesized - primer
ataaatcataaaatcaaaaaatcgaa

SEQ ID NO: 21 - DNA, 25 bp, Artificial Sequence
  Chemically Synthesized - primer
tttttgagtagttgggattataggc

SEQ ID NO: 22 - DNA, 23 bp, Artificial Sequence
  Chemically Synthesized - primer
acactttaaaaaaccgaaacgaa

SEQ ID NO: 23 - DNA, 26 bp, Artificial Sequence
  Chemically Synthesized - primer
ttttttgagtagttgggattataggt

SEQ ID NO: 24 - DNA, 25 bp, Artificial Sequence
  Chemically Synthesized - primer
caacactttaaaaaaccaaaacaaa

SEQ ID NO: 25 - DNA, 24 bp, Artificial Sequence
  Chemically Synthesized - primer
aacactttaaaaaaccaaaacaaa

SEQ ID NO: 26 - DNA, 23 bp, Artificial Sequence
  Chemically Synthesized - primer
aacactttaaaaaaccgaaacga

SEQ ID NO: 27 - DNA, 27 bp, Artificial Sequence
  Chemically Synthesized - primer
gttttttgagtagttgggattataggt

SEQ ID NO: 28 - DNA, 24 bp, Artificial Sequence
  Chemically Synthesized - primer
gagtagttgggattataggcgttc

SEQ ID NO: 29 - DNA, 25 bp, Artificial Sequence
  Chemically Synthesized - primer
taaatcataaaatcaaaaaatcgaa

SEQ ID NO: 30 - DNA, 25 bp, Artificial Sequence
  Chemically Synthesized - primer
agtagttgggattataggtgtttgt

SEQ ID NO: 31 - DNA, 25 bp, Artificial Sequence
  Chemically Synthesized - primer
taaatcataaaatcaaaaaatcaaa

SEQ ID NO: 32 - DNA, 25 bp, Artificial Sequence
  Chemically Synthesized - primer
ttgtaattttagtattttgggaggt

SEQ ID NO: 33 - DNA, 25 bp, Artificial Sequence
  Chemically Synthesized - primer
ctcttattacccaaactaaacccaa

SEQ ID NO: 34 - DNA, 24 bp, Artificial Sequence
  Chemically Synthesized - primer
tgttgtttaggttggagtgtaatg

SEQ ID NO: 35 - DNA, 26 bp, Artificial Sequence
  Chemically Synthesized - primer
ctataatcccaacactttaaaaaaac

SEQ ID NO: 36 - DNA, 25 bp, Artificial Sequence
  Chemically Synthesized - primer
tttgttgtttaggttggagtgtaat

SEQ ID NO: 37 - DNA, 27 bp, Artificial Sequence
  Chemically Synthesized - primer
cacctataatcccaacactttaaaaaa

SEQ ID NO: 38 - DNA, 24 bp, Artificial Sequence
  Chemically Synthesized - primer
gtaagtttcgtttttcgggtttac

SEQ ID NO: 39 - DNA, 26 bp, Artificial Sequence
  Chemically Synthesized - primer
cctaactaacataataaaaccccgtc

SEQ ID NO: 40 - DNA, 25 bp, Artificial Sequence
  Chemically Synthesized - primer
taagttttgttttttgggtttatgt

SEQ ID NO: 41 - DNA, 25 bp, Artificial Sequence
  Chemically Synthesized - primer
ctaactaacataataaaaccccatc

SEQ ID NO: 42 - DNA, 24 bp, Artificial Sequence
  Chemically Synthesized - primer
tttcgagtagttgggattataggc

SEQ ID NO: 43 - DNA, 23 bp, Artificial Sequence
  Chemically Synthesized - primer
aacactttaaaaaaccgaaacga

SEQ ID NO: 44 - DNA, 26 bp, primer
ttttttgagtagttgggattataggt

SEQ ID NO: 45 - DNA, 25 bp, Artificial Sequence
  Chemically Synthesized - primer
caacactttaaaaaaccaaaacaaa

SEQ ID NO: 46 - DNA, 25 bp, Artificial Sequence
  Chemically Synthesized - primer
ttttttgagtagttgggattatagg

SEQ ID NO: 47 - DNA, 30 bp, Artificial Sequence
  Chemically Synthesized - primer
aaaacaaactaacaatatccttaaaaatta

SEQ ID NO: 48 - DNA, 25 bp, Artificial Sequence
  Chemically Synthesized - primer
attgtgggttagttataatggttta

SEQ ID NO: 49 - DNA, 23 bp, Artificial Sequence
  Chemically Synthesized - primer
actcactacaaactctacctccc

SEQ ID NO: 50 - DNA, 25 bp, Artificial Sequence
  Chemically Synthesized - primer
gtgggttagttataatggtttacgt

SEQ ID NO: 51 - DNA, 22 bp, Artificial Sequence
  Chemically Synthesized - primer
actacaaactctacctcccgaa

SEQ ID NO: 52 - DNA, 25 bp, Artificial Sequence
  Chemically Synthesized - primer
gtgggttagttataatggtttatgt

SEQ ID NO: 53 - DNA, 24 bp, Artificial Sequence
  Chemically Synthesized - primer
ctcactacaaactctacctcccaa

SEQ ID NO: 54 - DNA, 26 bp, Artificial Sequence
  Chemically Synthesized - primer
tgtagtggtgtgatttttgtttattg

SEQ ID NO: 55 - DNA, 25 bp, Artificial Sequence
  Chemically Synthesized - primer
tataatcccaacaccttaaaaaacc

SEQ ID NO: 56 - DNA, 24 bp, Artificial Sequence
  Chemically Synthesized - primer
gtttaggatggagtgtagtggtgt

SEQ ID NO: 57 - DNA, 25 bp, Artificial Sequence
  Chemically Synthesized - primer
tgggattataggcgtttgtaattac

SEQ ID NO: 58 - DNA, 22 bp, Artificial Sequence
  Chemically Synthesized - primer
acaccttaaaaaaccgaaacga

SEQ ID NO: 59 - DNA, 27 bp, Artificial Sequence
  Chemically Synthesized - primer
tgggattataggtgtttgtaattatgt

SEQ ID NO: 60 - DNA, 23 bp, Artificial Sequence
  Chemically Synthesized - primer
acaccttaaaaaaccaaaacaaa

SEQ ID NO: 61 - DNA, 25 bp, Artificial Sequence
  Chemically Synthesized - primer
tgtaagtttcgttttttgggtttac

SEQ ID NO: 62 - DNA, 21 bp, Artificial Sequence
  Chemically Synthesized - primer
aaccgaacgctatatctcacg

SEQ ID NO: 63 - DNA, 25 bp, Artificial Sequence
  Chemically Synthesized - primer
taagttttgttttttgggtttatgt

SEQ ID NO: 64 - DNA, 24 bp, Artificial Sequence
  Chemically Synthesized - primer
aaaccaaacactatatctcacacc

SEQ ID NO: 65 - DNA, 26 bp, Artificial Sequence
  Chemically Synthesized - primer
tagggttatttgggttatagatttag

SEQ ID NO: 66 - DNA, 25 bp, Artificial Sequence
  Chemically Synthesized - primer
cctaactaaatcacatttaccattc

SEQ ID NO: 67 - DNA, 30 bp, Artificial Sequence
  Chemically Synthesized - primer
attatagtaataggtgataatttaagagaa

SEQ ID NO: 68 - DNA, 29 bp, Artificial Sequence
  Chemically Synthesized - primer
aaatacctcttattaaacactcaaattat

SEQ ID NO: 69 - DNA, 29 bp, Artificial Sequence
  Chemically Synthesized - primer
gagaaattatgtttggttagatttttaat

SEQ ID NO: 70 - DNA, 29 bp, Artificial Sequence
  Chemically Synthesized - primer
ataaccaacccaatactactttaatatac

SEQ ID NO: 71 - RNA, 25 nt, Artificial Sequence
  Chemically Synthesized - PPP1R8 gene
ggaacucgaaccuccacgaacaauu

SEQ ID NO: 72 - RNA, 21 nt, Artificial Sequence
  Chemically Synthesized - EZH2 siRNA
aagacucugaaugcaguugcu

SEQ ID NO: 73 - RNA, 21 nt, Artificial Sequence
  Chemically Synthesized
aacuggacuuccagaagaaca

SEQ ID NO: 74 - DNA, 21 bp, Artificial Sequence
  Chemically Synthesized - primer sense
gtttaggttggagtgtagtgg

SEQ ID NO: 75 - DNA, 25 bp, Artificial Sequence
  Chemically Synthesized - primer antisense
atcctaactaacataataaaacccc

SEQ ID NO: 76 - DNA, 27 bp, Artificial Sequence
  Chemically Synthesized - primer sense
agtttttttatttaggggtggatttta

SEQ ID NO: 77 - DNA, 27 bp, Artificial Sequence
  Chemically Synthesized - primer antisense
ccaaactaatctcaaatacctaacctc

(19) United States
(12) Patent Application Publication
(10) Pub. No.: US 2009/0203010 A1
(43) Pub. Date: Aug. 13, 2009

(54) MSMB-gene based diagnosis, staging and prognosis of prostate cancer

(75) Inventors: Lijs Beke, Heverlee (BE); Monique Beullens, Leuven (BE); Mathieu Bollen, Haasrode (BE); Kyrylo Litovkin, Leuven (BE); Mieke Nuytten, Holsbeek (BE); Aleyde van Eynde, Pellenberg (BE)

Correspondence Address: TRASKBRITT, P.C., P.O. BOX 2550, SALT LAKE CITY, UT, 84110, US

(73) Assignee: Katholieke Universiteit Leuven, K.U. Leuven R&D, Leuven (BE)

(21) Appl. No.: 12/321,583

(22) Filed: Jan. 21, 2009

Related U.S. Application Data
(60) Provisional application No. 61/011,537, filed on Jan. 18, 2008.

(51) Int. Cl. C12Q 1/68; C07H 21/04

(52) U.S. Cl. 435/6; 536/23.1

(57) ABSTRACT

This invention relates generally to a method of diagnosis for distinguishing between a benign prostate hyperplasia and a prostate cancer and between a hormone-sensitive and a hormone-refractory prostate cancer condition and specifically to identification of a hypermethylated (on CpG and non-CpG dinucleotides) CpG island in the beta-microseminoprotein (MSMB) regulatory regions surrounding the transcriptional start site of the MSMB gene as a diagnostic indicator of prostate cancer (PrCa) and for distinguishing androgen-refractory from androgen-sensitive prostate cancer.

### CROSS-REFERENCE TO RELATED APPLICATION

This application claims the benefit, under 35 U.S.C. § 119(e) to U.S. Provisional Patent Appln. Ser. No. 61/011,537, filed Jan. 18, 2008, the contents of the entirety of which are incorporated herein by this reference.

### STATEMENT ACCORDING TO 37 C.F.R. §1.52(e)(5)—SEQUENCE LISTING SUBMITTED ON COMPACT DISC

Pursuant to 37 C.F.R. §1.52(e)(1)(ii), a compact disc containing an electronic version of the Sequence Listing has been submitted concomitant with this application, the contents of which are hereby incorporated by reference. A second compact disc is submitted and is an identical copy of the first compact disc. The discs are labeled “copy 1” and “copy 2,” respectively, and each disc contains one file entitled “Sequence_listing.txt” which is 174 KB and created on Jan. 20, 2009.

### TECHNICAL FIELD

The invention generally relates to medicine and biotechnology.

### BACKGROUND

Several documents are cited throughout the text of this specification. Each of the documents herein (including any manufacturer's specifications, instructions etc.) are hereby incorporated by reference; however, there is no admission that any document cited is indeed prior art of the invention.

Prostate cancer (PrCa) is the second most common malignancy in males worldwide after lung cancer, and the third leading cause of cancer death in men. Early detection greatly improves survival rates. If the malignant prostate tumor is still local, PrCa can be successfully treated by radiation therapy, surgery, hormone therapy and/or chemotherapy.

Unfortunately, if the PrCa invades other parts of the body like bones, lymph nodes, rectum and bladder (metastatic PrCa), it becomes refractory to hormone therapy. For this advanced PrCa the prognosis is poor. Currently, PrCa is detected by an elevated level of Prostate-Specific Antigen (PSA) in the blood, along with a digital rectal exam. The PSA test is also used to monitor patients for the recurrence of PrCa following surgery or other treatments. However, although the PSA test has greatly improved the detection of PrCa, its usefulness is still controversial. A recent study by Concato et al. shows that PSA screening is not associated with lower mortality (J. Concato et al. (2006), Arch. Intern. Med. 166:38-43). Moreover, the serum PSA level is also elevated in non-cancerous prostate disorders such as benign prostate hyperplasia and infection.

Initial tests for suspected prostate cancer are done by analysis of blood levels of protein like PSA or, for instance, PSP94 protein (under development). Positive tests are followed by a conformational diagnosis. The only test which can fully confirm the diagnosis of prostate cancer is a biopsy, the removal of small pieces of the prostate for microscopic examination. The invention provides a novel diagnostic test of prostatic tissue or cells obtainable from prostatic tissue.

A condition of benign prostatic hyperplasia (BPH) or benign prostatic hypertrophy is common as a man ages. It is thus very important to distinguish between a PrCa and a BPH. Moreover hormone-refractory prostate cancers are more aggressive and need specific treatments such as apoptosis and regression induction of the tumors and/or antimetastasis.

The latter type of cancer will remain localized in a person's lifetime and is unlikely to reduce life expectancy. In contrast, an aggressive cancer is more lethal, due to metastasis, and requires immediate intervention. Therefore, there is an unmet need for a reliable diagnostic assay and biomarker to distinguish between these two types. The invention provides such.

PrCa can be distinguished from BPH by diagnosis of prostate cells or tissues, for instance, from prostate tissue biopsy or prostate cells in seminal fluids by assessing the DNA methylation status of non-CpGs (in particular, of CpA and CpT) upstream of or in the promoter region of the MSMB gene as novel biomarker to diagnose for prostate cancer.

PrCa diagnosis is based on newly identified DNA-methylation markers in the DNA methylome of the MSMB gene and, in particular, the DNA methylation status of specific CpG islands upstream of or in the promoter region of the MSMB gene as novel biomarker.

The CpG methylation status of specific CpG islands upstream or in the promoter region of the MSMB gene can be used to diagnose for prostate cancer and to distinguish between hormone-refractory and hormone-sensitive prostate cancer, in particular between an androgen hormone-refractory prostate cancer and an androgen hormone-sensitive prostate cancer. Moreover the methylation status of the non CpGs upstream of or in the promoter region of the MSMB gene can be used to distinguish between benign prostate hyperplasia (BPH) and prostate cancer.

The MSMB gene has a role as an autocrine paracrine factor in uterine, breast and other female reproductive tissues (M. Baijal-Gupta et al., J. Endocrinol. 2000 May, 165(2):425-33). Transcriptional silencing of the MSMB gene is known to be associated with prostate-cancer progression. Expression profiling revealed that the expression of the MSMB gene gradually decreases during the development of PrCa, i.e., from primary PrCa to the late, highly invasive, androgen-independent state (E. S. LaTulippe et al. (2002), Cancer Res. 62:4499-506; D. K. Vanaja et al. (2003), Cancer Res. 63:3877-82; and M. Stanbrough et al. (2006), Cancer Res. 66:2815-25). Actually, the MSMB gene was the most down-regulated of all genes and its expression level decreased about 100-fold in metastatic prostate tissue, as compared to its expression in adjacent benign tissue (D. K. Vanaja et al. (2003), Cancer Res. 63:3877-82).

### SUMMARY OF THE INVENTION

This invention relates generally to the identification of a prostate cell proliferative disorder and method and a diagnostic assay to distinguish between benign prostate hyperplasia (BPH) and prostate cancer. Moreover it relates to a diagnostic assay or method to distinguish between a hormone-sensitive and a hormone-refractory prostate cell proliferative disorder. More specifically this invention relates to analyzing the methylation status of non CpG dinucleotides in the regulatory region surrounding the transcription start site (TSS) of a beta-microseminoprotein (MSMB) gene to distinguish between BPH and prostate cancer. Furthermore the methylation status of CpG dinucleotides regulatory region surrounding the transcription start site (TSS) of a beta-microseminoprotein (MSMB) gene has been proven to provide a power to distinguish between a hormone-refractory prostate cancer (aggressive) and a hormone-sensitive prostate cancer. This is also part of the invention. Both diagnostic assays and methods can be combined to diagnose (together or in sequence) for the diseased status of the prostate. Moreover the invention provides methods and compositions for the diagnosis and for deciding the proper treatment of such prostate cellular proliferation disorders.

In a particular embodiment this invention relates to a diagnosis for a prostate proliferation disorder specifically by identification hypermethylations of non CpG dinucleotides (for instance, hypermethylation of CpA, CpT or CpC dinucleotides) in particular in a regulatory region surrounding the transcriptional start site of the beta-microseminoprotein (MSMB) gene or in particular upstream of the promoter region or in the promoter region of the beta-microseminoprotein (MSMB) gene. Such diagnosis on cell or tissue samples of the prostate allows specific distinguishing between tissues of benign prostate hyperplasia and prostate cancer.

More specifically, this invention also relates to a diagnosis for a prostate proliferation disorder specifically by identification hypermethylations in genomic regions that contain a high frequency of CG dinucleotides (CpG islands) and in particular in a regulatory region surrounding the transcriptional start site of the beta-microseminoprotein (MSMB) gene. Hypermethylations in genomic regions that contain a high frequency of CG dinucleotides (CpG islands) and in particular upstream of the promoter region or in the promoter region of the beta-microseminoprotein (MSMB) gene are indicative of a prostate-proliferative disorder that allows to distinguish between a hormone-sensitive and a hormone-refractory prostate cell proliferative disorder.

A more particular aspect of the invention relates to a diagnostic indicator of an androgen hormone-refractory prostatic tissue cellular proliferative disorder, for instance, an androgen hormone-refractory prostate cancer (PrCa).

Another aspect of the invention relates to a diagnostic indicator of 1) a benign prostate hyperplasia or a prostate cancer and 2) in case of a prostate cancer of an androgen hormone-sensitive prostatic tissue cancer or an androgen hormone-refractory prostatic tissue cancer.

The invention allows one to distinguish between hormone-refractory and hormones-sensitive cancer, particularly in prostatic tissues or cells originating from prostatic tissues. However, the test could also be used on body fluids.

The invention is broadly drawn to methods and assays for detecting a prostate proliferative disorder, in particular for identifying prostate tumor cells that have become refractory or resistant to hormone therapy, and thus allowing to identify the prostate cancer or/and to distinguish hormone-sensitive from hormone-refractory prostate cancers.

The invention relates generally to the identification of the distinguishing difference between a hormone-refractory prostate tissue cellular proliferative disorder and a hormone-sensitive prostate tissue cellular proliferative disorder in a subject, preferably a human subject. The distinguishing difference relies on the identification of one or more hypermethylated CpG islands surrounding the transcription start site (TSS) of the human gene for beta-microseminoprotein (MSMB), more in particular hypermethylated CpG islands are found in regions upstream of the TSS or in the promoter region of the human gene for beta-microseminoprotein (MSMB).

The prognostic methods that detect whether a prostate cancer in subjects, preferably human, comprises an androgen-refractory cancer and/or an androgen-sensitive cancer can be carried by biopsy and analysis of the hypermethylation status of the MSMB gene.

Thus, in a first aspect, the invention provides methods for detecting in a subject of prostate cell proliferative disorder, which methods comprise the steps of:
(a) obtaining a biological sample from the subject;
(b) determining the methylation state of CpG island upstream and/or downstream of the TSS and/or in the promoter region of the MSMB gene, for instance, the CpG islands about 3.5 kb upstream and about 2.2 kb downstream of the transcriptional start site in the MSMB gene in the subject's sample; and wherein detection of hypermethylation is indicative of a predisposition to, or the incidence of, prostate cancer.

In a similar aspect, the invention provides methods for detecting in a subject an androgen-refractory prostate cancer, which methods comprise the steps of:
(a) obtaining a biological sample from the subject;
(b) determining the methylation state of CpG island upstream and/or downstream of the TSS region and/or in the promoter region of the MSMB gene, for instance, the CpG island about 3 kb upstream and about 2.2 kb downstream of the transcriptional start site in the MSMB gene in the subject's sample; and wherein detection of hypermethylation is indicative of a predisposition to, or the incidence of, androgen-sensitive prostate cancer.

Preferably, both the methods of the invention comprise a further step as follows:
(c) identifying hypermethylation of region(s), wherein hypermethylation is identified as being different when compared to the same region(s) of the gene or associated regulatory region in a subject having an androgen-sensitive prostate cancer.

Another aspect of the invention is that it provides methylation conditions of regulatory regions of the MSMB gene, such as in the CpG islands surrounding the TSS of the human gene for beta-microseminoprotein (MSMB), which can be used (a) to analyze the presence of cancer cells in prostate tissue and/or in prostatic secretions, for instance, in seminal plasma, (b) to define patients that have a prostate cancer or alternatively patients that have a normal prostate, and (c) to define which patients with a prostate cancer have an androgen-refractory prostate cancer or alternatively to define which patients with a prostate cancer have a hormone-sensitive prostate cancer.

Such test provides an accurate means or tool to decide about the suitable treatment of the prostate cancer; in particular if the MSMB gene is methylated/hypermethylated the need for chemotherapy, surgery or radiation therapy is identified. The methods of the invention can also be used to predict effectiveness of such chemotherapies applicable on a prostate cancer.

Patients affected by a condition of hypermethylation of regulatory regions of the MSMB gene such as in the CpG islands surrounding the TSS of the MSMB gene, and/or CpG islands upstream of the TSS or in the promoter region of the MSMB gene can, for instance, be treated by DNA methyltransferase (DNMT) inhibitors or can be treated with inhibitors of the EZH2 gene expression or inhibitors of the function of the polycomb protein EZH2 to induce a repair of abnormal methylation.

Still another aspect of the invention relates to the observation that due to the fact that the MSMB gene, which encodes PSP94 (beta-microsemenoprotein or beta-inhibin), a prostatic secretory protein of 94 amino acids, or PSP57 (lacking an internal exon of 106 bases in the coding region resulting in a frameshift at the 3′ end, compared to PSP94) is repressed in hormone-refractory cancer cells, by the hypermethylation of a CpG island in the regulatory regions surrounding the transcriptional start site of the MSMB gene or in the promoter region that the encoding by the MSMB gene or expression of PSP94, known to be a suppressor of tumor growth and metastasis and to be secreted by the prostate gland and functions, is lost in advanced hormone-refractory cancer, for instance, advanced hormone-refractory prostate cancer. PSP57 mRNA is in prostate tumor cell lines, aberrantly spliced and localized in the nuclear fraction of the cell. (J. W. Xuan et al., Oncogene 1995 Sep. 21, 11(6):1041-7.) PSP57 mRNA has been also detected in other urogenital tissues (kidney, bladder) and in most tumor cell lines tested, but was not detectable in other tissues such as breast and lung. (R. Hoffmann et al., Nature Genetics 36:664 (2004).)

Hypermethylation can be detected by restriction endonuclease treatment and Southern blot analysis. Therefore, in a method of the invention, when the cellular component detected is DNA, restriction endonuclease analysis is preferable to detect hypermethylation of the MSMB regulatory region, in the promoter or upstream of the promoter. Any restriction endonuclease that includes CG as part of its recognition site and that is inhibited when the C is methylated can be utilized. Preferably, the methylation-sensitive restriction endonuclease is BssHII, MspI, or HpaII, used alone or in combination. Other methylation-sensitive restriction endonucleases will be known to those of skill in the art.

Additional indicators can be part of the diagnostic method of the invention; Moreover the MSMB gene can in androgen-refractory prostate cancer cells, but not in androgen-sensitive prostate cancer cells be trimethylated on histone H3 K27 and the MSMB can be additionally repressed in androgen-refractory prostate cancer cells by the hypoacetylation of H3K9. Assaying for this trimethylation status or this hypoacetylation status can be an additional part of the diagnostic assay or the diagnostic method to distinguish between an androgen-refractory and an androgen-sensitive prostate cancer.

By the invention, MSMB has been demonstrated and validated to be a true target for repression by the histone methyltransferase EZH2. The identification of MSMB as an EZH2 target gene can explain why the expression of this tumor suppressor gene is lost in advanced stages of prostate cancer. We demonstrated that the increased expression of EZH2 in metastatic prostate cancer results in H3K27 trimethylation of the MSMB gene. This leads to the recruitment of the PRC1 complex and MSMB silencing. In addition, EZH2 binds to DNA methyltransferases and, indirectly, histone deacetylases and these enzymes also contribute to the maintenance of MSMB silencing. Our data demonstrate that specific inhibitors of EZH2 are useful for the treatment of metastatic prostate cancer, at least in part because such inhibitors are expected to reverse the down-regulation of the tumor suppressor PSP94.

Furthermore, the invention relates generally to the demonstration that the expression of the tumor suppressor PSP94 by MSMB is silenced by EZH2 in advanced prostate cancer cells and that an increased expression of the polycomb protein EZH2 (enhancer of zeste homolog 2), represses transcription via trimethylation of histone H3 on Lys27 (H3K27). The RNAi-mediated knockdown of EZH2 resulted in a loss of H3K27 trimethylation and an increased expression of the MSMB gene. Conversely, the overexpression of EZH2 was associated with a decreased expression of the MSMB gene. PSP94, for prostatic secretory protein of 94 amino acids, is secreted by the prostate gland and functions as a suppressor of tumor growth and metastasis. The expression of PSP94 is lost in advanced, hormone-refractory prostate cancer. Present invention now demonstrates that this decrease of PSP94 expression correlates with an increased expression of the polycomb protein EZH2 (enhancer of zeste homolog 2), which represses transcription via trimethylation of histone H3 on Lys27 (H3K27) and that these events are causally related and that the MSMB gene, which encodes PSP94, is trimethylated on H3K27 in androgen-refractory, but not in androgen-sensitive prostate cancer cells.

By the invention, it has been demonstrated and validated that the gene encoding the prostatic tumor suppressor PSP94 is a target for repression by the polycomb group protein EZH2. For instance, chromatin immunoprecipitation experiments confirmed an association of EZH2 with the MSMB gene. The RNAi-mediated knockdown of EZH2 resulted in a loss of H3K27 trimethylation and an increased expression of the MSMB gene. Conversely, the overexpression of EZH2 was associated with a decreased expression of the MSMB gene. We also demonstrate that MSMB is additionally repressed in androgen-refractory prostate cancer cells by the hypoacetylation of histone H3K9 and the hypermethylation of a CpG island in the promoter region. Present invention demonstrates a hitherto unexplored link between the putative oncogene EZH2 and the tumor suppressor PSP94, and show that MSMB is silenced by EZH2 in advanced prostate cancer cells.

The diagnostic method and assay of the invention is thus indicative for the suitability of a treatment of a prostate cell proliferation disorder and in particular a prostate cancer, for instance, an anti-EZH2 treatment by a therapeutically effective amount of an EZH2 inhibitor to prevent the transition of an androgen-refractory prostate cancer to an androgen insensitive prostate cancer. Anti-EZH2 treatment in the art is, for instance, EZH2 siRNA, for instance, the Small interfering RNA (siRNA) duplexes4 targeted against EZH2 reduce the amounts of EZH2 protein present in prostate cells (S. M. Elbashir et al., Nature 411:494-498 (2001)).

Diagnosis of hypermethylation of the CpG island in the regions surrounding the TSS or in the promoter of the MSMB gene and preferably about 3 kb upstream of the transcriptional start site in the MSMB gene can thus be used as a decision toll for treatment of a patient affected with such hypermethylation with a therapeutically effective amount of an DNA methyltransferase (DNMT) inhibitor for treating the prostate cancer or for preventing that a androgen sensible prostate cancer evolves into an androgen-refractory prostate cancer. MGI Pharma developed small molecule DNA methyltransferase (DNMT) inhibitors for the treatment of cancer. Short oligonucleotide DNA methylation inhibitors in the art are Decitabine 5-Aza-CdR, S110 AzapG, S53 GpAza, S54 GpAzapG, S55 AzapGpAzapG, S56 pGpAzapAzapG, S52R AzapsG, Zebularine and S112 HEGpAzapG. A specific DNMT inhibitor is, for instance, the compound with the structure D in FIG. 6 called S110 or S110 of the company SuperGen which is a dinucleotide containing decitabine, S110, which has superior activity due to increased stability because of less degradation by hydrolytic cleavage and deamination. This is a DNA demethylating agent with a similar activity as decitabine (5-aza-2′-deoxycytidine) or its derivatives. Decitabine is a potent DNA methylation inhibitor which is approved in the US for the treatment of myelodysplastic syndromes (D. B. Yoo et al., Cancer Research 67:6400-6408, No. 13, 1 Jul. 2007). Another DNA methyltransferase (DNMT) inhibitor is MG 98 (HYB 101584) is described in U.S. Pat. No. 6,953,783 and U.S. Pat. No. 6,506,735. MG 98 is a second generation antisense oligonucleotide that selectively targets DNA methyltransferase 1 (DNMT1) mRNA. By inhibiting the production of DNMT, the methylation of DNA is reversed and leads to re-expression of the tumor suppression genes. MG 98 is created by MethylGene Inc. (D. Stewart et al., 11th NCI-EORTC-AACR symposium on new drugs in cancer therapy, 148, 7 Nov. 2000; E. Winquist et al., European Journal of Cancer 38 (Suppl. 7):141, Nov. 2002; D. J. Stewart et al., Annals of Oncology 14:766-774, May 2003; S. Ramchandani et al., Proceedings of the National Academy of Sciences of the United States of America 94:684-689, January 1997; and A. J. Davis et al., 11th NCI-EORTC-AACR symposium on new drugs in cancer therapy, 94, 7 Nov. 2000. These compounds can be administered in a therapeutically efficient amount to patients that have been identified by the diagnostic method of the invention to be in need thereof.

Thus, epigenetic loss of gene function due to hypermethylation can be rescued by the use of DNA demethylating agents and/or DNA methyltransferase inhibitors and/or HDAC inhibitors. Accordingly, the invention also provides for a method for predicting the likelihood of successful treatment of prostate proliferative disorder or prostate cancer, with a DNA demethylating agent and/or a DNA methyltransferase inhibitor and/or HDAC inhibitor comprising detecting a methylation change in the region surrounding the TSS or the promotor region of the MBMS gene wherein detection of the methylation change is indicative of successful treatment to a higher degree than if the methylation modification is not detected.

Also provided is a kit for detecting a predisposition to, or the incidence of, prostate cancer in a sample comprising:
(a) means for detecting a methylation change in the region surrounding the TSS or the promotor region of the MBMS gene
(b) means for processing a sample derived from the prostate.

In certain embodiments, a method of diagnosing a disease state or cell proliferative disorder in the prostate of a subject, the method comprising: (a) analyzing the level DNA methylation of regulatory region surrounding the transcription start site (TSS) of a beta-microseminoprotein (MSMB) gene or a homologous sequence in a biological sample isolated from the subject, and (b) comparing the DNA methylation with the DNA methylation in a control sample and/or a benign prostate hyperplasia sample; whereby non-CpG methylation or increased non-CpG methylation relative to the control sample or the benign prostate hyperplasia sample in the regulatory regions surrounding the transcriptional start site of the MSMB gene is an indication for prostate cancer and whereby methylation of the CpG dinucleotides or an increased level methylation of the CpG dinucleotides in the regulatory region surrounding the transcriptional start site of the MSMB gene relative to a control sample or relative to the benign prostate hyperplasia sample is an indication of a hormone-refractory prostate cancer, androgen-independent prostate cancer (AIPC) or androgen-independent metastatic prostate cancer.

In certain embodiments, disclosed is a method of diagnosing a disease state or cell proliferative disorder in the prostate in a subject, the method comprising: (a) analyzing the level DNA methylation of the CpG island in the promoter and upstream of the promoter of a beta-microseminoprotein (MSMB) gene or a homologous sequence in a biological sample isolated from the subject, and (b) comparing the DNA methylation with the DNA methylation in a control sample and/or a benign prostate hyperplasia sample; whereby non-CpG methylation or increased non-CpG methylation relative to the control sample or the benign prostate hyperplasia sample in the regulatory regions surrounding the transcriptional start site of the MSMB gene is an indication for prostate cancer and whereby methylation of the CpG dinucleotides or an increased level methylation of the CpG dinucleotides in the regulatory region surrounding the transcriptional start site of the MSMB gene relative to a control sample or relative to the benign prostate hyperplasia sample is an indication of a hormone-refractory prostate cancer, androgen-independent prostate cancer (AIPC) or androgen-independent metastatic prostate cancer.

In certain embodiments, disclosed is a method of diagnosing a disease state or cell proliferative disorder in the prostate in a subject, the method comprising: (a) analyzing the level DNA methylation of the transcriptional start site (TSS) of the beta-microseminoprotein (MSMB) gene or a homologous sequence, in particular in regions upstream the TSS or in its promoter region, in a biological sample isolated from the subject, and (b) comparing the DNA methylation with the DNA methylation in a control sample and/or a benign prostate hyperplasia sample; whereby non-CpG methylation or increased non-CpG methylation relative to the control sample or the benign prostate hyperplasia sample in the regulatory regions surrounding the transcriptional start site of the MSMB gene is an indication for prostate cancer and whereby methylation of the CpG dinucleotides or an increased level methylation of the CpG dinucleotides in the regulatory region surrounding the transcriptional start site of the MSMB gene relative to a control sample or relative to the benign prostate hyperplasia sample is an indication of a hormone-refractory prostate cancer, androgen-independent prostate cancer (AIPC) or androgen-independent metastatic prostate cancer.

In certain embodiments, a method of diagnosing a disease state or cell proliferative disorder in the prostate in a subject, the method comprising: (a) analyzing the level DNA methylation of regulatory regions surrounding the transcriptional start site of the beta-microseminoprotein (MSMB) gene in a biological sample isolated from the subject, and (b) comparing the DNA methylation with the DNA methylation in a control sample and/or a benign prostate hyperplasia sample; whereby non-CpG methylation or increased non-CpG methylation relative to the control sample or the benign prostate hyperplasia sample in the regulatory regions surrounding the transcriptional start site of the MSMB gene is an indication for prostate cancer and whereby methylation of the CpG dinucleotides or an increased level methylation of the CpG dinucleotides in the regulatory region surrounding the transcriptional start site of the MSMB gene relative to a control sample or relative to the benign prostate hyperplasia sample is an indication of an androgen-independent metastatic prostate cancer.

In certain embodiments, a method of diagnosing a disease state or cell proliferative disorder in the prostate in a subject, the method comprising: (a) analyzing the level DNA methylation in the CpG4-5 region of the MSMB gene of a beta-microseminoprotein (MSMB) gene or a homologous sequence in a biological sample isolated from the subject, and (b) comparing the DNA methylation with the DNA methylation in a control sample and/or a benign prostate hyperplasia sample; whereby non-CpG methylation or increased non-CpG methylation relative to the control sample or the benign prostate hyperplasia sample in the regulatory regions surrounding the transcriptional start site of the MSMB gene is an indication for prostate cancer and whereby methylation of the CpG dinucleotides or an increased level methylation of the CpG dinucleotides in the regulatory region surrounding the transcriptional start site of the MSMB gene relative to a control sample or relative to the benign prostate hyperplasia sample is an indication of a hormone-refractory prostate cancer, androgen-independent prostate cancer (AIPC) or androgen-independent metastatic prostate cancer.

Further specific embodiments of these previous methods of diagnosis can be:

The previous method of diagnosing further comprising a step of analyzing histone (de)acetylation of the MSMB gene in the sample.

The previous method of diagnosing whereby the disease state or cell proliferative disorder is a cancer.

The previous method of diagnosing to distinguish between a healthy prostate and a disordered or diseased prostate.

The previous method of diagnosing to distinguish between a benign prostate hyperplasia and a prostate cancer.

The previous method of diagnosing to distinguish between a hormone-sensitive prostate cancer and a hormone-refractory prostate cancer.

The previous method of diagnosing to distinguish between an androgen-sensitive prostate cancer or androgen dependent prostate cancer and androgen-independent prostate cancer (AIPC).

The previous method of diagnosing to discover an androgen-independent metastatic prostate cancer in a prostate cell or prostate tissue.

The previous method of diagnosing to carry out a prostate cancer grading or prostate cancer staging.

The previous method of diagnosing to decide on the proper treatment or proper medicament of the prostate disease state.

The previous method of diagnosing to decide on the treatment with a pharmaceutically acceptable DNA methylation inhibitor.

The previous method of diagnosing to decide on the treatment with a pharmaceutically acceptable HDAC inhibitor.

The previous method of diagnosing to decide on the treatment to decrease the activity of the EZH2 protein.

The previous method of diagnosing to decide on the treatment with a DNA demethylating agent and/or a DNA methyltransferase inhibitor and/or HDAC inhibitor.

The previous method of diagnosing to decide on a prophylactically effective amount of a nutraceutical. To treat a subject with a prostate disease status.

Further scope of applicability of the invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

### DESCRIPTION OF THE DRAWINGS

FIG. 1 demonstrates MSMB and EZH2 expression in prostatic epithelial cells and trimethylation of the MSMB gene on H3K27. (a) The relative amounts of EZH2 (left panel) and MSMB transcripts (right panel) in the indicated prostate cell lines were determined by quantitative RT-PCR with intron-spanning primers. Human prostate PC-3 cells (adenocarcinoma), LNCaP cells (carcinoma) and DU 145 cells (carcinoma) were cultured as monolayers in 50% Dulbecco's modified Eagle's medium (DMEM) and 50% Ham's F12, RPMI1640 and DMEM, respectively, supplemented with 10% fetal calf serum. PZ-HPV-7 cells, an immortalized cell line derived from normal human prostate cells, were cultured in keratinocyte-serum free medium supplemented with 5 ng/ml human recombinant epidermal growth factor and 0.05 mg/ml bovine pituitary extract. Total RNA was isolated using the Genelute Mammalian Total RNA Miniprep kit (Sigma, St. Louis, Mo., USA). A total of 1-5 mg RNA was reverse-transcribed with oligo dT primer (Sigma) and the M-MulV reverse transcriptase (Fermentas GMBH, St. Leon-Rot, Germany). cDNA (1.5%) was analyzed by real-time PCR in triplicate using a Platinum SYBR Green qPCR SuperMix-UDG (Invitrogen, Paisly, UK) in a Rotorgene detection system (Corbett Research, Cambridge, UK) and normalized to the housekeeping gene hypoxanthine-guanine-phosphoribosyl-transferase (HPRT). All used primer sequences are available on request. The data represent the means±s.e. of at least three independent experiments. (b) Schematic representation of the MSMB gene on scale. The four exons are indicated by the black boxes. The black lines below the MSMB locus represent the fragments (1-5) amplified by ChIP analysis. (c) ChIPs on PC-3 (left panel) and LNCaP (right panel) cells were performed with 10 μg of control antibodies (rabbit anti-mouse IgGs, Dakocytomation, Gostrup, Denmark) or 5 μg of antibodies against H3K27me3 (polyclonal anti-H3K27, Upstate, Dundee, UK). ChIP reactions were performed according to the protocol of Upstate, Dundee, UK. The DNA was recovered with the Genelute PCR clean-up kit (Sigma) and analyzed by real-time PCR. Numbers 1-5 refer to the MSMB fragments that were amplified (see panel b). The data represent the means±s.e. of at least three independent experiments in duplicate and indicate the fold enrichment as compared to the negative control (IgGs). The enrichment of DNA was calculated using the formula:fold enrichment=^{2(CtlgG-CtAb)}, where Ct is the threshold cycle, IgG is the normal rabbit IgG and Ab is the specific antibody.

FIG. 2 concerns how EZH2 is recruited to the MSMB gene and causes trimethylation of H3K27. (a) ChIP assays were performed in PC-3 cells using rabbit antibodies against a synthetic peptide of human EZH2 (E. Viré et al. (2006), Nature 439:871-874), trimethylated H3K27 (Upstate), trimethylated H3K9 (Upstate) and control IgGs. ChIP results were revealed by EtBr staining of agarose gels containing PCR-amplified ChIP DNA. (b) Immunoblotting (upper panel) and quantitative RT-PCR analysis (lower panel) of PC-3 cell lysates, obtained after transfection with either a control siRNA (Ctr), that is, a scrambled version of a siRNA duplex for the housekeeping gene PPP1R8 (GGAACUCGAACCUCCACGAACAAUU (SEQ ID NO:71), Invitrogen) or an EZH2 siRNA (KD; AAGACUCUGAAUGCAGUUGCU (SEQ ID NO:72), Dharmacon, Chicago, Ill., USA). The PC-3 cells were plated at 1.2×10⁶ cells in a 10 cm plate. At 24 hours after plating, the cells were transfected with 300 nM of siRNA duplex using Lipofectamine 2000 (Invitrogen). At 48 hours after transfection, the cells were harvested. SIPP1 served as a loading control for the immunoblotting and ACTIN was used as control for normalization in the quantitative RT-PCR. (c and d) ChIP assays were performed on chromatin obtained from PC-3 cells transfected with either control (Ctr) or EZH2 siRNAs, using control antibodies (rabbit IgGs), antibodies against EZH2 (c) or antibodies against H3K27me3 (d). The immunoprecipitated DNA was analyzed by quantitative PCR using primers specific for the MSMB gene (FIG. 1, Panel b). The enrichment on MSMB is expressed as a %±s.e. of the control value (n=3-4). (e) PC-3 cells were transfected with control (A lamin A/C siRNA duplex (AACUGGACUUCCAGAAGAACA (SEQ ID NO:73), Dharmacon) or EZH2 siRNAs for 48 hours. The steady-state transcript levels of MSMB, EZH2 and the housekeeping gene PPP1R8 were determined by quantitative RT-PCR analysis with intron-spanning primers specific for the indicated genes and were expressed relative to the transcript level in the control condition. ACTIN was used as a control for normalization. (f) PZ-HPV7 cells were transiently transfected with an expression vector encoding either Gal4-tag alone or its fusion with EZH2 using lipofectamine plus (Invitrogen), according to manufacturer's instructions. The analysis was carried out as described in (e), with the housekeeping gene HPRT as negative control.

FIG. 3 demonstrates that the MSMB gene is regulated by histone (de)acetylation. (a) PC-3 cells were treated for nine hours with 50 ng/ml of the histone deacetylase inhibitor trichostatin A (TSA, Sigma). Subsequently, the steady-state levels of the MSMB and EZH2 transcripts were determined by quantitative RT-PCR analysis with intron-spanning primers specific for the indicated genes. The data are expressed relative to the transcript level in the control condition. HPRT was used as a control for normalization. (b) ChIPs on PC-3 (left panel) and LNCaP (right panel) cells were performed with 10 μg of control antibodies (rabbit anti-mouse IgGs, Dakocytomation) or 5 μg of antibodies against H3K9ac (polyclonal anti-H3K9ac, Upstate). The data represent the means±s.e. of three independent experiments in duplicate and indicate the fold enrichment as compared to the negative control with IgG.

FIG. 4 demonstrates that the MSMB gene is regulated by methylation of a CpG island in the promoter region. (a) PC-3 cells were treated for 48 hours with 10 mM of the DNA methyltransferase inhibitor 50-azacytidine. Subsequently, the steady-state levels of the MSMB and EZH2 transcripts were determined by quantitative RT-PCR analysis with intron-spanning primers specific for the indicated genes. The data were expressed relative to the transcript level in the control condition. HPRT was used as a control for normalization. (b) A schematic representation of the MSMB gene on scale. The four exons are indicated by the black boxes and the two analyzed CpG regions by black stars. TSS, transcriptional start site. Methylated CG dinucleotides are denoted underneath by closed circles and unmethylated CGs by open circles. Genomic DNA of PC-3 cells or LNCaP cells was purified with the GenElute Mammalian Genomic DNA Miniprep kit of Sigma. Two microgram was digested overnight with BglII. The DNA was denatured with 0.3 M NaOH at 421° C. for 30 minutes. Sodium bisulfite (3.3 M) and hydroquinone (0.5 mM) were added to the DNA and the mixture was incubated overnight at 55° C.

The DNA was purified with the PCR purification kit of Sigma, St Louis, Mo., USA. The DNA was desulfonated with 0.3 M NaCl for 15 minutes at 37° C. and precipitated by adding NH4Ac and ethanol. The pellet was air-dried and dissolved in 10 mM Tris and 1 mM ethylene-diaminete-traacetic acid at pH 8. PCR was performed with Jumpstart Taq Polymerase (Sigma). The primers GTTTAGGTTGGAGTGTAGTGG (SEQ ID NO:74) (sense) and ATCCTAACTAACATAATAAAACCCC (SEQ ID NO:75) (antisense) were used to amplify the first CpG island and the primers AGTTTTTTTATTTAGGGGTGGATTTTA (SEQ ID NO:76) (sense) and CCAAACTAATCTCAAATACCTAACCTC (SEQ ID NO:77) (antisense) were used to amplify the second CpG island. The PCR products were subcloned in the pGem-T vector of Promega according to the manufacturer's protocol. At least ten clones for each condition were sequenced. The plasmids were sequenced on a MegaBace sequencer. The percentages of methylated CpG dinucleotides are indicated in the bar diagrams.

FIG. 5 displays chemical structures of DNA methylation inhibitors: 5-aza-CdR (A), S52 (B), S53 (C), S110 (D), and S112 (E). 5-Aza-CdR is a deoxycytidine with an extra nitrogen at the 5-position of the pyrimidine ring. S52 is a phosphorothioate analogue of S110. There are two optical isomers of S52: S52S and S52R. S53 is a dinucleotide with a guanosine at the 5′-end and 5-aza-CdR at the 3′-end. S110 is a reverse dinucleotide of S53 containing a 5-aza-CdR at the 5′-end followed by a guanosine. S112 is a triethylamine salt of 5′-AzapG-3′ dinucleotide with a hexaethylene glycol phosphate at the 5′-end.

FIG. 6 provides a schematic representation of SEQ ID NO:5: 15000 bp upstream and downstream of the transcriptional start site (TSS) (first nucleotide of exon 1) of the MSMB gene. The MSMB gene is located on the forward strand of chromosome 10 from 51219559 to 51232596 with transcriptional start site (TSS)=51219559. The sequence (SEQ ID NO:5) was obtained from the Homo sapiens chromosome 10 genomic contig with the accession number NT_{—}008583.16 (Hs10_{—}8740:85708-115707). All CpG islands, except for CpG5 and 7, are predicted by the program Newcpgreport, with the following parameters: window=50, window shift=1, Island size>200, GC %>0.2 and O/E (observed/predicted>0.2. CpG5 and CpG7 islands are predicted by the program Methprimer with the following parameters: Island size>100, GC %>0.5 and O/E>0.6. The exact location of the CpG islands are described in Table 1. The predicted CpG islands (CpG 1-CpG 10) are shown by Shaded arrows and exons (E1-E4) by shaded boxes.

SEQ ID NO:5 represents genomic sequences of the forward strand of chromosome 10 from 15000 nt upstream and downstream of the transcriptional start site (TSS) of the MSMB gene and SEQ ID NO:6 represent the bisulfite converted sequence thereof. The TSS of the MSMB gene is located at position 51219559 on the forward strand of chromosome 10.

SEQ ID NO:7 represents genomic sequences of the reverse strand of chromosome 10 from 15000 nt upstream and downstream of the transcriptional start site (TSS) of the MSMB gene and SEQ ID NO:8 represent the bisulfite converted sequence thereof. The TSS of the MSMB gene is located at position 51219559 on the forward strand of chromosome 10.

### DETAILED DESCRIPTION OF THE INVENTION

The invention demonstrates that the MSMB gene is silenced by DNA methylation of regulatory regions surrounding the transcriptional start site (TSS) of the concerned gene, in particular in regions upstream the TSS or in its promoter region. Novel specific CpG islands have been such as the CpG islands about 3 kb upstream and about 2.2 kb downstream of the transcriptional start site in the MSMB gene and showed by bisulphite sequencing that in the androgen-independent metastatic PrCa PC-3 cells the CpG dinucleotides were methylated.

Furthermore, we could show that the MSMB gene can be reactivated in PC-3 cells by the addition of the DNA methyltransferase inhibitor 5′ azacytidine and that this was associated with a decreased methylation of the upstream CpG island.

Importantly, the CpG island about 3 kb upstream of the transcriptional start site in the MSMB gene was hypomethylated in the androgen-sensitive LNCaP cells as compared to its methylation status in the androgen-refractory PC-3 cells. This agrees with the higher expression of the MSMB gene in LNCaP cells as compared to its expression in PC-3 cells.

It has been demonstrated that the methylation of CpG sites is the major factor underlying the transcriptional silencing of the MSMB gene in PrCa cell lines and this suggests that the DNA methylation state of the MSMB gene can be used as a biomarker for PrCa.

“Disease state” as used herein means any disease, disorder, condition, symptom, or indication.

As used herein, the term “cell proliferative disorder” refers to conditions in which the unregulated and/or abnormal growth of cells can lead to the development of an unwanted condition or disease, which can be cancerous or non-cancerous. The detection of the cell proliferative disorder may be by way of routine examination, screening for a cell proliferative disorder or pre-stadia such cell proliferative disorder, monitoring and/or staging the state and/or progression of the cell proliferative disorder, assessing for recurrence following treatment, and monitoring the success of a treatment regimen. In certain embodiments, the cell proliferation disorder is cancer.

As used herein, the term “cancer” as used herein concerns malignant neoplasm, malignant tumor or invasive tumor and also can include solid neoplasm or solid tumors cancers. Cancers are classified by the type of cell that resembles the tumor and, therefore, the tissue presumed to be the origin of the tumor. Examples of general categories include: Carcinoma: Malignant tumors derived from epithelial cells. This group represents the most common cancers, including the common forms of breast, prostate, lung and colon cancer. Sarcoma: Malignant tumors derived from connective tissue, or mesenchymal cells. Lymphoma and leukemia: Malignancies derived from hematopoietic (blood-forming) cells Germ cell tumor: Tumors derived from totipotent cells. In adults most often found in the testicle and ovary; in fetuses, babies, and young children most often found on the body midline, particularly at the tip of the tailbone; in horses most often found at the poll (base of the skull). Blastic tumor: A tumor (usually malignant) which resembles an immature or embryonic tissue.

“Hormone-refractory prostate cancer” and in particular “androgen-independent prostate cancer (AIPC)” has to be understood for the meaning of this invention as prostate cancer that has become refractory, that is, it no longer responds to hormone therapy.

“Prostate cancer grading” as used herein means describing how abnormal or aggressive the cancer cells appear. The grade helps to predict long-term results, response to treatment and survival. In the art there is, for instance, the Gleason scale that is the most common scale used for grading prostate cancer. This system assigns cancer cells a score from 1 to 10, by combining the two most common patterns of cells to give a total score (i.e., 3+4=grade 7). Scores generally range between 4 and, most commonly, 6 or 7. These scores are broken down into three main levels: Low-grade (well differentiated): This type of slow-growing cancer has an appearance most like normal prostate cells and is the least dangerous. It has a Gleason score of 4 or less. Intermediate grade (moderately differentiated): This type is somewhere between the low- and high-grade cancers and the most common of the three. Depending on PSA level and tumor volume, it can act like a high- or low-grade cancer. It has Gleason score between 4 and 7. High-grade (poorly differentiated). This type of cancer has an appearance least like normal prostate cells. It is the most deadly since it is very aggressive and grows very fast—even into surrounding areas such as lymph nodes and bones. These cancer cells also tend to be large, hard to treat, and reappear more frequently. They have a Gleason score between 8 and 10 (A. S. Perry et al., Endocrine-Related Cancer 13 (2) 357-377).

“Prostate cancer staging” as used herein concerns how much and where the cancer is located. The more cancer there is in the body, the more likely it is to spread and less likely that treatments will work. Therefore, the more advanced stages can affect long-term results and survival. According an older prostate cancer staging the prostate cancer is broken down into four primary stages, for instance, the four ABCD stages of staging to gauge the severity of prostate cancer to describe the detection and location of the cancer. Stage A: Cancer found when not suspected or due to a high PSA level, Stage B: Cancer found due to abnormal digital rectal exam and is held in the prostate, Stage C: Cancer that has spread to the tissues outside of the prostate, Stage D: Cancer that has spread to the lymph nodes or bone. A particular system in the art which replaced the ABCD staging system of prostate cancer to give an even more accurate description of the cancer is the TNM grading system. “T” describes the tumor and uses different numbers to explain how large it is; “N” stands for nodes and tells whether the cancer has spread to the lymph nodes; “M” means metastatic, and tells whether the cancer has spread throughout the body. There are various T Status stages: Stage T1: Microscopic tumor confined to prostate and undetectable by a digital rectal exam (DRE) or ultrasound; Stage T1a: Tumor found in 5% or less of prostate tissue sample; Stage T1b: Tumor found in more than 5% of a prostate tissue sample; Stage T1c: Tumor is identified by needle biopsy as a follow-up to screening that detected elevated PSA results; Stage T2: Tumor confined to prostate and can be detected by DRE or ultrasound; Stage T2a: Tumor involves less than half of one lobe of the prostate, and can usually be discovered during DRE exam; Stage T2b: Tumor involves more than half of one lobe of the prostate, and can usually be felt during DRE exam; Stage T2c: Tumor involves both lobes of the prostate and is felt during a DRE exam; Stage T3: Tumor has spread to surrounding tissues or to the seminal vesicles; Stage T3a: Tumor has spread to outside of the prostate on only one side; Stage T3b: Tumor has spread to outside of the prostate on both sides; Stage T3c: Tumor has spread to one or both of the seminal tubes; Stage T4: Tumor is still within the pelvic region but may have spread to organs near the prostate, such as the bladder; Stage T4a: Tumor has spread beyond the prostate to any or all of the bladder neck, the external sphincter, and/or the rectum and Stage T4b: Tumor has spread beyond the prostate and may affect the levator muscles (the muscles that help to raise and lower the organ) and/or the tumor may be attached to the pelvic wall and various N Status stages: Stage N0: Cancer cells have spread, but not yet to pelvic lymph nodes; Stage N1: Cancer cells have spread to a single lymph node in the pelvic area and are 2 cm (approximately ¾ of one inch) or less in size; Stage N2: Cancer cells have spread either to a single lymph node and are more than 2 cm but less than 5 cm (approximately 2 inches) in size, or the prostate cancer cells are found in more than one lymph node and are no larger than 5 cm in size; Stage N3: Cancer cells have spread to the lymph nodes and are larger than 5 cm in size and various M Status stages: Stage M0: Cancer cells have spread, but only regionally in the pelvic area & Stage M1: Cancer cells have spread beyond the pelvic area to other parts of the body (Dr. F. H. Schröder et al., The Prostate Volume 21, Issue S4, pp. 129-138, 20 Jul. 2006).

As used herein, the term “effective amount” refers to an amount of a compound, or a combination of compounds, of the invention effective when administered alone or in combination as an anti-proliferative agent. For example, an effective amount refers to an amount of the compound present in a formulation or on a medical device given to a recipient patient or subject sufficient to elicit biological activity, for example, anti-proliferative activity, such as e.g., anti-cancer activity or anti-neoplastic activity. The combination of compounds optionally is a synergistic combination. Synergy, as described, for example, by Chou and Talalay, Adv. Enzyme Regul. vol. 22, pp. 27-55 (1984), occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, or increased anti-proliferative effect, or some other beneficial effect of the combination compared with the individual components.

A “therapeutically effective amount” as used herein means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated. A therapeutically effective amount of one or more of the compounds can be formulated with a pharmaceutically acceptable carrier for administration to a human or an animal. Accordingly, the compounds or the formulations can be administered, for example, via oral, parenteral, or topical routes, to provide an effective amount of the compound. In alternative embodiments, the compounds prepared in accordance with the invention can be used to coat or impregnate a medical device.

The term “prophylactically effective amount” as used herein means an effective amount of a compound or compounds, of the invention that is administered to prevent or reduce the risk of unwanted cellular proliferation.

“Pharmacological effect” as used herein encompasses effects produced in the subject that achieve the intended purpose of a therapy. In one preferred embodiment, a pharmacological effect means that primary indications of the subject being treated are prevented, alleviated, or reduced. For example, a pharmacological effect would be one that results in the prevention, alleviation or reduction of primary indications in a treated subject. In another preferred embodiment, a pharmacological effect means that disorders or symptoms of the primary indications of the subject being treated are prevented, alleviated, or reduced. For example, a pharmacological effect would be one that results in the prevention or reduction of primary indications in a treated subject.

“Prostate biopsy” as used herein is a procedure in which small samples are removed from a man's prostate gland to be tested for the presence of cancer. It is typically performed when the scores from a PSA blood test rise to a level that is associated with the possible presence of prostate cancer.

“Treating” includes any effect, e.g., lessening, reducing, modulating, or eliminating, that results in the improvement of the condition, disease, disorder, etc. “Treating” or “treatment” of a disease state includes: (1) preventing the disease state, i.e., causing the clinical symptoms of the disease state not to develop in a subject that may be exposed to or predisposed to the disease state, but does not yet experience or display symptoms of the disease state; (2) inhibiting the disease state, i.e., arresting the development of the disease state or its clinical symptoms; or (3) relieving the disease state, i.e., causing temporary or permanent regression of the disease state or its clinical symptoms.

By “homologous sequence” is meant, a nucleotide sequence that is shared by one or more polynucleotide sequences, such as genes, gene transcripts and/or non-coding polynucleotides. For example, a homologous sequence can be a nucleotide sequence that is shared by two or more genes encoding related but different proteins, such as different members of a gene family, different protein epitopes, different protein isoforms or completely divergent genes, such as a cytokine and its corresponding receptors. A homologous sequence can be a nucleotide sequence that is shared by two or more non-coding polynucleotides, such as noncoding DNA or RNA, regulatory sequences, introns, and sites of transcriptional control or regulation. Homologous sequences can also include conserved sequence regions shared by more than one polynucleotide sequence. Homology does not need to be perfect homology (e.g., 100%), as partially homologous sequences are also contemplated by the instant invention (e.g., 99%, 98%, 97%, 96%, 95%, 94%, 93%, 92%, 91%, 90%, 89%, 88%, 87%, 86%, 85%, 84%, 83%, 82%, 81%, 80% etc.).

With the “regulatory region surrounding the transcription start site (TSS)” is meant a regulatory region located upstream or 5′ to the TSS and/or a regulatory region around the TSS and/or a regulatory region located downstream or 3′ to the TSS of the concerned gene. The location of the concerned region can vary from 15 Kbp upstream to 15 Kbp downstream of the TSS. Thus the region under investigation may correspond to all or part of the promotor region of the MSMB gene. Alternatively, the region under investigation corresponds with an exon and/or intron region and/or TSS region of the MSMB gene. The up stream region of the MSMB gene is preferably between −13877 and −13583 or between −10528 and −10254 or between −3920 to −3673 or between −3471 and −3141 or −3128 and −2817 or between −3533 and −2734 base pairs from the transcription start site, and/or preferably between −452 bp upstream and +152 bp downstream from the TSS and/or between 1671 and 1996 or between 2180 and 2390 between 5236 and 5616 or between 7670 and 8030 or between 11432 and 11754 base pairs downstream from the transcription start site of the MSMB gene is analyzed for hypermethylation. Most preferably, the region extends from −3128 bp to −2817 bp upstream from the transcription start site of the MSMB gene and/or between −452 bp upstream and +152 bp downstream from the TSS and/or extends from 2180 bp to 2390 bp downstream from the transcription start site of the MSMB gene.

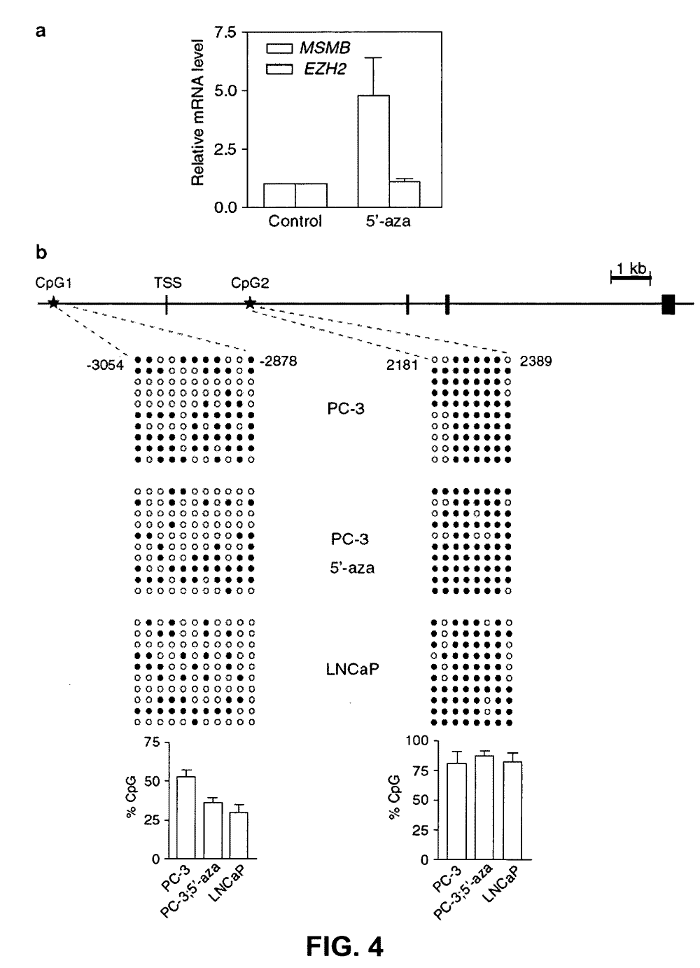

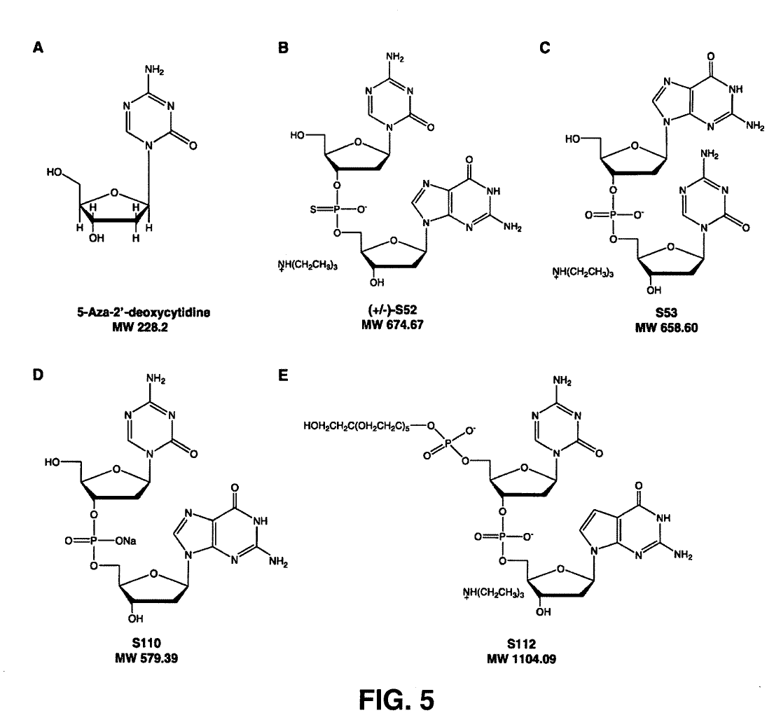

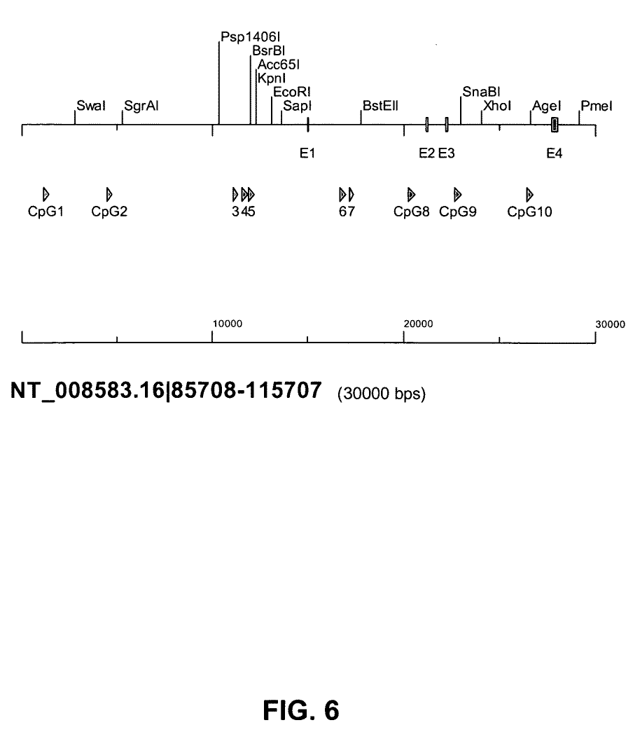

The term “promoter” refers to the regulatory region located upstream, or 5′ to the structural gene and/or TSS. Such a region extends typically between approximately 5 Kb, 500 bp or 150 to 300 bp upstream from the transcription start site of the concerned gene. For the MSMB gene, we identified at least two CpG islands (genomic regions that contain a high frequency of CG dinucleotides) surround the transcriptional start site, by the use of the bioinformatics program Methprimer (parameters: Island size>100 nt, GC %>0.5 and Observed/expected (O/E)>0.6; FIG. 4) but a more extended bioinformatics approach analysis (program Newcpgreport, with parameters: window=50, window shift=1, Island size>200, GC %>0.2 and O/E>0.2) predicted 8 additional islands as shown in Table 1.

By “conserved sequence region” is meant, a nucleotide sequence of one or more regions in a polynucleotide does not vary significantly between generations or from one biological system, subject, or organism to another biological system, subject, or organism. The polynucleotide can include both coding and non-coding DNA and RNA.

By “EZH2” as used herein is meant, the EZH2 gene and any polycomb group protein EZH2 protein, peptide, or polypeptide having any polycomb group protein EZH2 activity, such as encoded by EZH2 or any other polycomb group protein EZH2 transcript derived from an EZH2 gene. The term EZH2 also refers to nucleic acid sequences encoding any polycomb group protein EZH2 protein, peptide, or polypeptide having EZH2 activity. The term “EZH2” is also meant to include other EZH2 encoding sequence, such as other EZH2 isoforms, mutant EZH2 genes, splice variants of EZH2 genes, and EZH2 gene polymorphisms. The polycomb group protein enhancer of zeste homolog 2 (EZH2) is overexpressed in hormone-refractory, metastatic prostate cancer (Varambally et al., 2002, Nature 419, 624-629). An example of such EZH2 is, for instance, the unprocessed precursor with entry in the UniProtKB/Swiss-Prot and with primary accession number Q15910, Protein name Enhancer of zeste homolog 2 Synonym ENX-1 Gene name Name: EZH2 and the sequence of the unprocessed precursor (Length: 746 AA (This is the length of the unprocessed precursor) Molecular weight: 85363 Da as demonstrated by SEQ ID NO: 1.

A published sequence of the EZH2 mRNA is, for instance, Homo sapiens enhancer of zeste homolog 2 (Drosophila) (EZH2), transcript variant 1, mRNA. as published in NMCI with SEQ ID NO:2 and published in G. Laible et al., EMBO J. 16 (11), 3219-3232 (1997); H. Chen et al., Genomics 38 (1), 30-37 (1996); K. J. Abel et al., Genomics 37 (2), 161-171 (1996); and Cardoso et al., European Journal of Human Genetics vol. 8, January 2000, pages 174-180.

The gene MSMB encodes, microseminoprotein, beta (also known as MSP; PSP; IGBF; MSPB; PN44; PRPS; PSP57; PSP94; PSP-94) which is a member of the immunoglobulin binding factor family. PSP94 encoded by the MSMB gene is a tumor suppressor. The Prostate secretory protein of 94 amino acids (PSP94), encoded by the highly prostate-specific MSMB gene, is one of the three major proteins secreted in the seminal fluid, together with PSA and Prostatic Acid Phosphatase (PAP). It has been shown that PSP94 decreases tumor growth in a syngenic in vivo model of PrCa (et al. (2003) Cancer Res. 63:2072-8) and suppresses PC-3 cell clonogenic growth as well as the growth of PC-3 xenografts (S. V. Garde et al. (1999), Prostate 38:118-25). Interestingly, the peptide PCK3145, derived from PSP94 and patented by Ambrilia (Biopharmaceutical company, Quebec, Canada), is currently being clinically tested for the treatment of advanced PrCa (R. E. Hawkins, L. Daigneault, R. Cowan, R. Griffiths, C. Panchal et al. (2005), Clin. Prostate Cancer 4:91-9).

The MSMB gene is approximately 13 kb in length, comprises 4 exons and 3 introns, and encodes a transcript of 572 nucleotides (FIG. 6). It is synthesized by the epithelial cells of the prostate gland and secreted into the seminal plasma. This protein has inhibin-like activity. It may have a role as an autocrine paracrine factor. The expression of the encoded protein is found to be decreased in prostate cancer. Two alternatively spliced transcript variants encoding different isoforms are described for this gene. One transcript variant (Homo sapiens microseminoprotein, beta-MSMB, transcript variant PSP94) has been deposited in NCBI under the accession number NM_{—}002443.2 of which the 572 bp mRNA has been described as in SEQ ID NO:3.

The other variant (Homo sapiens microseminoprotein, beta-(MSMB), transcript variant PSP57, mRNA) has been deposited in CBI under the accession number NM_{—}138634.1 of which the 466 bp mRNA has been described as in SEQ ID NO:4.

The tumor suppressor PSP94, also known as b-microseminoprotein or prostatic inhibin, is a small (10.7 kDa), non-glycosylated and cysteine-rich protein that is abundantly secreted by the prostate gland and is found in both seminal fluid and blood (S. V. Garde et al. (1999), Prostate 38:118-125; Shukeir et al., 2003; Annahi et al., 2005; and S. Lamy et al. (2006), Int. J. Cancer 118:2350-2358). It is also know that the expression of PSP94 progressively decreases during the development of prostate cancer from an early, low-invasive, androgen-dependent state to a late, highly invasive, androgen-refractory state (E. LaTulippe, J. Satagopan et al. (2002), Cancer Res. 62:4499-4506; D. K. Vanaja et al. (2003), Cancer Res. 63:3877-3882; M. Stanbrough et al. (2006), Cancer Res. 66:2815-2825). The gradual loss of PSP94 is likely to contribute to the development of prostate cancer because PSP94 impedes prostate cancer growth and metastasis (S. V. Garde et al. (1999), Prostate 38:118-125; N. Shukeir, A. Arakelian et al. (2004), Cancer Res. 64:5370-5377; and N. Shukeir et al. (2003), Cancer Res. 63:2072-2078). It is not known how the expression of the PSP94-encoding MSMB gene is regulated.

The molecular basis for the tumor-suppressor function of PSP94 is complex as this protein has been found to promote tumor cell apoptosis (S. V. Garde et al. (1999), Prostate 38:118-125), to inhibit the secretion of a matrix metalloproteinase that is implicated in tumor metastasis (B. Annahi et al. (2005), Clin. Exp. Mestas. 22:429-439), and to decrease tumor-associated, vascular endothelial growth factor (VEGF)-mediated vascularization (S. Lamy et al. (2006), Int. J. Cancer 118:2350-2358).

Interestingly, the antitumor effects of PSP94 can be recapitulated with a synthetic peptide comprising an N-terminal fragment of PSP94 and this peptide is currently clinically tested for the treatment of metastatic prostate cancer (S. Lamy et al. (2006), Int. J. Cancer 118:2350-2358).

As aforementioned, the invention is based on the unexpected discovery that the level of methylation of the MSMB gene promoter and/or the region upstream from the transcription start site of the MSMB gene is different in androgen-sensitive when compared to androgen-refractory prostate cancers. Hypermethylation of the MSMB gene promoter and/or region results in a reduced transcription of MSMB and encoding of PS94 which is regulated by EZH2.

Accordingly, in a first aspect, provided is a method that identifies a prostate cell proliferative disorder in a human male subject, the method comprises:
providing a sample of prostatic tissue and/or biological fluid of the prostate from a human patient susceptible to a prostate cancer and,
analyzing the sample for the presence of hypermethylation (on CpG and/or non-CpG dinucleotides) in a regulatory region surrounding the transcription start site or promoter region of the MSMB gene,
wherein the presence of hypermethylation in this region is indicative of prostate cancer.

Preferably, the method comprise the steps of:
(a) obtaining a biological sample from the subject;
(b) determining the methylation state of CpG island upstream and/or downstream of the TSS region and/or in the promoter region of the MSMB gene, for instance, the CpG islands about 3 kb upstream and about 2.2 kb downstream of the transcriptional start site in the MSMB gene in the subject's sample; and
(c) identifying hypermethylation of the region(s), wherein hypermethylation (on CpG and/or non-CpG dinucleotides) is identified as being different when compared to the same region(s) of the gene or associated regulatory region in a subject not having the prostate cellular proliferative disorder,
wherein detection of hypermethylation is indicative of a predisposition to, or the incidence of, prostate cancer.

The sample for use in such methods is preferably a tissue sample. Prostate biopsy is a procedure in which small samples are removed from a man's prostate gland to be tested for the presence of cancer. It is typically performed when the scores from a PSA blood test rise to a level that is associated with the possible presence of prostate cancer. A biopsy thus provides a specific example of a biological sample for use in present methods. Examination of the condition of the prostate may be performed transrectally, through the ureter or through the perineum. The most common procedure is transrectal, and may be done with tactile finger guidance (M. Ghei, S. Pericleous, et al. (2005 September), Ann. R. Coll. Surg. Engl. 87 (5):386-7) or with ultrasound guidance. If cancer is suspected, a biopsy is offered. During a biopsy tissue samples from the prostate are obtained, for instance, via the rectum. A biopsy gun can be used to insert and remove special hollow-core needles (usually three to six on each side of the prostate) in less than a second.

Suitable samples for diagnostic, prognostic, or personalized medicinal uses can be obtained from surgical samples, such as biopsies or surgical resection. However, other suitable samples for use in the methods of the invention comprise fine needle aspirates, paraffin embedded tissues, frozen tumor tissue samples, fresh tumor tissue samples, fresh or frozen body fluid. Examples of body fluids include prostatic fluids, blood samples, serum, plasma, urine, ejaculate, wash or lavage fluid. In fact, any tissue or fluid containing cells or nucleic acid, preferably DNA, derived from cells of the prostate is a suitable reagent for use in the methods of the invention. Present methods preferably also include the step of obtaining the suitable sample. Cells may need to be lysed for release of the nucleic acid. The nucleic acid may need to be cleared of proteins or other contaminants, e.g., by treatment with enzymes. The nucleic acid may also need to be concentrated prior to further use in the method of the invention, in particular when the nucleic acid is derived from bodily fluids.

As shown herein, the above mentioned methods for identifying prostate tumor cells also allow distinguishing hormone-sensitive from hormone-refractory prostate cancers.

Thus, in a particular aspect, the invention provides for an in vitro method for distinguishing a hormone independent proliferative disorder or hormone-refractory proliferative disorder from a hormone-sensitive proliferative disorder in tissue and/or in at least one cell obtainable from tissue of the prostate from a subject. Such diagnostic method comprises contacting a DNA of a tissue or a DNA of a biological fluid with a reagent which detects the methylation status of the promoter region of the MSMB gene, wherein hypermethylation, as compared to the methylation status of the MSMB promoter region or upstream of the promoter region from a normal cell or compared to the methylation status of the MSMB promoter region or upstream of the promoter region from cells of tissue of a prostate with steroidal hormone-sensitive proliferative disorder, is indicative of the steroidal hormone-refractory proliferative disorder.

The test is particularly suitable to distinguish between hormone-refractory and hormone-sensitive and in particular for androgen-sensitive and androgen-refractory prostate proliferative disorders and to distinguish between benign prostate hyperplasia and prostate cancer.

In certain embodiments, provided is a method for distinguishing between androgen-sensitive and androgen-refractory prostate cancer by contacting a cellular component of a prostate tissue sample or another sample with a reagent which detects the methylation status of the MSMB promoter or upstream of the MSMB promoter region.

As aforementioned, methylation-sensitive restriction endonuclease can be utilized to identify a hypermethylated MSMB promoter or upstream region, for example.

Other approaches for detecting methylated CpG dinucleotide motifs use chemical reagents. In particular chemical reagents that selectively modify the methylated or non-methylated form of CpG dinucleotide motifs can be used in the methods of the invention. Such chemical reagents include bisulphite ions. Sodium bisulphite converts unmethylated cytosine to uracil but methylated cytosines remain unconverted. Analysis of the nucleic acid sequence after bisulfite conversion indicates if the original nucleic acid was all or not methylated.

Multiple techniques for analyzing the methylation status of CpG dinucleotide motifs in CpG islands are known in the art. They comprise without limitation sequencing, methylation-specific PCR (MS-PCR), McMS-PCR, MLPA, QAMA, MSRE-PCR, MethyLight, HeavyMethyl, ConLight-MSP, BS-MSP, COBRA, McCOBRA, MS-SNuPE, MS-SSCA, PyroMethA, MALDI-TOF, MassARRAY, ERMA, QBSUPT, MethylQuant, Quantitative PCR sequencing, oligonucleotide-based microarray systems, Pyrosequencing, and Meth-DOP-PCR. A review of techniques for the detection of the methylation state of a gene is given, for instance, in Oral Oncology 2006, Vol. 42, 5-13 and references cited therein.

A preferred technique for the detection and/or quantification of methylated DNA is the Methylation Specific PCR (MSP) technique. This technique can be used in end-point format, wherein the presence of methylated DNA is, for instance, detected by electrophoresis or by the use of dyes such as SYBR Green I or Ethidium Bromide that bind double-stranded DNA that accumulates during the amplification reaction. Alternatively, the method is based on the continuous optical monitoring of an amplification process and utilizes fluorescently labeled reagents. Their incorporation in a product can be quantified as the reaction processes and is used to calculate the copy number of that gene or sequence region in the sample. The quantification of the amplification product may require the use of controls to avoid false negativity/positivity of the reaction. Particularly suitable for the quantification of the amplification product are reference genes (e.g., beta-actin) whose methylation status is known, and/or DNA standards (e.g., methylated or unmethylated standards).

Accumulation of an amplification product can be monitored through the incorporation of labeled reagents. Some techniques use labeled primers; others rely upon the use of labeled probes to monitor the amplification product. Real-time quantitative methylation specific PCR techniques comprise the use of Amplifluor primers and/or Molecular Beacon probes and/or Fret probes and/or Scorpion primers and/or Taqman probes and/or oligonucleotide blockers (e.g., HeavyMethyl approach) and/or DzyNA primers. All these probes and primers have been described and their mode of action is well known in the art.

In certain embodiments, the methods of the invention use unmethylated specific primers indicated by SEQ ID NOS:15, 16, 18, 23, 24, 25, 27, 30, 31, 40, 41, 44, 45, 52, 53, 59, 60, 63, 64 and/or methylated specific primers indicated by SEQ ID NOS:13, 14, 17, 19, 20, 21, 22, 26, 28, 29, 38, 39, 42, 43, 50, 51, 57, 58, 61, 62.

Alternatively to PCR, other amplification methods such as NASBA, 3SR, TMA, LCR, selective amplification of target polynucleotide sequences (U.S. Pat. No. 6,410,276), consensus sequence primed polymerase chain reaction (U.S. Pat. No. 5,437,975), arbitrarily primed polymerase chain reaction (WO 90/06995), invader technology, strand displacement technology, and nick displacement amplification (WO 2004/067726) may be used to amplify the appropriate nucleic acid.

In certain embodiments, bisulphite sequencing is utilized in order to determine the methylation status of the MSMB gene. Primers may be designed in both the sense and antisense orientation to direct sequencing across the relevant region of the MSMB gene. In one embodiment, bisulphite sequencing may be carried out by using at least one the following sequencing primers. SEQ ID NOS: 9, 10, 11, 12, 32, 33, 34, 35, 36, 37, 46, 47, 48, 49, 54, 55, 56, 65, 66, 67, 68, 69 and 70.

These amplification primers, amplification probes and sequencing primers form a further aspect of the invention.

Revealed is that a hypermethylated promoter for the regulatory regions of the human MSMB gene, CpG island in the promoter and upstream of the promoter, positively correlates with androgen-insensitivity in prostatic carcinogenesis. This invention provides a diagnostic tools or means to determine a prostate cancer and to distinguish between androgen sensitivity and androgen independency of such prostate cancer. Methylation changes are not only ideal for screening purposes, but also interesting targets for monitoring staging or grading of the cancer. Methods for identifying a prostate cell proliferative disorder in a subject, can comprise the steps of:
(a) obtaining a biological sample from the subject;
(b) determining the methylation state of CpG island upstream and/or downstream of the TSS region and/or in the promoter of the MSMB gene, for instance, the CpG islands about 3 kb upstream and about 2.2 kb downstream of the transcriptional start site in the MSMB gene in the subject's sample; and
(c) identifying hypermethylation of the region(s), wherein hypermethylation on CpG and/or non-CpG dinucleotides is identified as being different when to the same region(s) of the gene or associated regulatory region in a subject not having the prostate cellular proliferative disorder, wherein detection of hypermethylation is indicative for the stage or grade of the prostate cancer.

This unexpected finding allows diagnosis of hormone-independent cancers by a simple assay that detects the hypermethylated GCP islands in the promoter region or upstream of the promoter region directly by, for instance, restriction endonuclease analysis to select the proper treatment for subjects with a prostate cancer, depending on the fact of the prostate cancer is hormone-refractory or hormone-sensitive or depending on the stage or grade of prostate cancer as can be indicated by the hypermethylation status. This is more reliable than detecting levels of MSMB mRNA or MSMB gene products. The diagnostic methods will also allow indication of the proper treatment for hormone-refractory cancers or avoid giving subjects with a hormone-sensitive cancer an inadequate treatment or assure that they can be treated differently. For instance, patients by the diagnosis of the invention to have hypermethylation of a CpG island in the promoter region or upstream of the promoter region of the MSMB can be subjected to an antimitotic drug therapy methods of treatment or the treatment can now adequately be directed to replacing the hypermethylated CpG islands with a non-methylated islands, which, for instance, is possible by a treatment with a therapeutically sufficient dosage of a pharmaceutically acceptable DNA methylation inhibitor. A particular treatment selected based on the conclusion of the diagnosis method of the invention can also be a treatment to decrease the expression of the histone modifier gene, EZH2, or a treatment to decrease the activity of the EZH2 protein. Such treatments are available in the art. For instance, Chroma Therapeutics developed a series of compounds that inhibit specifically EZH2.

Methylation and hypermethylation of the non −CpG of the MSMB gene was found to be a biomarker to distinguish between benign prostate hyperplasia (BPH) and prostate cancer. Human samples 5 prostate cancer samples and 4 benign prostate hyperplasia (BPH) and one whole blood sample (Control) have been screened for methylations and sequence variations to map CpG methylation in the CpG4-5 region of the MSMB gene, to map non-CpG methylation in the CpG4-5 region of the MSMB gene and to map sequence variations in CpG4-5 region of the MSMB gene.

A weak hypermethylation on some CpG dinucleotides in the CpG4-5 island of the MSMB gene was found in the human prostate cancer samples in comparison to benign prostatic hyperplasia (Table 2). Indeed, after bisulphite sequencing, one out of eleven analyzed clones of benign prostate hyperplasia sample B3 was completely unmethylated, and five out of eighteen analyzed clones of benign prostate hyperplasia sample B4 was methylated less than 50%. In contrast, the average CpG methylation level for all clones from prostate cancer samples was about 90% (Table 2). No significant difference for CpG methylation in CpG4-5 island of the MSMB gene was found between human prostate cancer sample and human genomic DNA isolated from whole blood (Table 2).

A clear hypermethylation on some non-CpG dinucleotides in the CpG4-5 island of the MSMB gene was found in the human prostate cancer samples in comparison to benign prostatic hyperplasia (Table 3). Indeed, after bisulphite sequencing, four out of five prostate cancer samples contained clones with non-CpG methylation on position −2973 bp, −2958 bp, −2944 bp, −2885 bp towards the TSS of the MSMB gene, which was not observed in the clones from benign prostatic hyperplasia samples B1-5. Prostate Cancer sample C4 showed another non-CpG methylation that was located on position −3328 bp, −3194 bp, −3111 bp towards the TSS of the MSMB gene and which was also not observed in benign prostatic hyperplasia samples B1-5. CA-dinucleotide methylation at position −3223 bp and −3100 bp was found only in some clones from the benign prostate hyperplasia samples B4. CT-dinucleotide at position −2992 was methylated in some clones of both prostate cancer (C1, C5) and benign (B1, B2) samples. Importantly, non-CpG methylation was not at all detected in human genomic DNA isolated from whole Blood (sample H1) (Table 3).

All different sequence alterations which are detected in the CpG4-5 island of the MSMB gene in prostate cancer (C1-4), benign prostatic hyperplasia (B1-5) samples and whole blood (H1) are shown in Table 4.

In conclusion, analysis of DNA samples of five prostate cancer samples (C1-5), four benign prostatic hyperplasia (B1-4) and whole blood (H1) on differential non-CpG methylation and sequence alterations in CpG4-5 island of the MSMB gene resulted in total 10 new haplotypes (Table 5). Haplotype 0 corresponds to a reference sequence of the corresponding fragment of the MSMB gene without any alteration. Haplotypes 3, 4 and 6 are only found in prostate cancer and not in benign prostatic hyperplasia. Haplotype 3, present in C1, C2, C3 and C5, is characterized by non-CpG methylation at position −2973 bp, −2958 bp, −2944 bp and −2885 bp towards TSS of MSMB gene. Haplotype 4, present in sample C4, is characterized by CA methylation at position −3352 bp and sequence alteration at position −3166 bp, −2956 bp, −2782 bp towards TSS of MSMB gene. Haplotype 6, present in sample C4, is characterized by non-CpG methylation at position −3328 bp, −3194 bp and −3111 bp towards TSS of the MSMB gene. Haplotypes 7-10 were present only in benign prostate hyperplasia samples, while haplotypes 0, 1, 2 and 5 could be found both in cancer and benign prostate samples.

This unexpected finding allows to diagnose prostatic cells or tissues for prostate cancer and to distinguish between a condition of benign prostate hyperplasia and prostate cancer.

The unexpected findings of the invention now specifically allows to diagnose for androgen-independent prostate cancer (AIPC) by a simple assay that detects the hypermethylated promoter or upstream region of the promoter directly of the MSMB gene and to select the proper treatment for subjects with this AIPC or avoid that subjects with an androgen-sensitive cancer will receive an inadequate treatment or allow that a such subject will be treated differently than subjects with androgen-sensitive prostate cancer.

For instance, patients by the diagnosis of the invention found to have hypermethylation of a CpG island in the promoter region of the MSMB gene or in the regulatory regions surrounding the transcriptional start site of the MSMB gene can be subjected to an antimitotic drug therapy methods or the treatment can now focus on replacing the hypermethylated promoter with a non-methylated promoter.

Moreover, the unexpected findings of the invention now specifically allows to define of a patients has a prostate cancer or a benign prostate hyperplasia by a simple assay that detects methylated non CpG dinucleotides in the promoter or upstream region of the promoter or in the regulatory regions surrounding the transcriptional start site of the MSMB gene and to select the proper treatment for subjects with this benign prostate hyperplasia or prostate cancer or avoid that subjects with benign prostate hyperplasia will receive an inadequate anti-cancer treatment or allow that a such subject will be treated differently than subjects with prostate cancer.

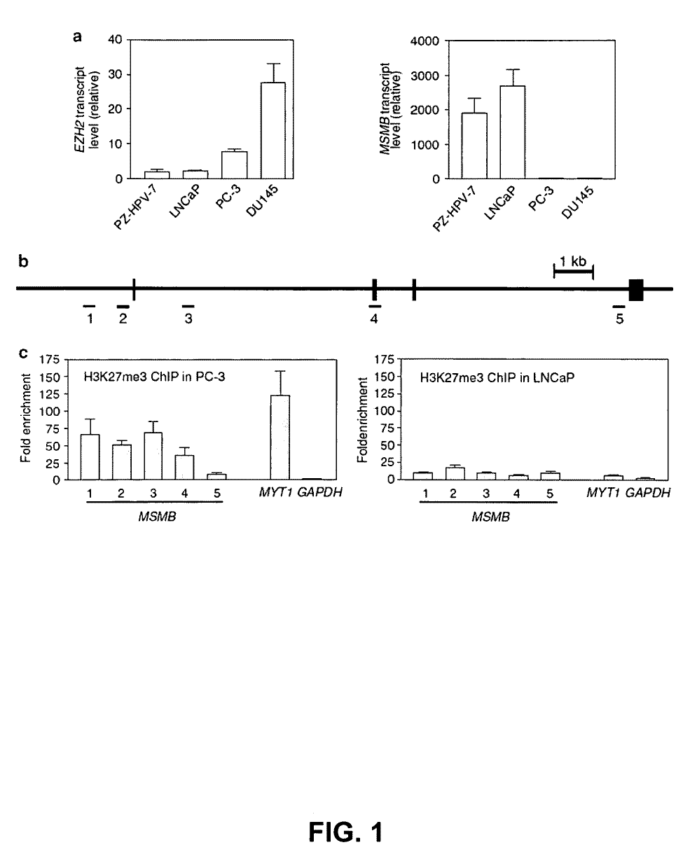

Moreover, in the case of trimethylation of the histones, the treatment can focus on decreasing the expression of the EZH2 gene or a treatment to decrease the activity of the EZH2 protein. We have used quantitative reverse transcriptase-polymerase chain reaction (RT-PCR) to examine whether these changes can also be detected in prostate-derived cell lines. The EZH2 transcript was indeed four- to 14-fold over-expressed in the androgen-refractory PC-3 and DU 145 cell lines, as compared to the EZH2 expression level in the androgen-sensitive PZ-HPV-7 and LNCaP cells (FIG. 1, Panel a). Furthermore, the PSP94 transcript was readily detected in the PZ-HPV-7 and LNCaP cells, but was at least three orders of magnitude less abundant in the PC-3 and DU 145 cells. Having confirmed an inverse relationship between the transcript levels of EZH2 and MSMB, we subsequently examined whether these changes are causally related and whether the MSMB gene is a target for H3K27 trimethylation. Using a chromatin immunoprecipitation procedure (ChIP) with anti-H3K27me3 antibodies, we found that the MSMB gene in PC-3 cells was heavily trimethylated on H3K27 in nucleosomes that were associated with the promoter region (primer set 2) and with flanking sequences (primer sets 1, 3 and 4) (FIG. 1, Panels b and c). In contrast, nucleosomes from a fragment of intron 3 of the MSMB gene (primer set 5) were much less trimethylated on H3K27. In these experiments MYT1 (myelin-transcription-factor 1), a well-established Polycomb target gene (Kirmizis et al., 2004), served as a positive control and glyceraldehyde-3 phosphate dehydrogenase (GAPDH) as a negative control.

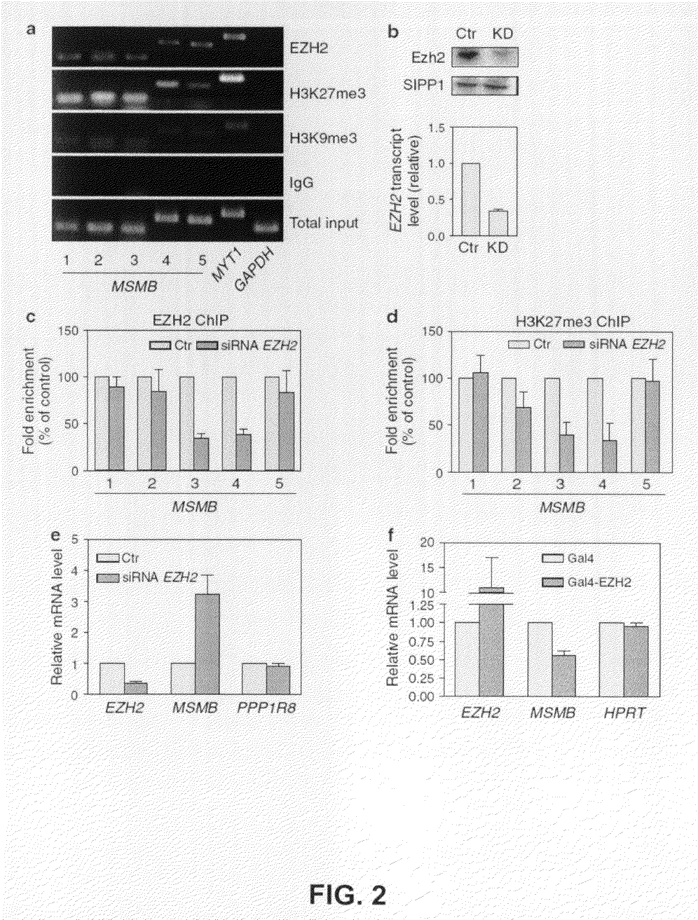

Importantly, whereas the MSMB gene was heavily trimethylated on H3K27 in PC-3 cells, which hardly express PSP94, this gene was only mildly trimethylated on H3K27 in LNCaP cells, which express a lot of PSP94 (FIG. 1, Panels a and c). As EZH2 is the major histone methyltransferase known to trimethylate H3K27 in vivo, the above data suggested that the MSMB gene is a target for repression by EZH2. Consistent with this notion, ChIP experiments with anti-EZH2 antibodies showed an association of EZH2 with all the analyzed regions of the MSMB gene (FIG. 2, Panel a). Binding of EZH2 to the MSMB gene was as robust as its binding to the MYT1 gene, a well-known EZH2 target gene. Interestingly, neither MSMB nor MYT1 was abundantly trimethylated on H3K9. As H3K9 trimethylation also correlates with transcriptional repression but is EZH2-independent, these data attest to the specificity of the detected EZH2-H3K27me3 association. Little or no enrichment of GAPDH DNA was observed in the ChIP experiments with the different antibodies (FIG. 2, Panel a). To obtain more direct evidence for a role of EZH2 in the transcriptional repression of MSMB, we have subsequently examined the effect of the RNAi-mediated knockdown of EZH2 on the H3K27 trimethylation of the MSMB gene in PC-3 cells. As expected, less EZH2 was associated with the MSMB gene (FIG. 2, Panel c) following the knockdown of EZH2 (FIG. 2, Panel b). Within the time frame of the experiment (48 hours), the loss was only evident in intron I (primer sets 3 and 4) and was not detected in upstream (primer sets 1 and 2) or downstream (primer set 5) sequences. This is reminiscent of the local loss of the association of PRC2 component SUZ12 with the MYT1 gene following the knockdown of SUZ12 (Cao and Zhang, 2004). Importantly, the loss of the targeting of EZH2 to intron 1 was associated with a loss of H3K27 trimethylation in this region (FIG. 2. Panel d). We have also investigated whether a change in the level of EZH2 affects the expression of MSMB. In FIG. 2, Panel e, it is shown that the knockdown of the EZH2 transcript in PC-3 cells by about 70% was associated with a threefold increase in the expression of MSMB. The transcript level of a control housekeeping gene, PPP1R8, was not affected. As the knockdown of EZH2 only affected the targeting of EZH2 to intron 1 (FIG. 2, Panel c), this suggests that intron 1 harbors important regulatory elements of MSMB expression. Conversely, the overexpression of EZH2, fused to a Gal4-tag, in PZ-HPV-7 cells resulted in a 50% drop of the MSMB transcript level but was without effect on the transcript level of the housekeeping gene HPRT (FIG. 2, Panel f). Collectively, the above data demonstrate that MSMB is a canonical EZH2 target gene and that repression of MSMB is associated with trimethylation of H3K27. Polycomb target genes are often additionally silenced through histone deacetylation and DNA methylation of CpG islands (van der Vlag and Otte, 1999; Viré et al., 2006). This is explained by the ability of PcG proteins to bind histone deacetylases and to recruit DNA methyltransferases.

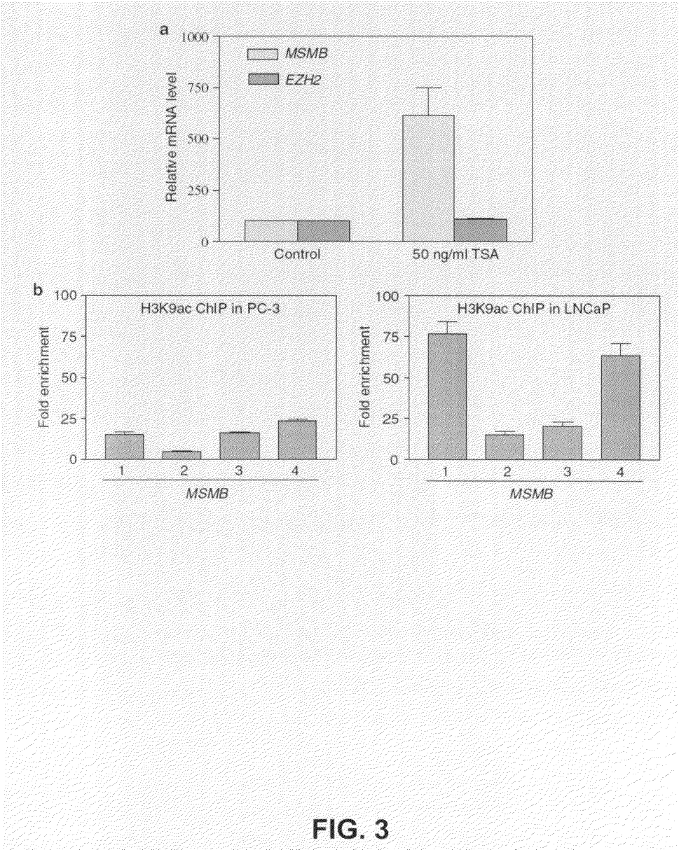

We used trichostatin A (TSA), a cell permeable inhibitor of histone deacetylases, to examine whether the MSMB gene is also controlled by histone (de)acetylation. The addition of TSA (50 ng/ml) to PC-3 cells for nine hours indeed resulted in a six-fold increase of the MSMB transcript level (FIG. 3, Panel a). As TSA did not affect the expression level of EZH2, these data strongly indicate that the MSMB gene is additionally silenced by histone deacetylation.

To examine our findings in more detail, we performed ChIP experiments with antibodies against acetylated Lys 9 of Histone H3 (H3K9ac). Three of the four examined regions of the MSMB gene were hypoacetylated in PC-3 cells, as compared to their acetylation status in LNCaPs (FIG. 3, Panel b). This fits nicely with the decreased expression of the MSMB gene in PC-3 cells (FIG. 1, Panel a) and is further evidence for a role of deacetylation in the repression of this gene in PC-3 cells. Finally, we have found that 50-azacytidine, an inhibitor of DNA methyltransferases, promotes the expression of the MSMB gene in PC-3 cells by about fivefold (FIG. 4, Panel a), indicating that DNA methylation also contributes to the repression of MSMB. In further agreement with this notion, we found that the MSMB gene harbors two CpG islands (FIG. 4, Panel b). DNA bisulfite sequencing revealed that these islands are indeed methylated, both in PC-3 and in LNCaP cells. Interestingly, the methylation of the CpG island in the promoter region was significantly more pronounced in PC-3 cells, as compared to its methylation in LNCaP cells, in agreement with the lesser expression of the MSMB gene in PC-3 cells. In addition, the methylation of this CpG island in PC-3 was decreased following the addition of 50-azacytidine, which is additional evidence that MSMB is controlled by DNA methylation.

Diagnosing the acetylation status of MSMB gene histones can be a further diagnostic tool to decide which of the prostate cancer patients should be treated by histone deacetylase inhibitors (HDAC inhibitors). Various histone deacetylase inhibitor are available in the art for and structurally defined for the man skilled in the art from several companies such as EntreMed, Merck & Co, Karus Therapeutics, Kalypsys (US20070123580 and US20070135438), Johnson & Johnson (JNJ 26481585, a second-generation, oral, pan-HDAC inhibitor with broad-spectrum preclincial antitumor activity), MethylGene/EnVivo (U.S. Pat. No. 7,288,567 B2, U.S. Pat. No. 6,946,441 B2, US20070155730 and U.S. Pat. No. 6,897,220 B2), ArQule, Sulfidris and Pharmacyclics (US20070281934), the compounds hereby incorporated by reference.

The HDAC inhibitors of Pharmacyclics comprise, for instance, a compound selected from among: 1-(3,4-dichloro-phenylmethyl)-1H-indole-6-carboxylic acid hydroxyamide; 1-(2-methyl-phenylmethyl)-1H-indole-6-carboxylic acid hydroxyamide; 1-(3,4,5-trimethoxy-phenylmethyl)-1H-indole-6-carboxylic acid hydroxyamide; 1-(3-fluoro-phenylmethyl)-1H-indole-6-carboxylic acid hydroxyamide; 1-(3-methyl-phenylmethyl)-1H-indole-6-carboxylic acid hydroxyamide; 1-(benzyl)-1H-indole-6-carboxylic acid hydroxyamide; 1-(3,5-dimethoxy-phenylmethyl)-1H-indole-6-carboxylic acid hydroxyamide; 1-(1-methyl-1-phenylmethyl)-1H-indole-6-carboxylic acid hydroxyamide; 1-(4-fluoro-phenylmethyl)-1H-indole-6-carboxylic acid hydroxyamide; 1-(2-fluoro-phenylmethyl)-1H-indole-6-carboxylic acid hydroxyamide; 1-(2-chloro-phenylmethyl)-1H-indole-6-carboxylic acid hydroxyamide; 1-(3-methoxy-phenylmethyl)-1H-indole-6-carboxylic acid hydroxyamide; 1-(naphth-2-ylmethyl)-1H-indole-6-carboxylic acid hydroxyamide; 1-(3-phenylpropyl)-1H-indole-6-carboxylic acid hydroxyamide; 1-(cyclohexylmethyl)-1H-indole-6-carboxylic acid hydroxyamide; 1-[1-(phenyl)-propen-3-yl]-1H-indole-6-carboxylic acid hydroxyamidel-[4-(trifluoromethoxy)-phenylmethyl]-1H-indole-6-carboxylic acid hydroxyamide; 1-(4-chloro-phenylmethyl)-1H-indole-6-carboxylic acid hydroxyamide; 1-(benzo[2,1,3]oxadiazol-5-ylmethyl)-1H-indole-6-carboxylic acid hydroxyamide; 1-(4-methyl-phenylmethyl)-1H-indole-6-carboxylic acid hydroxyamide; 1-(3-fluoro-4-methoxy-phenylmethyl)-1H-indole-6-carboxylic acid hydroxyamide; 1-[4-(difluoromethoxy)-phenylmethyl]-1H-indole-6-carboxylic acid hydroxyamide; 1-(4-methoxy-phenylmethyl)-1H-indole-6-carboxylic acid hydroxyamide; 1-(phenethyl)-1H-indole-6-carboxylic acid hydroxyamide; 1-(3-chloro-phenylmethyl)-1H-indole-6-carboxylic acid hydroxyamide; 1-[N-(t-butoxycarbonyl)piperidin-4-ylmethyl]-1H-indole-6-carboxylic acid hydroxyamide; 1-(piperidin-4-ylmethyl)-1H-indole-6-carboxylic acid hydroxyamide; 1-(N-methylsulfonyl-3-aminobenzyl)-1H-indole-6-carboxylic acid hydroxyamide; 3-(Dimethylaminomethyl)-1-(4-methoxybenzyl)-1H-indole-6-carboxylic acid hydroxyamide; 3-(N-Morpholinomethyl)-1-(4-methoxybenzyl)-1H-indole-6-carboxylic acid hydroxyamide; 3-(N-Pyrrolidinomethyl)-1-(4-methoxybenzyl)-1H-indole-6-carboxylic acid hydroxyamide; 3-(N-Benzylaminomethyl)-1-(4-methoxybenzyl)-1H-indole-6-carboxylic acid hydroxyamide; and 3-(Ethyl)-1-(4-methoxybenzyl)-1H-indole-6-carboxylic acid hydroxyamide.

The HDAC inhibitors of EntreMed (EntreMed. Inc. EntreMed Completes Acquisition of Miikana Therapeutics, Media Release, 11 Jan. 2006. Available from: World Wide Web entremed.com have been developed by Miikana (US20050197336, US20050250784 and US20060199829) as series of mercaptoamide-based, non-hydroxamate inhibitors of HDACs. The structure of this series was designed to be as close as possible to that of vorinostat, but having a mercaptoamide replacing the hydroxamate moiety. Particular compounds are for instance the compounds of the group consisting of 1,3,4,9-tetrahydro-2H-b-carbolin-2-yl, 1,3,4,4a,9,9a-hexahydro-2H-b-carbolin-2-yl, 1,3,4,5-tetrahydro-2H-pyrido[4,3-b]indol-2-yl, 1,1a,3,4,4a,5-hexahydro-2H-pyrido[4,3-b]indol-2-yl, 1,4,5,6-tetrahydroazepino[4,5-b]indol-3(2H)-yl, 3,4-dihydro[1]benzothieno[2,3-c]pyridin-2(1H)-yl, 3,4-dihydro[1]benzofuro[2,3-c]pyridin-2(1H)-yl and 10-oxo-3,4,5,10-tetrahydrobenzo[b]-1,6-naphthyridin-2(1H)-yl. Other such inhibitors of HDACs are, for instance, -(2-naphthylsulfonyl)-4-(5-hydroxyaminocarbonylthiazol-2-yl)piperazine; 1-(2-naphthylsulfonyl)-4-(5-hydroxyamino-carbonylthiazol-2-yl)-1,4-diazepane; 1-(2-naphthylsulfonyl)-4-(4-hydroxyaminocarbonylthiazol-2-yl)piperazine; 1-(2-naphthylsulfonyl)-4-[(5-(2-hydroxyaminocarbonylethen-1(Z)-yl-thiazol-2-yl)piperazine; 4-(2-naphthylsulfonylamino)-1-[(5-(2-hydroxyaminocarbonyl-thiazol-2-yl)-piperadine; 2-[4-(3,4-dimethoxy-benzene sulfonyl)-piperazin-1-yl]-thiazole-5-carboxylic acid hydroxyamide; 2-[4-(4-trifluoromethoxy-benzene sulfonyl)-piperazin-1-yl]-thiazole-5-carboxylic acid hydroxyamide; 2-[4-(4-toluene-4-sulfonyl)-piperazin-1-yl]-thiazole-5-carboxylic acid hydroxyamide; 2-[4-(4-trifluoromethyl-benzenesulfonyl)-piperazin-1-yl]-thiazole-5-carboxylic acid hydroxyamide; 2-[4-(4-nitro-benzenesulfonyl)-piperazin-1-yl]-thiazole-5-carboxylic acid hydroxyamide; 2-[4-(4-acetyl-benzenesulfonyl)-piperazin-1-yl]-thiazole-5-carboxylic acid hydroxyamide; 2-[4-(thiophene-2-sulfonyl)-piperazin-1-yl]-thiazole-5-carboxylic acid hydroxyamide; 2-[4-(biphenyl-4-sulfonyl)-piperazin-1-yl]-thiazole-5-carboxylic acid hydroxyamide; 2-[4-(5-dimethylamino-naphthalene-1-sulfonyl)-piperazin-1-yl]-thiazole-5-carboxylic acid hydroxyamide; 2-[4-(4-fluoro-benzenesulfonyl)-piperazin-1-yl]-thiazole-5-carboxylic acid hydroxyamide; 2-(4-methyl-piperazin-1-yl)-thiazole-5-carboxylic acid hydroxyamide; 2-(4-Benzyl-piperazin-1-yl)-thiazole-5-carboxylic acid hydroxyamide; 2-(4-(2-hydroxyethyl)-piperazin-1-yl)-thiazole-5-carboxylic acid hydroxyamide; 2-(4-(2-aminoethyl)-piperazin-1-yl)-thiazole-5-carboxylic acid hydroxyamide; 2-(4-phenylethyl-piperazin-1-yl)-thiazole-5-carboxylic acid hydroxyamide; 2-(4-acetyl-piperazin-1-yl)-thiazole-5-carboxylic acid hydroxamide; 2-(4-benzoyl-piperazin-1-yl)-thiazole-5-carboxylic acid hydroxamide; 2-(4-phenylacetyl-piperazin-1-yl)-thiazole-5-carboxylic acid hydroxamide; N-(2-naphthylsulfonyl)-N′-{2-[5-(N-hydroxycarboxamido)]thiazolyl}-piperazine; and pharmaceutically acceptable salts, isomers, tautomers, and prodrugs thereof. 2-[4-(naphtha-2-yl-4-sulfonyl)-piperazin-1-yl]-thiazole-5-carboxylic acid hydroxyamide; 2-[4-(4-trifluoromethoxy-benzene sulfonyl)-piperazin-1-yl]-thiazole-5-carboxylic acid hydroxyamide; 2-[4-(4-toluene-4-sulfonyl)-piperazin-1-yl]-thiazole-5-carboxylic acid hydroxyamide; 2-[4-(biphenyl-4-sulfonyl)-piperazin-1-yl]-thiazole-5-carboxylic acid hydroxyamide; 2-[4-(4-trifluoromethyl-benzenesulfonyl)-piperazin-1-yl]-thiazole-5-carboxylic acid hydroxyamide; 2-[4-(3,4-dimethoxy-benzene sulfonyl)-piperazin-1-yl]-thiazole-5-carboxylic acid hydroxyamide; 2-[4-(5-dimethylamino-naphthalene-1-sulfonyl)-piperazin-1-yl]-thiazole-5-carboxylic acid hydroxyamide; 2-[4-(4-acetyl-benzenesulfonyl)-piperazin-1-yl]-thiazole-5-carboxylic acid hydroxyamide; 2-[4-(benzenesulfonyl)-piperazin-1-yl]-thiazole-5-carboxylic acid hydroxyamide; 2-[4-(4-nitro-benzenesulfonyl)-piperazin-1-yl]-thiazole-5-carboxylic acid hydroxyamide; 2-[4-(4-fluoro-benzenesulfonyl)-piperazin-1-yl]-thiazole-5-carboxylic acid hydroxyamide; 2-[4-(4-pyrolidinylbenzenesulfonyl)-piperazin-1-yl]-thiazole-5-carboxylic acid hydroxyamide; 2-[4-(thiophene-2-benzenesulfonyl)-piperazin-1-yl]-thiazole-5-carboxylic acid hydroxyamide; 2-[4-(N-methyl-2,3-dihydrobenzisoxazinylsulfonyl) piperazin-1-yl]-thiazole-5-carboxylic acid hydroxyamide; 2-[4-(4-isopropylphenylsulfonyl)-piperazin-1-yl]-thiazole-5-carboxylic acid hydroxyamide; 2-[4-(trans-2-phenylethanelsulfonyl)-piperazin-1-yl]-thiazole-5-carboxylic acid hydroxyamide; 2-[4-(2-chlorophenylsulfonyl)-piperazin-1-yl]-thiazole-5-carboxylic acid hydroxyamide; 2-[4-(3-chlorophenylsulfonyl)-piperazin-1-yl]-thiazole-5-carboxylic acid hydroxyamide; 2-[4-(3,4-dichlorophenylsulfonyl)-piperazin-1-yl]-thiazole-5-carboxylic acid hydroxyamide; 2-[4-(N,N-dimethylsulfonyl)-piperazin-1-yl]-thiazole-5-carboxylic acid hydroxyamide; 2-[4-(4-methylsulfonylphenylsulfonyl)-piperazin-1-yl]-thiazole-5-carboxylic acid hydroxyamide; 2-[4-(pyridine-3-sulfonyl)-piperazin-1-yl]-thiazole-5-carboxylic acid hydroxyamide; 2-[4-(2-methylphenylsulfonyl)-piperazin-1-yl]-thiazole-5-carboxylic acid hydroxyamide; 2-[4-(3-methylphenylsulfonyl)-piperazin-1-yl]-thiazole-5-carboxylic acid hydroxyamide; 2-{4-[(3′-[(dimethylamino)methyl]-1,1′-biphenylsulfonyl]piperazin-1-yl)}-1,3-thiazole-5-carboxylic acid hydroxyamide; 2-[4-(4-n-propylphenylsulfonyl)-piperazin-1-yl]-thiazole-5-carboxylic acid hydroxyamide; 2-[4-(3,5-dimethylphenylsulfonyl)-piperazin-1-yl]-thiazole-5-carboxylic acid hydroxyamide; 2-[4-(4-t-butylphenylsulfonyl)-piperazin-1-yl]-thiazole-5-carboxylic acid hydroxyamide; 2-[4-(benzylsulfonyl)piperazin-1-yl]-thiazole-5-carboxylic acid hydroxyamide; 2-[4-(4′-N,N-dimethylcarboxamido-1,1′-biphenylsulfonyl) piperazin-1-yl]-1,3-thiazole-5-carboxylic acid hydroxyamide; 2-[4-(4′-methylsulfonylamino-1,1′-biphenylsulfonyl)piperazin-1-yl]-1,3-thiazole-5-carboxylic acid hydroxyamide; 2-{4-[(4′-((dimethylamino)methyl)-1,1′-biphenylsulfonyl]piperazin-1-yl}-1,3-thiazole-5-carboxylic acid hydroxyamide; 2-[4-(3,5-dimethylisoxazolesulfonyl)piperazin-1-yl]-thiazole-5-carboxylic acid hydroxyamide; 2-[4-({4-morpholino}-3-pyridylsulfonyl)-piperazin-1-yl]-thiazole-5-carboxylic acid hydroxyamide; 2-[4-(3′-(dimethylamino)-1,1′-biphenylsulfonyl)piperazin-1-yl]-1,3-thiazole-5-carboxylic acid hydroxyamide; 2-{4-[(3′-(pyrrolidin-1-ylmethyl 1,1′-biphenylsulfonyl]piperazin-1-yl}-1,3-thiazole-5-carboxylic acid hydroxyamide; 2-[4-(4-dimethylaminophenylsulfonyl)-piperazin-1-yl]-thiazole-5-carboxylic acid hydroxyamide; 2-[4-(3,4-dimethylphenylsulfonyl)-piperazin-1-yl]-thiazole-5-carboxylic acid hydroxyamide; 2-[4-(4′-(morpholin-4-ylcarbonyl 1,1′-biphenylsulfonyl)piperazin-1-yl]-1,3-thiazole-5-carboxylic acid hydroxyamide; 2-(4-{4-[(2-((dimethylamino)-methyl)thien-3-yl]phenylsulfonyl}piperazin-1-yl)-1,3-thiazole-5-carboxylic acid hydroxyamide; 2-{4-[(4′-fluoro-1,1′-biphenyl-4-yl)sulfonyl]piperazin-1-yl}-1,3-thiazole-5-carboxylic acid hydroxyamide; 2-[4-(3-chloro-2-methylphenylsulfonyl)-piperazin-1-yl]-thiazole-5-carboxylic acid hydroxyamide; 2-[4-(3-chloro-4-methylphenylsulfonyl)-piperazin-1-yl]-thiazole-5-carboxylic acid hydroxyamide; 2-[4-(4-methoxyphenylsulfonyl)-piperazin-1-yl]-thiazole-5-carboxylic acid hydroxyamide; 2-{4-[4-(pyridin-4-yl)phenylsulfonyl]piperazin-1-yl}-1,3-thiazole-5-carboxylic acid hydroxyamide; 2-[4-(3-fluorophenylsulfonyl)-piperazin-1-yl]-thiazole-5-carboxylic acid hydroxyamide; 2-[4-(2-fluorophenylsulfonyl)-piperazin-1-yl]-thiazole-5-carboxylic acid hydroxyamide; 2-[4-(2-methyl-5-fluorophenylsulfonyl)-piperazin-1-yl]-thiazole-5-carboxylic acid hydroxyamide; 2-[4-(2-trifluoromethylphenylsulfonyl)-piperazin-1-yl]-thiazole-5-carboxylic acid hydroxyamide; 2-{4-[(3′-chloro-1,1′-biphenyl-4-yl)sulfonyl]piperazin-1-yl}-1,3-thiazole-5-carboxylic acid hydroxyamide; 2-{4-[(2′-chloro-1,1′-biphenyl-4-yl)sulfonyl]piperazin-1-yl}-1,3-thiazole-5-carboxylic acid hydroxyamide; 2-(4-{[4-(2-furyl)phenyl]sulfonyl}piperazin-1-yl)-1,3-thiazole-5-carboxylic acid hydroxyamide; 2-[4-(3,4-difluorophenylsulfonyl)-piperazin-1-yl]-thiazole-5-carboxylic acid hydroxyamide; 2-[4-(4-fluoro-2-methylphenylsulfonyl)-piperazin-1-yl]-thiazole-5-carboxylic acid hydroxyamide; 2-[4-(3-fluoro-4-methylphenylsulfonyl)-piperazin-1-yl]-thiazole-5-carboxylic acid hydroxyamide; 2-[4-(2-methyl-6-chlorophenylsulfonyl)-piperazin-1-yl]-thiazole-5-carboxylic acid hydroxyamide; 2-[4-(2,5-dimethyl-4-chlorophenylsulfonyl)-piperazin-1-yl]-thiazole-5-carboxylic acid hydroxyamide; 2-[4-(2,1,3-benzothiadiazole-5-sulfonyl)-piperazin-1-yl]-thiazole-5-carboxylic acid hydroxyamide; 2-[4-(2-benzothiphenesulfonyl)-piperazin-1-yl]-thiazole-5-carboxylic acid hydroxyamide; 2-[4-(2,3-dihydrobenzofuransulfonyl)-piperazin-1-yl]-thiazole-5-carboxylic acid hydroxyamide; 2-[4-(3,4-benzodioxansulfonyl)-piperazin-1-yl]-thiazole-5-carboxylic acid hydroxyamide; 2-[4-(3-biphenylsulfonyl)-piperazin-1-yl]-thiazole-5-carboxylic acid hydroxyamide; 2-[4-(2-phenoxypyridine-5-sulfonyl)-piperazin-1-yl]-thiazole-5-carboxylic acid hydroxyamide; 2-[4-(2-{2-methylthiopyrimidine-4-yl}-5-thiophenesulfonyl)-piperazin-1-yl]-thiazole-5-carboxylic acid hydroxyamide; 2-(4-[4-{(2-(pyrrolidin-1-ylmethyl)-thien-3-yl) phenylsulfonyl}]-piperazin-1-yl)-1,3-thiazole-5-carboxylic acid hydroxyamide; 2-(4-{4-[(5-(pyrrolidin-1-ylmethyl)-thien-2-yl]phenylsulfonyl}-piperazin-1-yl)-1,3-thiazole-5-carboxylic acid hydroxyamide; 2-{4-[(3′-((4-methylpiperazin-1-yl)methyl)-1,1′-biphenylsulfonyl]-piperazin-1-yl}-1,3-thiazole-5-carboxylic acid hydroxyamide; 2-{4-[3′-{[(2-(dimethylamino)ethyl)(methyl)amino]methyl}-1,1′-biphenylsulfonyl]piperazin-1-yl}-1,3-thiazole-5-carboxylic acid hydroxyamide; 2-[4-(3,5-difluorophenylsulfonyl)-piperazin-1-yl]-thiazole-5-carboxylic acid hydroxyamide; 2-(4-{4-[(3-(pyrrolidin-1-ylmethyl)-thien-2-yl]phenylsulfonyl}-piperazin-1-yl)-1,3-thiazole-5-carboxylic acid hydroxyamide; 2-{4-[(3′-[isopropyl(methyl)amino]methyl]-1,1′-biphenylsulfonyl]-piperazin-1-yl}-1,3-thiazole-5-carboxylic acid hydroxyamide; 2-{4-[(3′-[ethyl(methyl)amino]methyl]-1,1′-biphenylsulfonyl]-piperazin-1-yl}-1,3-thiazole-5-carboxylic acid hydroxyamide; 2-{4-[(3′-fluoro-1,1′-biphenylsulfonyl]piperazin-1-yl})-1,3-thiazole-5-carboxylic acid hydroxyamide; 2-(4-{[4-(1,3-benzodioxol-5-yl)phenyl]sulfonyl}piperazin-1-yl)-1,3-thiazole-5-carboxylic acid hydroxyamide; 2-[4-(n-butylsulfonyl)-piperazin-1-yl]-thiazole-5-carboxylic acid hydroxyamide; 2-[4-(chlorophenylsulfonyl)-piperazin-1-yl]-thiazole-5-carboxylic acid hydroxyamide; 2-{4-[(5-bromothien-2-yl)sulfonyl]piperazin-1-yl}-N-hydroxy-1,3-thiazole-5-carboxamide; 2-{4-[(4′-chloro-1,1′-biphenylsulfonyl]piperazin-1-yl}-1,3-thiazole-5-carboxylic acid hydroxyamide; 2-{4-[(4′-methoxy-1,1′-biphenylsulfonyl]piperazin-1-yl}-1,3-thiazole-5-carboxylic acid hydroxyamide; 2-{4-[(4′-(2,2-dimethylpropyl)-1,1′-biphenylsulfonyl]piperazin-1-yl}-1,3-thiazole-5-carboxylic acid hydroxyamide; 2-{4-[(4-thien-2-ylphenyl)sulfonyl]piperazin-1-yl}-1,3-thiazole-5-carboxylic acid hydroxamide; 2-(4-{[4-(1-(2,2-dimethylprop-oxycarbonyl)-1H-pyrrol-2-yl)phenyl]-sulfonyl}-piperazin-1-yl)-1,3-thiazole-5-carboxylic acid hydroxyamide; 2-(4-{[4-(1H-pyrrol-2-yl)phenyl]sulfonyl}piperazin-1-yl)-1,3-thiazole-5-carboxylic acid hydroxyamide; 2-{4-[(3′-(piperidin-1-ylmethyl)-1,1′-biphenylsulfonyl]piperazin-1-yl}-1,3-thiazole-5-carboxylic acid hydroxyamide; 2-{4-[(3′-(4-methylpiperidin-1-ylmethyl 1,1′-biphenylsulfonyl]-piperazin-1-yl}-1,3-thiazole-5-carboxylic acid hydroxyamide; 2-{4-[(3′-(hexahydroazepin-1-ylmethyl)-1,1′-biphenylsulfonyl]-piperazin-1-yl}-1,3-thiazole-5-carboxylic acid hydroxyamide; 2-{4-[(3′-((diethylamino)methyl)-1,1′-biphenylsulfonyl]piperazin-1-yl}-1,3-thiazole-5-carboxylic acid hydroxyamide; 2-{4-[(3′-((methyl(3-propenyl)amino)methyl)phenylsulfonyl]piperazin-1-yl}-1,3-thiazole-5-carboxylic acid hydroxyamide; 2-(4-{4-[(3-(pyrrolidin-1-ylmethyl)-2-furyl]phenylsulfonyl}piperazin-1-yl)-1,3-thiazole-5-carboxylic acid hydroxyamide; 2-(4-{5-[3-(pyrrolidin-1-ylmethyl)phenyl]thiophene-2-sulfonyl}piperazin-1-yl)-1,3-thiazole-5-carboxylic acid hydroxyamide; 2-[4-(4-pyrzaol-1-yl-4-sulfonyl)-piperazin-1-yl]-thiazole-5-carboxylic acid hydroxyamide; 2-[4-(4-{1-methylimidazol-4-yl}-4-sulfonyl)-piperazin-1-yl]-thiazole-5-carboxylic acid hydroxyamide; 2-[4-(4-methoxyphenyl-4-sulfonyl)-piperazin-1-yl]-thiazole-5-carboxylic acid hydroxyamide; 2-[4-{3-trifluoromethylphenyl}-4-sulfonyl)-piperazin-1-yl]-thiazole-5-carboxylic acid hydroxyamide; 2-[4-(4-{N,N-dimethylaminomethylphenyl}-4-sulfonyl)-piperazin-1-yl]-thiazole-5-carboxylic acid hydroxyamide; 2-[4-(4-{N-morpholinomethylphenyl}-4-sulfonyl)-piperazin-1-yl]-thiazole-5-carboxylic acid hydroxyamide; 2-[4-(4-{N-pyrrolidinylmethylphenyl}-4-sulfonyl)-piperazin-1-yl]-thiazole-5-carboxylic acid hydroxyamide; 2-[4-(4-phenethylphenyl-4-sulfonyl)-piperazin-1-yl]-thiazole-5-carboxylic acid hydroxyamide; 2-[4-(4-ethylphenyl-4-sulfonyl)-piperazin-1-yl]-thiazole-5-carboxylic acid hydroxyamide; 2-[4-(3-hydroxymethylphenyl-4-sulfonyl)-piperazin-1-yl]-thiazole-5-carboxylic acid hydroxyamide; 2-[4-(3-pyrrolidin-1-ylmethylphenyl-4-sulfonyl)-piperazin-1-yl]-thiazole-5-carboxylic acid hydroxyamide; 2-[4-(4-pyrimid-5-ylphenyl-4-sulfonyl)-piperazin-1-yl]-thiazole-5-carboxylic acid hydroxyamide; 2-{4-[(4′-(acetamidophenyl)-phenylsulfonyl]piperazin-1-yl}-1,3-thiazole-5-carboxylic acid hydroxyamide; 2-[4-(3-pyridylphenyl-4-sulfonyl)-piperazin-1-yl]-thiazole-5-carboxylic acid hydroxyamide; 2-[4-(3-{N,N-dimethylaminomethylphenyl}-4-sulfonyl)-piperazin-1-yl]-thiazole-5-carboxylic acid hydroxyamide; 2-[4-(3-methoxymethylbenzenesulfonyl)-piperazin-1-yl]-thiazole-5-carboxylic acid hydroxyamide; 2-[4-(4-acetamido-4-sulfonyl)-piperazin-1-yl]-thiazole-5-carboxylic acid hydroxyamide; 2-[4-(3-cyanophenyl}-4-sulfonyl)-piperazin-1-yl]-thiazole-5-carboxylic acid hydroxyamide; 2-{4-[(2-chloro-5-methoxyphenyl)sulfonyl]piperazin-1-yl}-1,3-thiazole-5-carboxylic acid hydroxyamide; 2-(4-{[3-(difluoromethoxy)phenyl]sulfonyl}piperazin-1-yl)-1,3-thiazole-5-carboxylic acid hydroxyamide; 2-{4-[(4-methyl-3-nitrophenyl)sulfonyl]piperazin-1-yl}-1,3-thiazole-5-carboxylic acid hydroxyamide; 2-{4-[(2,5-dimethoxyphenyl)sulfonyl]piperazin-1-yl}-1,3-thiazole-5-carboxylic acid hydroxyamide; 2-{4-[(2,5-dimethylphenyl)sulfonyl]piperazin-1-yl}-1,3-thiazole-5-carboxylic acid hydroxyamide; 2-(4-{[2-(pyrrolidin-1-ylmethyl-4-methylphenyl]sulfonyl}piperazin-1-yl)-1,3-thiazole-5-carboxylic acid hydroxyamide; 2-(4-{[3-fluoro-4-(pyrrolidin-1-yl)phenyl]sulfonyl}piperazin-1-yl)-1,3-thiazole-5-carboxylic acid hydroxyamide; 2-(4-{[4-(piperidin-1-yl)phenyl]sulfonyl}piperazin-1-yl)-1,3-thiazole-5-carboxylic acid hydroxyamide; 2-(4-{[4-(morpholin-4-yl)phenyl]sulfonyl}piperazin-1-yl)-1,3-thiazole-5-carboxylic acid hydroxyamide; 2-{4-[(trifluoromethyl)sulfonyl]piperazin-1-yl}-1,3-thiazole-5-carboxylic acid hydroxyamide; 2-{4-[ethylsulfonyl]piperazin-1-yl}-1,3-thiazole-5-carboxylic acid hydroxyamide; 2-{4-[3′-{[N-acetylamino]methyl}-1,1′-biphenylsulfonyl]piperazin-1-yl}-1,3-thiazole-5-carboxylic acid hydroxyamide; 2-{4-[3′-{methoxymethyl)}-1,1′-biphenylsulfonyl]piperazin-1-yl}-1,3-thiazole-5-carboxylic acid hydroxyamide; 2-{4-[4-(2-(pyrrolidin-1-ylmethyl)-thien-4-yl)phenylsulfonyl]piperazin-1-yl}-1,3-thiazole-5-carboxylic acid hydroxyamide; 2-(4-{4-[3-((dimethylamino)-methyl)-2-furyl]phenylsulfonyl}piperazin-1-yl)-1,3-thiazole-5-carboxylic acid hydroxyamide; 2-(4-{4-[(4-(pyrrolidin-1-ylmethyl-2-furyl]phenylsulfonyl}piperazin-1-yl)-1,3-thiazole-5-carboxylic acid hydroxyamide; 2-(4-{4-[(3-((dimethylamino)methyl)thien-2-yl]phenylsulfonyl}piperazin-1-yl)-1,3-thiazole-5-carboxylic acid hydroxyamide; 2-{4-[3′-{[(2-hydroxyethyl)(methyl)amino]methyl}-1,1′-biphenylsulfonyl]piperazin-1-yl}-1,3-thiazole-5-carboxylic acid hydroxyamide; 2-{4-[3′-{[bis(2-hydroxyethyl)amino]methyl}-1,1′-biphenylsulfonyl]piperazin-1-yl}-1,3-thiazole-5-carboxylic acid hydroxyamide; 2-{4-[3′-(3,6-dihydropyridin-1(2H)-ylmethyl)-1,1′-biphenylsulfonyl]piperazin-1-yl}-1,3-thiazole-5-carboxylic acid hydroxyamide; 2-{4-[3′-{[(butyl)(methyl)amino]methyl}-1,1′-biphenylsulfonyl]piperazin-1-yl}-1,3-thiazole-5-carboxylic acid hydroxyamide; 2-{4-[3′-((piperazin-1-yl)methyl)-1,1′-biphenylsulfonyl]piperazin-1-yl}-1,3-thiazole-5-carboxylic acid hydroxyamide; 2-{4-[3′-{[(2-methoxyethyl)(methyl)amino]methyl}-1,1′-biphenylsulfonyl]piperazin-1-yl}-1,3-thiazole-5-carboxylic acid hydroxyamide; 2-{4-[3′-{[bis(3-propenyl))amino]methyl}-1,1′-biphenylsulfonyl]piperazin-1-yl}-1,3-thiazole-5-carboxylic acid hydroxyamide; 2-{4-[3′-{[methylamino]methyl}-1,1′-biphenylsulfonyl]piperazin-1-yl}-1,3-thiazole-5-carboxylic acid hydroxyamide and 2-{4-[2′-{pyrrolidin-1-ylmethyl}-1,1′-biphenylsulfonyl]piperazin-1-yl}-1,3-thiazole-5-carboxylic acid hydroxyamide; and tautomers, isomers, prodrugs and pharmaceutically acceptable salts thereof. Yet other HDAC inhibitors are compounds selected from the group consisting of: 2-[4-(naphtha-2-yl-4-sulfonyl)-piperazin-1-yl]-thiazole-5-carboxylic acid hydroxyamide 2-[4-(4-trifluoromethoxy-benzene sulfonyl)-piperazin-1-yl]-thiazole-5-carboxylic acid hydroxyamide 2-[4-(4-toluene-4-sulfonyl)-piperazin-1-yl]-thiazole-5-carboxylic acid hydroxyamide 2-[4-(biphenyl-4-sulfonyl)-piperazin-1-yl]-thiazole-5-carboxylic acid hydroxyamide 2-[4-(4-trifluoromethyl-benzenesulfonyl)-piperazin-1-yl]-thiazole-5-carboxylic acid hydroxyamide 2-[4-(3,4-dimethoxy-benzene sulfonyl)-piperazin-1-yl]-thiazole-5-carboxylic acid hydroxyamide 2-[4-(5-dimethylamino-naphthalene-1-sulfonyl)-piperazin-1-yl]-thiazole-5-carboxylic acid hydroxyamide 2-[4-(4-acetyl-benzenesulfonyl)-piperazin-1-yl]-thiazole-5-carboxylic acid hydroxyamide 2-[4-(benzenesulfonyl)-piperazin-1-yl]-thiazole-5-carboxylic acid hydroxyamide 2-[4-(4-nitro-benzenesulfonyl)-piperazin-1-yl]-thiazole-5-carboxylic acid hydroxyamide 2-[4-(4-fluoro-benzenesulfonyl)-piperazin-1-yl]-thiazole-5-carboxylic acid hydroxyamide 2-[4-(4-pyrrolidinylbenzenesulfonyl)-piperazin-1-yl]-thiazole-5-carboxylic acid hydroxyamide 2-[4-(thiophene-2-benzenesulfonyl)-piperazin-1-yl]-thiazole-5-carboxylic acid hydroxyamide 2-[4-(N-methyl-2,3-dihydrobenzisoxazinylsulfonyl)-piperazin-1-yl]-thiazole-5-carboxylic acid hydroxyamide 2-[4-(4-isopropylphenylsulfonyl)-piperazin-1-yl]-thiazole-5-carboxylic acid hydroxyamide 2-[4-(trans-2-phenylethanelsulfonyl)-piperazin-1-yl]-thiazole-5-carboxylic acid hydroxyamide 2-[4-(2-chlorophenylsulfonyl)-piperazin-1-yl]-thiazole-5-carboxylic acid hydroxyamide 2-[4-(3-chlorophenylsulfonyl)-piperazin-1-yl]-thiazole-5-carboxylic acid hydroxyamide 2-[4-(3,4-dichlorophenylsulfonyl)piperazin-1-yl]-thiazole-5-carboxylic acid hydroxyamide 2-[4-(N,N-dimethylsulfonyl)-piperazin-1-yl]-thiazole-5-carboxylic acid hydroxyamide 2-[4-(4-methylsulfonylphenylsulfonyl)-piperazin-1-yl]-thiazole-5-carboxylic acid hydroxyamide 2-[4-(pyridine-3-sulfonyl)-piperazin-1-yl]-thiazole-5-carboxylic acid hydroxyamide 2-[4-(2-methylphenylsulfonyl)-piperazin-1-yl]-thiazole-5-carboxylic acid hydroxyamide 2-[4-(3-methylphenylsulfonyl)-piperazin-1-yl]-thiazole-5-carboxylic acid hydroxyamide 2-{4-[(3′-[(dimethylamino)methyl]-1,1′-biphenylsulfonyl]piperazin-1-yl}-1,3-thiazole-5-carboxylic acid hydroxyamide 2-[4-(4-n-propylphenylsulfonyl)-piperazin-1-yl]-thiazole-5-carboxylic acid hydroxyamide 2-[4-(3,5-dimethylphenylsulfonyl)-piperazin-1-yl]-thiazole-5-carboxylic acid hydroxyamide 2-[4-(4-t-butylphenylsulfonyl)-piperazin-1-yl]-thiazole-5-carboxylic acid hydroxyamide 2-[4-(benzylsulfonyl)-piperazin-1-yl]-thiazole-5-carboxylic acid hydroxyamide 2-[4-(4′-N,N-dimethylcarboxamido-1,1′-biphenylsulfonyl)piperazin-1-yl]-1,3-thiazole-5-carboxylic acid hydroxyamide 2-[4-(4′-methylsulfonylamino-1,1′-biphenylsulfonyl)piperazin-1-yl]-1,3-thiazole-5-carboxylic acid hydroxyamide 2-{4-[(4′-((dimethylamino)methyl}1,1′-biphenylsulfonyl]piperazin-1-yl}-1,3-thiazole-5-carboxylic acid hydroxyamide 2-[4-(3,5-dimethylisoxazolesulfonyl) piperazin-1-yl]-thiazole-5-carboxylic acid hydroxyamide 2-[4-({4-morpholino}-3-pyridylsulfonyl)-piperazin-1-yl]-thiazole-5-carboxylic acid hydroxyamide 2-[4-(3′-(dimethylamino)-1,1′-biphenylsulfonyl)piperazin-1-yl]-1,3-thiazole-5-carboxylic acid hydroxyamide 2-{4-[(3′-(pyrrolidin-1-ylmethyl 1,1′-biphenylsulfonyl]piperazin-1-yl}-1,3-thiazole-5-carboxylic acid hydroxyamide 2-[4-(4-dimethylaminophenylsulfonyl)-piperazin-1-yl]-thiazole-5-carboxylic acid hydroxyamide 2-[4-(3,4-dimethylphenylsulfonyl)-piperazin-1-yl]-thiazole-5-carboxylic acid hydroxyamide 2-[4-(4′-(morpholin-4-ylcarbonyl)-1,1′-biphenylsulfonyl)piperazin-1-yl]-1,3-thiazole-5-carboxylic acid hydroxyamide 2-(4-{4-[(2-((dimethylamino)-methyl)thien-3-yl]phenylsulfonyl}piperazin-1-yl)-1,3-thiazole-5-carboxylic acid hydroxyamide 2-{4-[(4′-fluoro-1,1′-biphenyl-4-yl)sulfonyl]piperazin-1-yl}-1,3-thiazole-5-carboxylic acid hydroxyamide 2-[4-(3-chloro-2-methylphenylsulfonyl)-piperazin-1-yl]-thiazole-5-carboxylic acid hydroxyamide 2-[4-(3-chloro-4-methylphenylsulfonyl)-piperazin-1-yl]-thiazole-5-carboxylic acid hydroxyamide 2-[4-(4-methoxyphenylsulfonyl)-piperazin-1-yl]-thiazole-5-carboxylic acid hydroxyamide 2-{4-[4-(pyridin-4-yl)phenylsulfonyl]piperazin-1-yl}-1,3-thiazole-5-carboxylic acid hydroxyamide 2-[4-(3-fluorophenylsulfonyl)-piperazin-1-yl]-thiazole-5-carboxylic acid hydroxyamide 2-[4-(2-fluorophenylsulfonyl)-piperazin-1-yl]-thiazole-5-carboxylic acid hydroxyamide 2-[4-(2-methyl-5-fluorophenylsulfonyl)-piperazin-1-yl]-thiazole-5-carboxylic acid hydroxyamide 2-[4-(2-trifluoromethylphenylsulfonyl)-piperazin-1-yl]-thiazole-5-carboxylic acid hydroxyamide 2-{4-[(3′-chloro-1,1′-biphenyl-4-yl)sulfonyl]piperazin-1-yl}-1,3-thiazole-5-carboxylic acid hydroxyamide 2-{4-[(2′-chloro-1,1′-biphenyl-4-yl)sulfonyl]piperazin-1-yl}-1,3-thiazole-5-carboxylic acid hydroxyamide 2-(4-{[4-(2-furyl)phenyl]sulfonyl}piperazin-1-yl)-1,3-thiazole-5-carboxylic acid hydroxyamide 2-[4-(3,4-difluorophenylsulfonyl)-piperazin-1-yl]-thiazole-5-carboxylic acid hydroxyamide 2-[4-(4-fluoro-2-methylphenylsulfonyl)-piperazin-1-yl]-thiazole-5-carboxylic acid hydroxyamide 2-[4-(3-fluoro-4-methylphenylsulfonyl)-piperazin-1-yl]-thiazole-5-carboxylic acid hydroxyamide 2-[4-(2-methyl-6-chlorophenylsulfonyl)piperazin-1-yl]-thiazole-5-carboxylic acid hydroxyamide 2-[4-(2,5-dimethyl-4-chlorophenylsulfonyl)-piperazin-1-yl]-thiazole-5-carboxylic acid hydroxyamide 2-[4-(2,1,3-benzothiadiazole-5-sulfonyl)-piperazin-1-yl]-thiazole-5-carboxylic acid hydroxyamide 2-[4-(2-benzothiphenesulfonyl)-piperazin-1-yl]-thiazole-5-carboxylic acid hydroxyamide 2-[4-(2,3-dihydrobenzofuransulfonyl)-piperazin-1-yl]-thiazole-5-carboxylic acid hydroxyamide 2-[4-(3,4-benzodioxansulfonyl)-piperazin-1-yl]-thiazole-5-carboxylic acid hydroxyamide 2-[4-(3-biphenylsulfonyl)piperazin-1-yl]-thiazole-5-carboxylic acid hydroxyamide 2-[4-(2-phenoxypyridine-5-sulfonyl)-piperazin-1-yl]-thiazole-5-carboxylic acid hydroxyamide 2-[4-(2-{2-methylthiopyrimidine-4-yl}-5-thiophenesulfonyl)-piperazin-1-yl]-thiazole-5-carboxylic acid hydroxyamide 2-(4-[4-{(2-(pyrrolidin-1-ylmethyl)-thien-3-yl)phenylsulfonyl}]-piperazin-1-yl)-1,3-thiazole-5-carboxylic acid hydroxyamide 2-(4-{4-[(5-(pyrrolidin-1-ylmethyl)-thien-2-yl]phenylsulfonyl}-piperazin-1-yl)-1,3-thiazole-5-carboxylic acid hydroxyamide 2-{4-[(3′-((4-methylpiperazin-1-yl)methyl)-1,1′-biphenylsulfonyl]-piperazin-1-yl}-1,3-thiazole-5-carboxylic acid hydroxyamide 2-{4-[3′-{[(2-(dimethylamino) ethyl)(methyl)amino]methyl}-1,1′-biphenylsulfonyl]piperazin-1-yl}-1,3-thiazole-5-carboxylic acid hydroxyamide 2-[4-(3,5-difluorophenylsulfonyl)-piperazin-1-yl]-thiazole-5-carboxylic acid hydroxyamide 2-(4-{4-[(3-(pyrrolidin-1-ylmethyl)-thien-2-yl]phenylsulfonyl}-piperazin-1-yl)-1,3-thiazole-5-carboxylic acid hydroxyamide 2-{4-[(3′-[isopropyl(methyl)amino]methyl]-1,1′-biphenylsulfonyl]-piperazin-1-yl}-1,3-thiazole-5-carboxylic acid hydroxyamide 2-{4-[(3′-[ethyl(methyl)amino]methyl]-1,1′-biphenylsulfonyl]-piperazin-1-yl}-1,3-thiazole-5-carboxylic acid hydroxyamide 2-{4-[(3′-fluoro-1,1′-biphenylsulfonyl]piperazin-1-yl}-1,3-thiazole-5-carboxylic acid hydroxyamide 2-(4-{[4-(1,3-benzodioxol-5-yl)phenyl]sulfonyl}piperazin-1-yl)-1,3-thiazole-5-carboxylic acid hydroxyamide 2-[4-(n-butylsulfonyl)-piperazin-1-yl]-thiazole-5-carboxylic acid hydroxyamide 2-[4-(chlorophenylsulfonyl)-piperazin-1-yl]-thiazole-5-carboxylic acid hydroxyamide 2-{4-[(5-bromothien-2-yl)sulfonyl]piperazin-1-yl}-N-hydroxy-1,3-thiazole-5-carboxamide 2-{4-[(4′-chloro-1,1′-biphenylsulfonyl]piperazin-1-yl}-1,3-thiazole-5-carboxylic acid hydroxyamide 2-{4-[(4′-methoxy-1,1′-biphenylsulfonyl]piperazin-1-yl}-1,3-thiazole-5-carboxylic acid hydroxyamide 2-{4-[(4′-(2,2-dimethylpropyl)-1,1′-biphenylsulfonyl]piperazin-1-yl}-1,3-thiazole-5-carboxylic acid hydroxyamide 2-{4-[(4-thien-2-ylphenyl)sulfonyl]piperazin-1-yl}-1,3-thiazole-5-carboxylic acid hydroxamide 2-(4-{[4-(1-(2,2-dimethylprop-oxycarbonyl 1H-pyrrol-2-yl)phenyl]-sulfonyl}-piperazin-1-yl)-1,3-thiazole-5-carboxylic acid hydroxyamide 2-(4-{[4-(1H-pyrrol-2-yl)phenyl]sulfonyl}piperazin-1-yl)-1,3-thiazole-5-carboxylic acid hydroxyamide 2-{4-[(3′-(piperidin-1-ylmethyl)-1,1′-biphenylsulfonyl]piperazin-1-yl}-1,3-thiazole-5-carboxylic acid hydroxyamide 2-{4-[(3′-(4-methylpiperidin-1-ylmethyl)-1,1′-biphenylsulfonyl]-piperazin-1-yl}-1,3-thiazole-5-carboxylic acid hydroxyamide 2-{4-[(3′-(hexahydroazepin-1-ylmethyl)-1,1′-biphenylsulfonyl]-piperazin-1-yl}-1,3-thiazole-5-carboxylic acid hydroxyamide 2-{4-[(3′-((diethylamino)methyl} 1,1′-biphenylsulfonyl]piperazin-1-yl}-1,3-thiazole-5-carboxylic acid hydroxyamide 2-{4-[(3′-((methyl(3-propenyl)amino)methyl)phenylsulfonyl]piperazin-1-yl}-1,3-thiazole-5-carboxylic acid hydroxyamide 2-(4-{4-[(3-(pyrrolidine-1-ylmethyl)-2-furyl]phenylsulfonyl}piperazin-1-yl)-1,3-thiazole-5-carboxylic acid hydroxyamide 2-(4-{5-[3-(pyrrolidin-1-ylmethyl)phenyl]thiophene-2-sulfonyl}piperazin-1-yl)-1,3-thiazole-5-carboxylic acid hydroxyamide 2-[4-(4-pyrzaol-1-yl-4-sulfonyl)-piperazin-1-yl]-thiazole-5-carboxylic acid hydroxyamide 2-[4-(4-{1-methylimidazol-4-yl}-4-sulfonyl)-piperazin-1-yl]-thiazole-5-carboxylic acid hydroxyamide 2-[4-(4-methoxyphenyl-4-sulfonyl)-piperazin-1-yl]-thiazole-5-carboxylic acid hydroxyamide 2-[4-{3-trifluoromethylphenyl}-4-sulfonyl)-piperazin-1-yl]-thiazole-5-carboxylic acid hydroxyamide 2-[4-(4-{N,N-dimethylaminomethylphenyl}-4-sulfonyl)-piperazin-1-yl]-thiazole-5-carboxylic acid hydroxyamide 2-[4-(4-{N-morpholinomethylphenyl}-4-sulfonyl)-piperazin-1-yl]-thiazole-5-carboxylic acid hydroxyamide 2-[4-(4-{N-pyrrolidinylmethylphenyl}-4-sulfonyl)-piperazin-1-yl]-thiazole-5-carboxylic acid hydroxyamide 2-[4-(4-phenethylphenyl-4-sulfonyl)-piperazin-1-yl]-thiazole-5-carboxylic acid hydroxyamide 2-[4-(4-ethylphenyl-4-sulfonyl)-piperazin-1-yl]-thiazole-5-carboxylic acid hydroxyamide 2-[4-(3-hydroxymethylphenyl-4-sulfonyl)-piperazin-1-yl]-thiazole-5-carboxylic acid hydroxyamide 2-[4-(3-pyrrolidin-1-ylmethylphenyl-4-sulfonyl)-piperazin-1-yl]-thiazole-5-carboxylic acid hydroxyamide 2-[4-(4-pyrimid-5-ylphenyl-4-sulfonyl)-piperazin-1-yl]-thiazole-5-carboxylic acid hydroxyamide 2-{4-[(4′-(acetamidophenyl)-phenylsulfonyl]piperazin-1-yl}-1,3-thiazole-5-carboxylic acid hydroxyamide 2-[4-(3-pyridylphenyl-4-sulfonyl)-piperazin-1-yl]-thiazole-5-carboxylic acid hydroxyamide 2-[4-(3-{N,N-dimethylaminomethylphenyl}-4-sulfonyl)-piperazin-1-yl]-thiazole-5-carboxylic acid hydroxyamide 2-[4-(3-methoxymethylbenzenesulfonyl)-piperazin-1-yl]-thiazole-5-carboxylic acid hydroxyamide 2-[4-(4-acetamido-4-sulfonyl)-piperazin-1-yl]-thiazole-5-carboxylic acid hydroxyamide 2-[4-(3-cyanophenyl}-4-sulfonyl)-piperazin-1-yl]-thiazole-5-carboxylic acid hydroxyamide or tautomers, isomers, prodrugs and pharmaceutically acceptable salts thereof.

### EXAMPLES AND DRAWING DESCRIPTION

### Example 1

### DNA Methylation Analysis

DNA methylation is an epigenetic event that affects cell function by altering gene expression and refers to the covalent addition of a methyl group, catalyzed by DNA methyltransferase (DNMT), to the 5-carbon of cytosine in a CpG dinucleotide. Methods for DNA methylation analysis can be divided roughly into two types: global and gene-specific methylation analysis. For global methylation analysis, there are methods which measure the overall level of methyl cytosines in genome such as chromatographic methods and methyl accepting capacity assay. For gene-specific methylation analysis, a large number of techniques have been developed. Most early studies used methylation-sensitive restriction enzymes to digest DNA followed by Southern detection or PCR amplification. Recently, bisulfite reaction-based methods have become very popular such as methylation specific PCR (MSP), bisulfite genomic sequencing PCR. Additionally, in order to identify unknown methylation hot-spots or methylated CpG islands in the genome, several of genome-wide screen methods have been invented such as Restriction Landmark Genomic Scanning for Methylation (RLGS-M), and CpG island microarray. For the various aspects of this technology is available in the art. The bisulfite modification (conversion) of DNA is obtainable using sodium bisulfite to convert unmethylated cytosines to uracils and subsequently detecting methylated cytosines using methylation specific PCR (MSP) technique or bisulfite genomic sequencing after PCR amplification with or without cloning. Protocols or procedures for handling such nanogram quantities of DNA have been published in technical manuals and journals (H. Hayatsu et al., Biochemistry 1970, 9:2858; K. D. Tremblay, 1998 Technical Tips Online; S. J. Clark et al., Nucleic Acids Res. 1994, 22:1827; M. Frommer et al., PNAS 1992, 89:1827; E. J. Oakeley, Pharmacology & Therapeutics 1999, 84:389; M. F. Fraga and M. Esteller, Biotechniques 2002, 33:632; L. C. Li and R. Dahiya, Bioinformatics 2002, 18(11):1427; and UCLA Fan Lab Bisulfite Treatment, by Shaun Fouse, online or via Dr. Guoping Fan, Department of Human Genetics, David Geffen School of Medicine, Gonda BLDG Rm. 6554, P.O. Box 957088, University of California Los Angeles, Los Angeles, Calif. 90095-7088). The analysis of DNA methylation can be carried out by bisulphite sequencing. This method allows precise analysis of methylation in a certain region by converting all nonmethylated cytosines into tymines, while methylated cytosines remain unchanged. This method requires small amount of genomic DNA and therefore seems to be very useful for the analysis of clinical samples, where the material amount is limited. Such method can be optimizes the method using genomic DNA from a cell line and then apply it to valuable samples. Primers have to be developed for bisulphite converted DNA. One can generate a model of bisulphite treated DNA by substituting all cytosines which are not in CG context into tymines. And then design the primers in the way that they don not contain any CG. If this is impossible, one can use C/T at the place of C in CG context. Usually primer selection is the most critical in bisulphite-based methylation analysis, since the complexity of DNA is reduced. Therefore the skilled man can select two to three pairs of primers, check them on bisulphite modified DNA, and use the most specific ones. Protocols and procedures of such analysis of DNA methylation by bisulphite sequences are available for the man skilled in the art (A. Kaneda et al., Cancer Lett. 2004, 212:203-210; and A. Kaneda et al., Cancer Sci. 2004, 95:58-64).

### Example 2

### Quantitative Methyl-Specific PCR (qMSP)

Quantitative Methyl-Specific PCR (qMSP) (J. G. Herman et al., Proc. Natl. Acad. Sci. USA Vol. 93, pp. 9821-9826, September 1996) is the ideal technology for early detection of PrCa. The detection of DNA methylation is based on bisulphite treatment of DNA, which reproducibly converts unmethylated cytosine into uracil. In this assay, the methylated cytosines remain unchanged. The bisulphite conversion is combined with a PCR-based approach known as Methylation-Specific PCR (MSP). This technique has been described enabling in U.S. Pat. Nos. 5,786,146, 6,017,704, 6,265,171, and 6,200,756 and is hereby incorporated by reference. It has major advantages over other PrCa screening methods:

The qMSP technology is extremely sensitive and can detect one to ten tumor cells among thousands of healthy cells.

The qMSP technology is highly reproducible, quick and easy to perform.

It is a low-cost assay only requiring standard qPCR equipment.

Since DNA is very stable (more stable than proteins), the qMSP test can be performed on many sample types, including DNA isolated from paraffin embedded, formalin fixed prostatectomy samples as well as body fluids such as blood plasma, urine or prostatic secretions (M. L. Gonzalgo et al. (2004), Urology 63:414-418).

### Example 3

### Predicted CpG Islands from the MSMB Gene

A detailed bioinformatical analysis of 15 kbp region upstream and downstream of the transcriptional start site of the MSMB gene resulted in the prediction of 10 CpG islands. All CpG islands, except for CpG5 and 7, are predicted by the program Newcpgreport, with the following parameters: window=50, window shift=1, Island size>200, GC %>0.2 and O/E (observed/predicted>0.2. CpG5 and 7 island are predicted by the program Methprimer with the following parameters: Island size>100, GC %>0.5 and O/E>0.6 A schematic representation and localization of the predicted CpG islands has been displayed in FIG. 6 and Table 1.

### Example 4

### Non-CpG Methylation in CpG4-5 Islands of the MSMB Gene

### Location, See Table 1

Genomic DNA has been isolated from five prostate cancer samples, indicated as C1-5, and four Benign Prostatic Hyperplasia, indicated as B1-4 with GenElute Mammalian Genomic DNA Miniprep kit of Sigma. Human genomic DNA from whole blood, indicated as H, was purchased from Clonetech (cat no. 636401). Total genomic DNA of all samples, as well as the human genomic DNA from the whole blood, was bisulphite treated converting unmethylated cytosines to uracil. Methylated cytosines remained conserved. Bisulphite treatment was performed using the Zymo gold kit of Zymo Research (USA) according to the protocol of the manufacturer. After bisulphite conversion 10 ng of each DNA sample was used in a subsequent PCR to amplify the CpG islands 4 and 5 as one 799 bp fragment (sense primer is SEQ ID NO:67 and antisense primer is SEQ ID NO:68). Each reaction contained the following: 400 μM dNTPs, 10 pmol each primer, 2 U JumpStart™ Taq DNA Polymerase (Sigma), 10 ng DNA (bisulphite treated). Denaturation at 95° C. for 4 minutes was followed by five cycles (95° C. for 30 seconds, annealing at 53° C. for 90 seconds, elongation at 68° C. for 120 seconds) and then by 30 cycles (95° C. for 30 seconds, annealing at 53° C. for 90 seconds, elongation at 68° C. for 90 seconds). A final elongation at 68° C. was carried out for five minutes. PCR products were subcloned in the pGem-T vector (Promega) according the manufacturer's protocol. The plasmids were sequenced using ABI Big Dye Terminator Cycle Sequencing Kit (Applied Biosystems). We determined the methylation status of all CG dinucleotides, known as CpG methylation, and of some CA, CT, CC dinucleotides, known as non-CpG methylation.

Table 2. Map of the methylation state of all CG dinucleotides present in CpG 4-5 island of the MSMB gene. The CpG 4-5 island is located at −3533 nt upstream to −2734 nt upstream of the transcriptional start site (TSS) of the MSMB gene. The TSS is located at position 51219559 on the forward strand of chromosome 10 (SEQ ID NO:5). The numbers in the first row represent the location of all CG dinucleotides towards the TSS of the MSMB gene (SEQ ID NO:5). Methylated CG dinucleotides are denoted by C and unmethylated CGs by T. Deleted CG-dinucleotides in some clones are denoted by DEL. A CG-dinucleotide in a position −3234 towards the TSS that is mutated in some DNA samples (CG/GG mutation), is denoted by GG. Non-available data for the methylation status of some CG-dinucleotides in some clones are denoted by hyphen. In the first column, the name of the analyzed biological sample followed by the number of the analyzed clone is indicated. C1-5, prostate cancer samples; B1-4, Benign Prostatic Hyperplasia; H, human genomic DNA.

Table 3. Map of the non-CG dinucleotides which are present in CpG4-5 island of the MSMB gene and were methylated in at least one of the analyzed samples. The CpG4-5 island is located at −3533 nt upstream to −2734 nt upstream of the transcriptional start site (TSS) of the MSMB gene. The TSS is located at position 51219559 on the forward strand of chromosome 10 (SEQ ID NO:5). The numbers in first row are the location of the non-CG dinucleotides towards the TSS of the MSMB gene (SEQ ID NO:5). Methylated non-CG dinucleotides are denoted by C, followed by the next nucleotide (A or T) and indicated by grey boxes. Unmethylated non-CG dinucleotides are denoted by T followed by the next nucleotide (A or T). In the first column, you find the name of the analyzed biological sample followed by the number of the analyzed clone. C1-5, prostate cancer sample; B1-4, Benign Prostatic Hyperplasia; H, human genomic DNA.

Table 4. Map of the sequence alterations unrelated to DNA methylation present in CpG4-5 island of the MSMB gene and detected in at least one analyzed sample. The CpG4-5 island is located at −3533 nt upstream to −2734 nt upstream of the transcriptional start site (TSS) of the MSMB gene. The TSS is located at position 51219559 on the forward strand of chromosome 10 (SEQ ID NO:5). The numbers in the first row refer to the location of the nucleotide towards the TSS of the MSMB gene (SEQ ID NO:5). Letter in the table indicates the nucleotide (A, T, G or C) detected in the indicate clone; all sequence alterations are marked by grey color. Deleted AGT-trinucleotide at position −2969 towards TSS of the MSMB gene in some clones is denoted by DEL. In the first column, the name of the analyzed biological sample followed by the number of the analyzed clone is indicated. C1-5, prostate cancer sample; B1-4, Benign Prostatic Hyperplasia; H, human genomic DNA.

Table 5. Summary of all detected haplotypes (differential non-CpG methylation plus sequence alterations unrelated to DNA methylation) of CpG4-5 island of the MSMB gene in all analyzed DNA samples. The CpG4-5 island is located at −3533 nt upstream to −2734 nt upstream of the transcriptional start site (TSS) of the MSMB gene. The TSS is located at position 51219559 on the forward strand of chromosome 10 (SEQ ID NO:5). The 11 haplotypes (numbered in the first column) are generated based on data of differential non-CpG methylation (Table 3) and sequence alterations from Table 4. The numbers in first row are the location of the nucleotide towards the TSS of the MSMB gene (SEQ ID NO:5). In the first row the non-CpG dinucleotides are denoted in grey boxes and the sequence alterations unrelated to DNA methylation in dotted boxes. A star in the two last columns indicates the presence of the haplotype in at least one cancer and/or benign samples; a hyphen, correspondingly, the absence of the haplotype in all analyzed cancer and/or benign samples. A star in the two last rows indicates the presence of the definite nucleotide modification in at least one cancer and/or benign samples; a hyphen, correspondingly, the absence of the definite nucleotide modification in all analyzed cancer and/or benign samples.

It will be apparent to those skilled in the art that various modifications and variations can be made in defining the methylation levels of the promoter region of the MSMB gene of the invention and in construction of the system and method without departing from the scope or spirit of the invention. Examples of such modifications have been previously provided. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

TABLE 1
Location of the predicted CpG islands towards the
transcriptional start site of the MSMB gene.

| | location towards TSS of the MSMB |
|---|---|
| CpG island | gene (SEQ ID NO: 5) |
| CpG1 | −13877 to −13583 |
| CpG2 | −10528 to −10254 |
| CpG3 | −3920 to −3673 |
| CpG4 | −3471 to −3141 |
| CpG5* | −3128 to −2817 |
| CpG4-5** | −3533 to −2734 |
| CpG6 | −1671 to 1996 |
| CpG7*** | 2180 to 2390 |
| CpG8 | 5236 to 5616 |
| CpG9 | 7670 to 8030 |
| CpG10 | 11432 to 11754 |

*corresponds to CpG1 island in FIG. 4
**CpG4 island and CpG5 island are analyzed together by one PCR reaction and is indicated as CpG4-5 island
***corresponds to CpG2 island in FIG. 4

TABLE 2
CpG-methylation in CpG-island 4-5 of the MSMB gene

| | | | | | | | | | | −3234 | |
|---|---|---|---|---|---|---|---|---|---|---|---|
| | −3408 | −3383 | −3376 | −3345 | −3312 | −3308 | −3281 | −3250 | −3244 | CG-44 | −3174 |
| Clone | CG-53 | CG-52 | CG-51 | CG-50 | CG-49 | CG-48 | CG-47 | CG-46 | CG-45 | C-G | GC-41 |
| C1/24 | C | C | C | C | C | C | C | C | C | GG | C |
| C1/7 | C | C | C | C | C | C | C | C | C | GG | C |
| C1/1 | C | C | C | C | C | C | C | C | C | GG | C |
| C1/3 | C | C | C | C | C | C | C | C | C | GG | C |
| C1/8 | C | C | C | C | C | C | C | C | C | GG | C |
| C1/5 | C | C | C | C | C | C | C | C | C | GG | C |
| C1/11 | C | C | C | C | C | C | C | C | C | GG | C |
| C1/10 | C | C | C | C | C | C | C | C | C | CG | C |
| C1/2 | C | C | C | C | C | C | C | C | C | CG | C |
| C1/12 | C | C | C | C | C | C | C | C | C | CG | C |
| C1/15 | C | C | C | C | C | C | C | C | C | CG | C |
| C1/21 | C | C | C | C | C | C | C | C | C | CG | C |
| C1/18 | C | C | C | C | C | C | C | C | C | CG | C |
| C1/22 | C | C | C | C | C | C | C | C | C | CG | C |
| C1/4 | C | C | C | C | C | C | C | C | C | CG | C |
| C1/9 | C | C | C | C | C | C | C | C | C | CG | C |
| C1/16 | C | C | C | C | C | C | C | C | C | CG | C |
| C1/13 | C | C | C | C | C | C | C | C | C | CG | C |
| C1/23 | C | C | C | C | C | C | C | C | C | GC | C |
| C1/14 | C | C | C | C | C | C | C | C | C | CG | C |
| C1/20 | C | C | C | T | C | C | C | C | C | CG | C |
| C1/17 | C | C | C | T | C | C | C | C | C | CG | C |
| C1/6 | C | C | C | T | C | C | C | C | C | CG | C |
| C2/8 | — | — | — | — | — | — | C | C | C | CG | C |
| C2/4 | C | C | C | C | C | C | C | C | C | CG | C |
| C2/11 | C | C | C | C | C | C | C | C | C | CG | C |
| C2/19 | C | C | C | C | C | C | C | C | C | CG | C |
| C2/12 | C | C | C | C | C | C | C | C | C | CG | C |
| C2/2 | C | C | C | C | C | C | C | C | C | CG | C |
| C2/6 | C | C | C | C | C | C | C | C | C | CG | C |
| C2/10 | C | C | C | C | C | C | C | C | C | CG | C |
| C2/3 | C | C | C | C | C | C | C | C | C | CG | C |
| C2/13 | C | C | C | C | C | C | C | C | C | CG | C |
| C2/14 | C | C | C | C | C | C | C | C | C | CG | C |
| C2/16 | C | C | C | C | C | C | C | C | C | CG | C |
| C2/15 | C | C | C | C | C | C | C | C | C | CG | C |
| C2/17 | C | C | C | C | C | C | C | C | C | CG | C |
| C2/9 | C | C | C | C | C | C | C | C | C | CG | C |
| C2/24 | T | C | C | C | C | C | C | C | C | CG | C |
| C2/5 | C | C | C | C | C | C | C | C | C | CG | C |
| C2/7 | C | C | C | C | C | C | C | C | C | CG | C |
| C2/18 | T | C | C | C | C | C | C | C | C | CG | C |
| C2/22 | T | C | C | C | C | C | C | C | C | CG | C |
| C2/23 | C | C | C | C | C | C | C | C | C | CG | C |
| C3/22 | C | C | C | C | C | C | C | C | C | CG | C |
| C3/19 | C | C | C | C | C | C | C | C | C | CG | C |
| C3/11 | C | C | C | C | C | C | C | C | C | CG | C |
| C3/2 | — | C | C | C | C | C | C | C | C | CG | C |
| C3/7 | C | C | C | C | C | C | C | C | C | CG | C |
| C3/10 | C | C | C | C | C | C | C | C | C | CG | C |
| C3/12 | C | C | C | C | C | C | C | C | C | CG | C |
| C3/9 | C | C | C | C | C | C | C | C | C | CG | C |
| C3/1 | C | C | C | C | C | C | C | C | C | CG | C |
| C3/13 | C | C | C | C | C | C | C | C | C | CG | C |
| C3/18 | C | C | C | C | C | C | C | C | C | CG | C |
| C3/16 | C | C | C | C | C | C | C | C | C | CG | C |
| C3/14 | C | C | C | C | C | C | C | C | C | CG | C |
| C3/23 | C | C | C | C | C | C | C | C | C | CG | C |
| C3/15 | C | C | C | C | C | C | C | C | T | CG | C |
| C3/20 | C | C | C | C | C | C | C | C | C | CG | C |
| C3/17 | C | C | C | C | C | C | C | C | C | CG | C |
| C3/24 | C | C | C | C | C | C | C | C | C | CG | C |
| C3/3 | C | C | C | C | C | C | C | C | C | CG | C |
| C3/8 | C | C | C | C | C | C | C | C | C | CG | C |
| C3/6 | T | C | C | C | C | C | C | C | C | CG | C |
| C4/11 | C | C | C | C | C | C | C | C | C | CG | C |
| C4/10 | C | C | C | C | C | C | C | C | C | CG | C |
| C4/4 | C | C | C | C | C | C | C | C | C | CG | C |
| C4/3 | C | C | C | C | C | C | C | C | C | CG | C |
| C4/1 | C | C | C | C | C | C | C | C | C | CG | C |
| C4/17 | C | C | C | C | C | C | C | C | C | CG | C |
| C4/14 | C | C | C | C | C | C | C | C | C | CG | C |
| C4/20 | C | C | C | C | C | C | C | C | C | CG | C |
| C4/18 | C | C | C | C | C | C | C | C | C | CG | C |
| C4/2 | C | C | C | C | C | C | C | C | C | CG | C |
| C4/12 | C | C | C | C | C | C | C | C | C | CG | C |
| C4/5 | C | C | C | C | C | C | C | C | C | CG | C |
| C4/8 | C | C | C | C | C | C | C | C | C | CG | C |
| C4/9 | C | C | C | C | C | C | C | C | C | CG | C |
| C4/21 | C | C | C | C | C | C | C | C | C | CG | C |
| C4/22 | C | C | C | C | C | C | C | C | C | CG | C |
| C4/13 | C | C | C | C | C | C | C | C | C | CG | C |
| C4/23 | C | C | C | C | C | C | C | C | C | CG | C |
| C4/15 | C | C | C | C | C | C | C | C | C | CG | C |
| C4/24 | C | C | C | T | C | C | C | C | C | CG | C |
| C4/16 | C | C | C | C | C | C | C | C | C | CG | C |
| C4/19 | C | C | C | C | C | C | C | T | C | CG | C |
| C5/7 | C | C | C | C | C | C | C | C | C | GG | C |
| C5/5 | C | C | C | C | C | C | C | C | C | GG | C |
| C5/1 | C | C | C | C | C | C | C | C | C | GG | C |
| C5/6 | C | C | C | C | C | C | C | C | C | GG | C |
| C5/11 | C | C | C | C | C | C | C | C | C | GG | C |
| C5/8 | C | C | C | C | C | C | C | C | C | GG | C |
| C5/10 | C | C | C | C | C | C | C | C | C | CG | C |
| C5/9 | C | C | C | C | C | C | C | C | C | CG | C |
| C5/2 | C | C | C | C | C | C | C | C | C | CG | C |
| C5/3 | C | C | C | C | C | C | C | C | C | CG | C |
| C5/4 | — | — | — | C | — | — | C | T | — | CG | C |
| C5/12 | C | C | C | C | C | C | C | C | C | CG | C |
| B1/2 | T | C | C | C | C | C | C | C | C | CG | C |
| B1/4 | T | C | C | C | C | C | C | C | C | CG | C |
| B1/15 | T | C | C | C | C | C | C | C | C | CG | C |
| B1/8 | T | C | C | C | C | C | C | C | C | CG | C |
| B1/14 | T | C | C | C | C | C | C | C | C | CG | C |
| B1/9 | T | C | C | C | C | C | C | C | C | CG | C |
| B1/13 | C | C | C | C | C | C | C | C | C | GG | C |
| B1/11 | C | C | C | C | C | C | C | C | C | GG | C |
| B1/12 | C | C | C | C | C | C | C | C | C | GG | C |
| B1/16 | C | C | C | C | C | C | C | C | C | GG | C |
| B1/6 | C | C | C | C | C | C | C | C | C | GG | C |
| B1/1 | C | C | C | C | C | C | C | C | C | GG | C |
| B1/3 | C | C | C | C | C | C | C | C | C | GG | C |
| B1/5 | C | C | C | C | C | C | C | C | C | GG | C |
| B1/7 | C | C | C | C | C | C | C | C | C | GG | C |
| B2/10 | C | C | C | C | C | C | C | C | C | GG | C |
| B2/5 | C | C | C | C | C | C | C | C | C | GG | C |
| B2/11 | C | C | C | C | C | C | C | C | C | GG | C |
| B2/7 | C | C | C | C | C | C | C | C | C | GG | C |
| B2/1 | C | C | C | C | C | C | C | C | C | GG | C |
| B2/2 | C | C | C | C | C | C | C | C | C | GG | C |
| B2/12 | C | C | C | C | C | C | C | C | C | GG | C |
| B2/3 | C | C | C | C | C | C | C | C | C | GG | C |
| B2/13 | C | C | C | C | C | C | C | C | C | CG | T |
| B2/4 | C | C | C | C | C | C | C | C | C | CG | T |
| B2/6 | T | C | C | C | C | C | C | C | T | CG | C |
| B2/9 | C | C | C | C | C | C | C | C | C | TG | C |
| B3/9 | T | C | C | C | C | C | C | C | C | CG | C |
| B3/8 | C | C | C | C | C | C | C | C | C | CG | C |
| B3/4 | — | — | — | — | — | — | — | — | — | — | — |
| B3/10 | T | T | T | T | T | T | T | T | T | TG | T |
| B3/5 | C | C | C | T | C | C | C | C | C | CG | C |
| B3/6 | C | C | C | T | C | C | C | C | C | CG | C |
| B3/3 | C | C | C | T | C | C | C | C | C | CG | C |
| B3/1 | C | C | C | T | C | C | C | T | C | CG | C |
| B3/11 | C | C | C | T | C | C | C | C | C | CG | C |
| B3/2 | C | C | C | T | C | C | C | C | C | CG | C |
| B3/12 | C | C | — | T | C | C | C | — | C | CG | C |
| B4/10 | — | — | T | C | C | T | T | T | T | CG | C |
| B4/7 | T | C | T | C | C | T | T | T | T | CG | C |
| B4/2 | T | C | T | C | C | T | T | T | T | CG | C |
| B4/3 | T | C | T | C | C | T | T | T | T | CG | C |
| B4/17 | T | C | T | C | C | T | T | T | T | CG | C |
| B4/14 | T | C | C | C | C | T | C | C | T | TG | C |
| B4/18 | T | C | C | C | C | T | C | C | T | TG | C |
| B4/11 | C | C | C | C | C | C | C | C | C | CG | C |
| B4/13 | C | C | C | C | C | C | C | C | C | CG | C |
| B4/12 | C | C | C | C | C | C | C | C | C | CG | C |
| B4/1 | C | C | C | C | C | C | C | C | C | CG | C |
| B4/16 | C | C | C | C | C | C | C | C | C | CG | C |
| B4/4 | C | T | C | C | C | C | C | C | C | CG | C |
| B4/6 | C | T | C | C | C | C | C | C | C | CG | C |
| B4/5 | C | T | C | C | C | C | C | C | C | CG | C |
| B4/20 | C | T | C | C | C | C | C | C | C | CG | C |
| B4/21 | C | T | C | C | C | C | C | C | C | CG | C |
| B4/9 | C | C | C | C | C | C | C | C | C | CG | C |
| H1/1 | C | C | C | C | C | C | C | C | C | CG | C |
| H1/9 | C | C | T | C | C | C | C | C | C | CG | C |
| H1/5 | C | C | C | C | C | C | C | T | C | CG | C |
| H1/10 | C | C | C | C | C | C | C | C | C | CG | C |
| H1/2 | C | C | C | C | C | C | C | C | C | CG | C |
| H1/11 | C | C | C | T | C | C | C | C | C | CG | C |
| H1/8 | T | T | C | C | C | C | C | C | C | CG | C |
| H1/7 | C | C | C | C | C | C | C | C | C | CG | C |
| H1/12 | C | C | C | C | C | C | C | C | C | CG | C |
| H1/6 | C | C | C | C | C | C | C | C | C | CG | C |
| H1/4 | C | C | C | C | C | C | C | C | C | CG | C |
| H2/1 | C | C | C | C | C | C | C | C | C | CG | C |
| H2/12 | C | C | C | C | C | C | C | C | C | CG | C |
| H2/2 | C | C | C | C | C | C | C | C | C | CG | C |
| H2/5 | C | C | C | C | C | C | C | C | C | CG | C |
| H2/3 | C | C | C | T | C | C | C | C | C | CG | C |
| H2/8 | C | C | C | C | C | C | C | C | C | CG | C |
| H2/4 | C | C | C | C | C | C | T | C | T | CG | C |
| H2/10 | C | T | C | C | C | C | C | C | C | TG | C |
| H2/11 | T | C | C | C | C | C | C | C | C | CG | C |
| H2/6 | C | C | C | C | C | C | C | C | C | CG | C |
| H2/7 | C | C | C | C | C | C | C | C | C | CG | C |
| H2/9 | C | C | T | C | C | C | C | T | T | CG | C |

| | −3097 | −3065 | −3057 | −3035 | −3033 | −3026 | 3010 | −3003 | −2995 | −2971 |
|---|---|---|---|---|---|---|---|---|---|---|
| Clone | CG-40 | CG-39 | CG-38 | CG-37 | CG-36 | CG-35 | CG-34C-A | CG-33 | CG-32 | CG-30 |
| C1/24 | C | C | C | C | C | C | GC | C | C | C |
| C1/7 | C | C | C | C | C | C | CG | C | C | C |
| C1/1 | C | C | C | C | C | C | CG | C | C | C |
| C1/3 | C | C | C | C | C | C | CG | C | C | C |
| C1/8 | C | C | C | C | C | C | CG | C | C | C |
| C1/5 | C | C | C | C | C | C | CG | C | C | C |
| C1/11 | C | C | C | C | C | C | CG | C | C | C |
| C1/10 | C | C | C | C | C | C | CG | C | C | C |
| C1/2 | C | C | C | C | C | C | CG | C | C | C |
| C1/12 | C | C | C | C | C | C | CG | C | C | C |
| C1/15 | C | C | C | C | C | C | CG | C | C | C |
| C1/21 | C | C | C | C | C | C | CG | C | C | C |
| C1/18 | C | C | C | C | C | C | CG | C | C | C |
| C1/22 | C | C | C | C | C | C | CG | C | C | C |
| C1/4 | C | C | C | C | C | C | CG | C | C | C |
| C1/9 | C | C | C | C | C | C | CG | C | C | C |
| C1/16 | C | C | C | C | C | C | CG | C | C | C |
| C1/13 | C | C | C | C | C | C | CG | C | C | C |
| C1/23 | C | C | C | C | C | C | CG | C | C | C |
| C1/14 | C | C | C | C | C | C | CG | C | C | C |
| C1/20 | C | C | C | C | C | C | CG | C | C | C |
| C1/17 | C | C | C | C | C | C | CG | C | C | C |
| C1/6 | C | C | C | C | C | C | CG | C | C | C |
| C2/8 | C | C | C | C | C | C | CG | C | C | C |
| C2/4 | C | C | C | C | C | C | — | C | C | C |
| C2/11 | C | C | C | C | C | C | CG | C | C | C |
| C2/19 | C | C | C | C | C | C | CG | C | C | C |
| C2/12 | C | C | C | C | C | C | CG | C | C | C |
| C2/2 | C | C | C | C | C | C | CG | C | C | C |
| C2/6 | C | C | C | C | C | C | CG | C | C | C |
| C2/10 | C | C | C | C | C | C | CG | C | C | C |
| C2/3 | C | C | C | C | C | C | CG | C | C | C |
| C2/13 | C | C | C | C | C | C | CG | C | C | C |
| C2/14 | C | C | C | C | C | C | CG | C | C | C |
| C2/16 | C | C | C | C | C | C | CG | C | C | C |
| C2/15 | C | C | C | C | C | C | CG | C | C | C |
| C2/17 | C | C | C | C | C | C | CG | C | C | C |
| C2/9 | C | C | C | C | C | C | CG | C | C | C |
| C2/24 | C | C | C | C | C | C | CG | C | C | C |
| C2/5 | C | C | C | C | C | C | CG | C | C | C |
| C2/7 | C | C | C | C | C | C | CG | C | C | C |
| C2/18 | C | C | C | C | C | C | CG | C | C | C |
| C2/22 | C | C | C | C | C | C | CG | C | C | C |
| C2/23 | C | C | C | C | C | C | CG | C | C | C |
| C3/22 | C | C | C | C | C | C | C | C | C | C |
| C3/19 | C | C | C | C | C | C | C | C | C | C |
| C3/11 | C | C | C | C | C | C | C | C | C | C |
| C3/2 | C | C | C | C | C | C | C | C | C | C |
| C3/7 | C | C | C | C | C | C | C | C | C | C |
| C3/10 | C | C | C | C | C | C | C | C | C | C |
| C3/12 | C | C | C | C | C | C | C | C | C | C |
| C3/9 | C | C | C | C | C | C | C | C | C | C |
| C3/1 | C | C | C | C | C | C | C | C | C | C |
| C3/13 | C | C | C | c | C | C | C | C | C | C |
| C3/18 | C | C | C | T | C | C | C | C | C | C |
| C3/16 | C | C | C | C | C | C | C | C | C | C |
| C3/14 | C | C | C | C | C | C | C | C | C | C |
| C3/23 | C | C | C | C | C | C | C | C | C | C |
| C3/15 | C | C | C | C | C | C | C | C | C | C |
| C3/20 | C | C | C | C | C | C | C | C | C | C |
| C3/17 | C | C | C | C | C | C | C | C | C | C |
| C3/24 | C | C | C | C | C | C | C | C | C | C |
| C3/3 | C | C | C | C | C | C | C | C | C | C |
| C3/8 | C | C | C | C | C | C | C | C | C | C |
| C3/6 | C | C | C | C | C | C | C | C | C | C |
| C4/11 | C | C | C | C | C | C | C | C | C | C |
| C4/10 | C | C | C | C | C | C | C | C | C | C |
| C4/4 | C | C | C | C | C | C | C | C | C | C |
| C4/3 | C | C | C | C | C | C | C | C | C | C |
| C4/1 | C | C | C | C | C | C | C | C | C | C |
| C4/17 | C | C | C | C | C | C | C | C | C | C |
| C4/14 | C | C | C | C | C | C | C | C | C | C |
| C4/20 | C | C | C | C | C | C | C | C | C | C |
| C4/18 | C | C | C | C | C | C | C | C | C | C |
| C4/2 | C | C | C | C | C | C | C | C | C | C |
| C4/12 | C | C | C | C | C | C | C | C | C | C |
| C4/5 | C | C | C | C | C | C | C | C | C | C |
| C4/8 | C | C | C | C | C | C | C | C | C | C |
| C4/9 | C | C | C | C | C | C | C | C | C | C |
| C4/21 | C | C | C | C | C | C | C | C | C | C |
| C4/22 | C | C | C | C | C | C | C | C | C | C |
| C4/13 | C | C | C | C | C | C | C | C | C | C |
| C4/23 | C | C | C | C | C | C | C | C | C | C |
| C4/15 | C | C | C | C | C | C | C | C | C | C |
| C4/24 | C | C | C | C | C | C | C | C | C | C |
| C4/16 | C | C | C | C | C | C | C | C | C | C |
| C4/19 | C | C | C | C | C | C | C | C | C | C |
| C5/7 | C | C | C | C | C | C | C | C | C | C |
| C5/5 | C | C | C | C | C | C | C | C | C | C |
| C5/1 | C | C | C | C | C | C | C | C | C | C |
| C5/6 | C | C | C | C | C | C | C | C | C | C |
| C5/11 | C | C | C | C | C | C | C | C | C | C |
| C5/8 | C | C | C | C | C | C | C | C | C | C |
| C5/10 | C | C | C | C | C | C | C | C | C | C |
| C5/9 | C | C | C | C | C | C | C | C | C | C |
| C5/2 | C | C | C | C | C | C | C | C | C | C |
| C5/3 | C | C | C | C | C | C | C | C | C | C |
| C5/4 | C | C | C | C | C | C | C | C | C | C |
| C5/12 | C | C | C | C | C | C | C | C | C | C |
| B1/2 | C | C | C | C | C | C | C | C | C | C |
| B1/4 | C | C | C | C | C | C | C | C | C | C |
| B1/15 | C | C | C | C | C | C | C | C | C | C |
| B1/8 | C | C | C | C | C | C | C | C | C | C |
| B1/14 | C | C | C | C | C | C | C | C | C | C |
| B1/9 | C | C | C | C | C | C | C | C | C | C |
| B1/13 | C | C | C | C | C | C | C | C | C | C |
| B1/11 | C | C | C | C | C | C | C | C | C | C |
| B1/12 | C | C | C | C | C | C | C | C | C | C |
| B1/16 | C | C | C | C | C | C | C | C | C | C |
| B1/6 | C | C | C | C | C | C | C | C | C | C |
| B1/1 | C | C | C | C | C | C | C | C | C | C |
| B1/3 | C | C | C | C | C | C | C | C | C | C |
| B1/5 | C | C | C | C | C | C | C | C | C | C |
| B1/7 | C | C | C | C | C | C | C | C | C | C |
| B2/10 | C | C | C | C | C | C | C | C | C | C |
| B2/5 | C | C | C | C | C | C | C | C | C | C |
| B2/11 | C | C | C | C | C | C | C | C | C | C |
| B2/7 | C | C | C | C | C | C | C | C | C | C |
| B2/1 | C | C | C | C | C | C | C | C | C | C |
| B2/2 | C | C | C | C | C | C | C | C | C | C |
| B2/12 | C | C | C | C | C | C | C | C | C | C |
| B2/3 | C | C | C | C | C | C | C | C | C | C |
| B2/13 | C | C | C | C | C | C | C | C | C | C |
| B2/4 | C | C | C | C | C | C | C | C | C | C |
| B2/6 | C | C | C | C | C | C | C | C | C | C |
| B2/9 | C | C | C | C | C | C | C | C | C | C |
| B3/9 | C | C | C | C | C | C | C | C | C | C |
| B3/8 | C | C | C | C | C | C | C | C | C | C |
| B3/4 | C | C | C | C | C | C | C | C | C | C |
| B3/10 | T | T | T | T | T | T | T | T | T | T |
| B3/5 | C | C | C | C | C | C | C | C | C | C |
| B3/6 | C | C | C | C | C | C | C | C | C | C |
| B3/3 | C | C | C | C | C | C | C | C | C | C |
| B3/1 | C | C | C | C | C | C | C | C | C | C |
| B3/11 | C | C | C | C | C | C | C | C | C | C |
| B3/2 | C | C | C | C | C | C | C | C | C | C |
| B3/12 | C | C | C | C | C | C | C | C | C | C |
| B4/10 | T | T | T | T | T | C | C | C | C | T |
| B4/7 | T | T | T | T | T | C | C | C | C | T |
| B4/2 | T | T | T | T | T | C | C | C | C | T |
| B4/3 | T | T | T | T | T | C | C | C | C | T |
| B4/17 | T | T | T | T | T | C | C | C | C | T |
| B4/14 | C | C | C | C | C | C | C | C | C | C |
| B4/18 | C | C | C | C | C | C | C | C | C | C |
| B4/11 | C | C | C | C | C | C | C | C | C | C |
| B4/13 | C | C | C | C | C | C | C | C | C | C |
| B4/12 | C | C | C | C | C | C | C | C | C | C |
| B4/1 | C | C | C | C | C | C | C | C | C | C |
| B4/16 | C | C | C | C | C | C | C | C | C | C |
| B4/4 | C | C | C | C | C | C | C | C | C | C |
| B4/6 | C | C | C | C | C | C | C | C | C | C |
| B4/5 | C | C | C | C | C | C | C | C | C | C |
| B4/20 | C | C | C | C | C | C | C | C | C | C |
| B4/21 | C | C | C | C | C | C | C | C | C | C |
| B4/9 | C | C | C | C | C | C | C | C | C | C |
| H1/1 | C | C | C | C | C | C | C | C | C | C |
| H1/9 | C | C | C | C | C | C | C | C | C | C |
| H1/5 | C | C | C | C | C | C | C | C | C | C |
| H1/10 | C | C | C | C | C | C | C | C | C | C |
| H1/2 | C | C | C | C | C | C | C | C | C | C |
| H1/11 | C | T | C | C | C | C | C | C | C | C |
| H1/8 | C | C | C | C | C | C | C | C | C | T |
| H1/7 | C | C | T | C | C | C | C | C | C | C |
| H1/12 | C | C | C | C | C | C | C | C | C | C |
| H1/6 | C | C | C | C | C | C | C | C | C | C |
| H1/4 | C | C | C | C | C | C | C | C | C | C |
| H2/1 | C | C | C | C | C | C | C | C | C | DEL |
| H2/12 | C | C | C | C | C | C | C | C | C | C |
| H2/2 | C | C | C | C | C | C | C | C | C | C |
| H2/5 | C | C | C | C | C | C | C | C | C | C |
| H2/3 | C | C | T | C | C | C | C | C | C | C |
| H2/8 | C | C | C | C | C | C | C | C | C | T |
| H2/4 | C | C | C | C | C | C | C | C | C | C |
| H2/10 | C | C | C | C | C | C | C | C | C | C |
| H2/11 | C | C | C | C | C | C | C | C | C | C |
| H2/6 | C | C | C | C | C | C | C | C | C | DEL |
| H2/7 | C | C | C | C | C | C | C | C | C | DEL |
| H2/9 | T | C | C | C | C | C | C | C | C | DEL |

| | −2951 | −2947 | −2939 | −2935 | −2905 | −2874 | −2860 | −2853 | −2849 | −2844 |
|---|---|---|---|---|---|---|---|---|---|---|
| Clone | CG-29 | CG-28 | CG-27 | CG-26 | CG-25 | CG-24 | CG-23 | GC-22 | GC-21 | GC-20 |
| C1/24 | C | C | C | C | C | C | C | C | C | C |
| C1/7 | C | C | C | C | C | C | C | T | C | C |
| C1/1 | C | C | C | C | C | C | C | C | C | C |
| C1/3 | C | C | C | C | C | C | C | C | C | C |
| C1/8 | C | C | C | C | C | C | C | C | C | C |
| C1/5 | C | C | C | C | C | C | C | C | C | C |
| C1/11 | C | C | C | C | C | C | C | C | C | C |
| C1/10 | C | C | C | C | C | C | C | C | C | C |
| C1/2 | C | C | C | C | C | C | C | C | C | C |
| C1/12 | C | C | C | C | C | C | C | C | C | C |
| C1/15 | C | C | C | C | C | C | C | C | C | C |
| C1/21 | C | C | C | C | C | C | C | C | C | C |
| C1/18 | C | C | C | C | C | C | C | C | C | C |
| C1/22 | C | C | C | C | C | C | C | C | C | C |
| C1/4 | C | C | C | C | C | C | C | C | C | C |
| C1/9 | C | C | C | C | C | C | C | C | C | C |
| C1/16 | C | C | C | C | C | C | C | C | C | C |
| C1/13 | C | C | C | C | C | C | C | C | C | C |
| C1/23 | C | C | C | C | C | C | C | C | C | C |
| C1/14 | C | C | C | C | C | C | C | C | C | C |
| C1/20 | C | C | C | C | C | C | C | C | C | C |
| C1/17 | C | C | C | C | C | C | C | C | C | C |
| C1/6 | C | C | C | C | C | C | C | C | C | C |
| C2/8 | C | C | C | C | C | C | C | C | C | C |
| C2/4 | C | C | C | C | C | C | C | C | C | C |
| C2/11 | C | C | C | C | C | C | C | C | C | C |
| C2/19 | C | C | C | C | C | C | C | C | C | C |
| C2/12 | C | C | C | C | C | C | C | C | C | C |
| C2/2 | C | C | C | C | C | C | C | C | C | C |
| C2/6 | C | C | C | C | C | C | C | C | C | C |
| C2/10 | C | C | C | C | C | C | C | C | C | C |
| C2/3 | C | C | C | C | C | C | C | C | C | C |
| C2/13 | C | C | C | C | C | C | C | C | C | C |
| C2/14 | C | C | C | C | C | C | C | C | C | C |
| C2/16 | C | C | C | C | C | C | C | C | C | C |
| C2/15 | C | C | C | C | C | C | C | C | C | C |
| C2/17 | C | C | C | C | C | C | C | C | C | C |
| C2/9 | C | C | C | C | C | C | C | C | C | C |
| C2/24 | C | C | C | C | C | C | C | C | C | C |
| C2/5 | C | C | C | C | C | C | C | C | C | T |
| C2/7 | C | C | C | C | C | C | C | C | C | C |
| C2/18 | C | C | C | C | C | C | C | C | C | C |
| C2/22 | C | C | C | C | C | C | C | C | C | C |
| C2/23 | C | C | C | C | C | C | C | C | C | C |
| C3/22 | C | C | C | C | C | C | C | C | C | C |
| C3/19 | C | C | C | C | C | C | C | C | C | C |
| C3/11 | C | C | C | C | C | C | C | C | C | C |
| C3/2 | C | C | C | C | C | C | C | C | C | C |
| C3/7 | C | C | C | C | C | C | C | C | C | C |
| C3/10 | C | C | C | C | C | C | C | C | C | C |
| C3/12 | C | C | C | C | C | C | C | C | C | C |
| C3/9 | C | C | C | C | C | C | C | C | C | C |
| C3/1 | C | C | C | C | C | C | C | C | C | C |
| C3/13 | C | C | C | C | C | C | C | C | C | C |
| C3/18 | C | C | C | C | C | C | C | C | C | C |
| C3/16 | C | C | C | C | C | C | C | C | C | C |
| C3/14 | C | C | C | C | C | C | C | C | C | C |
| C3/23 | C | C | C | C | C | C | C | C | C | C |
| C3/15 | C | C | C | C | C | C | C | C | C | C |
| C3/20 | C | C | C | C | C | C | C | C | C | C |
| C3/17 | C | C | C | C | C | C | C | C | C | C |
| C3/24 | C | C | C | C | C | C | C | C | C | C |
| C3/3 | C | C | C | C | C | C | C | C | C | C |
| C3/8 | C | C | C | C | C | C | C | C | C | C |
| C3/6 | C | C | C | C | C | C | C | C | C | C |
| C4/11 | C | C | C | C | C | C | C | C | C | C |
| C4/10 | C | C | C | C | C | C | C | C | C | C |
| C4/4 | C | C | C | C | C | C | C | C | C | C |
| C4/3 | C | C | C | C | C | C | C | C | C | C |
| C4/1 | C | C | C | C | C | C | C | C | C | C |
| C4/17 | C | C | C | C | C | C | C | C | C | C |
| C4/14 | C | C | C | C | C | C | C | C | C | C |
| C4/20 | C | C | C | C | C | C | C | C | C | C |
| C4/18 | C | C | C | C | C | C | C | C | C | C |
| C4/2 | C | C | C | C | C | C | C | C | C | T |
| C4/12 | C | C | C | C | C | C | C | C | C | T |
| C4/5 | C | C | C | C | C | C | C | C | C | T |
| C4/8 | C | C | C | C | C | C | C | C | C | T |
| C4/9 | C | C | C | C | C | C | C | C | C | T |
| C4/21 | C | C | C | C | C | C | C | C | C | T |
| C4/22 | C | C | C | C | C | C | C | C | C | T |
| C4/13 | C | C | C | C | C | C | C | T | T | C |
| C4/23 | C | C | C | C | C | C | C | T | T | C |
| C4/15 | C | C | C | C | C | C | C | C | C | C |
| C4/24 | C | C | C | C | C | C | C | C | C | C |
| C4/16 | C | C | C | C | C | C | C | C | C | C |
| C4/19 | C | C | C | C | C | C | C | C | C | C |
| C5/7 | C | C | C | C | C | C | C | C | C | C |
| C5/5 | C | C | C | C | C | C | C | C | C | C |
| C5/1 | C | C | C | C | C | C | C | C | C | C |
| C5/6 | C | C | C | C | C | C | C | C | C | C |
| C5/11 | C | C | C | C | C | C | C | C | C | C |
| C5/8 | C | C | C | C | C | C | C | C | C | C |
| C5/10 | C | C | C | C | C | C | C | C | C | C |
| C5/9 | C | C | C | C | C | C | C | C | C | C |
| C5/2 | C | C | C | C | C | C | C | C | C | C |
| C5/3 | C | C | C | C | C | C | C | C | C | C |
| C5/4 | C | C | C | C | C | C | C | C | C | C |
| C5/12 | C | C | C | C | C | C | C | C | C | C |
| B1/2 | C | C | C | C | C | C | C | C | C | C |
| B1/4 | C | C | C | C | C | C | C | C | C | C |
| B1/15 | C | C | C | C | C | C | C | C | C | C |
| B1/8 | C | C | C | C | C | C | C | C | C | C |
| B1/14 | C | C | C | C | C | C | C | C | C | C |
| B1/9 | C | C | C | C | C | C | C | C | C | C |
| B1/13 | C | C | C | C | C | C | C | C | C | C |
| B1/11 | C | C | C | C | C | C | C | C | C | C |
| B1/12 | C | C | C | C | C | C | C | C | C | C |
| B1/16 | C | C | C | C | C | C | C | C | C | C |
| B1/6 | C | C | C | C | C | C | C | C | C | C |
| B1/1 | C | C | C | C | C | C | C | C | C | C |
| B1/3 | C | C | C | C | C | C | C | C | C | C |
| B1/5 | C | C | C | C | C | C | C | C | C | C |
| B1/7 | C | C | C | C | C | C | C | C | C | C |
| B2/10 | C | C | C | C | C | C | C | C | C | C |
| B2/5 | C | C | C | C | C | C | C | C | C | C |
| B2/11 | C | C | C | C | C | C | C | C | C | C |
| B2/7 | C | C | C | C | C | C | C | C | C | C |
| B2/1 | C | C | C | C | C | C | C | C | C | C |
| B2/2 | C | C | C | C | C | C | C | C | C | C |
| B2/12 | C | C | C | C | C | C | C | C | C | C |
| B2/3 | C | C | C | C | C | C | C | C | C | C |
| B2/13 | C | C | C | C | C | C | C | C | C | C |
| B2/4 | C | C | C | C | C | C | C | C | C | C |
| B2/6 | C | C | C | C | C | C | C | C | C | C |
| B2/9 | C | C | C | C | C | C | C | C | C | C |
| B3/9 | C | C | C | C | C | C | C | C | C | C |
| B3/8 | C | C | C | C | C | C | C | C | C | C |
| B3/4 | C | C | C | C | C | C | C | C | C | C |
| B3/10 | T | T | T | T | T | T | T | T | T | T |
| B3/5 | C | C | C | C | C | C | C | C | C | C |
| B3/6 | C | C | C | C | C | C | C | C | C | C |
| B3/3 | C | C | C | C | C | C | C | C | C | C |
| B3/1 | C | C | C | C | C | C | C | C | C | C |
| B3/11 | C | C | C | C | C | C | C | C | C | C |
| B3/2 | C | C | C | C | C | C | C | C | C | C |
| B3/12 | C | C | C | C | C | C | C | C | C | C |
| B4/10 | T | C | T | C | T | C | T | T | T | C |
| B4/7 | T | C | T | C | T | C | T | T | T | C |
| B4/2 | T | C | T | C | T | C | T | T | T | C |
| B4/3 | T | C | T | C | T | C | T | T | T | C |
| B4/17 | T | C | C | C | T | C | T | T | T | C |
| B4/14 | T | C | T | C | C | C | C | T | C | C |
| B4/18 | T | C | T | C | C | C | C | T | C | C |
| B4/11 | C | C | C | C | C | C | C | C | C | C |
| B4/13 | C | C | C | C | C | C | C | C | C | C |
| B4/12 | C | C | C | C | C | C | C | C | C | C |
| B4/1 | C | C | C | C | C | C | C | C | C | C |
| B4/16 | C | C | C | C | C | C | C | C | C | C |
| B4/4 | C | C | C | C | C | C | C | C | C | C |
| B4/6 | C | C | C | C | C | C | C | C | C | C |
| B4/5 | C | C | C | C | C | C | C | C | C | C |
| B4/20 | C | C | C | C | C | C | C | C | C | C |
| B4/21 | C | C | C | C | C | C | C | C | C | C |
| B4/9 | C | C | C | C | C | C | C | C | C | C |
| H1/1 | C | C | C | C | C | C | C | C | C | C |
| H1/9 | C | C | C | C | C | C | C | C | C | T |
| H1/5 | C | C | C | C | C | C | C | C | C | C |
| H1/10 | C | C | C | C | C | C | C | C | C | C |
| H1/2 | C | C | C | C | C | C | C | C | C | C |
| H1/11 | C | C | T | C | C | C | C | C | C | C |
| H1/8 | C | C | C | C | C | T | T | T | T | T |
| H1/7 | T | C | C | C | C | T | C | C | C | C |
| H1/12 | C | C | C | C | C | C | C | C | C | C |
| H1/6 | C | C | C | C | C | T | C | C | C | C |
| H1/4 | C | C | C | C | C | C | C | C | C | C |
| H2/1 | C | C | C | C | C | C | C | C | C | C |
| H2/12 | C | C | C | C | C | C | C | C | C | C |
| H2/2 | C | C | C | C | C | C | T | C | C | C |
| H2/5 | C | C | C | C | C | C | C | C | C | C |
| H2/3 | C | C | T | C | C | C | C | C | C | C |
| H2/8 | C | C | C | C | C | C | C | C | C | C |
| H2/4 | C | C | C | C | C | C | C | C | C | C |
| H2/10 | C | C | C | C | C | T | C | C | C | C |
| H2/11 | C | C | C | C | C | C | C | C | C | C |
| H2/6 | C | C | C | C | C | C | C | C | C | C |
| H2/7 | C | C | C | C | C | C | C | C | C | T |
| H2/9 | C | C | C | C | C | C | C | C | C | C |

TABLE 3
Non-CpG methylation in CpG-island 4-5 of the MSMB gene

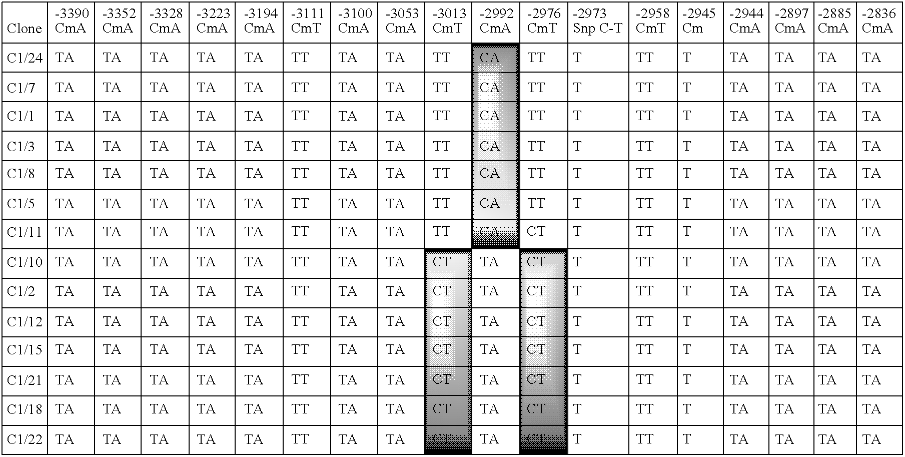

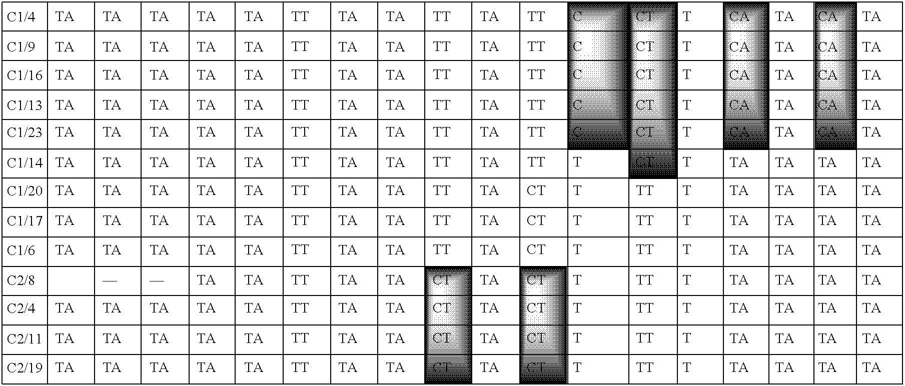

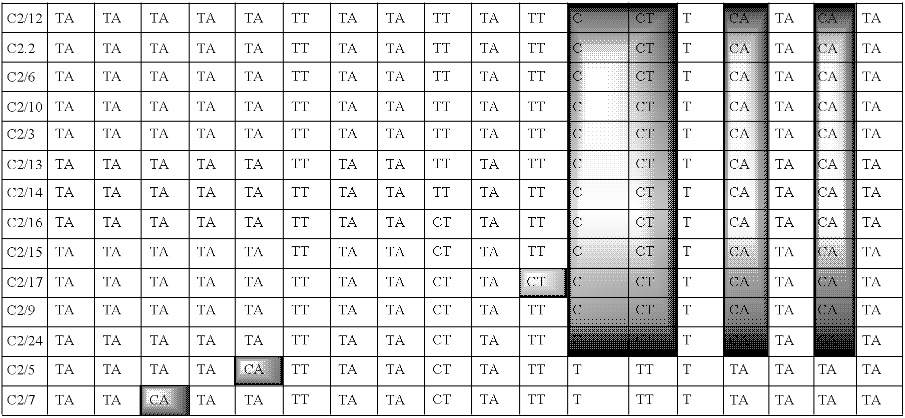

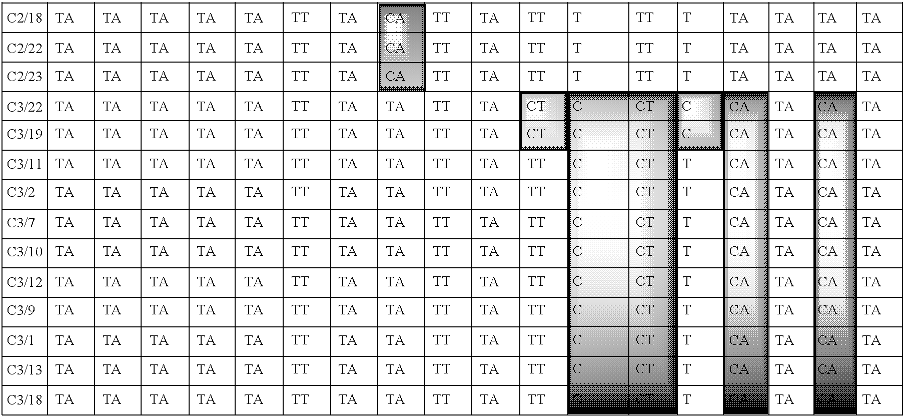

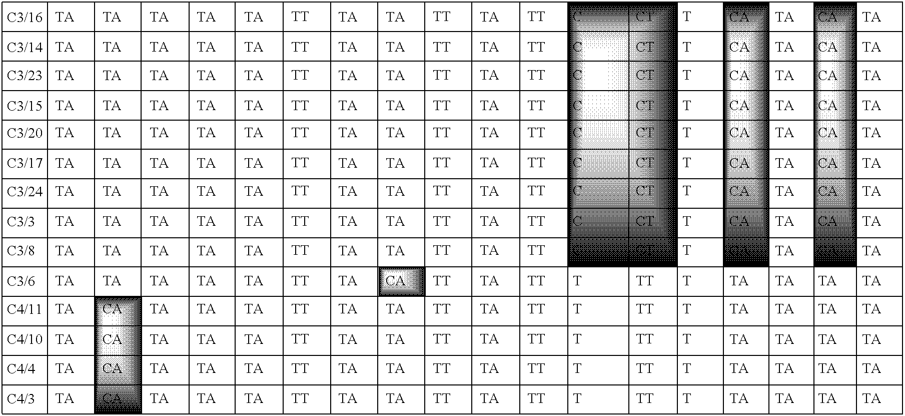

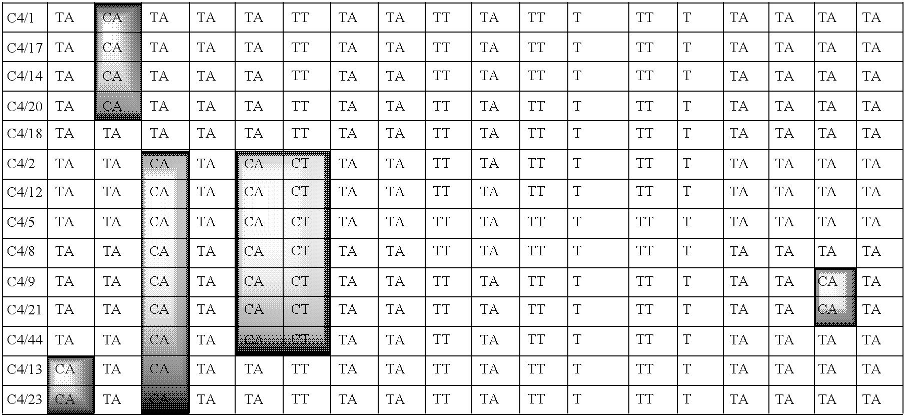

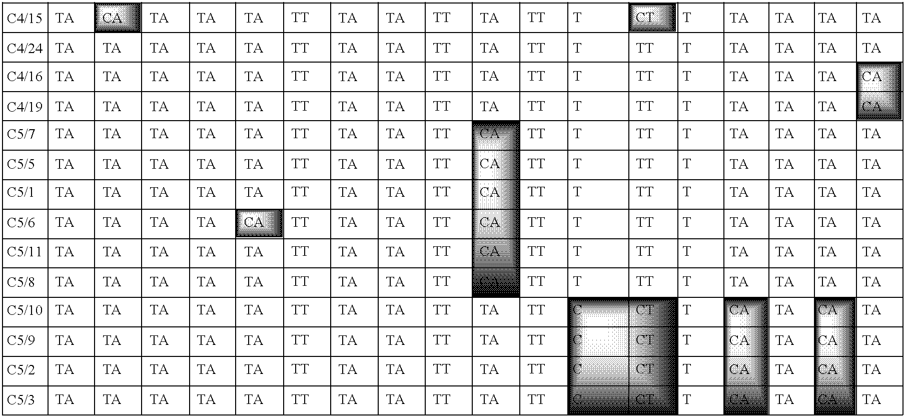

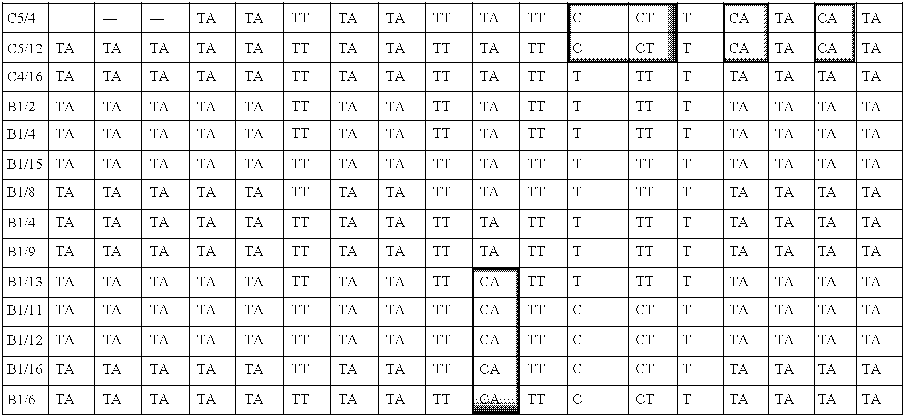

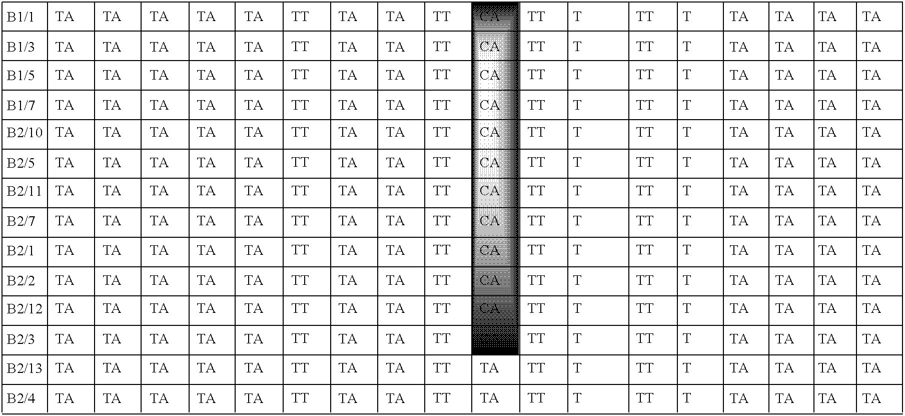

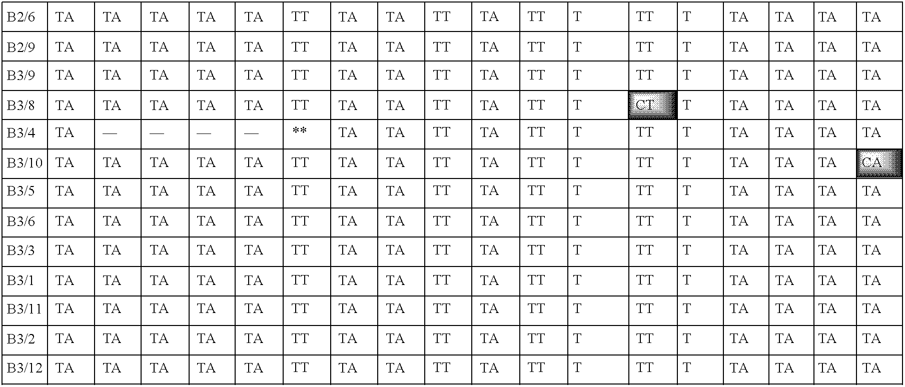

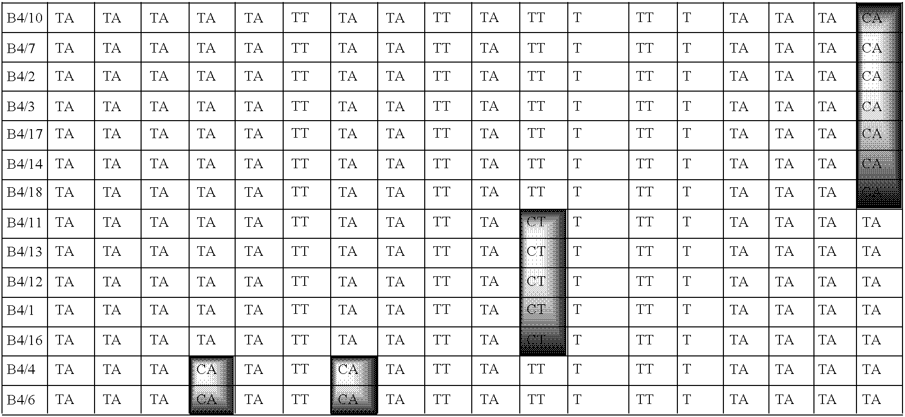

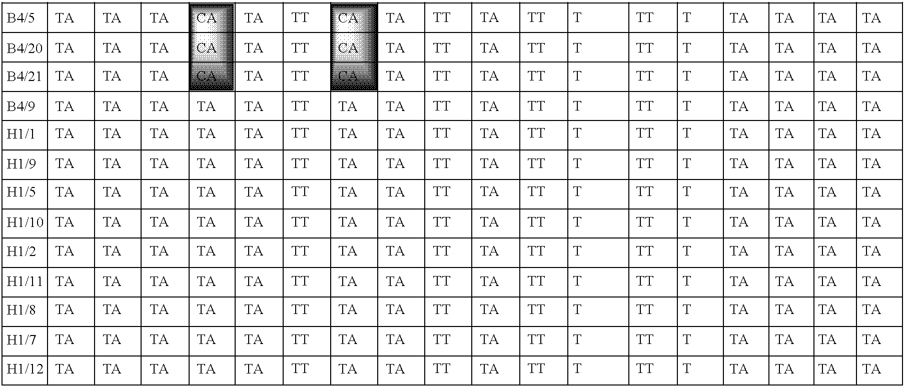

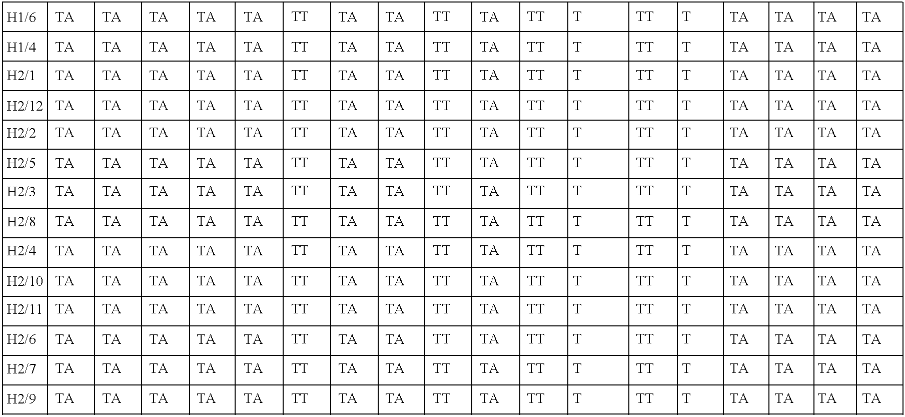

TABLE 4
Sequence alterations in CpG-island 4-5 of the MSMB gene

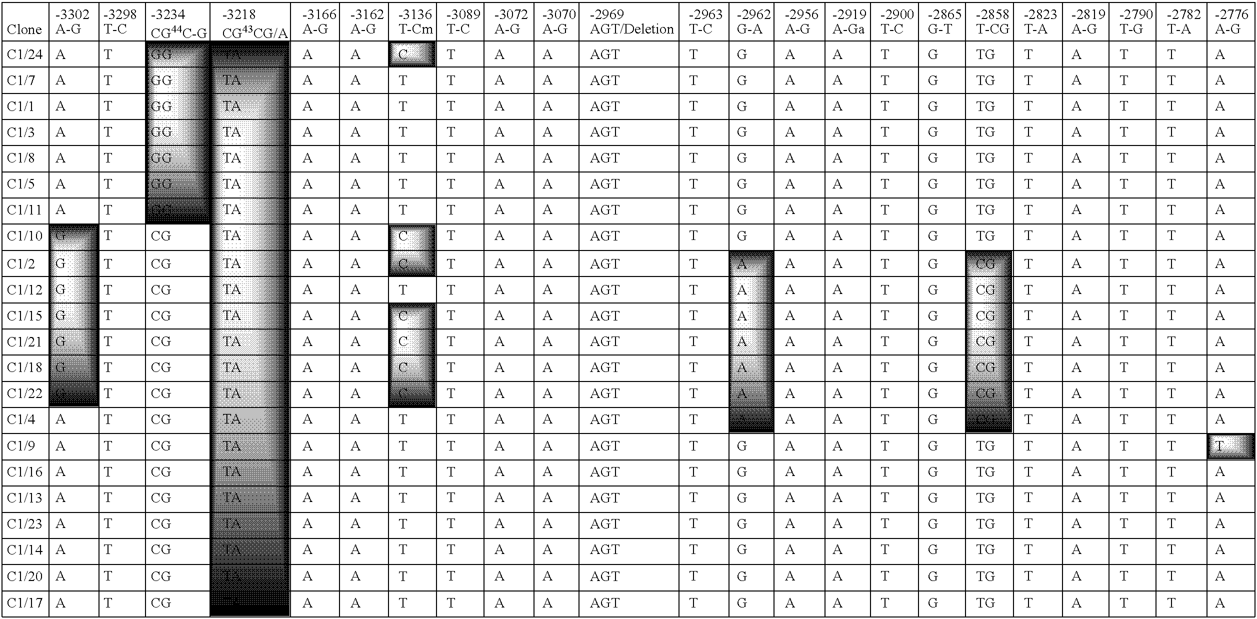

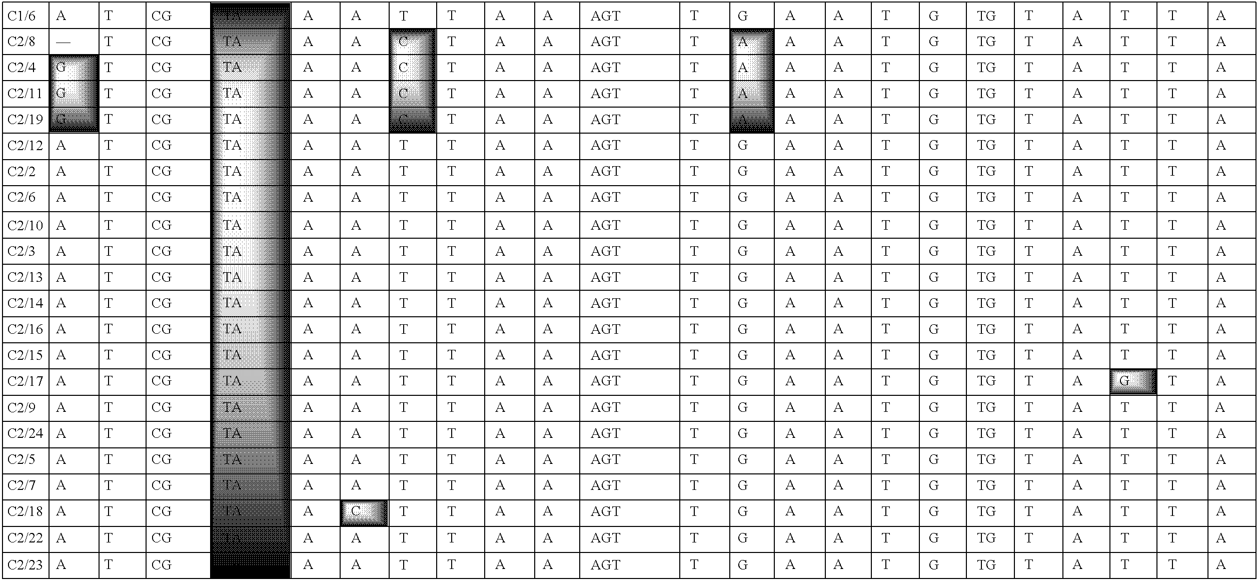

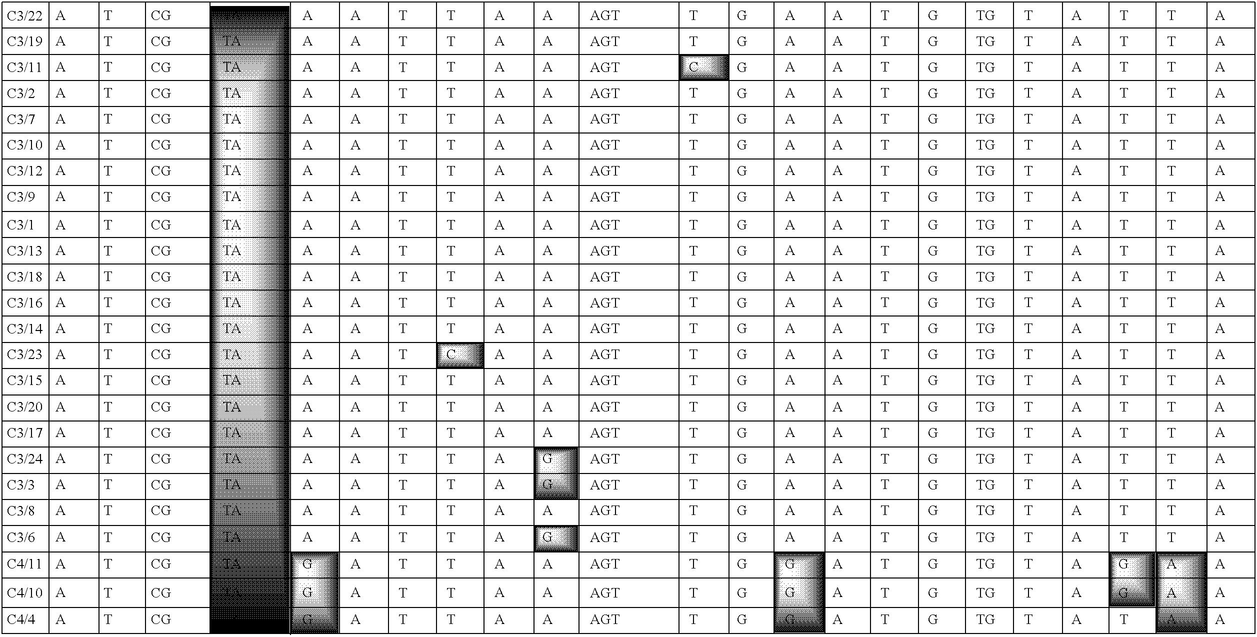

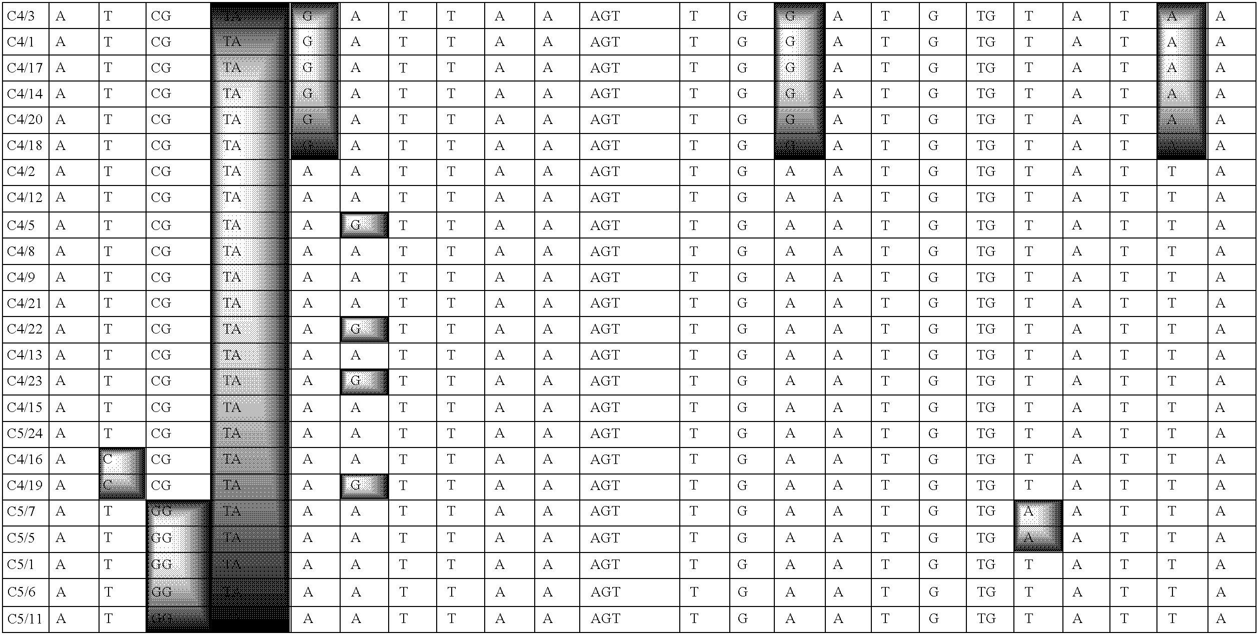

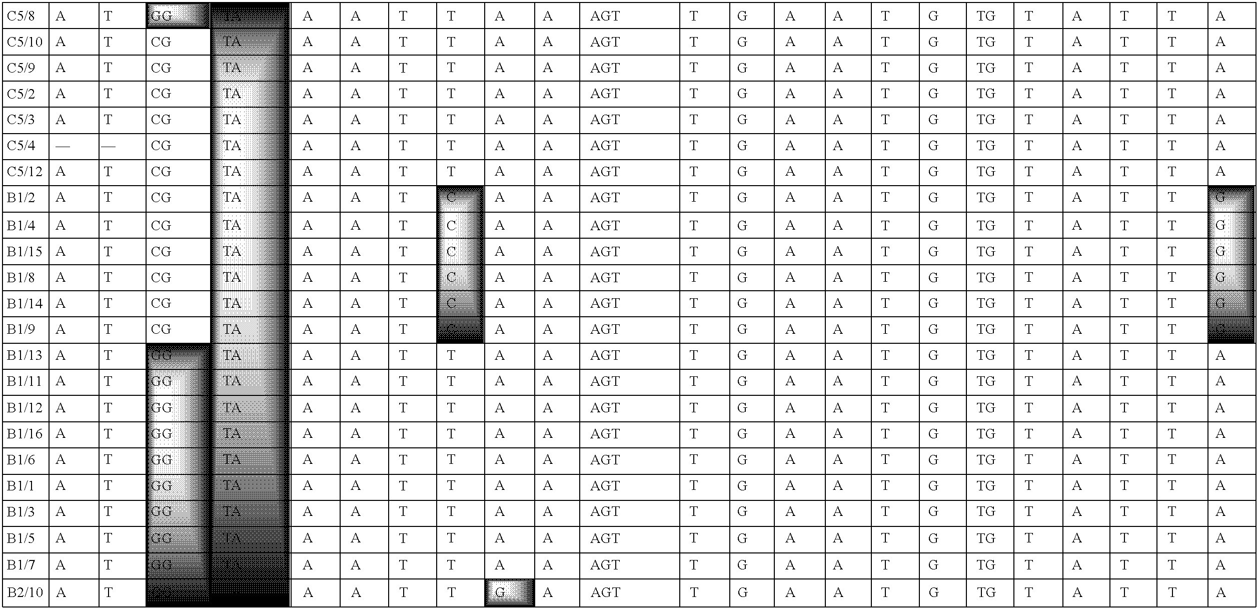

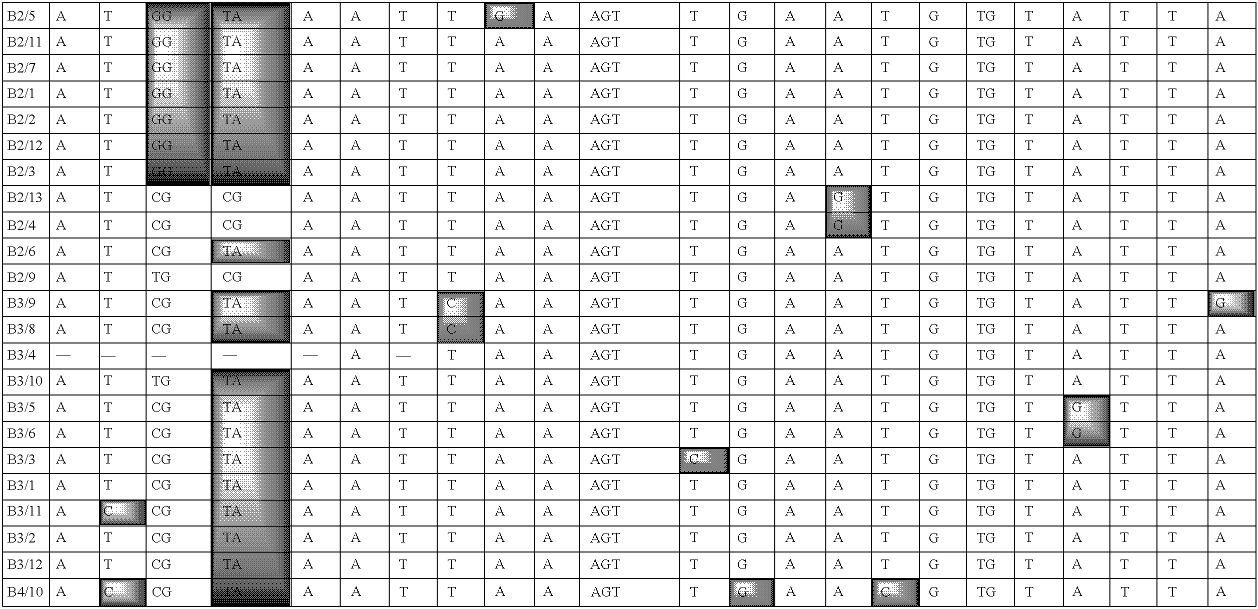

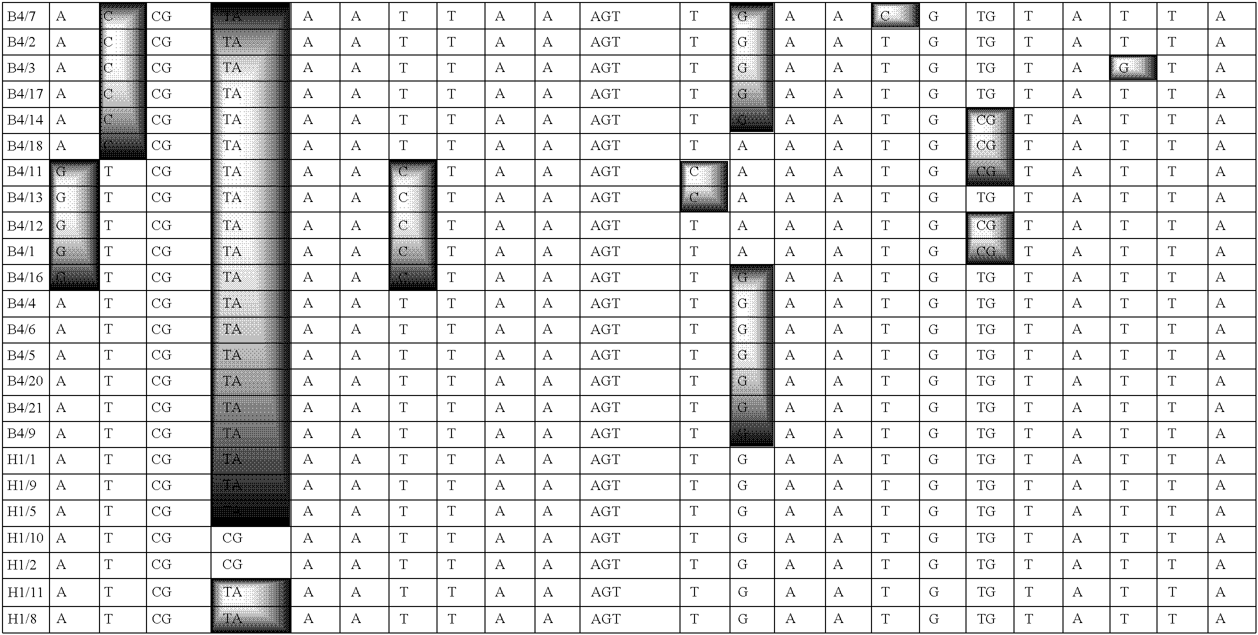

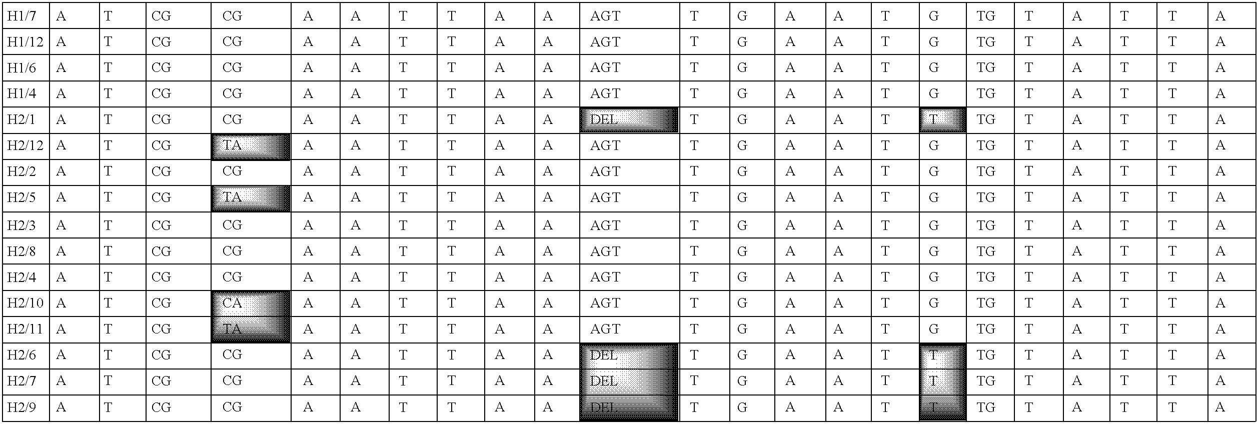

TABLE 5
Summary of all detected haplotypes of CpG island 4-5 of the MSMB gene

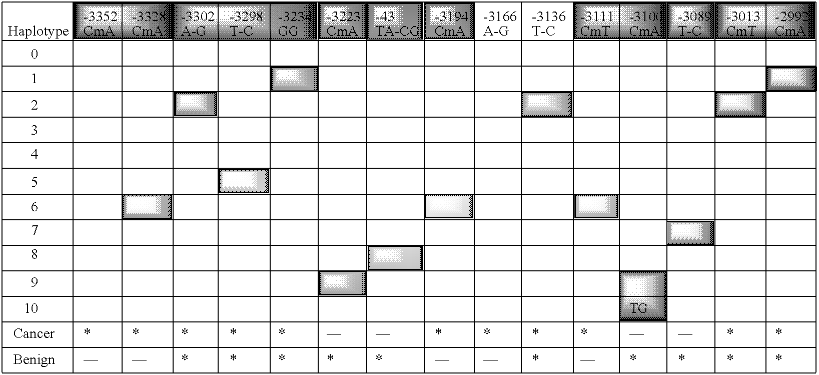

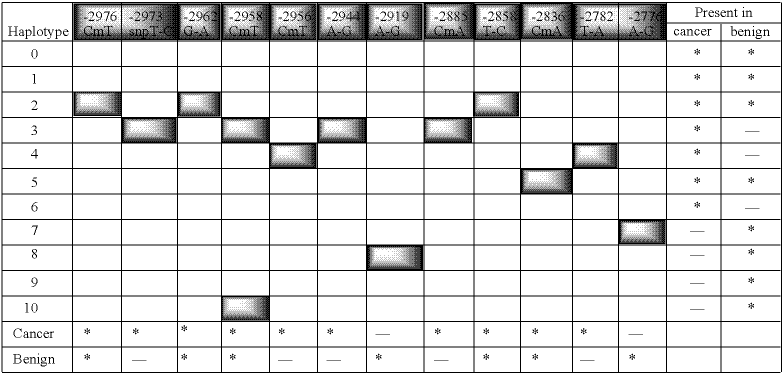

What is claimed is:

1. A method of identifying a prostate cell proliferative disorder in a human male subject, the method comprising:
providing a sample comprising prostatic tissue, prostatic cells, fluid of the prostate from a human patient susceptible to a prostate cancer, and
analyzing the sample for the level DNA methylation of the regulatory region surrounding the transcription start site (TSS) of a beta-microseminoprotein gene (MSMB gene), wherein hypermethylation in this region indicates the presence of prostate cancer cells or is indicative of prostate cancer.

2. The method according to claim 1, wherein the region comprises one or more CpG islands and extends from about 3.0 kb upstream to about 2.2 kb downstream from the TSS of the MSMB gene.

3. The method according to claim 1, wherein the region extends from −3128 bp to −2817 bp upstream from the transcription start site of the MSMB gene.

4. The method according to claim 1, wherein the region extends from −3533 bp to −2734 bp upstream from the transcription start site of the MSMB gene.

5. The method according to claim 1, wherein the region extends from −452 bp upstream to +150 bp downstream from the transcription start site of the MSMB gene.

6. The method according to claim 1, wherein the region extends from 2180 bp to 2390 bp downstream from the transcription start site of the MSMB gene.

7. The method according to claim 1, wherein the region between −13877 and −13583 or between −10528 and −10254 or between −3920 to −3673 or between −3471 and −3141 or −3128 and −2817 base pairs upstream from the transcription start site of the MSMB gene is analyzed for hypermethylation.

8. The method according to claim 1, wherein the region between 1671 and 1996 or between 2180 and 2390 between 5236 and 5616 or between 7670 and 8030 or between 11432 and 11754 or base pairs downstream from the transcription start site of the MSMB gene is analysed for hypermethylation.

9. The method according to claim 1, wherein the presence of hypermethylation of CpG and non-CpG dinucleotides in this regulatory region surrounding the transcription start site indicates the presence of prostate cancer cells or is indicative of prostate cancer.

10. The method according to claim 1, wherein the method comprises: analyzing the level DNA methylation of the regulatory region surrounding the TSS of a beta-MSMB gene in a biological sample isolated from the subject; whereby non-CpG methylation or non-CpG hypermethylation in the regulatory regions surrounding the transcriptional start site of the MSMB gene is an indication of prostate cancer or for prostate cancer cells' presence.

11. The method according to claim 1, wherein the method comprises: analyzing the level DNA methylation of the regulatory region surrounding the TSS of a beta-MSMB gene in a biological sample isolated from the subject, and further comprises comparing the DNA methylation with the DNA methylation in a control sample and/or a benign prostate hyperplasia sample; whereby increased non-CpG methylation in the regulatory regions surrounding the transcriptional start site of the MSMB gene is an indication for prostate cancer or for the presence of prostate cancer cells.

12. The method according to claim 1, wherein the method comprises: analyzing the level DNA methylation of the regulatory region surrounding the TSS of a beta-MSMB gene in a biological sample isolated from the subject, and further comprises comparing the DNA methylation with the DNA methylation in a control sample; whereby increased CpG methylation in the regulatory regions surrounding the transcriptional start site of the MSMB gene is an indication for prostate cancer or for the presence of prostate cancer cells.

13. The method according to claim 1, wherein the method comprises: analyzing the level DNA methylation of the regulatory region surrounding the TSS of a beta-MSMB gene in a biological sample isolated from the subject, and further comprises comparing the DNA methylation with the DNA methylation in a sample of androgen sensitive prostate cancer; whereby increased level methylation of the CpG dinucleotides in the regulatory region surrounding the transcriptional start site of the MSMB gene relative to the androgen sensitive prostate cancer is an indication of an hormone refractory prostate cancer, androgen-independent prostate cancer (AIPC) or androgen-independent metastatic prostate cancer.

14. The method according to claim 1, wherein the method comprises: analyzing the level DNA methylation of regulatory region surrounding the TSS of a beta-MSMB gene in a biological sample isolated from the subject, and further comprises comparing the DNA methylation with the DNA methylation in a control sample or a benign prostate hyperplasia sample; whereby increased non-CpG methylation in the regulatory regions surrounding the transcriptional start site of the MSMB gene relative to a control sample is an indication for prostate cancer and comparing the DNA methylation with the DNA methylation in a androgen sensitive prostate cancer sample; and whereby an increased level methylation of the CpG dinucleotides in the regulatory region surrounding the transcriptional start site of the MSMB gene relative to a control sample is an indication of an hormone refractory prostate cancer, androgen-independent prostate cancer (AIPC) or androgen-independent metastatic prostate cancer.

15. The method according to claim 1, wherein the method comprises:
analyzing histone acetylation or deacetylation of the MSMB gene in the sample.

16. The method according to claim 14, wherein the method comprises:
analyzing histone acetylation or deacetylation of the MSMB gene in the sample.

17. The method according to claim 1, wherein an androgen-independent metastatic prostate cancer in a prostate cell or prostate tissue is diagnosed.

18. The method according to claim 14, wherein an androgen-independent metastatic prostate cancer in a prostate cell or prostate tissue is diagnosed.

19. The method according to claim 1, further comprising:
carrying out a prostate cancer grading or prostate cancer staging.

20. The method according to claim 14, further comprising:
carrying out a prostate cancer grading or prostate cancer staging.

21. The method according to claim 1, further comprising:
deciding on a treatment or medicament of the prostate disease state.

22. The method according to claim 14, further comprising:
deciding on a treatment or medicament of the prostate disease state.

23. The method according to claim 1, wherein hypermethylation is determined using PCR or other amplification technique.

24. The method according to claim 1, wherein hypermethylation is determined by bisulfite genomic sequencing PCR analysis.

25. The method according to claim 1, wherein hypermethylation is determined by Methylation-Specific PCR analysis or other amplification technique.

26. The method according to claim 1, wherein hypermethylation is determined by a diagnostic array, the array comprising primers for assessing the presence of hypermethylation in a regulatory region surrounding the TSS of the MSMB gene.

27. The method according to claim 1 which utilizes at least one primer of the group consisting of methylated specific primers (SEQ ID NOs: 13, 14, 17, 19, 20, 21, 22, 26, 28, 29, 38, 39, 42, 43, 50, 51, 57, 58, 61, and 62) and of the group consisting of unmethylated specific primers (SEQ ID NO's 15, 16, 18, 23, 24, 25, 27, 30, 31, 40, 41, 44, 45, 52, 53, 59, 60, 63 and 64).

28. The method according to claim 1 which utilizes at least one primer of the group consisting of bisulfite sequencing primers (SEQ ID NO: 9, 10, 11, 12, 32, 33, 34, 35, 36, 37, 46, 47, 48, 49, 54, 55, 56, 65, 66, 67, 68, 69 and 70).

29. The method according to claim 1, wherein distinguishing between methylated and non-methylated CpG dinucleotide sequences within the target sequence comprises utilizing at least one primer in each case a contiguous sequence at least 16 nucleotides in length that is complementary to, or hybridizes under stringent conditions to a bisulfite-converted sequence (SEQ ID NO:6 and 8) derived from a sequence selected from the SEQ ID NO:5 and 7.

30. The method according to claim 1 wherein PCR analysis is performed on polynucleotide materials of cells derived from prostatic tissue.

31. The method according to claim 1 wherein PCR analysis is performed on polynucleotide materials of the cells derived from seminal fluid or from ejaculate.

32. The method according to claim 1 wherein PCR analysis is performed on polynucleotide materials of the cells derived from body fluids selected from the group consisting of blood, urine, ejaculates, prostate secretions, and combinations thereof.

33. The method according to claim 1 wherein PCR analysis is performed on polynucleotide materials of the cells derived from prostate tissue from histological slides or biopsies or paraffin-embedded tissue.

34. The method according to claim 1 wherein PCR analysis is performed on polynucleotide materials of the cells of prostatic tissue from biopsy or from surgical resection.

35. The method according to claim 1, comprising: obtaining a biological sample from the subject; determining the methylation state of CpG island upstream and/or downstream of the transcriptional start site of the MSMB gene in the subject's sample; and identifying hypermethylation of one or more CpG islands of the regulatory region surrounding the transcription start site of the MSMB gene, wherein detection of hypermethylation is indicative of a predisposition to, or the incidence of prostate cancer.

36. The method according to claim 1, wherein differential methylation is observed when compared to the methylation status of the regulatory region surrounding the transcription start site of the MSMB gene from a androgen-sensitive prostate cancer cell or a normal cell, which differential methylation is hypermethylation and is indicative for androgen-refractory prostate cancer.

37. The method according to claim 1, wherein detection of hypermethylation is indicative for the grade and/or stage of the prostate proliferative disorder.

38. The method according to claim 1, wherein detection of hypermethylation is indicative for decided about a treatment with a DNA methylation inhibitor.

39. The method according to claim 1, wherein detection of hypermethylation is indicative for decided about the initiation or continuation of treating with a compound in an effective amount to reduce male hormones.

40. The method according to claim 1, further comprising analyzing acetylation of the MSMB gene histones for distinguishing between an androgen-independent or androgen-refractory prostate cancer and/or an androgen-sensitive prostate cancer.

41. A nucleic acid molecule consisting essentially of a sequence at least 16 continuous bases in length of a sequence selected from the sequence group consisting of SEQ ID NOS: 6 and 8.
